(19) United States
(12) Patent Application Publication
(10) Pub. No.: US 2025/0206761 A1
(43) Pub. Date: Jun. 26, 2025

(54) HALOINDOLE MACROCYCLIC COMPOUNDS FOR THE TREATMENT OF CANCER

(71) Applicant: Hoffmann-La Roche Inc., Little Falls, NJ (US)

(72) Inventors: Jianguo CHEN, Shanghai (CN); Lei GUO, Shanghai (CN); Haixia LIU, Shanghai (CN); Hong SHEN, Shanghai (CN); Jin XIE, Shanghai (CN); Weixing ZHANG, Shanghai (CN); Dan ZHAO, Shanghai (CN); Wei ZHU, Shanghai (CN)

(73) Assignee: Hoffmann-La Roche Inc., Little Falls, NJ (US)

(21) Appl. No.: 18/869,390

(22) PCT Filed: May 30, 2023
(86) PCT No.: PCT/EP2023/064389

§ 371 Date: Nov. 26, 2024

(30) Foreign Application Priority Data
Jun. 1, 2022 (WO) PCT/CN2022/096622
Oct. 11, 2022 (WO) PCT/CN2022/124651
Jan. 5, 2023 (WO) PCT/CN2023/070763

(51) Int. Cl. C07D 519/00; A61P 35/00; C07D 498/14; C07D 513/22

(52) U.S. Cl. CPC C07D 519/00; A61P 35/00; C07D 498/14; C07D 513/22

(57) ABSTRACT

The present invention relates to compounds of formula (I), wherein R¹ to R⁷, A¹ and A² are as described herein, and their pharmaceutically acceptable salt thereof, and compositions including the compounds and methods of using the compounds.

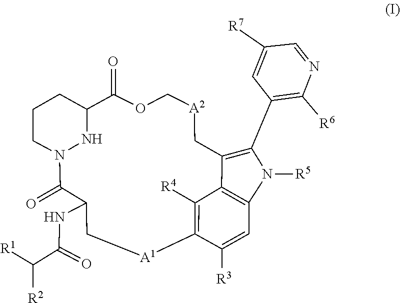

The present invention relates to organic compounds useful for therapy and/or prophylaxis in a mammal, and in particular to inhibition of KRAS G12C useful for treating cancers.

### FIELD OF THE INVENTION

RAS is one of the most well-known proto-oncogenes. Approximately 30% of human cancers contain mutations in three most notable members, KRAS, HRAS, and NRAS, making them the most prevalent oncogenic drivers. KRAS mutations are generally associated with poor prognosis especially in colorectal cancer, pancreatic cancer, lung cancers. As the most frequently mutated RAS isoform, KRAS has been intensively studied in the past years. Among the most commonly occurring KRAS alleles (including G12D, G12V, G12C, G13D, G12R, G12A, G12S, Q61H, etc), G12C, G12D, G12V represent more than half of all K-RAS-driven cancers across colorectal cancer (CRC), pancreatic ductal adenocarcinoma (PDAC), lung adenocarcinoma (LUAD). Of note, KRAS wild-type amplifications are also found in around 7% of all KRAS-altered cancers (ovarian, esophagogastric, uterine), ranking among the top alterations.

All RAS proteins belong to a protein family of small GTPases that hydrolyze GTP to GDP. KRAS is structurally divided into an effector binding lobe followed by the allosteric lobe and a carboxy-terminal region that is responsible for membrane anchoring. The effector lobe comprises the P-loop, switch I, and switch II regions. The switch I/II loops play a critical role in KRAS downstream signaling through mediating protein-protein interactions with effector proteins that include RAF in the mitogen-activated protein kinase (MAPK) pathway or PI3K in the phosphatidylinositol 3-kinase (PI3K)/protein kinase B (AKT) pathway.

KRAS protein switches between an inactive to an active form via binding to GTP and GDP, respectively. Under physiological conditions, the transition between these two states is regulated by guanine nucleotide exchange factors (GEFs), such as Son Of Sevenless Homolog 1 (SOS1), or GTPase-activating proteins (GAPs) that involve catalyzing the exchange of GDP for GTP, potentiating intrinsic GTPase activity or accelerating RAS-mediated GTP hydrolysis. In response to extracellular stimuli, the inactive RAS-GDP is converted to active RAS-GTP which directly binds to RAF RAS binding domains (RAF^{RBD}), recruiting RAF kinase family from cytoplasm to membranes, where they dimerize and become active. The activated RAF subsequently carries out a chain of phosphorylation reactions to its downstream Mitogen-activated protein kinase (MEK) and extracellular signal-regulated kinase (ERK), and propagates the growth signal. Of the RAF family of protein kinases (three known isoforms ARAF, BRAF, CRAF/RAF1), BRAF is most frequently mutated and remains the most potent activator of MEK. Despite that individual RAS and RAF family members revealed distinct binding preferences, all RAFs possess the conserved RBD for forward transmission of MAPK signaling, frequently used for characterize KRAS inhibition (e.g. KRAS-BRAF^{RBD} herein). For KRAS, mutations at positions 12, 13, 61, and 146 lead to a shift toward the active KRAS form through impairing nucleotide hydrolysis or activating nucleotide exchange, leading to hyper-activation of the MAPK pathway that results in tumorigenesis.

Despite its well-recognized importance in cancer malignancy, continuous efforts in the past failed to develop approved therapies for KRAS mutant cancer until recently, the first selective drug AMG510 has fast approval as second line treatment in KRAS G12C driven non-small cell lung cancer (NSCLC). Nevertheless, the clinical acquired resistance to KRAS G12C inhibitors emerge rigorously with disease progresses after around 6 month of treatment. All of the mutations converge to reactivate RAS-MAPK signaling, with secondary RAS mutants at oncogenic hotspots (e.g. G12/G13/Q61) and within the switch II pocket (e.g. H95, R68, and Y96) have been observed; moreover, over 85% of all KRAS-mutated or wild-type amplified driven cancers still lack novel agents. Altogether, both the myriad of escape mechanism and various oncogenic alleles, highlight the urgent medical need for additional KRAS therapies. As such, we invented oral compounds that target and inhibit KRAS alleles for the treatment of KRAS mutant driven cancers.

### SUMMARY OF THE INVENTION

The present invention relates to novel compounds of formula (I),

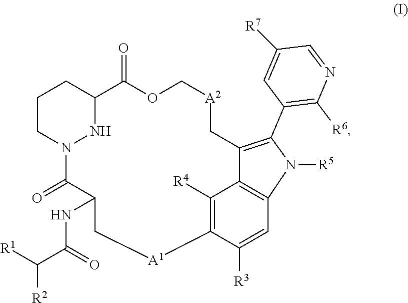

wherein
R¹

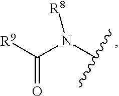

1-oxo-2,7-diazaspiro[4.4]nonanyl substituted by (dihaloC₁₋₆alkyl)carbonyl,
1-oxo-2,8-diazaspiro[4.5]decanyl substituted by (dihaloC₁₋₆alkyl)carbonyl,
1-oxo-2,9-diazaspiro[5.5]undecanyl substituted by (dihaloC₁₋₆alkyl)carbonyl,
5-oxo-2,6-diazaspiro[3.4]octanyl substituted by (dihaloC₁₋₆alkyl)carbonyl,
5-oxo-2,6-diazaspiro[3.5]nonanyl substituted by (dihaloC₁₋₆alkyl)carbonyl,
6-oxo-2,7-diazaspiro[4.5]decanyl substituted by (dihaloC₁₋₆alkyl)carbonyl or
azetidinyloxyC₁₋₆alkyl substituted by (dihaloC₁₋₆alkyl)carbonyl;
wherein R⁸ is C₁₋₆alkyl;
   R⁹ is piperidinyl twice substituted by halogen and (dihaloC₁₋₆alkyl)carbonyl, or pyrrolidinyl substituted by (dihaloC₁₋₆alkyl)carbonyl;
R² is C₁₋₆alkyl;
R³ is H, C₁₋₆alkyl or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene or phenylene substituted by hydroxy;
A² is C₁₋₆alkylene;
with the proviso that R³ and R⁴ are not H simultaneously;
or a pharmaceutically acceptable salt thereof.

The invention also relates to their manufacture, medicaments based on a compound in accordance with the invention and their production as well as the use of compounds of formula (I) or (Ia) thereof as inhibitor of KRAS.

The compounds of formula (I) or (Ia) show good KRAS inhibition for G12C, G12D and G12V. In another embodiment, the compounds of this invention showed superior cancer cell inhibition and human hepatocyte stability. In addition, the compounds of formula (I) or (Ia) also show good or improved cytotoxicity, solubility profiles. Furthermore, the compound of current invention addressed GSH toxicity issue comparing with the reference compounds.

### BRIEF DESCRIPTION OF THE FIGURE

FIG. 1. X-ray crystallographic analysis of compound 4f 1.

FIG. 2. X-ray crystallographic analysis of compound 4f 2.

FIG. 3. X-ray crystallographic analysis of compound M2.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkylene” denotes a linear or branched saturated divalent hydrocarbon group of 1 to 6 carbon atoms or a divalent branched saturated divalent hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “halogen” and “halo” are used interchangeably herein and denote fluoro, chloro, bromo, or iodo.

The term “haloindole” denotes an indole wherein one or more of the hydrogen atoms have been replaced by same or different halogen atoms.

The term “dihaloC₁₋₆alkyl” denotes a C₁₋₆alkyl group wherein two of the hydrogen atoms of the C₁₋₆alkyl group have been replaced by same or different halogen atoms. Examples of dihaloC₁₋₆alkyl include, difluoro- or chloro(fluoro)-methyl, -ethyl or -propyl, particularly difluoropropyl, difluoromethyl, difluoroethyl or chloro(fluoro)methyl.

The term “haloC₁₋₆alkyl” denotes a C₁₋₆alkyl group wherein at least one of the hydrogen atoms of the C₁₋₆alkyl group have been replaced by same or different halogen atoms. Examples of haloC₁₋₆alkyl include fluoro, difluoro- or chloro(fluoro)-methyl, -ethyl or -propyl, for example fluoromethyl, difluoropropyl, difluoromethyl, difluoroethyl, chloro(fluoro)methyl, trifluoroethyl, or trifluoromethyl.

The term “phenylene” denotes a divalent phenyl group.

The term “piperidinylene” denotes a divalent piperidinyl group.

The term “pyrrolidinylene” denotes a divalent pyrrolidinyl group.

The term “thiazolylene” denotes a divalent thiazolyl group.

The term “oxo” denotes a divalent oxygen atom ═O.

The term “azetidinyloxy” denotes azetidinyl-O—.

The term “dimethylmethylene” denotes

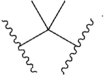 [SMILES: *C(*)(C)C]

The term “protecting group” denotes the group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups.

The skilled of the art would understand that the following structures of compounds of formula (Ia) and (Ia′) are equal especially for the chiral centers:

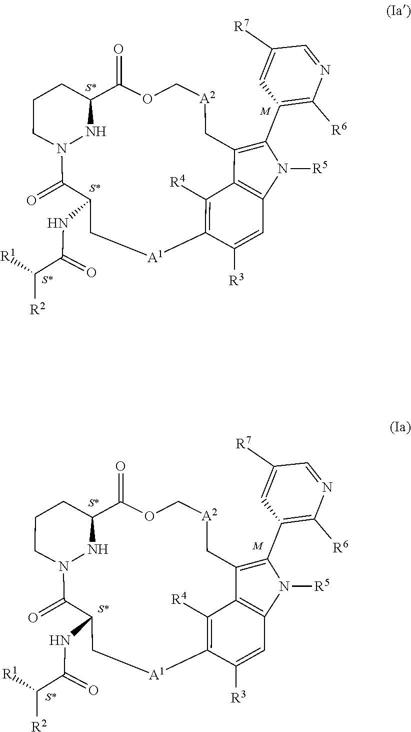

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### Inhibitor of KRAS

The present invention relates to (i) a compound of formula (I),

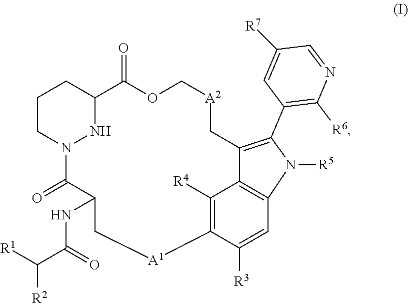

wherein
R¹ is

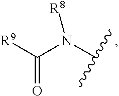

1-oxo-2,7-diazaspiro[4.4]nonanyl substituted by (dihaloC₁₋₆alkyl)carbonyl,
1-oxo-2,8-diazaspiro[4.5]decanyl substituted by (dihaloC₁₋₆alkyl)carbonyl,
1-oxo-2,9-diazaspiro[5.5]undecanyl substituted by (dihaloC₁₋₆alkyl)carbonyl,
5-oxo-2,6-diazaspiro[3.4]octanyl substituted by (dihaloC₁₋₆alkyl)carbonyl,
5-oxo-2,6-diazaspiro[3.5]nonanyl substituted by (dihaloC₁₋₆alkyl)carbonyl,
6-oxo-2,7-diazaspiro[4.5]decanyl substituted by (dihaloC₁₋₆alkyl)carbonyl or
azetidinyloxyC₁₋₆alkyl substituted by (dihaloC₁₋₆alkyl)carbonyl;
wherein R⁸ is C₁₋₆alkyl;
   R⁹ is piperidinyl twice substituted by halogen and (dihaloC₁₋₆alkyl)carbonyl, or pyrrolidinyl substituted by (dihaloC₁₋₆alkyl)carbonyl;
R² is C₁₋₆alkyl;
R³ is H, C₁₋₆alkyl or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene or phenylene substituted by hydroxy;
A² is C₁₋₆alkylene;
with the proviso that R³ and R⁴ are not H simultaneously;
or a pharmaceutically acceptable salt thereof.

Another embodiment of present invention is (ii) a compound of formula (Ia),

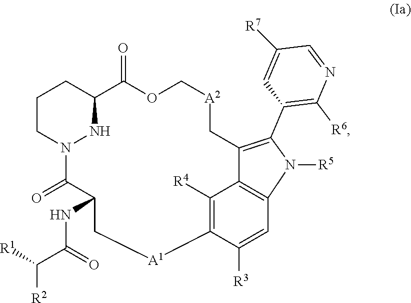

wherein
R¹ is

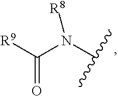

1-oxo-2,7-diazaspiro[4.4]nonanyl substituted by (dihaloC₁₋₆alkyl)carbonyl,
1-oxo-2,8-diazaspiro[4.5]decanyl substituted by (dihaloC₁₋₆alkyl)carbonyl,
1-oxo-2,9-diazaspiro[5.5]undecanyl substituted by (dihaloC₁₋₆alkyl)carbonyl,
5-oxo-2,6-diazaspiro[3.4]octanyl substituted by (dihaloC₁₋₆alkyl)carbonyl,
5-oxo-2,6-diazaspiro[3.5]nonanyl substituted by (dihaloC₁₋₆alkyl)carbonyl,
6-oxo-2,7-diazaspiro[4.5]decanyl substituted by (dihaloC₁₋₆alkyl)carbonyl or
azetidinyloxyC₁₋₆alkyl substituted by (dihaloC₁₋₆alkyl)carbonyl;
wherein R⁸ is C₁₋₆alkyl;
   R⁹ is piperidinyl twice substituted by halogen and (dihaloC₁₋₆alkyl)carbonyl, or pyrrolidinyl substituted by (dihaloC₁₋₆alkyl)carbonyl;
R² is C₁₋₆alkyl;
R³ is H, C₁₋₆alkyl or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene or phenylene substituted by hydroxy;
A² is C₁₋₆alkylene;
with the proviso that R³ and R⁴ are not H simultaneously;
or a pharmaceutically acceptable salt thereof.

A further embodiment of present invention is (iii) a compound of formula (I) or (Ia) according to (i) or (ii), or a pharmaceutically acceptable salt thereof, wherein
R¹ is

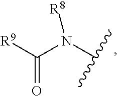

1-oxo-2,7-diazaspiro[4.4]nonanyl substituted by (dihaloC₁₋₆alkyl)carbonyl,
1-oxo-2,8-diazaspiro[4.5]decanyl substituted by (dihaloC₁₋₆alkyl)carbonyl,
1-oxo-2,9-diazaspiro[5.5]undecanyl substituted by (dihaloC₁₋₆alkyl)carbonyl, or
5-oxo-2,6-diazaspiro[3.4]octanyl substituted by (dihaloC₁₋₆alkyl)carbonyl;
wherein R⁸ is C₁₋₆alkyl;
R⁹ is piperidinyl twice substituted by halogen and (dihaloC₁₋₆alkyl)carbonyl, or pyrrolidinyl substituted by (dihaloC₁₋₆alkyl)carbonyl.

A further embodiment of present invention is (iv) a compound of formula (I) or (Ia), according to any one of (i) to (iii), or a pharmaceutically acceptable salt thereof, wherein
R¹ is

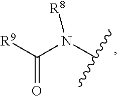

1-oxo-2,7-diazaspiro[4.4]nonan-2-yl substituted by chloro(fluoro)acetyl,
1-oxo-2,8-diazaspiro[4.5]decan-2-yl substituted by chloro(fluoro)acetyl,
1-oxo-2,9-diazaspiro[5.5]undecan-2-yl substituted by chloro(fluoro)acetyl, or
5-oxo-2,6-diazaspiro[3.4]octan-6-yl substituted by chloro(fluoro)acetyl;
wherein R⁸ is methyl;
R⁹ is piperidinyl twice substituted by fluoro and chloro(fluoro)acetyl, or pyrrolidinyl substituted by chloro(fluoro)acetyl.

A further embodiment of present invention is (v) a compound of formula (I) or (Ia) according to any one of (i) to (iv), wherein R¹ is (5R)-7-[(2R)-2-chloro-2-fluoro-acetyl]-1-oxo-2,7-diazaspiro[4.4]nonan-2-yl, (5S)-7-[(2R)-2-chloro-2-fluoro-acetyl]-1-oxo-2,7-diazaspiro[4.4]nonan-2-yl, 8-[(2R)-2-chloro-2-fluoro-acetyl]-1-oxo-2,8-diazaspiro[4.5]decan-2-yl, 9-[(2R)-2-chloro-2-fluoro-acetyl]-1-oxo-2,9-diazaspiro[5.5]undecan-2-yl, 2-[(2R)-2-chloro-2-fluoro-acetyl]-5-oxo-2,6-diazaspiro[3.4]octan-6-yl, 1-[(2R)-2-chloro-2-fluoro-acetyl]-4-fluoro-4-piperidinyl or 1-[(2R)-2-chloro-2-fluoro-acetyl]pyrrolidin-3-yl.

A further embodiment of present invention is (vi) a compound of formula (I) or (Ia), or a pharmaceutically acceptable salt thereof, according to any one of (i) to (v), wherein R² is isopropyl.

A further embodiment of present invention is (vii) a compound of formula (I) or (Ia), or a pharmaceutically acceptable salt thereof, according to any one of (i) to (vi), wherein R³ is H, methyl or fluoro.

A further embodiment of present invention is (viii) a compound of formula (I) or (Ia), or a pharmaceutically acceptable salt thereof, according to any one of (i) to (vii), wherein R³ is fluoro.

A further embodiment of present invention is (ix) a compound of formula (I) or (Ta), or a pharmaceutically acceptable salt thereof, according to any one of (i) to (viii), wherein R⁴ is H or fluoro.

A further embodiment of present invention is (x) a compound of formula (I) or (Ta), or a pharmaceutically acceptable salt thereof, according to any one of (i) to (ix), wherein R⁴ is H.

A further embodiment of present invention is (xi) a compound of formula (I) or (Ta), or a pharmaceutically acceptable salt thereof, according to any one of (i) to (x), wherein R⁵ is ethyl or 2,2,2-trifluoroethyl.

A further embodiment of present invention is (xii) a compound of formula (I) or (Ta), or a pharmaceutically acceptable salt thereof, according to any one of (i) to (xi), wherein 1-methoxyethyl.

A further embodiment of present invention is (xiii) a compound of formula (I) or (Ta), or a pharmaceutically acceptable salt thereof, according to any one of (i) to (xii), wherein R⁷ is morpholinyl, 4-(2,2,2-trifluoroethyl)piperazin-1-yl or 4-methylpiperazin-1-yl.

A further embodiment of present invention is (xiv) a compound of formula (I) or (Ia), or a pharmaceutically acceptable salt thereof, according to any one of (i) to (xiii), wherein A¹ is

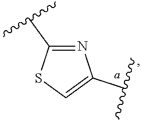 [SMILES: *c1csc(*)n1]

wherein bond “a” connects to indole ring.

A further embodiment of present invention is (xv) a compound of formula (I) or (Ia), or a pharmaceutically acceptable salt thereof, according to any one of (i) to (xiv), wherein A² is dimethylmethylene.

Another embodiment of present invention is (xvi) a compound of formula (I) or (Ia), according to (i) or (ii), wherein
R¹ is

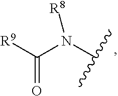

1-oxo-2,7-diazaspiro[4.4]nonanyl substituted by (dihaloC₁₋₆alkyl)carbonyl,
1-oxo-2,8-diazaspiro[4.5]decanyl substituted by (dihaloC₁₋₆alkyl)carbonyl,
1-oxo-2,9-diazaspiro[5.5]undecanyl substituted by (dihaloC₁₋₆alkyl)carbonyl, or
5-oxo-2,6-diazaspiro[3.4]octanyl substituted by (dihaloC₁₋₆alkyl)carbonyl;
wherein R⁸ is C₁₋₆alkyl;
   R⁹ is piperidinyl twice substituted by halogen and (dihaloC₁₋₆alkyl)carbonyl, or pyrrolidinyl substituted by (dihaloC₁₋₆alkyl)carbonyl;
R² is C₁₋₆alkyl;
R³ is halogen;
R⁴ is H;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is

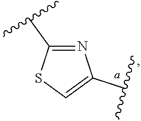 [SMILES: *c1csc(*)n1]

wherein bond “a” connects to indole ring;
A² is C₁₋₆alkylene;
or a pharmaceutically acceptable salt thereof.

Another embodiment of present invention is (xvii) a compound of formula (I) or (Ia), according to (xvi), wherein
R¹ is (5R)-7-[(2R)-2-chloro-2-fluoro-acetyl]-1-oxo-2,7-diazaspiro[4.4]nonan-2-yl, (5S)-7-[(2R)-2-chloro-2-fluoro-acetyl]-1-oxo-2,7-diazaspiro[4.4]nonan-2-yl, 8-[(2R)-2-chloro-2-fluoro-acetyl]-1-oxo-2,8-diazaspiro[4.5]decan-2-yl, 9-[(2R)-2-chloro-2-fluoro-acetyl]-1-oxo-2,9-diazaspiro[5.5]undecan-2-yl, 2-[(2R)-2-chloro-2-fluoro-acetyl]-5-oxo-2,6-diazaspiro[3.4]octan-6-yl, 1-[(2R)-2-chloro-2-fluoro-acetyl]-4-fluoro-4-piperidinyl or 1-[(2R)-2-chloro-2-fluoro-acetyl]pyrrolidin-3-yl;
R² is isopropyl;
R³ is fluoro;
R⁴ is H;
R⁵ is ethyl or 2,2,2-trifluoroethyl;
R⁶ is (1S)-1-methoxyethyl;
R⁷ is morpholinyl, 4-(2,2,2-trifluoroethyl)piperazin-1-yl or 4-methylpiperazin-1-yl;
A¹ is

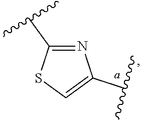 [SMILES: *c1csc(*)n1]

wherein bond “a” connects to indole ring;
A² is dimethylmethylene;
or a pharmaceutically acceptable salt thereof.

Another embodiment of present invention is (xviii) a compound of formula (I) or (Ia) selected from the following:
(3S)-1-[(2R)-2-chloro-2-fluoro-acetyl]-N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(7S,13S)-(20M)-20-[2-[(1S)-1-methoxyethyl]-5-(4-methylpiperazin-1-yl)-3-pyridyl]-17,17,24-trimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(2S)-2-[(5R)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[9-[(2R)-2-chloro-2-fluoro-acetyl]-1-oxo-2,9-diazaspiro[5.5]undecan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(3S)-1-[(2R)-2-chloro-2-fluoro-acetyl]-N-[(1S)-1-[[(8S,14S)-25-fluoro-4-hydroxy-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl]-2-methyl-propyl]-N-methyl-pyrrolidine-3-carboxamide;
1-[(2R)-2-chloro-2-fluoro-acetyl]-4-fluoro-N-[(1S)-1-[[(7S,13S)-(20M)-20-[2-[(1S)-1-methoxyethyl]-5-(4-methylpiperazin-1-yl)-3-pyridyl]-17,17,24-trimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-piperidine-4-carboxamide;
1-[(2R)-2-chloro-2-fluoro-acetyl]-4-fluoro-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-piperidine-4-carboxamide;
(2S)-2-[2-[(2R)-2-chloro-2-fluoro-acetyl]-5-oxo-2,6-diazaspiro[3.5]nonan-6-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2R)-2-[[1-[(2R)-2-chloro-2-fluoro-acetyl]azetidin-3-yl]oxymethyl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo-[17.5.2.1^{2,5}.1^{9,13}.0^{22,26}]octacosa-1(25),2,5(28),19,22(26),23-hexaen-7-yl]-3-methyl-butanamide;
(2S)-2-[[1-[(2R)-2-chloro-2-fluoro-acetyl]azetidin-3-yl]oxymethyl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[8-[(2R)-2-chloro-2-fluoro-acetyl]-1-oxo-2,8-diazaspiro[4.5]decan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-2-[(2R)-2-chloro-2-fluoro-acetyl]-6-oxo-2,7-diazaspiro[4.5]decan-7-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5R)-2-[(2R)-2-chloro-2-fluoro-acetyl]-6-oxo-2,7-diazaspiro[4.5]decan-7-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[9-[(2R)-2-chloro-2-fluoro-acetyl]-1-oxo-2,9-diazaspiro[5.5]undecan-2-yl]-N-[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5R)-2-[(2S)-2-chloro-2-fluoro-acetyl]-6-oxo-2,7-diazaspiro[4.5]decan-7-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-2-[(2S)-2-chloro-2-fluoro-acetyl]-6-oxo-2,7-diazaspiro[4.5]decan-7-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(3S)-1-[(2R)-2-chloro-2-fluoro-acetyl]-N-[(1S)-1-[[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(2S)-2-[2-[(2R)-2-chloro-2-fluoro-acetyl]-5-oxo-2,6-diazaspiro[3.4]octan-6-yl]-N-[(7S,13S)-24-fluoro-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(3S)-1-[(2R)-2-chloro-2-fluoro-acetyl]-N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(8S,14S)-26-fluoro-4-hydroxy-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl]-2-methyl-propyl]-N-methyl-pyrrolidine-3-carboxamide;
(2S)-2-[8-[(2R)-2-chloro-2-fluoro-acetyl]-1-oxo-2,8-diazaspiro[4.5]decan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[2-[(2R)-2-chloro-2-fluoro-acetyl]-5-oxo-2,6-diazaspiro[3.4]octan-6-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5R)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(3S)-1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-4-fluoro-N-methyl-piperidine-4-carboxamide;
(2S)-2-[(5R)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[9-[(2R)-2-chloro-2-fluoro-acetyl]-1-oxo-2,9-diazaspiro[5.5]undecan-2-yl]-N-[(7S,13S)-21-ethyl-24-fluoro-(20M)-20-[2-1[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[2-[(2R)-2-chloro-2-fluoro-acetyl]-5-oxo-2,6-diazaspiro[3.4]octan-6-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[8-[(2R)-2-chloro-2-fluoro-acetyl]-1-oxo-2,8-diazaspiro[4.5]decan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2R)-2-[[1-[(2R)-2-chloro-2-fluoro-acetyl]azetidin-3-yl]oxymethyl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[[1-[(2R)-2-chloro-2-fluoro-acetyl]azetidin-3-yl]oxymethyl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[9-[(2R)-2-chloro-2-fluoro-acetyl]-1-oxo-2,9-diazaspiro[5.5]undecan-2-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-1[(5R)-7-[(2R)-2-chloro-2-fluoro-acetyl]-1-oxo-2,7-diazaspiro[4.4]nonan-2-yl]-N-[(7S,13S)-24-fluoro-20-[2-1[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
1-[(2R)-2-chloro-2-fluoro-acetyl]-4-fluoro-N-[(1S)-1-[[(7S,13S)-24-fluoro-(20M)-20-[2-[1(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-piperidine-4-carboxamide;
(2S)-2-[2-[(2R)-2-chloro-2-fluoro-acetyl]-5-oxo-2,6-diazaspiro[3.4]octan-6-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[8-[(2R)-2-chloro-2-fluoro-acetyl]-1-oxo-2,8-diazaspiro[4.5]decan-2-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[9-[(2R)-2-chloro-2-fluoro-acetyl]-1-oxo-2,9-diazaspiro[5.5]undecan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-4-fluoro-N-methyl-piperidine-4-carboxamide;
(2S)-2-[8-[(2R)-2-chloro-2-fluoro-acetyl]-1-oxo-2,8-diazaspiro[4.5]decan-2-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5R)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[2-[(2R)-2-chloro-2-fluoro-acetyl]-5-oxo-2,6-diazaspiro[3.4]octan-6-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-4-fluoro-N-methyl-piperidine-4-carboxamide;
(3S)-1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(2S)-2-[(5S)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide; and
(2S)-2-[2-[(2R)-2-chloro-2-fluoro-acetyl]-5-oxo-2,6-diazaspiro[3.4]octan-6-yl]-N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;

or a pharmaceutically acceptable salt thereof.

Another embodiment of present invention is related to (xix) a compound which has the following structure:

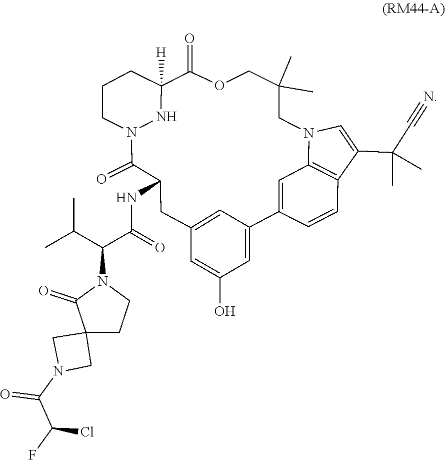 [SMILES: CC(C)[C@@H](C(=O)N[C@H]1Cc2cc(O)cc(c2)-c2ccc3c(C(C)(C)C#N)cn(c3c2)CC(C)(C)COC(=O)[C@@H]2CCCN(N2)C1=O)N1CCC2(CN(C(=O)[C@H](F)Cl)C2)C1=O]

Another embodiment of present invention is related to (xx) a process for the preparation of a compound according to any one of (i) to (xix) comprising any of the following steps:
a) coupling reaction between compound of formula (II),

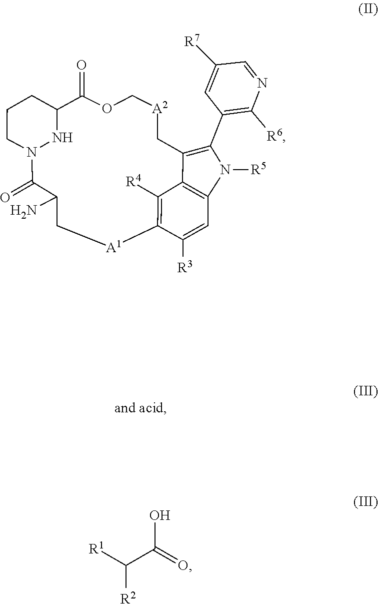

in the presence of a coupling reagent and a base to form the compound of formula (I);
b) coupling reaction between compound of formula (VI),

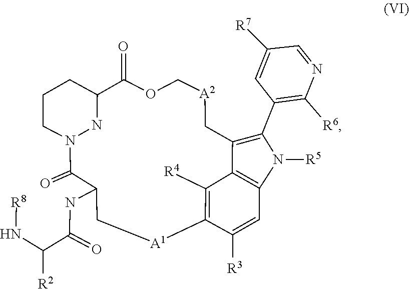

and acid (VII),

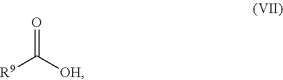

in the presence of a coupling reagent and a base to form the compound of formula (VIII),

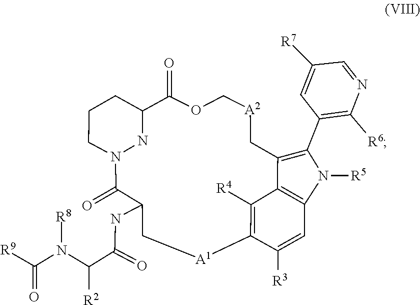

c) coupling reaction between compound of formula (XI),

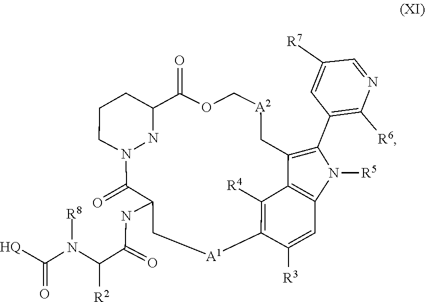

and acid (XII),

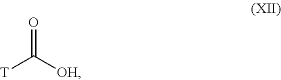

in the presence of a coupling reagent and a base to form the compound of formula (XIII),

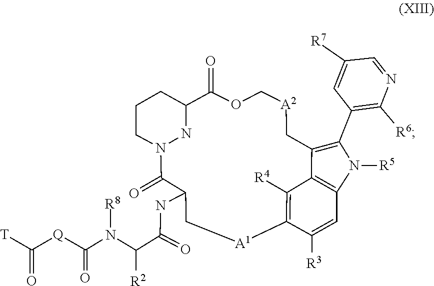

wherein Q is unsubstituted or substituted piperidinylene or pyrrolidinylene; T is dihaloC₁₋₆alkyl; R¹, R², R³, R⁴, R⁵, R⁶, R⁷, A¹ and A² are defined as in any one of (i) to (xvii); the coupling reagent in step a) to c) is T₃P, HATU, PyBOP or EDCl/HOBt; the base in step a) to c) is TEA, DIEPA or DMAP.

Another embodiment of present invention is (xxi) a compound or pharmaceutically acceptable salt according to any one of (i) to (xix) for use as therapeutically active substance.

Another embodiment of present invention is (xxii) a pharmaceutical composition comprising a compound in accordance with any one of (i) to (xix) and a pharmaceutically acceptable excipient.

Another embodiment of present invention is (xxiii) the use of a compound according to any one of (i) to (xix) for treating a KRAS G12C protein-related disease.

Another embodiment of present invention is (xxiv) the use of a compound according to any one of (i) to (xix) for treating a KRAS G12C, G12D and G12V protein-related disease.

Another embodiment of present invention is (xxv) the use of a compound according to any one of (i) to (xix) for inhibiting RAS interaction with downstream effectors, wherein the downstream effectors are RAF and PI3K.

Another embodiment of present invention is (xxvi) the use of a compound according to any one of (i) to (xix) for inhibiting the propagating oncogenic MAPK and PI3K signaling.

Another embodiment of present invention is (xxvii) the use of a compound according to any one of (i) to (xix) for the treatment or prophylaxis of KRAS mutation driven cancers, wherein the cancer is selected from pancreatic cancer, colorectal cancer, lung cancer, esophageal cancer, gallbladder cancer, melanoma ovarian cancer and endometrial cancer.

Another embodiment of present invention is (xxviii) the use of a compound according to any one of (i) to (xix) for the treatment or prophylaxis of KRAS mutation driven cancers, wherein the cancer is selected from pancreatic adenocarcinoma, colorectal cancer and non-small cell lung cancer.

Another embodiment of present invention is (xxix) a compound or pharmaceutically acceptable salt according to any one of (i) to (xix) for the treatment or prophylaxis of KRAS mutation driven cancers, wherein the cancer is selected from pancreatic adenocarcinoma, colorectal cancer and non-small cell lung cancer.

Another embodiment of present invention is (xxx) the use of a compound according to any one of (i) to (xix) for the preparation of a medicament for the treatment or prophylaxis of KRAS mutation driven cancers, wherein the cancer is selected from pancreatic adenocarcinoma, colorectal cancer and non-small cell lung cancer.

Another embodiment of present invention is (xxxi) a method for the treatment or prophylaxis of KRAS mutation driven cancers, wherein the cancer is selected from pancreatic adenocarcinoma, colorectal cancer and non-small cell lung cancer, which method comprises administering a therapeutically effective amount of a compound as defined in any one of (i) to (xix).

Another embodiment of present invention is (xxxii) a compound or pharmaceutically acceptable salt according to any one of (i) to (xix), when manufactured according to a process of (xx).

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit mutant RAS (e.g. KRAS G12C) interaction with RAF, blocking the oncogenic MAPK signaling. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 0.1 to 1000 mg/kg of patient body weight per day, with the typical initial range of compound used being 0.3 to 15 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 1 to 1000 mg of the compound of the invention compounded with about 1 to 1000 mg anhydrous lactose, about 1 to 1000 mg sodium croscarmellose, about 1 to 1000 mg polyvinylpyrrolidone (PVP) K30, and about 1 to 1000 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of mutant KRAS-driven cancers. Another embodiment includes a pharmaceutical composition comprising a compound of Formula (I) for use in the treatment of mutant KRAS-driven cancers.

The following example A and B illustrate typical compositions of the present invention, but serve merely as representative thereof.

### Example A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention induce a new binding pocket in KRAS by driving formation of a high affinity tri-complex between KRAS protein and the widely expressed cyclophilin A (CYPA), which inhibit KRAS interaction with downstream effectors, such as RAF and PI3K. Accordingly, the compounds of the invention are useful for inhibiting the propagating oncogenic MAPK and PI3K signaling, reducing cell proliferation, in particular cancer cells. Compounds of the invention are useful for termination of RAS signaling in cells that express RAS mutant, e.g. KRAS mutation driven pancreatic cancer, colorectal cancer, lung cancer, esophageal cancer, gallbladder cancer, melanoma ovarian cancer, endometrial cancer, etc. Alternatively, compounds of the invention are useful for termination of RAS signaling in malignant solid tumor where the oncogenic role of KRAS mutation is reinforced by dysregulation or mutation of effector pathways as MAPK, PI3K-AKT-mTOR (Mammalian target of rapamycin) driven signaling, for targeted therapy in pancreatic adenocarcinoma, colorectal cancer, non-small cell lung cancer, etc.

Another embodiment includes a method of treating or preventing cancer in a mammal in need of such treatment, wherein the method comprises administering to said mammal a therapeutically effective amount of a compound of formula (I), a stereoisomer, tautomer, prodrug or pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R⁷, A¹ and A² are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

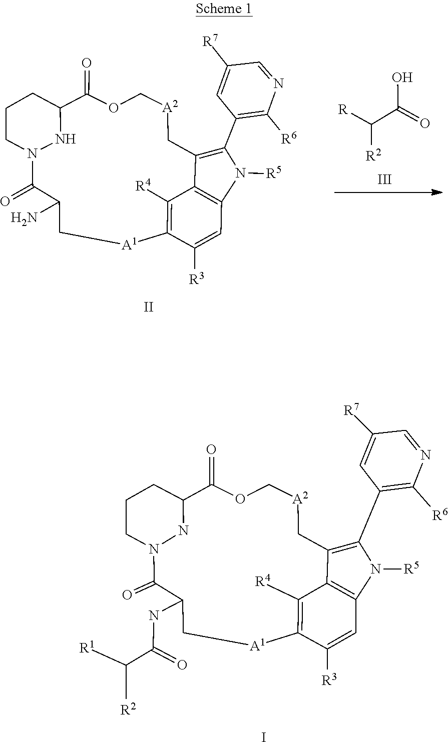

Compound of formula II was synthesized according to the procedure described in Intermediate A to L. Compound of formula (I) can be obtained by a coupling reaction between acid (III) and compound of formula (II) with coupling reagent(s), such as T₃P, HATU, PyBOP and EDCl/HOBt, in the presence of a base, such as TEA, DIEPA and DMAP.

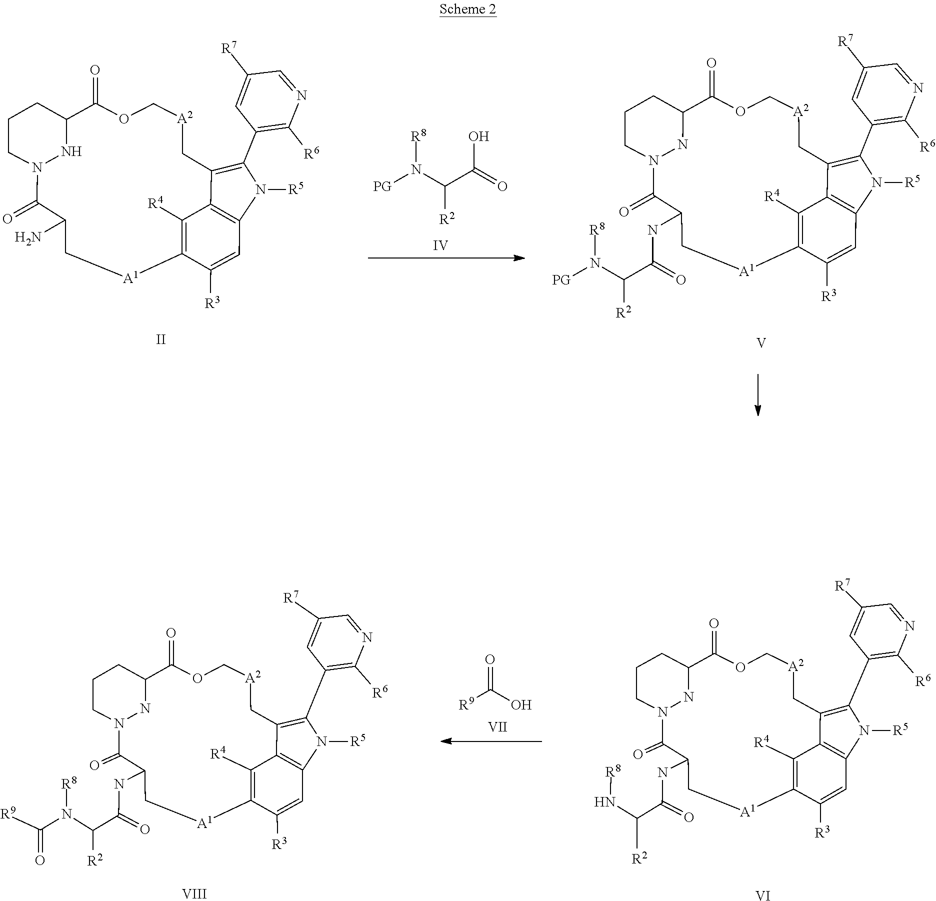

wherein PG is a protecting group, such as Boc and Cbz.

Alternatively compound of formula (V) can be obtained by a coupling reaction between acid (IV) and compound of formula (II) with coupling reagent(s), such as T₃P, HATU, PyBOP or EDCl/HOBt, in the presence of a base, such TEA, DIEPA or DMAP. Deprotection of compound of formula (V) can afford compound of formula (VI) in the presence of an acid, such as TFA, or under hydrogenation condition with a catalyst, such as Pd/C and Pd(OH)₂/C. Compound of formula (VIII) can be obtained by a coupling reaction between acid (VII) and compound of formula (VI) with coupling reagent(s), such as T₃P, HATU, PyBOP and EDCl/HOBt, in the presence of a base, such as TEA, DIEPA and DMAP.

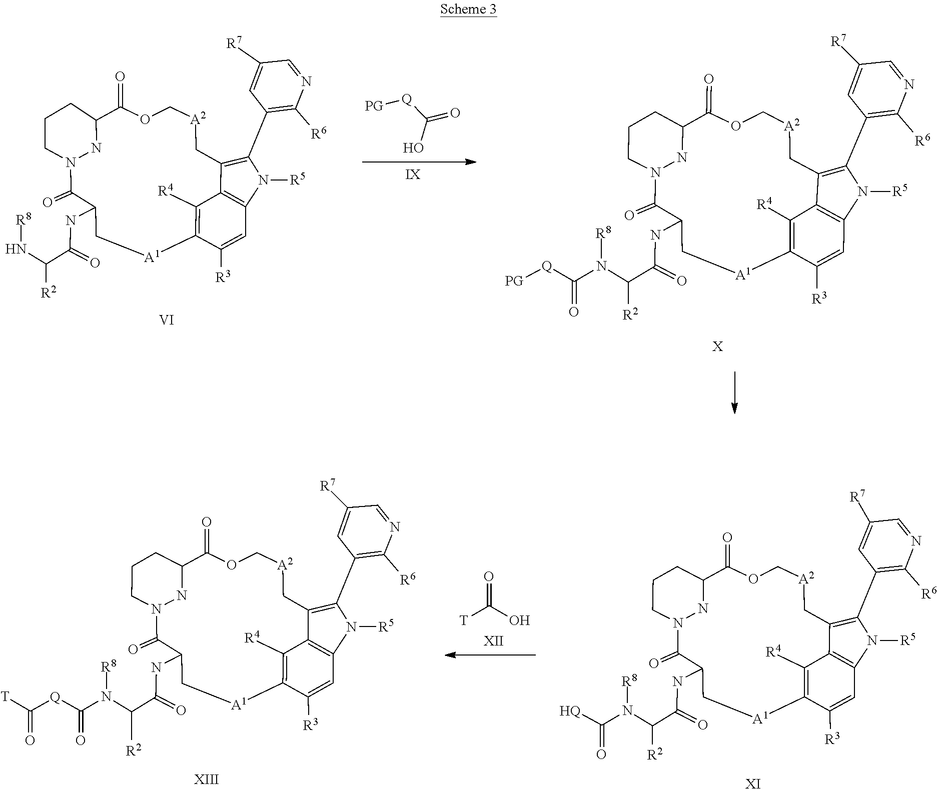

wherein PG is a protecting group, such as Boc and Cbz; T is dihaloC₁₋₆alkyl; Q is unsubstituted or substituted piperidinylene or pyrrolidinylene.

Alternatively compound of formula (X) can be obtained by a coupling reaction using acid (IX), compound of formula (VI) and coupling reagent(s), such as T₃P, HATU, PyBOP or EDCl/HOBt, in the presence of a base, such TEA, DIEPA or DMAP. Deprotection of compound of formula (X) can afford compound of formula (XI) in the presence of an acid, such as TFA, or under hydrogenation condition with a catalyst, such as Pd/C and Pd(OH)₂/C. Compound of formula (XIII) can be obtained by a coupling reaction between acid (XII) and compound of formula (XI) with coupling reagent(s), such as T₃P, HATU, PyBOP and EDCl/HOBt, in the presence of a base, such as TEA, DIEPA and DMAP.

Compounds of this invention can be obtained as mixtures of diastereomers or enantiomers, which can be separated by methods well known in the art, e.g. (chiral) HPLC or SFC. In another embodiment, compound of formula (I) can be obtained according to above scheme by using corresponding chiral starting materials.

This invention also relates to a process for the preparation of a compound of formula (I) comprising any of the following steps:
a) coupling reaction between compound of formula (II),

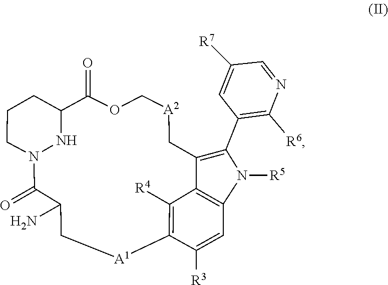

and acid (III),

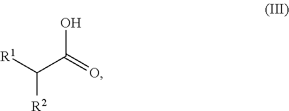

in the presence of a coupling reagent and a base to form the compound of formula (I);
b) coupling reaction between compound of formula (VI),

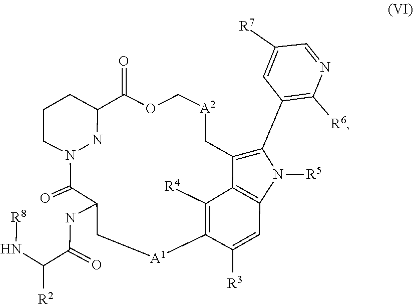

and acid (VII),

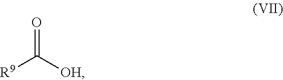

in the presence of a coupling reagent and a base to form the compound of formula (VIII),

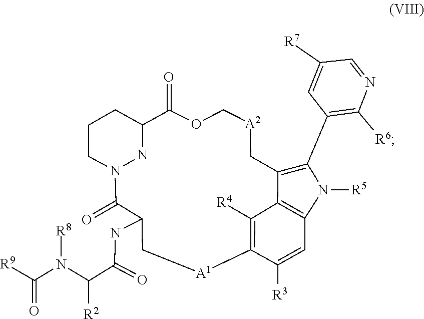

c) coupling reaction between compound of formula (XI),

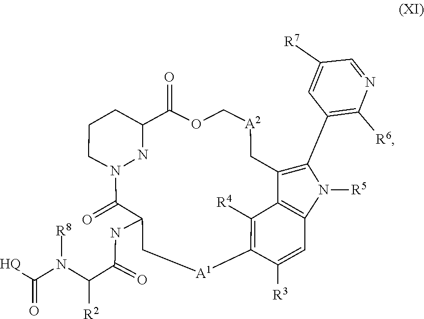

and acid (XII),

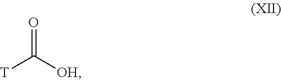

in the presence of a coupling reagent and a base to form the compound of formula (XIII),

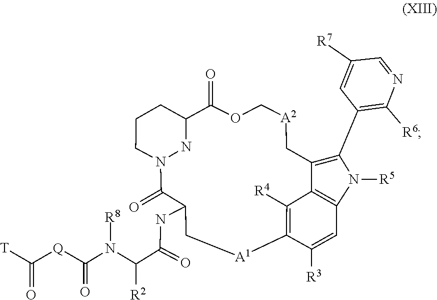

wherein
in step a), b) and c) the coupling reagent can be, for example, T₃P, HATU, PyBOP or EDCl/HOBt; the base can be, for example, TEA, DIEPA or DMAP; T is dihaloC₁₋₆alkyl; Q is unsubstituted or substituted piperidinylene or pyrrolidinylene.

A compound of formula (I) or (Ia) when manufactured according to the above process is also an object of the invention.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:

| | |
|---|---|
| ACN | acetonitrile |
| aq. | Aqueous |
| Boc-N-Me-Val-OH | N-(tert-Butoxycarbonyl)-N-methyl-L-valine |
| (Boc)₂O | Di-tert-butyldicarbonate |
| (R)-binap | (R)-(+)-2,2′-Bis(diphenylphosphino)-1,1′-binaphthyl |
| CDCl₃: | deuterated chloroform |
| CD₃OD: | deuterated methanol |
| COMU | (1-Cyano-2-ethoxy-2-oxoethylidenaminooxy)dimethylamino- |
| | morpholino-carbenium hexafluorophosphate |
| DIEPA: | N,N-diethylpropylamine |
| DIBAL-H | Diisobutylaluminium hydride |
| DMAP: | 4-Dimethylaminopyridine |
| DMF: | dimethyl formamide |
| DMSO: | dimethyl sulfoxide |
| EDCI: | N-Ethyl-N′-(3-dimethylaminopropyl)carbodiimide hydrochloride |
| EtOAc or EA: | ethyl acetate |
| FRET | fluorescence resonance energy transfer |
| HATU: | (1-[Bis(dimethylamino)methylene]-1H-1,2,3-triazolo[4,5- |
| | b]pyridinium 3-oxid hexafluorophosphate) |
| hr(s): | hour(s) |
| HPLC: | high performance liquid chromatography |
| HOBt: | N-hydroxybenzotriazole |
| H-VAL-OTBU HCl | (S)-tert-Butyl 2-amino-3-methylbutanoate hydrochloride |
| [Ir(OMe)(COD)]₂ | (1,5-Cyclooctadiene)(methoxy)iridium(I) dimer |
| LDA | Lithium diisopropylamide |
| MS: (ESI): | mass spectroscopy (electron spray ionization) |
| min(s) | minute(s) |
| NMM | N-Methylmorpholine |
| NMR: | nuclear magnetic resonance |
| NMO | 4-Methylmorpholine N-oxide |
| obsd. | Observed |
| Pd(dppf)Cl₂ | [1,1′-Bis(diphenylphosphino)ferrocene]dichloropalladium(II) |
| Pd(dtbpf)Cl₂ | [1,1′-Bis(di-tert-butylphosphino)ferrocene]dichloropalladium(II) |
| prep-HPLC | preparative high performance liquid chromatography |
| PyBOP: | benzotriazol-1-yloxytripyrrolidinophosphonium |
| | hexafluorophosphate |
| RT or rt: | room temperature |
| sat. | saturated |
| SFC | supercritical fluid chromatography |
| TEA: | triethylamine |
| TFA: | trifluoroacetic acid |
| THF: | tetrahydrofuran |
| TEA: | triethylamine |
| T₃P: | propylphosphonic anhydride |

### GENERAL EXPERIMENTAL CONDITIONS

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃—H₂O in MeOH), back pressure 100 bar, detection UV @254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃—H₂O in H₂O; B: acetonitrile;
Basic condition II: A: 0.025% NH₃—H₂O in H₂O; B: acetonitrile;
Neutral condition: A: H₂O; B: acetonitrile.
Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### Preparative Examples

### Preparation of Intermediate

### Intermediate A

### 1-[6-[(1S)-1-methoxyethyl]-5-(4,4,5,5-tetramethyl-1,3,2-dioxaborolan-2-yl)-3-pyridyl]-4-methyl-piperazine

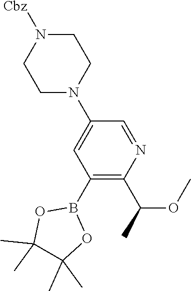

The title intermediate A was prepared according to the following scheme:

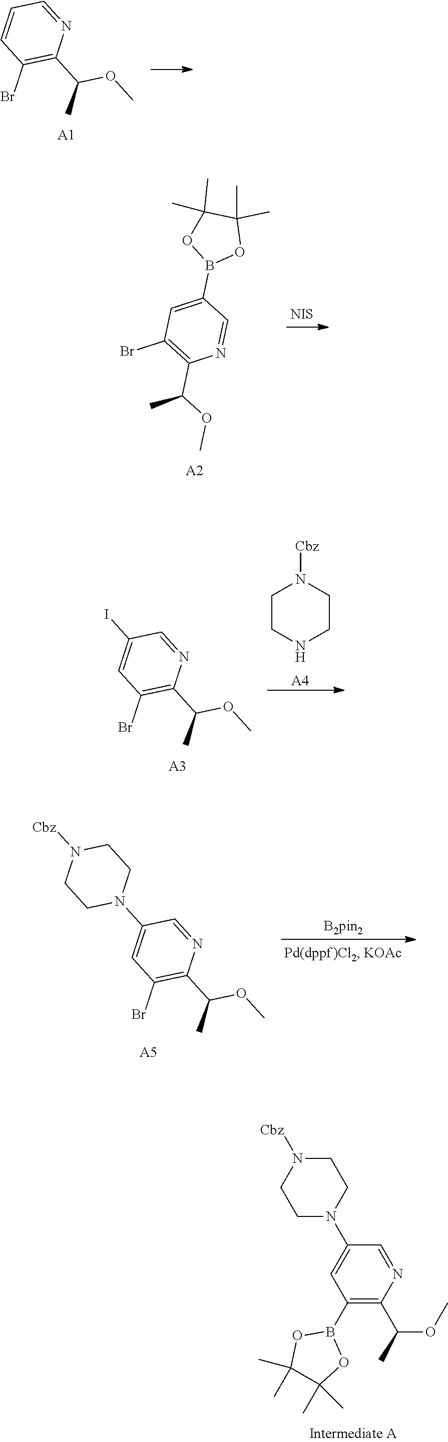

### Step 1: Preparation of 3-bromo-2-[(1S)-1-methoxyethyl]-5-(4,4,5,5-tetramethyl-1,3,2-dioxaborolan-2-yl)pyridine (compound A2)

To a solution of 3-bromo-2-[(1S)-1-methoxyethyl]pyridine (compound A1, 2.0 g, 9.26 mmol) and bis(pinacolato)diboron (3.5 g, 13.9 mmol) in THF (30 mL) were added 4,4′-di-tert-butyl-2,2′-bipyridin (372.7 mg, 1.39 mmol) and [Ir(OMe)(COD)]₂ (306.3 mg, 0.460 mmol). The mixture was stirred at 75° C. for 16 hours under N₂ protection. The mixture was filtrated and the filtrate was concentrated in vacuo. The residue was purified by silica gel chromatography (EA/PE: 0-20%) to afford 3-bromo-2-[(1S)-1-methoxyethyl]-5-(4,4,5,5-tetramethyl-1,3,2-dioxaborolan-2-yl)pyridine (compound A2, 2.4 g) as yellow oil. ¹H NMR (400 MHz, CDCl3) δ ppm 8.91 (d, J=1.4 Hz, 1H), 8.21 (d, J=1.4 Hz, 1H), 4.95 (q, J=6.5 Hz, 1H), 3.30 (s, 3H), 1.49 (d, J=6.5 Hz, 3H), 1.35 (s, 12H).

### Step 2: Preparation of 3-bromo-5-iodo-2-[(1S)-1-methoxyethyl]pyridine (compound A3)

To a solution of 3-bromo-2-[(1S)-1-methoxyethyl]-5-(4,4,5,5-tetramethyl-1,3,2-dioxaborolan-2-yl)pyridine (compound A2, 2.5 g, 7.3 mmol) in ACN (40 mL) was added N-iodosuccinimide (4.1 g, 18.27 mmol). The mixture was stirred at 90° C. for 40 hrs under N₂ protection. The reaction was quenched with saturated solution of Na₂SO₃ (40 mL) and the reaction mixture was extracted with EtOAc (30 mL, twice). The combined organic layer was washed with brine (50 mL), filtered and the filtrate was concentrated under vacuum. The residue was purified by silica gel chromatography (EA/PE: 0-20%) to afford 3-bromo-5-iodo-2-[(1S)-1-methoxyethyl]pyridine (compound A3, 660 mg) as yellow oil. MS calc'd 342 (MH⁺), measured 341.8 (MH⁺).

### Step 3: Preparation of benzyl 4-[5-bromo-6-[(1S)-1-methoxyethyl]-3-pyridyl]piperazine-1-carboxylate (Intermediate A5)

To a solution of 3-bromo-5-iodo-2-[(1S)-1-methoxyethyl]pyridine (compound A3, 660 mg, 1.9 mmol) and 1-Cbz-piperazine (compound A4, 425.1 mg, 1.9 mmol) in toluene (10 mL) were added cesium carbonate (1.6 g, 4.83 mmol), (R)-BINAP (60.1 mg, 0.1 mmol) and palladium (II) acetate (43.3 mg, 0.19 mmol). The mixture was stirred at 100° C. for 12 hours under N₂ protection. The mixture was filtered and the filtrate was concentrated under vacuum. The residue was purified by silica gel chromatography (EA/PE: 0-50%) to afford benzyl 4-[5-bromo-6-[(1S)-1-methoxyethyl]-3-pyridyl]piperazine-1-carboxylate (Intermediate A5, 740 mg) as a yellow solid. MS calc'd 434.1 (MH⁺), measured 434.1 (MH⁺).

### Step 4: Preparation of 1-[6-[(1S)-1-methoxyethyl]-5-(4,4,5,5-tetramethyl-1,3,2-dioxaborolan-2-yl)-3-pyridyl]-4-methyl-piperazine (Intermediate A)

To a solution of benzyl 4-[5-bromo-6-[(1S)-1-methoxyethyl]-3-pyridyl]piperazine-1-carboxylate (Intermediate A5, 740 mg, 1.7 mmol) and bis(pinacolato)diboron (519.2 mg, 2.04 mmol) in toluene (12 mL) were added KOAc (418.0 mg, 4.26 mmol) and Pd(dppf)Cl₂ (124.7 mg, 0.170 mmol). The reaction mixture was stirred at 90° C. for 12 hrs under N₂ protection. The mixture was filtered and the filtrate was concentrated in vacuo. The residue was purified by silica gel column to afford 1-[6-[(1S)-1-methoxyethyl]-5-(4,4,5,5-tetramethyl-1,3,2-dioxaborolan-2-yl)-3-pyridyl]-4-methyl-piperazine (Intermediate A, 470 mg) as a brown solid. MS calc'd 482.3 (MH⁺), measured 482.2 (MH⁺).

### Intermediate B

### Methyl (3S)-1-[(2S)-2-(tert-butoxycarbonylamino)-3-[3-(4,4,5,5-tetramethyl-1,3,2-dioxaborolan-2-yl)-5-triisopropylsilyloxy-phenyl]propanoyl]hexahydropyridazine-3-carboxylate

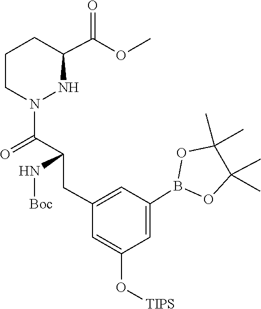

The intermediate B was prepared according to the following scheme:

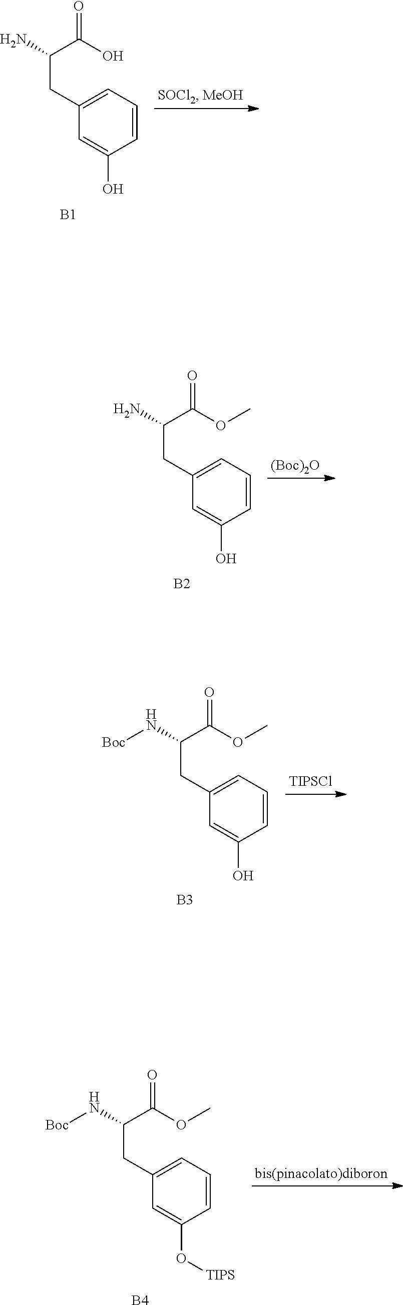

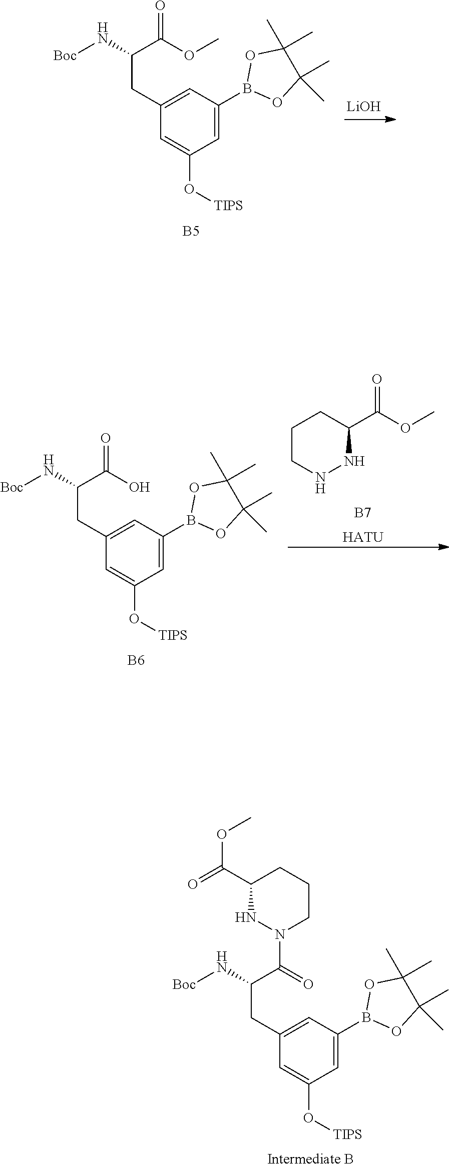

### Step 1: Preparation of methyl (2S)-2-amino-3-(3-hydroxyphenyl)propanoate (compound B2)

To a solution of L-M-tyrosine (compound B1, 5.0 g, 27.6 mmol) in methanol (80 mL) was added thionyl chloride (10 mL, 137.9 mmol). The mixture was stirred at 60° C. for 12 hrs. The reaction mixture was cooled to 20° C. and concentrated in vacuo to afford methyl (2S)-2-amino-3-(3-hydroxyphenyl)propanoate (compound B2, 6.2 g) as a yellow solid. ¹H NMR (400 MHz, CD3OD) δ ppm 7.18 (t, J=8.0 Hz, 1H), 6.78-6.66 (m, 3H), 4.29 (t, J=6.4 Hz, 1H), 3.82 (s, 3H), 3.23-3.05 (m, 2H).

### Step 2: Preparation of methyl (2S)-2-(tert-butoxycarbonylamino)-3-(3-hydroxyphenyl)propanoate (compound B3)

To a solution of methyl (2S)-2-amino-3-(3-hydroxyphenyl)propanoate (compound B2, 32.0 g, 138.1 mmol) in THF (80 mL) and water (20 mL) was added sodium bicarbonate (40.6 g, 483.4 mmol) followed by di-t-butyldicarbonate (33.1 g, 151.9 mmol) at 20° C. The mixture was stirred at 20° C. for 12 hours. The mixture was diluted with water (100 mL) and acidified by 1 M aq. solution of HCl until the pH=5. The mixture was extracted with ethyl acetate (100 mL, 3 times). The combined organic phase was washed with brine (80 mL), dried over anhydrous sodium sulfate, filtered and the filtrate was concentrated under vacuum to afford methyl (2S)-2-(tert-butoxycarbonylamino)-3-(3-hydroxyphenyl)propanoate (compound B3, 40 g) as colorless gum. MS: calc'd 318 (MNa⁺), measured 318.3 (MNa⁺).

### Step 3: Preparation of methyl (2S)-2-(tert-butoxycarbonylamino)-3-(3-triisopropylsilyloxyphenyl)propanoate (compound B4)

To a solution of methyl (2S)-2-(tert-butoxycarbonylamino)-3-(3-hydroxyphenyl)propanoate (compound B3, 40.0 g, 135.4 mmol) and 1H-imidazole (27.6 g, 406.3 mmol) in DMF (400 mL) was added triisopropylsilyl chloride (39.1 g, 203.1 mmol) dropwise at 0° C. After being stirred for 12 hrs at 25° C., the mixture was diluted with water (250 mL) at 0° C. and extracted with ethyl acetate (200 mL, 3 times). The combined organic phase was washed with brine (80 mL, four times), dried over anhydrous sodium sulfate, filtered and the filtrate was concentrated under vacuum to give a residue. The residue was purified by silica gel chromatography (EA/PE: 0-20%) to afford methyl (2S)-2-(tert-butoxycarbonylamino)-3-(3-triisopropylsilyloxyphenyl)propanoate (compound B4, 60 g) as yellow oil. MS: calc'd 474 (MNa⁺), measured 474.2 (MNa⁺).

### Step 4: Preparation of methyl (2S)-2-(tert-butoxycarbonylamino)-3-[3-(4,4,5,5-tetramethyl-1,3,2-dioxaborolan-2-yl)-5-triisopropylsilyloxy-phenyl]propanoate (compound B5)

To a solution of methyl (2S)-2-(tert-butoxycarbonylamino)-3-(3-triisopropylsilyloxyphenyl)propanoate (compound B4, 15.0 g, 33.2 mmol), 4,4′-di-tert-butyl-2,2′-bipyridine (2.6 g, 9.9 mmol) and bis(pinacolato)diboron (12.6 g, 49.8 mmol) in hexane (200 mL) was added [Ir(OMe)(COD)]_{b}(2.2 g, 3.3 mmol). The mixture was degassed and purged with N₂ for 3 times. The resulting mixture was stirred at 70° C. for 12 hrs. Then the reaction mixture was cooled to 20° C., diluted with petroleum ether (100 mL) and filtered. The filtrate was concentrated in vacuo to give a residue, which was purified by silica gel chromatography (EA/PE: 0-20%) to afford methyl (2S)-2-(tert-butoxycarbonylamino)-3-[3-(4,4,5,5-tetramethyl-1,3,2-dioxaborolan-2-yl)-5-triisopropylsilyloxy-phenyl]propanoate (compound B5, 21 g) as yellow oil. MS: calc'd 600 (MNa⁺), measured 600.3 (MNa⁺).

### Step 5: Preparation of (2S)-2-(tert-butoxycarbonylamino)-3-[3-(4,4,5,5-tetramethyl-1,3,2-dioxaborolan-2-yl)-5-triisopropylsilyloxy-phenyl]propanoic acid (compound B6)

To a solution of methyl (2S)-2-(tert-butoxycarbonylamino)-3-[3-(4,4,5,5-tetramethyl-1,3,2-dioxaborolan-2-yl)-5-triisopropylsilyloxy-phenyl]propanoate (compound B5, 40.0 g, 69.2 mmol) in methanol (300 mL) was added a solution of lithium hydroxide (3.2 mL, 346.2 mmol) in water (100 mL). After being stirred at 20° C. for 1 hour, the reaction mixture was diluted with water (200 mL) and MeOH was removed under vacuum. The resulting mixture was acidified by 1 M aq. solution of HCl until the pH=5. The resulting mixture was extracted with EtOAc (250 mL, 3 times). The organic phase was washed with brine (150 mL), dried over anhydrous sodium sulfate, filtered and the filtrate was concentrated in vacuo to afford (2S)-2-(tert-butoxycarbonylamino)-3-[3-(4,4,5,5-tetramethyl-1,3,2-dioxaborolan-2-yl)-5-triisopropylsilyloxy-phenyl]propanoic acid (compound B6, 33 g) as a white solid. MS: calc'd 586 (MNa⁺), measured 586.3 (MNa⁺).

### Step 6: Preparation of methyl (3S)-1-[(2S)-2-(tert-butoxycarbonylamino)-3-[3-(4,4,5,5-tetramethyl-1,3,2-dioxaborolan-2-yl)-5-triisopropylsilyloxy-phenyl]propanoyl]hexahydropyridazine-3-carboxylate (Intermediate B)

To a solution of (2S)-2-(tert-butoxycarbonylamino)-3-[3-(4,4,5,5-tetramethyl-1,3,2-dioxaborolan-2-yl)-5-triisopropylsilyloxy-phenyl]propanoic acid (compound B6, 8.0 g, 14.1 mmol) and O-(7-azabenzotriazol-1-yl)-N,N,N′,N′-tetramethyluronium hexafluorophosphate (5.6 g, 14.9 mmol) in DMF (100 mL) was added N,N-diisopropylethylamine (6.4 g, 49.6 mmol). The mixture was stirred at 0° C. for 10 min. Then methyl (3S)-hexahydropyridazine-3-carboxylate hydrochloride salt (compound B7, 2.6 g, 14.9 mmol) was added. The resulting mixture was stirred at 20° C. for 1.5 hrs. The mixture was diluted with water (200 mL) and extracted with EtOAc (100 mL, twice). The combined organic phase was washed with brine, dried over anhydrous sodium sulfate, filtrated and the filtrate was concentrated under vacuum. The residue was purified by silica gel chromatography to afford methyl (3S)-1-[(2S)-2-(tert-butoxycarbonylamino)-3-[3-(4,4,5,5-tetramethyl-1,3,2-dioxaborolan-2-yl)-5-triisopropylsilyloxy-phenyl]propanoyl]hexahydropyridazine-3-carboxylate (intermediate B, 7.8 g) as yellow oil. MS: calc'd 690 (MH⁺), measured 690.4 (MH⁺).

### Intermediate C

### Methyl (3S)-1-[(2S)-3-(4-bromothiazol-2-yl)-2-(tert-butoxycarbonylamino)-propanoyl]hexahydropyridazine-3-carboxylate

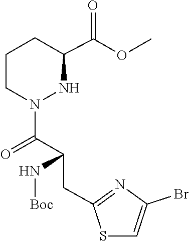 [SMILES: COC(=O)[C@@H]1CCCN(C(=O)[C@H](Cc2nc(Br)cs2)NC(=O)OC(C)(C)C)N1]

The intermediate C was prepared according to the following scheme:

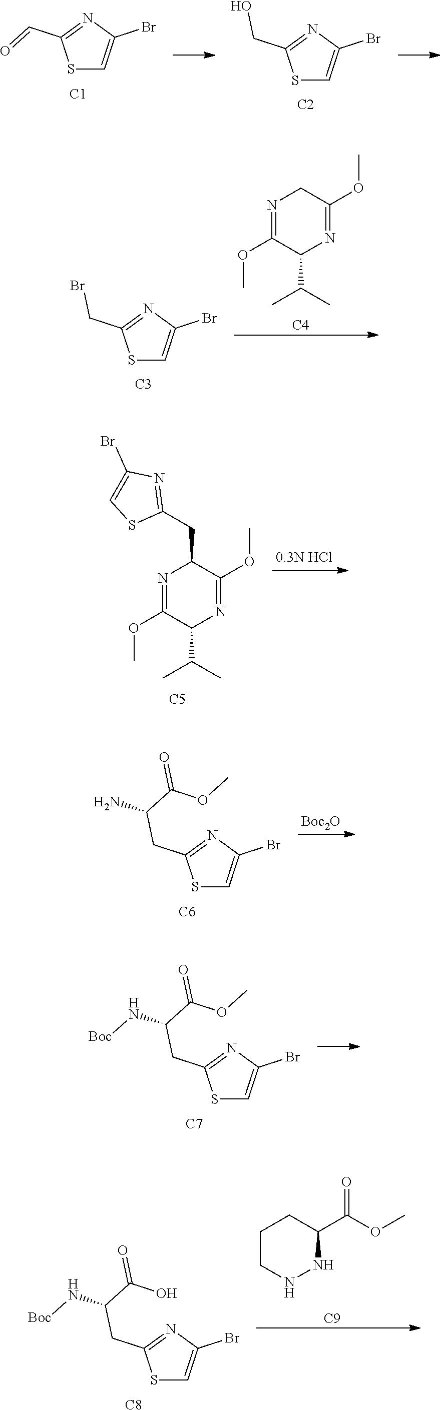

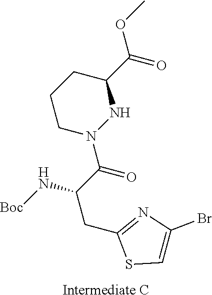 [SMILES: COC(=O)[C@@H]1CCCN(C(=O)[C@H](Cc2nc(Br)cs2)NC(=O)OC(C)(C)C)N1 ; "Intermediate C"]

### Step 1: Preparation of (4-bromothiazol-2-yl)methanol (compound C2)

To a solution of 4-bromothiazole-2-carboxaldehyde (6.0 g, 31.25 mmol) in methanol (70 mL) was added sodium borohydride (1.7 g, 46.87 mmol) at 0° C. The mixture was stirred at 25° C. for 1 hour. The reaction was quenched with water (300 mL) at 0° C. and the reaction mixture was extracted by ethyl acetate (200 mL, three times). The combined organic phase was washed with brine (150 mL, twice), dried over anhydrous sodium sulfate, filtered and the filtrate was concentrated under vacuum to afford (4-bromothiazol-2-yl)methanol (compound C2, 6 g) as colorless oil.

### Step 2: Preparation of 4-bromo-2-(bromomethyl)thiazole (compound C3)

To a solution of (4-bromothiazol-2-yl)methanol (compound C2, 6.0 g, 30.92 mmol) in DCM (80 mL) was added CBr₄ (15.4 g, 46.38 mmol) and triphenylphosphine (12.1 g, 46.38 mmol) at 0° C. After being stirred at 25° C. for 1 hour, the mixture was filtered and the filtrate was concentrated under vacuum. The residue was purified by silica gel column, eluted with ethyl acetate in petroleum ether=0˜10% to afford (4-bromothiazol-2-yl)methanol (compound C3, 6.0 g) as yellow oil. MS calc'd 255.9 (MH⁺), measured 255.9 (MH⁺).

### Step 3: Preparation of 4-bromo-2-[[(2S,5R)-5-isopropyl-3,6-dimethoxy-2,5-dihydropyrazin-2-yl]methyl]thiazole (compound C5)

To a mixture of (R)-2,5-dihydro-3,6-dimethoxy-2-isopropylpyrazine (compound C4, 4.3 g, 23.45 mmol) in THF (60 mL) was added n-butyllithium (10 ML, 25.22 mmol, 2.5 M) at −78° C. slowly. After addition, the mixture was stirred for 0.5 hour at −78° C. 4-bromo-2-(bromomethyl)thiazole (compound C3, 5.4 g, 21.02 mmol) was added into above mixture at −78° C. which was stirred for another 1 hour. The reaction was quenched with saturated solution of NH₄Cl (100 mL) and the reaction mixture was extracted with EtOAc (100 mL, twice). The combined organic layer was washed with brine (150 mL), dried over anhydrous sodium sulfate, filtered and the filtrate was concentrated under vacuum. The residue was purified by reversed-phase chromatography to afford 4-bromo-2-[[(2S,5R)-5-isopropyl-3,6-dimethoxy-2,5-dihydropyrazin-2-yl]methyl]thiazole (compound C5, 3.6 g) as yellow oil. MS calc'd 360 (MH⁺), measured 359.9 (MH⁺).

### Step 4: Preparation of methyl (2S)-2-amino-3-(4-bromothiazol-2-yl)propanoate (compound C6)

To a solution of 4-bromo-2-[[(2S,5R)-5-isopropyl-3,6-dimethoxy-2,5-dihydropyrazin-2-yl]methyl]thiazole (compound C5, 3.6 g, 10 mmol) in ACN (20 mL) was added hydrochloric acid (66.6 mL, 0.3 M). The mixture was stirred at 25° C. for 2 hours. The mixture was basified by saturated solution of NaHCO₃ until pH=8. The mixture was extracted with EtOAc (80 mL, six times). The combined organic layer was dried over anhydrous sodium sulfate, filtered and the filtrate was concentrated under vacuum to afford methyl (2S)-2-amino-3-(4-bromothiazol-2-yl)propanoate (compound C6, 3.1 g) as yellow oil. MS calc'd 264.9 (MH⁺), measured 264.9 (MH⁺).

### Step 5: Preparation of methyl (2S)-3-(4-bromothiazol-2-yl)-2-(tert-butoxycarbonylamino)propanoate (compound C7)

To a solution of methyl (2S)-2-amino-3-(4-bromothiazol-2-yl)propanoate (compound C6, 3.1 g, 11.69 mmol) in DCM (40 mL) were added triethylamine (2.9 g, 29.23 mmol) and (Boc)₂O (3.8 g, 17.54 mmol). After being stirred at 30° C. for 12 hours, the mixture was concentrated under vacuum. The residue was purified by silica gel column, eluted with ethyl acetate in petroleum ether=0˜30% to afford methyl (2S)-3-(4-bromothiazol-2-yl)-2-(tert-butoxycarbonylamino)propanoate (compound C7, 3.2 g) as yellow oil. MS calc'd 387 (MNa⁺), measured 386.9 (MNa⁺).

### Step 6: Preparation of (2S)-3-(4-bromothiazol-2-yl)-2-(tert-butoxycarbonylamino)-propanoic acid (compound C8)

To a solution of methyl (2S)-3-(4-bromothiazol-2-yl)-2-(tert-butoxycarbonylamino)propanoate (compound C7, 3.2 g, 8.76 mmol) in THF (30 mL), methanol (2 mL) and water (10 mL) was added lithium hydroxide (0.4 mL, 43.81 mmol). After being stirred at 25° C. for 1 hour, the reaction mixture was acidified by 1 M solution of HCl until pH=5. The mixture was extracted with EtOAc (40 mL, twice). The combined organic layer was washed with brine (100 mL), dried over anhydrous sodium sulfate, filtered and the filtrate was concentrated under vacuum to afford (2S)-3-(4-bromothiazol-2-yl)-2-(tert-butoxycarbonylamino)propanoic acid (compound C8, 3.1 g) as yellow oil. MS calc'd 373 (MNa⁺), measured 372.9 (MNa⁺).

### Step 7: Preparation of methyl (3S)-1-[(2S)-3-(4-bromothiazol-2-yl)-2-(tert-butoxycarbonylamino)propanoyl]hexahydropyridazine-3-carboxylate (Intermediate C)

To a solution of (2S)-3-(4-bromothiazol-2-yl)-2-(tert-butoxycarbonylamino)propanoic acid (compound C8, 3.1 g, 8.83 mmol) in DCM (50 mL) was added methyl (3S)-hexahydropyridazine-3-carboxylate;hydrochloride (compound C9, 2.4 g, 13.24 mmol), EDCl (3.4 g, 17.65 mmol), 1-Hydroxybenzotriazole (238.5 mg, 1.77 mmol) and NMM (9.92 mL, 88.26 mmol) at 0° C. After being stirred at 25° C. for 1 hour, the reaction mixture was diluted with water (60 mL) and extracted with EtOAc (60 mL, three times). The combined organic layer was washed with brine (100 mL), dried over anhydrous sodium sulfate, filtered and the filtrate was concentrated under vacuum. The residue was purified by silica gel column and eluted with ethyl acetate in petroleum ether=10-30% to afford methyl (3S)-1-[(2S)-3-(4-bromothiazol-2-yl)-2-(tert-butoxycarbonylamino)propanoyl]hexahydropyridazine-3-carboxylate (intermediate C, 2.4 g). MS calc'd 477 (MH⁺), measured 476.9 (MH⁺).

### Intermediate D

### (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

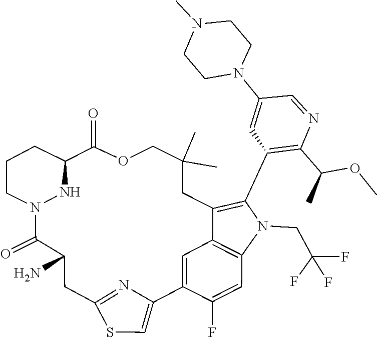

The title intermediate D was prepared according to the following scheme:

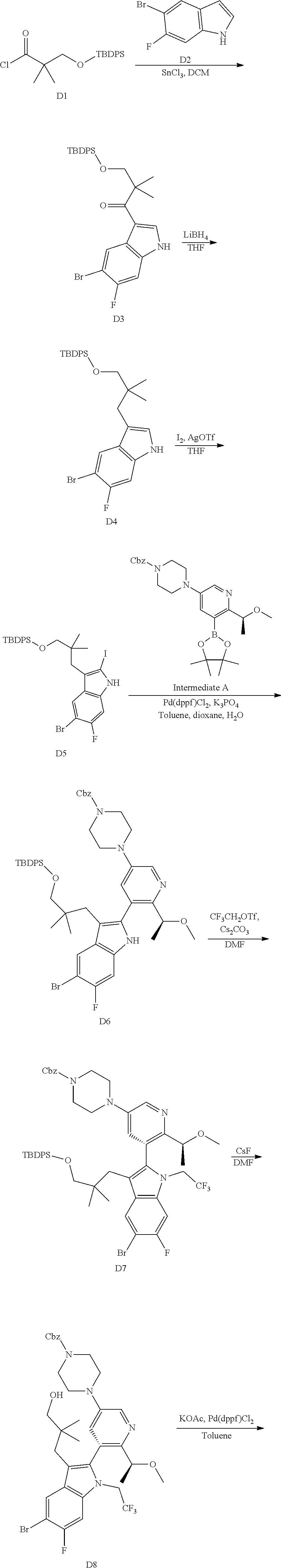

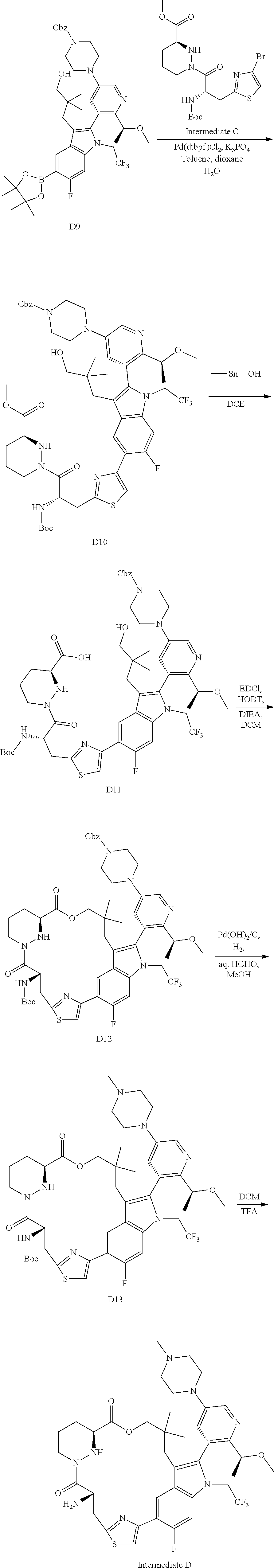

### Step 1: Preparation of 1-(5-bromo-6-fluoro-1H-indol-3-yl)-3-((tert-butyldiphenylsilyl) oxy)-2,2-dimethylpropan-1-one (compound D3)

To a mixture of 3-((tert-butyldiphenylsilyl)oxy)-2,2-dimethylpropanoyl chloride (compound D1, 35.0 g, 116.8 mmol) in DCM (400 mL) at 0° C. was added a solution of SnCl₄ (97.2 mL, 121.5 mmol) slowly. After being stirred at −40° C. for 0.5 hour, 5-bromo-6-fluoro-1H-indole (compound D2, 25.0 g, 116.8 mmol) in DCM (200 mL) was added dropwise and the mixture was stirred at −40° C. for 15 min. After the reaction was completed, it was quenched with sat. NaHCO₃ aq. (800 mL), and the reaction mixture was extracted with EtOAc (900 mL, twice). The combined organic layer was washed with brine (700 mL), dried over Na₂SO₄, filtered and concentrated in vacuo. The residue was triturated with the solution (100 mL, Petroleum ether:Ethyl acetate=8:1) and filtered. The filter cake was dried in vacuo to afford 1-(5-bromo-6-fluoro-1H-indol-3-yl)-3-((tertbutyldiphenylsilyl)oxy)-2,2-dimethylpropan-1-one (compound D3, 50.0 g) as a yellow solid. MS calc'd 552.1 (MH⁺), measured 552.1 (MH⁺).

### Step 2: Preparation of [3-(5-bromo-6-fluoro-1H-indol-3-yl)-2,2-dimethyl-propoxy]-tert-butyl-diphenyl-silane (compound D4)

To a mixture of 1-(5-bromo-6-fluoro-1H-indol-3-yl)-3-((tertbutyldiphenylsilyl)oxy)-2,2-dimethylpropan-1-one (compound D3, 50.0 g, 90.49 mmol) in THF (600 mL) was added LiBH₄ (48.4 mL, 193.49 mmol, 4 M in THF) dropwise at 0° C. The mixture was stirred at 70° C. for 24 hrs under nitrogen atmosphere. After the reaction was completed, it was quenched by addition of water (600 mL) at 0° C. slowly and the reaction mixture was extracted with EtOAc (600 mL, twice). The combined organic layer was washed with brine (600 mL), dried over Na₂SO₄, filtered and concentrated in vacuo. The residue was purified by silica column chromatography (EtOAc in PE=20%-33%) to afford [3-(5-bromo-6-fluoro-1H-indol-3-yl)-2,2-dimethyl-propoxy]-tert-butyl-diphenyl-silane (compound D4, 46.0 g) as a white solid. MS calc'd 538.1 (MH⁺), measured 538.2 (MH⁺).

### Step 3: Preparation of [3-(5-bromo-6-fluoro-2-iodo-1H-indol-3-yl)-2,2-dimethyl-propoxy]-tert-butyl-diphenyl-silane (compound D5)

To a mixture of [3-(5-bromo-6-fluoro-1H-indol-3-yl)-2,2-dimethyl-propoxy]-tert-butyl-diphenyl-silane (compound D4, 35.4 g, 65.73 mmol) and iodine (18.4 g, 72.3 mmol) in THF (400 mL) was added silver trifluoromethanesulfonate (20.3 g, 78.88 mmol) at 0° C. The mixture was stirred at 0° C. for 10 min. After the reaction was completed, it was quenched by sat. Na₂SO₃ aq. (400 mL) and EtOAc (400 mL) and the reaction mixture was filtered. The organic layer was washed with brine (100 mL), dried over Na₂SO₄, filtered and concentrated in vacuo. The residue was purified by silica column chromatography (EtOAc in PE=0%˜2.5%) to afford [3-(5-bromo-6-fluoro-2-iodo-1H-indol-3-yl)-2,2-dimethyl-propoxy]-tert-butyl-diphenyl-silane (compound D5, 43.0 g) as a yellow solid. MS calc'd 664.0 (MH⁺), measured 664.1 (MH⁺).

### Step 4: Preparation of benzyl 4-[5-[5-bromo-3-[3-[tert-butyl(diphenyl)silyl]oxy-2,2-dimethyl-propyl]-6-fluoro-1H-indol-2-yl]-6-[(1S)-1-methoxyethyl]-3-pyridyl]piperazine-1-carboxylate (compound D6)

To a mixture of [3-(5-bromo-6-fluoro-2-iodo-1H-indol-3-yl)-2,2-dimethyl-propoxy]-tert-butyl-diphenyl-silane (compound D5, 16.7 g, 25.13 mmol) and benzyl 4-[6-[(1S)-1-methoxyethyl]-5-(4,4,5,5-tetramethyl-1,3,2-dioxaborolan-2-yl)-3-pyridyl]piperazine-1-carboxylate (Intermediate A, 16.7 g, 34.69 mmol) in a mixed solution of 1,4-dioxane (270 mL)/Toluene (90 mL)/water (90 mL) were added potassium phosphate (15.7 g, 73.92 mmol) and Pd(dppf)Cl₂ (920 mg, 1.26 mmol). The mixture was stirred at 70° C. for 12 hrs under nitrogen atmosphere. After the reaction was completed, the mixture was filtered and concentrated in vacuo. The residue was purified by silica column chromatography (EtOAc in PE=20%-50%) to afford 4-[5-[5-bromo-3-[3-[tert-butyl(diphenyl)silyl]oxy-2,2-dimethyl-propyl]-6-fluoro-1H-indol-2-yl]-6-[(1S)-1-methoxyethyl]-3-pyridyl]piperazine-1-carboxylate (compound D6, 19.5 g) as a white solid. MS calc'd 891.3 (MH⁺), measured 891.3 (MH⁺).

### Step 5: Preparation of benzyl 4-[(5M)-5-[5-bromo-3-[3-[tert-butyl(diphenyl)silyl]oxy-2,2-dimethyl-propyl]-6-fluoro-1-(2,2,2-trifluoroethyl)indol-2-yl]-6-[(1S)-1-methoxyethyl]-3-pyridyl]piperazine-1-carboxylate (compound D7)

To a solution of 4-[5-[5-bromo-3-[3-[tert-butyl(diphenyl)silyl]oxy-2,2-dimethyl-propyl]-6-fluoro-1H-indol-2-yl]-6-[(1S)-1-methoxyethyl]-3-pyridyl]piperazine-1-carboxylate (compound D6, 14.5 g, 16.26 mmol) and Cs₂CO₃ (15.9 g, 48.77 mmol) in DMF (200 mL) was added 2,2,2-trifluoroethyl trifluoromethanesulfonate (37.7 g, 162.56 mmol) dropwise at 0° C., and the mixture was stirred at 20° C. for 12 hrs. After the reaction was completed, EtOAc (70 mL) and water (100 mL) were added and the layers were separated. The aqueous phase was extracted with EtOAc (70 mL, twice). Combined organic layer was washed with brine (100 mL, four times), dried over Na₂SO₄, filtered, and concentrated under vacuum to give a residue. The residue was purified by silica column chromatography to afford benzyl 4-[(5M)-5-[5-bromo-3-[3-[tert-butyl(diphenyl)silyl]oxy-2,2-dimethyl-propyl]-6-fluoro-1-(2,2,2-trifluoroethyl)indol-2-yl]-6-[(1S)-1-methoxyethyl]-3-pyridyl]piperazine-1-carboxylate (compound D7, 8.0 g, PEAK 1, faster eluted) as yellow oil. MS calc'd 973.3 (MH⁺1), measured 973.2 (MH⁺).

### Step 6: Preparation of benzyl 4-[(5M)-5-[5-bromo-6-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (compound D8)

To a solution of benzyl 4-[(5M)-5-[5-bromo-3-[3-[tert-butyl(diphenyl)silyl]oxy-2,2-dimethyl-propyl]-6-fluoro-1-(2,2,2-trifluoroethyl)indol-2-yl]-6-[(1S)-1-methoxyethyl]-3-pyridyl]piperazine-1-carboxylate (compound D7, 10.5 g, 10.78 mmol) in DMF (130 mL) was added cesium fluoride (8.2 g, 53.9 mmol) and the mixture was stirred at 60° C. for 24 hrs. After the reaction was completed, EtOAc (100 mL) and water (100 mL) were added and the layers were separated. The aqueous phase was extracted with EtOAc (100 mL, twice). The combined organic layer was washed with brine (80 mL, three times), dried over Na₂SO₄, filtered, and concentrated under vacuum to give a residue. The residue was purified by silica column chromatography (EtOAc in PE=25%-66%) to afford benzyl 4-[(5M)-5-[5-bromo-6-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (compound D8, 6.5 g) as a yellow solid. MS calc'd 735.2 (MH⁺), measured 735.1 (MH⁺).

### Step 7: Preparation of benzyl 4-[(5M)-5-[6-fluoro-3-(3-hydroxy-2,2-dimethyl-propyl)-5-(4,4,5,5-tetramethyl-1,3,2-dioxaborolan-2-yl)-1-(2,2,2-trifluoroethyl)indol-2-yl]-6-[(1S)-1-methoxyethyl]-3-pyridyl]piperazine-1-carboxylate (compound D9)

To a solution of benzyl 4-[(5M)-5-[5-bromo-6-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (compound D8, 5.4 g), bis(pinacolato)diboron (2.8 g, 11.01 mmol) and potassium acetate (1.2 mL, 18.35 mmol) in Toluene (70 mL) was added Pd(dppf)Cl₂ (537.1 mg, 0.73 mmol). The mixture was degassed and purged with nitrogen atmosphere for three times and the mixture was stirred at 90° C. for 12 hrs. After the reaction was completed, the mixture was cooled to room temperature. The reaction mixture was filtered and the filtrate was concentrated in vacuo to give a residue. The residue was purified by silica column chromatography (EtOAc in PE=25% 66%) to afford benzyl 4-[(5M)-5-[6-fluoro-3-(3-hydroxy-2,2-dimethyl-propyl)-5-(4,4,5,5-tetramethyl-1,3,2-dioxaborolan-2-yl)-1-(2,2,2-trifluoroethyl)indol-2-yl]-6-[(1S)-1-methoxyethyl]-3-pyridyl]piperazine-1-carboxylate (compound D9, 5.2 g) as yellow oil. MS calc'd 783.3 (MH⁺), measured 783.3 (MH⁺).

### Step 8: Preparation of methyl (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)-propanoyl]hexahydropyridazine-3-carboxylate (compound D10)

To a mixture of methyl (3S)-1-[(2S)-3-(4-bromothiazol-2-yl)-2-(tert-butoxycarbonylamino)propanoyl]hexahydropyridazine-3-carboxylate (intermediate C, 2.7 g, 5.69 mmol), benzyl 4-[(5M)-5-[6-fluoro-3-(3-hydroxy-2,2-dimethyl-propyl)-5-(4,4,5,5-tetramethyl-1,3,2-dioxaborolan-2-yl)-1-(2,2,2-trifluoroethyl)indol-2-yl]-6-[(1S)-1-methoxyethyl]-3-pyridyl]piperazine-1-carboxylate (compound D9, 4.9 g, 6.32 mmol) in toluene (60 mL)/1,4-dioxane (20 mL)/water (20 mL) were added K₃PO₄ (3.4 g, 15.81 mmol) and Pd(dtbpf)Cl₂ (412.2 mg, 0.63 mmol) under nitrogen atmosphere. The mixture was stirred at 70° C. for 12 hrs. After the reaction was completed, the mixture was concentrated in vacuo to give a residue. The residue was purified by silica column (EtOAc in PE=10%-75%) to afford methyl (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)-propanoyl]hexahydropyridazine-3-carboxylate (compound D10, 3.6 g) as a brown solid. MS calc'd 1053.4 (MH⁺), measured 1053.3 (MH⁺).

### Step 9: Preparation of (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)propanoyl]hexahydropyridazine-3-carboxylic acid (compound D11)

To a solution methyl (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)-propanoyl]-hexahydropyridazine-3-carboxylate (compound D10, 3.6 g, 3.42 mmol) in DCE (50 mL) was added trimethylstannanol (2.4 g, 13.67 mmol) and the mixture was stirred at 60° C. for 12 hrs.

After the reaction was completed, EtOAc (80 mL) and water (60 mL) were added and the layers were separated. The aqueous phase was extracted with EtOAc (80 mL, twice). The combined organic layer was washed with brine (100 mL), dried over Na₂SO₄, filtered, and concentrated under vacuum to give (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)propanoyl]hexahydropyridazine-3-carboxylic acid (compound D11, 4.3 g) as a brown solid. MS calc'd 1039.4 (MH⁺), measured 1039.2 (MH⁺).

### Step 10: Preparation of benzyl 4-[5-[(7S,13S)-7-(tert-butoxycarbonylamino)-24-fluoro-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-(20M)-20-yl]-6-[(1S)-1-methoxyethyl]-3-pyridyl]piperazine-1-carboxylate (compound D12)

To a mixture of (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)propanoyl]hexahydropyridazine-3-carboxylic acid (compound D11, 4.3 g, 4.14 mmol) in DCM (430 mL) was added DIEA (14.4 mL, 82.76 mmol), EDCl (11.9 g, 62.07 mmol) and 1-hydroxybenzotriazole (1.4 g, 10.35 mmol) at 0° C. The mixture was stirred at 15° C. for 12 hrs. After the reaction was completed, the mixture was concentrated in vacuo, then diluted with water (80 mL), extracted with EtOAc (80 mL, twice). The combined organic layer was washed with brine (80 mL), dried over Na₂SO₄, filtered and concentrated in vacuo. The residue was purified by silica column chromatography (EtOAc in PE=25%-66%) to afford benzyl 4-[5-[(7S,13S)-7-(tert-butoxycarbonylamino)-24-fluoro-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-(20M)-20-yl]-6-[(1S)-1-methoxyethyl]-3-pyridyl]piperazine-1-carboxylate (compound D12, 3.1 g) as yellow gum. MS calc'd 1021.4 (MH⁺), measured 1021.2 (MH⁺).

### Step 11: Preparation of tert-butyl N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound D13)

To a mixture of benzyl 4-[5-[(7S,13S)-7-(tert-butoxycarbonylamino)-24-fluoro-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-(20M)-20-yl]-6-[(1S)-1-methoxyethyl]-3-pyridyl]piperazine-1-carboxylate (compound D12, 3.1 g, 3.04 mmol) and formaldehyde aqueous (775.0 mg, 9.55 mmol) in methanol (150 mL) was added Pd(OH)₂ on activated carbon (2.79 g, 3.97 mmol). The mixture was degassed and purged with H₂ three times. The mixture was hydrogenated at 30° C. for 18 hrs. After the reaction was completed, the mixture was filtered and the filtrate was concentrated in vacuo to afford tert-butyl N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound D13, 2.6 g) as a brown solid. MS calc'd 901.3 (MH⁺), measured 901.3 (MH⁺).

### Step 12: Preparation of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D)

To a mixture of tert-butyl N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound D13, 2.6 g, 2.89 mmol) in DCM (18 mL) was added TFA (14.0 mL, 181.72 mmol). The mixture was stirred at 15° C. for 0.5 h. After the reaction was completed, the mixture was concentrated in vacuo and diluted with sat. NaHCO₃ (30 mL), extracted with EtOAc (30 mL, three times). The combined organic layer was washed with brine (50 mL), dried over Na₂SO₄, filtered and concentrated in vacuo to afford (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D, 2.0 g) as a yellow solid, which was used directly in the next step. MS calc'd 801.3 (MH⁺), measured 801.2 (MH⁺)

### Intermediate E

### (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

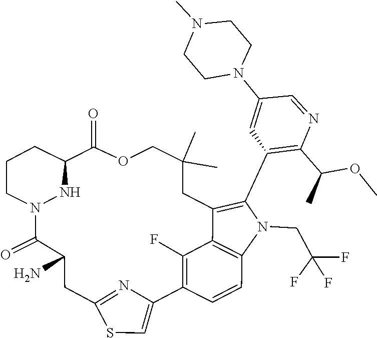

The title compound was prepared in analogy to the preparation of Intermediate D by using 5-bromo-4-fluoro-1H-indole instead of 5-bromo-6-fluoro-1H-indole (compound D2).

### Intermediate F

### (7S,13S)-7-amino-(20M)-20-[2-[(1S)-1-methoxyethyl]-5-(4-methylpiperazin-1-yl)-3-pyridyl]-17,17,24-trimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

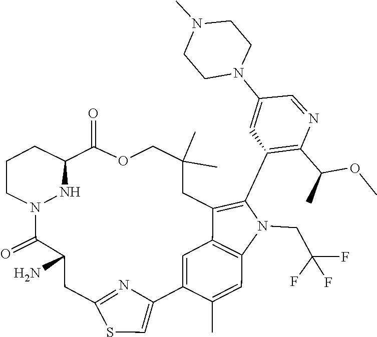

The title compound was prepared in analogy to the preparation of Intermediate D by using 5-bromo-6-methyl-1H-indole instead of 5-bromo-6-fluoro-1H-indole (compound D2).

### Intermediate G

### (8S,14S)-8-amino-25-fluoro-4-hydroxy-(21M)-21-[2-[(1S)-1-methoxyethyl]-5-(4-methylpiperazin-1-yl)-3-pyridyl]-18,18-dimethyl-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaene-9,15-dione

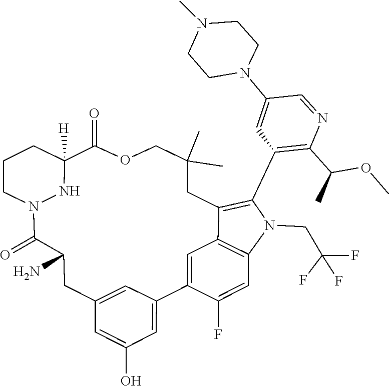

The compound Intermediate G was prepared according to the following scheme:

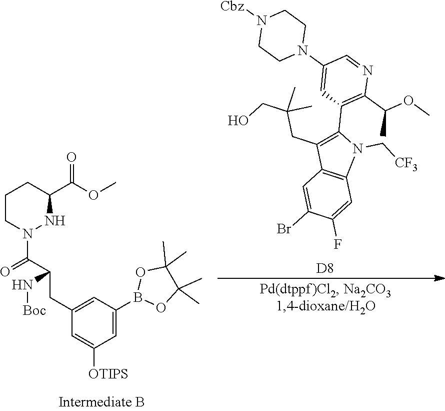

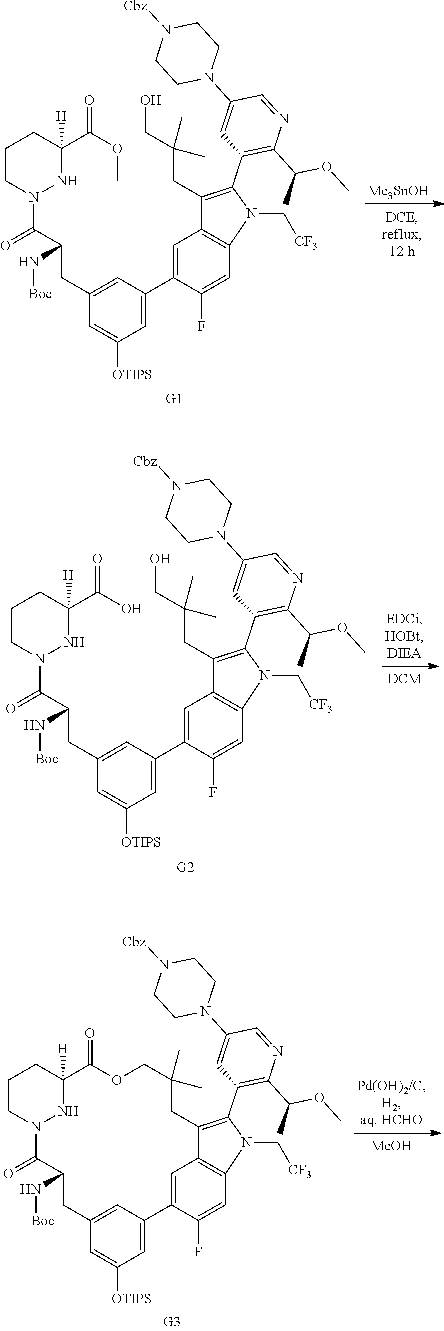

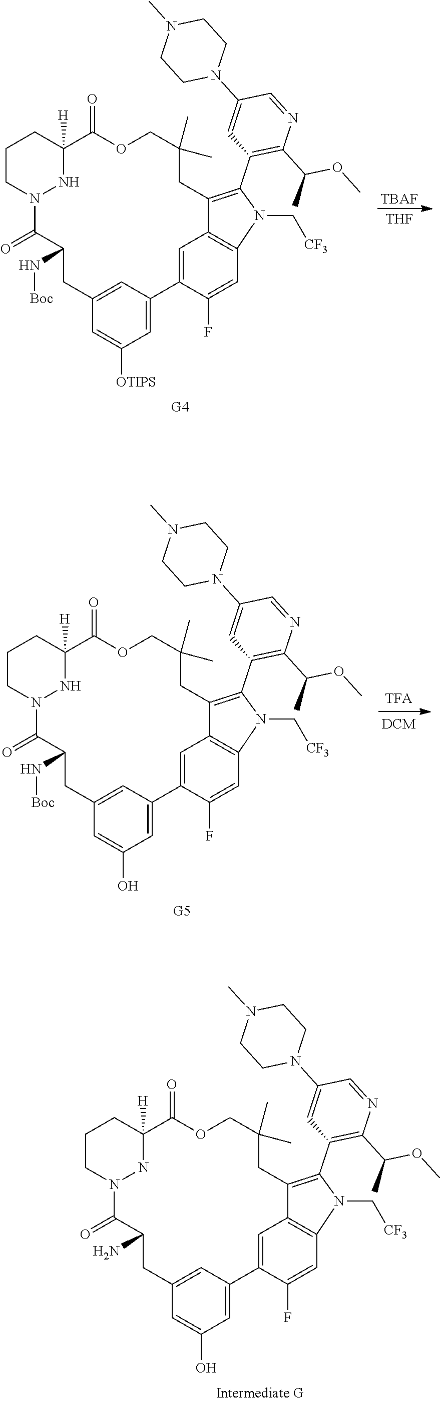

### Step 1: Preparation of methyl (3S)-1-[(2S)-3-[3-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]-5-triisopropylsilyloxy-phenyl]-2-(tert-butoxycarbonylamino)propanoyl]hexahydropyridazine-3-carboxylate (compound G1)

To a mixture of benzyl 4-[(5M)-5-[5-bromo-1-ethyl-6-fluoro-3-(3-hydroxy-2,2-dimethyl-propyl)indol-2-yl]-6-[(1S)-1-methoxyethyl]-3-pyridyl]piperazine-1-carboxylate (compound D8, 950.0 mg, 1.29 mmol) and methyl (3S)-1-[(2S)-2-(tert-butoxycarbonylamino)-3-[3-(4,4,5,5-tetramethyl-1,3,2-dioxaborolan-2-yl)-5-triisopropylsilyloxy-phenyl]propanoyl]hexahydropyridazine-3-carboxylate (intermediate B, 1.07 g, 1.55 mmol) in 1,4-dioxane (25 mL) and water (5 mL) were added sodium carbonate (342.2 mg, 3.23 mmol) and Pd(dtbpf)Cl₂ (84.2 mg, 0.13 mmol). The mixture was stirred at 85° C. for 12 hrs under nitrogen atmosphere. After the reaction was completed, the mixture was filtered and the filtrate was concentrated in vacuo. The residue was purified by reversed phase flash column and concentrated in vacuo to afford methyl (3S)-1-[(2S)-3-[3-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]-5-triisopropylsilyloxy-phenyl]-2-(tert-butoxycarbonylamino)propanoyl]hexahydropyridazine-3-carboxylate (compound G1, 1.3 g) as a brown solid. MS calc'd 1218.6 (MH⁺), measured 1218.5 (MH⁺).

### Step 2: Preparation of (3S)-1-[(2S)-3-[3-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]-5-triisopropylsilyloxy-phenyl]-2-(tert-butoxycarbonylamino)propanoyl]hexahydropyridazine-3-carboxylic acid (compound G2)

To a mixture of methyl (3S)-1-[(2S)-3-[3-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]-5-triisopropylsilyloxy-phenyl]-2-(tert-butoxycarbonylamino)-propanoyl]hexahydropyridazine-3-carboxylate (compound G1, 1.1 g, 0.9 mmol) in DCE (20 mL) was added Me₃SnOH (652.9 mg, 3.61 mmol). The mixture was stirred at 60° C. for 12 hrs. After the reaction was completed, the mixture was diluted with water (100 mL), extracted with EtOAc (100 mL, twice). The combined organic layer was washed with brine (150 mL), dried over Na₂SO₄, filtered and concentrated in vacuo to afford (3S)-1-[(2S)-3-[3-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]-5-triisopropylsilyloxy-phenyl]-2-(tert-butoxycarbonylamino)propanoyl]hexahydropyridazine-3-carboxylic acid (compound G2, 1.5 g) as a yellow solid, which was used directly in the next step. MS calc'd 1204.6 (MH⁺), measured 1204.5 (MH⁺).

### Step 3: Preparation of benzyl 4-[5-[(8S,14S)-8-(tert-butoxycarbonylamino)-25-fluoro-18,18-dimethyl-9,15-dioxo-22-(2,2,2-trifluoroethyl)-4-triisopropylsilyloxy-16-oxa-10,22,28-triazapentacyclo[18.5.2.1^{2,6}.1^{10,14}.0^{23,27}]nonacosa-1(26),2,4,6(29),20,23(27),24-heptaen-(21M)-21-yl]-6-[(1S)-1-methoxyethyl]-3-pyridyl]piperazine-1-carboxylate (compound G3)

To a mixture of (3S)-1-[(2S)-3-[3-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]-5-triisopropylsilyloxy-phenyl]-2-(tert-butoxycarbonylamino)-propanoyl]hexahydropyridazine-3-carboxylic acid (compound G2, 1.5 g, 1.25 mmol) in DCM (150 mL) were added DIEA (4.4 mL, 24.95 mmol), EDCl (3.6 g, 18.71 mmol) and HOBt (421.4 mg, 3.12 mmol) at 0° C. After being stirred at 15° C. for 12 hrs, the reaction mixture was concentrated in vacuo and diluted with water (50 mL), extracted with EtOAc (50 mL, three times). The combined organic layer was washed with brine (100 mL), dried over Na₂SO₄, filtered and concentrated in vacuo. The residue was purified by column chromatography (EtOAc in PE=25%-50%) to afford benzyl 4-[5-[(8S,14S)-8-(tert-butoxycarbonylamino)-25-fluoro-18,18-dimethyl-9,15-dioxo-22-(2,2,2-trifluoroethyl)-4-triisopropylsilyloxy-16-oxa-10,22,28-triazapentacyclo[18.5.2.1^{2,6}.1^{10,14}.0^{23,27}]nonacosa-1(26),2,4,6(29),20,23(27),24-heptaen-(21M)-21-yl]-6-[(1S)-1-methoxyethyl]-3-pyridyl]piperazine-1-carboxylate (compound G3, 1.1 g) as a yellow solid. MS calc'd 1186.4 (MH⁺), measured 1186.5 (MH⁺).

### Step 4: Preparation of tert-butyl N-[(8S,14S)-25-fluoro-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-4-triisopropylsilyloxy-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamate (compound G4)

To a mixture of benzyl 4-[5-[(8S,14S)-8-(tert-butoxycarbonylamino)-25-fluoro-18,18-dimethyl-9,15-dioxo-22-(2,2,2-trifluoroethyl)-4-triisopropylsilyloxy-16-oxa-10,22,28-triazapentacyclo[18.5.2.1^{2,6}.1^{10,14}.0^{23,27}]nonacosa-1(26),2,4,6(29),20,23(27),24-heptaen-(21M)-21-yl]-6-[(1S)-1-methoxyethyl]-3-pyridyl]piperazine-1-carboxylate (compound G3, 890.0 mg, 0.75 mmol) and formaldehyde (182.6 mg, 2.25 mmol) in methanol (15 mL) was added Pd(OH)₂ on activated carbon (700.0 mg, 1.0 mmol). The mixture was degassed and purged with H₂ three times. The mixture was hydrogenated at 30° C. for 18 hrs. The mixture was filtered, the filtrate was concentrated in vacuo to afford tert-butyl N-[(8S,14S)-25-fluoro-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-4-triisopropylsilyloxy-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamate (compound G4, 780.0 mg) as colorless gum, which was used directly in the next step. MS calc'd 1066.5 (MH⁺), measured 1066.7 (MH⁺).

### Step 5: Preparation of tert-butyl N-[(8S,14S)-25-fluoro-4-hydroxy-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamate (compound G5)

To a mixture of afford tert-butyl N-[(8S,14S)-25-fluoro-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-4-triisopropylsilyloxy-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamate (compound G4, 780.0 mg, 0.73 mmol) in THF (10 mL) was added tetrabutylammonium fluoride (0.8 mL, 0.8 mmol, 1 M in THF) at 0° C. After being stirred at 0° C. for 0.5 h, the reaction mixture was diluted with water (20 mL), extracted with EtOAc (20 mL, three times). The combined organic layer was washed with brine (30 mL, twice), dried over Na₂SO₄, filtered and concentrated in vacuo to afford tert-butyl N-[(8S,14S)-25-fluoro-4-hydroxy-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamate (compound G5, 700.0 mg, 0.77 mmol) as a yellow solid, which was directly used in the next step. MS calc'd 910.4 (MH⁺), measured 910.7 (MH⁺).

### Step 6: Preparation of (8S,14S)-8-amino-25-fluoro-4-hydroxy-(21M)-21-[2-[(1S)-1-methoxyethyl]-5-(4-methylpiperazin-1-yl)-3-pyridyl]-18,18-dimethyl-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaene-9,15-dione (intermediate G)

To a mixture of tert-butyl N-[(8S,14S)-25-fluoro-4-hydroxy-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamate (compound G5, 700.0 mg, 0.77 mmol) in DCM (10 mL) was added TFA (6.0 mL, 77.88 mmol). After being stirred at 15° C. for 0.5 h, the reaction mixture was concentrated in vacuo and diluted with sat. NaHCO₃ (15 mL), extracted with EtOAc (50 mL, twice). The combined organic layer was washed with brine (20 mL), dried over Na₂SO₄, filtered and concentrated in vacuo to afford (8S,14S)-8-amino-25-fluoro-4-hydroxy-(21M)-21-[2[(1S)-1-methoxyethyl]-5-(4-methylpiperazin-1-yl)-3-pyridyl]-18,18-dimethyl-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaene-9,15-dione (intermediate G, 600.0 mg) as a yellow solid, which was used directly in the next step. MS calc'd 810.3 (MH⁺), measured 810.4 (MH⁺).

### Intermediate H

### (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

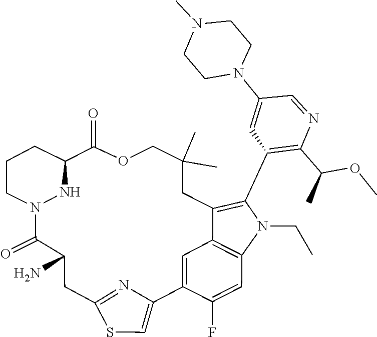

The title compound was prepared in analogy to the preparation of Intermediate D by using iodoethane instead of 2,2,2-trifluoroethyl trifluoromethanesulfonate.

### Intermediate I

### (7S,13S)-7-amino-24-fluoro-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

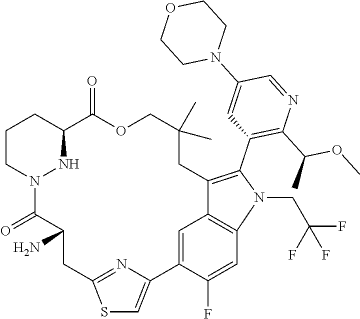

The compound was prepared according to the following scheme:

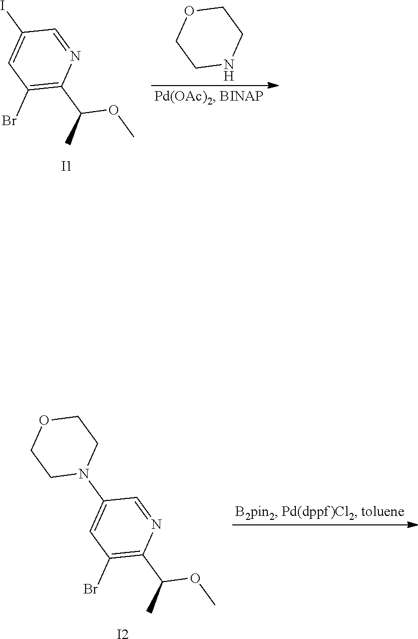

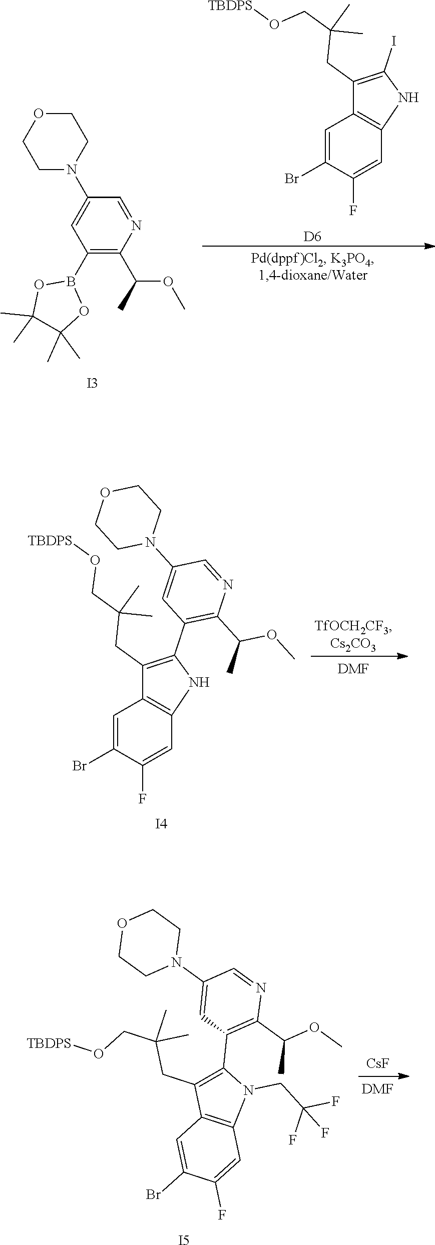

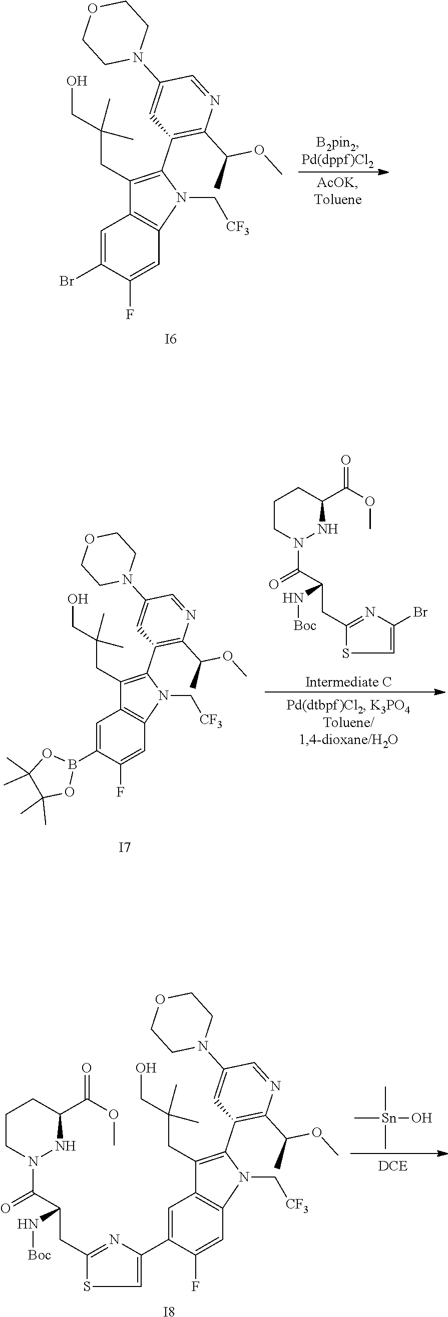

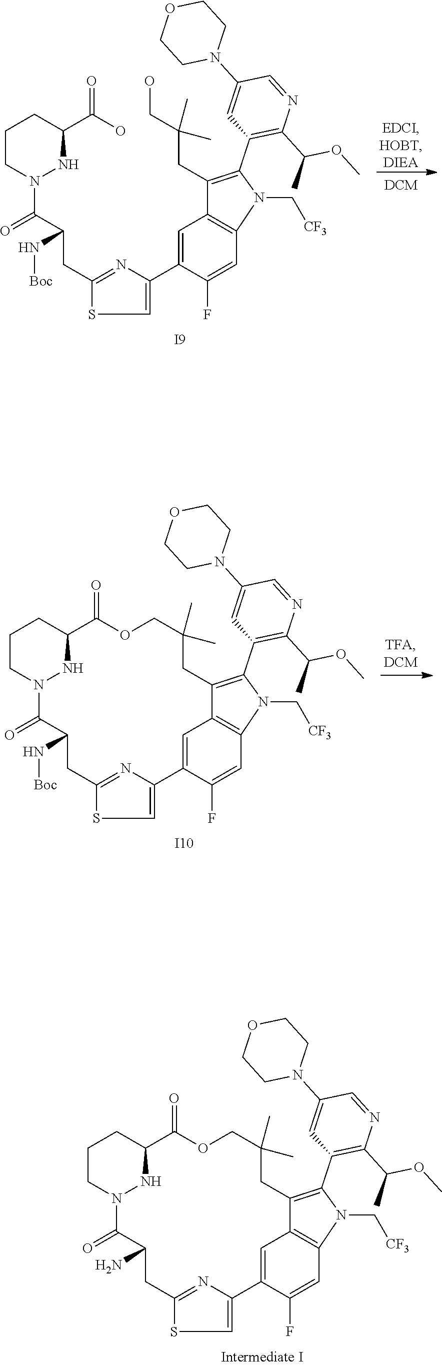

### Step 1: Preparation of 4-[5-bromo-6-[(1S)-1-methoxyethyl]-3-pyridyl]morpholine (compound 12)

To a mixture of 3-bromo-5-iodo-2-[(1S)-1-methoxyethyl]pyridine (compound I1, 3.4 g, 9.94 mmol) and morpholine (0.9 g, 9.94 mmol) in Toluene (50 mL) were added cesium carbonate (8.1 g, 24.87 mmol), (R)-binap (0.3 g, 0.5 mmol) and palladium (II) acetate (0.1 g, 0.5 mmol). The mixture was stirred at 100° C. for 12 hrs under nitrogen atmosphere. After the reaction was completed, the mixture was filtered and concentrated in vacuo. The residue was purified by column chromatography (EtOAc in PE=10%-25%) to afford 4-[5-bromo-6-[(1S)-1-methoxyethyl]-3-pyridyl]morpholine (compound 12, 2100.0 mg) as colorless oil. MS calc'd 301.1 (MH⁺), measured 301.1 (MH⁺).

### Step 2: Preparation of 4-[6-[(1S)-1-methoxyethyl]-5-(4,4,5,5-tetramethyl-1,3,2-dioxaborolan-2-yl)-3-pyridyl]morpholine (compound 13)

To a mixture of 4-[5-bromo-6-[(1S)-1-methoxyethyl]-3-pyridyl]morpholine (compound 12, 2.1 g, 6.97 mmol) and bis(pinacolato)diboron (2.1 g, 8.37 mmol) in 1,4-dioxane (25 mL) were added KOAc (1.7 g, 17.43 mmol) and Pd(dppf)Cl₂ (0.2 g, 0.35 mmol). The mixture was stirred at 90° C. for 12 hrs under nitrogen atmosphere. After the reaction was completed, the reaction mixture was filtered and the filtrate was concentrated in vacuo. The residue was purified by column chromatography (EtOAc in PE=10%-25%) to afford 4-[6-[(1S)-1-methoxyethyl]-5-(4,4,5,5-tetramethyl-1,3,2-dioxaborolan-2-yl)-3-pyridyl]morpholine (compound 13, 1100.0 mg) as a brown solid. MS calc'd 349.2 (MH⁺), measured 349.2 (MH⁺)

### Step 3: Preparation of [3-[5-bromo-6-fluoro-2-[2-[(1S)-1-methoxyethyl]-5-morpholino-3-pyridyl]-1H-indol-3-yl]-2,2-dimethyl-propoxy]-tert-butyl-diphenyl-silane (compound 14)

To a mixture of [3-(5-bromo-6-fluoro-2-iodo-1H-indol-3-yl)-2,2-dimethyl-propoxy]-tert-butyl-diphenyl-silane (compound D6, 1.9 g, 2.94 mmol) and 4-[6-[(1S)-1-methoxyethyl]-5-(4,4,5,5-tetramethyl-1,3,2-dioxaborolan-2-yl)-3-pyridyl]morpholine (compound 13, 1.0 g, 2.94 mmol) in 1,4-dioxane (25 mL) and water (5 mL) were added potassium phosphate (1.8 g, 8.64 mmol) and Pd(dppf)Cl₂ (0.22 g, 0.29 mmol). The mixture was stirred at 70° C. for 12 hrs under nitrogen atmosphere. After the reaction was completed, the reaction mixture was filtered and the filtrated was diluted with water (50 mL) and extracted with EtOAc (100 mL, three times). The combined organic phase was washed with brine (200 mL), dried over Na₂SO₄, filtered and concentrated in vacuo. The residue was purified by column chromatography to afford [3-[5-bromo-6-fluoro-2-[2-[(1S)-1-methoxyethyl]-5-morpholino-3-pyridyl]-1H-indol-3-yl]-2,2-dimethyl-propoxy]-tert-butyl-diphenyl-silane (compound 14, 1.4 g) as yellow gum. MS calc'd 758.2 (MH⁺), measured 758.2 (MH⁺).

### Step 4: Preparation of [3-[5-bromo-6-fluoro-(2M)-2-[2-[(1S)-1-methoxyethyl]-5-morpholino-3-pyridyl]-1-(2,2,2-trifluoroethyl)indol-3-yl]-2,2-dimethyl-propoxy]-tert-butyl-diphenyl-silane (compound 15)

To a solution of [3-[5-bromo-6-fluoro-2-[2-[(1S)-1-methoxyethyl]-5-morpholino-3-pyridyl]-1H-indol-3-yl]-2,2-dimethyl-propoxy]-tert-butyl-diphenyl-silane (compound 14, 1.4 g, 1.89 mmol) and cesium carbonate (1.8 g, 5.67 mmol) in DMF (30 mL) was added 2,2,2-trifluoroethyl trifluoromethanesulfonate (4.4 g, 18.9 mmol) dropwise at 0° C., and the mixture was stirred at 30° C. for 16 hrs. After the reaction was completed, EtOAc (200 mL) and water (200 mL) were added and the layers were separated. The aqueous phase was extracted with EtOAc (100 mL, twice). The combined organic layer was washed with brine (100 mL, three times), dried over Na₂SO₄, filtered, and concentrated under vacuum. The residue was purified by column chromatography (EtOAc in PE=25%-66%) to afford [3-[5-bromo-6-fluoro-(2M)-2-[2-[(1S)-1-methoxyethyl]-5-morpholino-3-pyridyl]-1-(2,2,2-trifluoroethyl)indol-3-yl]-2,2-dimethyl-propoxy]-tert-butyl-diphenyl-silane (compound 15, 800.0 mg, PEAK 1, faster eluted) as a brown solid. MS calc'd 840.1 (MH⁺), measured 840.1 (MH⁺).

### Step 5: Preparation of 3-[5-bromo-6-fluoro-(2M)-2-[2-[(1S)-1-methoxyethyl]-5-morpholino-3-pyridyl]-1-(2,2,2-trifluoroethyl)indol-3-yl]-2,2-dimethyl-propan-1-ol (compound 16)

To a solution of [3-[5-bromo-6-fluoro-(2M)-2-[2-[(1S)-1-methoxyethyl]-5-morpholino-3-pyridyl]-1-(2,2,2-trifluoroethyl)indol-3-yl]-2,2-dimethyl-propoxy]-tert-butyl-diphenyl-silane (compound 15, 0.8 g, 0.95 mmol) in DMF (10 mL) was added cesium fluoride (0.7 g, 4.93 mmol) and the mixture was stirred at 60° C. for 15 hrs. After the reaction was completed, the reaction mixture was cooled to room temperature. EtOAc (70 mL) and water (100 mL) were added and layers were separated. The aqueous phase was extracted with EtOAc (70 mL, twice). The combined organic layer was washed with brine (80 mL, four times), dried over Na₂SO₄, filtered, and concentrated under vacuum to give a residue. The residue was purified by column chromatography (EtOAc in PE=10%-66%) to afford 3-[5-bromo-6-fluoro-(2M)-2-[2-[(1S)-1-methoxyethyl]-5-morpholino-3-pyridyl]-1-(2,2,2-trifluoroethyl)indol-3-yl]-2,2-dimethyl-propan-1-ol (compound 16, 430.0 mg) as yellow gum. MS calc'd 602.1 (MH⁺1), measured 602.2 (MH⁺)

### Step 6: Preparation of 3-[6-fluoro-(2M)-2-[2-[(1S)-1-methoxyethyl]-5-morpholino-3-pyridyl]-5-(4,4,5,5-tetramethyl-1,3,2-dioxaborolan-2-yl)-1-(2,2,2-trifluoroethyl)indol-3-yl]-2,2-dimethyl-propan-1-ol (compound 17)

To a solution of 3-[5-bromo-6-fluoro-(2M)-2-[2-[(1S)-1-methoxyethyl]-5-morpholino-3-pyridyl]-1-(2,2,2-trifluoroethyl)indol-3-yl]-2,2-dimethyl-propan-1-ol (compound 16, 430.0 mg, 0.71 mmol) and bis(pinacolato)diboron (199.3 mg, 0.79 mmol) in toluene (5 mL) was added potassium acetate (0.1 mL, 1.78 mmol) and Pd(dppf)Cl₂ (52.2 mg, 0.07 mmol) and the mixture was degassed by bubbling nitrogen for 2 min and then the mixture was stirred at 90° C. for 15 hrs under nitrogen atmosphere. After the reaction was completed, the reaction mixture was filtered and the filtrate was concentrated in vacuo to give a residue. The residue was purified by column chromatography (EtOAc in PE=10%-66%) to afford 3-[6-fluoro-(2M)-2-[2-[(1S)-1-methoxyethyl]-5-morpholino-3-pyridyl]-5-(4,4,5,5-tetramethyl-1,3,2-dioxaborolan-2-yl)-1-(2,2,2-trifluoroethyl)indol-3-yl]-2,2-dimethyl-propan-1-ol (compound 17, 390.0 mg) as colorless gum. MS calc'd 650.3 (MH⁺), measured 650.3 (MH⁺)

### Step 7: Preparation of methyl (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-morpholino-3-pyridyl]-1-(2,2,2-trifluoroethyl)indol-5-yl]thiazol-2-yl]propanoyl]hexahydropyridazine-3-carboxylate (compound I8)

To a mixture of 3-[6-fluoro-(2M)-2-[2-[(1S)-1-methoxyethyl]-5-morpholino-3-pyridyl]-5-(4,4,5,5-tetramethyl-1,3,2-dioxaborolan-2-yl)-1-(2,2,2-trifluoroethyl)indol-3-yl]-2,2-dimethyl-propan-1-ol (compound 17, 390.0 mg, 0.6 mmol) and methyl (3S)-1-[(2S)-3-(4-bromothiazol-2-yl)-2-(tert-butoxycarbonylamino)propanoyl]hexahydropyridazine-3-carboxylate (intermediate C, 291.6 mg, 0.61 mmol) in toluene (4 mL), 1,4-dioxane (1.3 mL) and water (1.3 mL) were added K₃PO₄ (320.06 mg, 1.51 mmol) and Pd(dtbpf)Cl₂ (39.1 mg, 0.06 mmol). The mixture was stirred at 70° C. for 12 hrs under nitrogen atmosphere. After the reaction was completed, the reaction mixture was filtered and the filtrate was concentrated in vacuo. The residue was purified by column chromatography (EtOAc in PE=10%-75%) to afford (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-morpholino-3-pyridyl]-1-(2,2,2-trifluoroethyl)indol-5-yl]thiazol-2-yl]propanoyl]hexahydropyridazine-3-carboxylate (compound I8, 200.0 mg) as a brown solid. MS calc'd 920.3 (MH⁺), measured 920.3 (MH⁺).

### Step 8: Preparation of (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-morpholino-3-pyridyl]-1-(2,2,2-trifluoroethyl)indol-5-yl]thiazol-2-yl]propanoyl]hexahydropyridazine-3-carboxylic acid (compound 19)

To a solution of (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-morpholino-3-pyridyl]-1-(2,2,2-trifluoroethyl)indol-5-yl]thiazol-2-yl]propanoyl]hexahydropyridazine-3-carboxylate (compound 18, 170.0 mg, 0.18 mmol) in DCE (4 mL) was added trimethylstannanol (133.6 mg, 0.74 mmol).

The mixture was stirred at 60° C. for 15 hrs. After the reaction was completed, the reaction mixture was cooled to room temperature. The reaction mixture was concentrated, then EtOAc (40 mL) and water (50 mL) were added and the layers were separated. The aqueous phase was extracted with EtOAc (50 mL, twice). The combined organic layer was washed with brine (50 mL), dried over Na₂SO₄, filtered, and concentrated under vacuum to afford (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-morpholino-3-pyridyl]-1-(2,2,2-trifluoroethyl)indol-5-yl]thiazol-2-yl]propanoyl]hexahydropyridazine-3-carboxylic acid (compound 19, 160.0 mg) as a yellow solid. MS calc'd 906.3 (MH⁺), measured 906.3 (MH⁺).

### Step 9: Preparation of tert-butyl N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound 110)

To a solution of (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-morpholino-3-pyridyl]-1-(2,2,2-trifluoroethyl)indol-5-yl]thiazol-2-yl]propanoyl]hexahydropyridazine-3-carboxylic acid (compound 19, 160.0 mg, 0.18 mmol) in DCM (15 mL) was added EDCl (507.8 mg, 2.65 mmol) and DIEA (0.6 mL, 3.53 mmol), then followed by addition of HOBt (59.6 mg, 0.44 mmol) at 0° C., and then the mixture was stirred at 20° C. for 15 hrs. After the reaction was completed, EtOAc (50 mL) and water (80 mL) were added and the layers were separated. The aqueous phase was extracted with EtOAc (50 mL, twice). The combined organic layer was washed with brine (80 mL), dried over Na₂SO₄, filtered, and concentrated under vacuum to give a residue.

The residue was purified by column chromatography (EtOAc in PE=25%-66%) to afford tert-butyl N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound 110, 130.0 mg) as an off-white solid. MS calc'd 888.3 (MH⁺), measured 888.3 (MH⁺)

### Step 10: Preparation of (7S,13S)-7-amino-24-fluoro-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate I)

To a solution of tert-butyl N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound 110, 130.0 mg, 0.15 mmol) in DCM (1.5 mL) was added TFA (0.6 mL, 8.27 mmol) and the mixture was stirred at 20° C. for 1 h. After the reaction was completed, sat. NaHCO₃ solution (40 mL) was added and the mixture was extracted with EtOAc (50 mL, three times). The combined organic layer was washed with brine (50 mL), dried over anhydrous sodium sulfate, filtered and concentrated under vacuum to afford (7S,13S)-7-amino-24-fluoro-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate I, 105.0 mg) as a yellow solid. MS calc'd 788.3 (MH⁺), measured 788.3 (MH⁺).

### Intermediate J

### (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

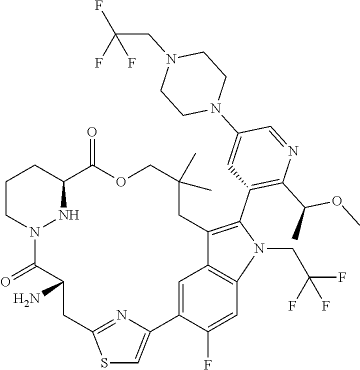

The title compound was prepared in analogy to the preparation of Intermediate I by using 1-(2,2,2-trifluoroethyl)piperazine instead of morpholine.

### Intermediate K

### (8S,14S)-8-amino-26-fluoro-4-hydroxy-(21M)-21-[2-[(1S)-1-methoxyethyl]-5-(4-methylpiperazin-1-yl)-3-pyridyl]-18,18-dimethyl-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaene-9,15-dione

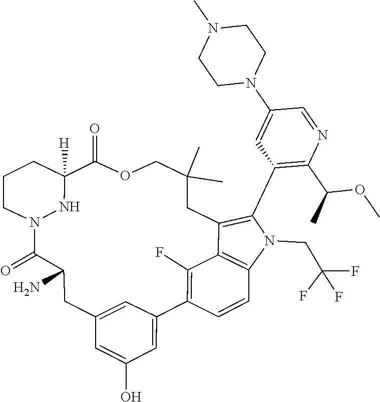

The title compound was prepared in analogy to the preparation of Intermediate G by using benzyl 4-[(5M)-5-[5-bromo-4-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (compound K8) instead of benzyl 4-[(5M)-5-[5-bromo-1-ethyl-6-fluoro-3-(3-hydroxy-2,2-dimethyl-propyl)indol-2-yl]-6-[(1S)-1-methoxyethyl]-3-pyridyl]piperazine-1-carboxylate (compound D8).

benzyl 4-[(5M)-5-[5-bromo-4-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (compound K8) was prepared in analogy to the preparation of benzyl 4-[(5M)-5-[5-bromo-1-ethyl-6-fluoro-3-(3-hydroxy-2,2-dimethyl-propyl)indol-2-yl]-6-[(1S)-1-methoxyethyl]-3-pyridyl]piperazine-1-carboxylate (compound D8) using 5-bromo-4-fluoro-1H-indole instead of 5-bromo-6-fluoro-1H-indole (compound D2).

### Intermediate L

### (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

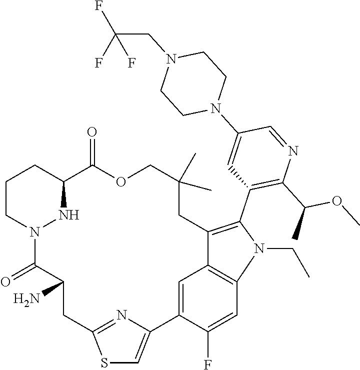

The title compound was prepared in analogy to the preparation of Intermediate I by using 1-(2,2,2-trifluoroethyl)piperazine and iodoethane instead of morpholine and 2,2,2-trifluoroethyl trifluoromethanesulfonate.

### Intermediate M

### (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo-[17.5.2.1^{2,5}.1^{9.13}.0^{23,26}]octacosa-1(25),2,5(28),19,22(26),23-hexaene-8,14-dione

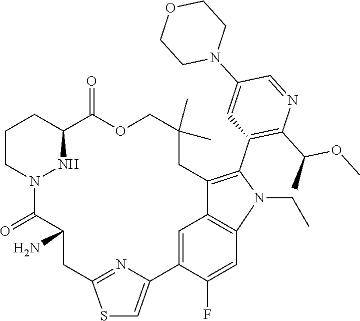

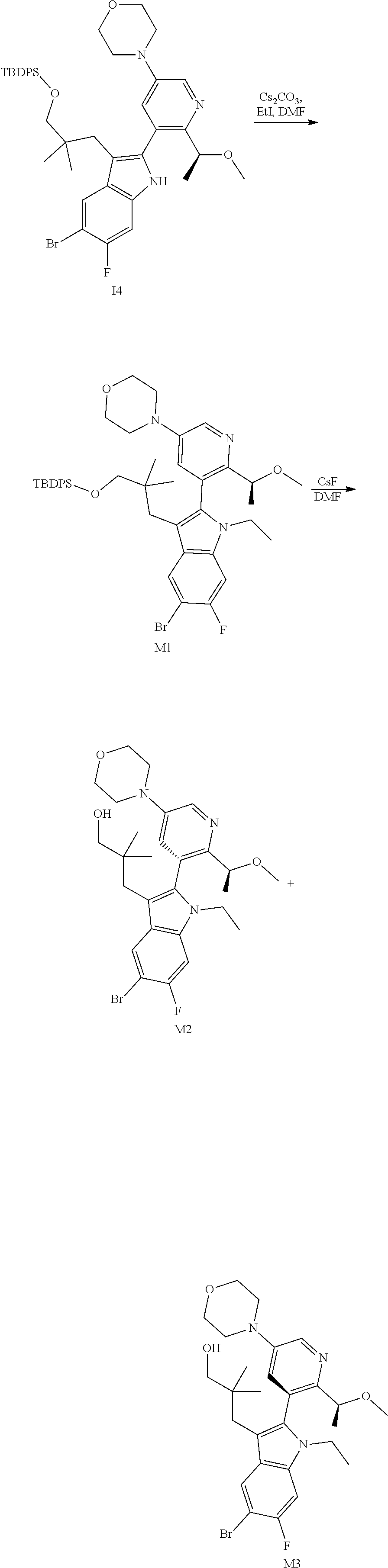

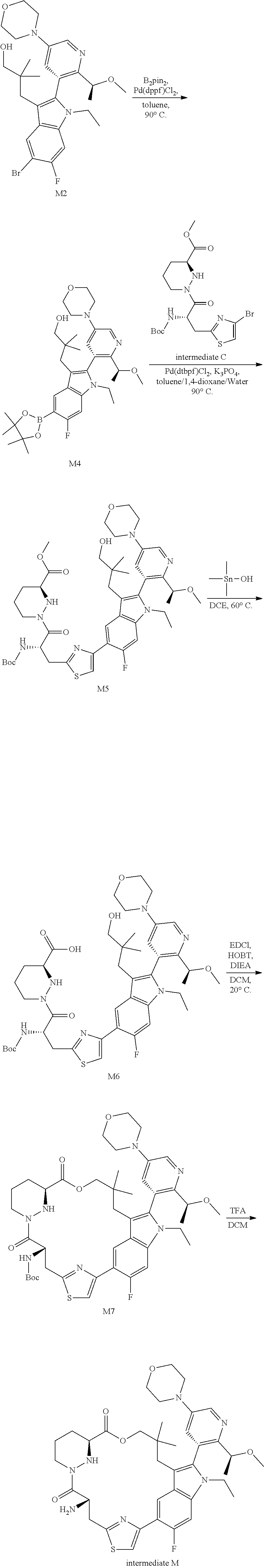

### Step 1: Preparation of [3-[5-bromo-1-ethyl-6-fluoro-2-[2-[(1S)-1-methoxyethyl]-5-morpholino-3-pyridyl]indol-3-yl]-2,2-dimethyl-propoxyl]-tert-butyl-diphenyl-silane (compound M1)

To a solution of [3-[5-bromo-6-fluoro-2-[2-11(1S)-1-methoxyethyl]-5-morpholino-3-pyridyl]-1H-indol-3-yl]-2,2-dimethyl-propoxy]-tert-butyl-diphenyl-silane (compound 14, 15 g, 19.77 mmol) in DMF (300 mL) was added Cs₂CO₃ (19.3 g, 59.3 mmol) and iodoethane (6.16 g, 39.53 mmol) at 0° C. After being stirred at 20° C. for 16 hrs, the reaction mixture was poured into water (200 mL), and extracted with EtOAc (200 mL, three times). The combined organic layer was washed with brine (10 mL, three times), dried over Na₂SO₄, filtered and concentrated under vacuum to give a residue. The residue was purified by column chromatography to afford [3-[5-bromo-1-ethyl-6-fluoro-2-[2-[(1S)-1-methoxyethyl]-5-morpholino-3-pyridyl]indol-3-yl]-2,2-dimethyl-propoxy]-tert-butyl-diphenyl-silane (compound M1, 14.7 g) as yellow oil. MS calc'd 786.3 (MH⁺), measured 786.4 (MH⁺).

### Step 2: Preparation of 3-[5-bromo-1-ethyl-6-fluoro-(2M)-2-[2-[(1S)-1-methoxyethyl]-5-morpholino-3-pyridyl]indol-3-yl]-2,2-dimethyl-propan-1-ol (compound M2) and 3-[5-bromo-1-ethyl-6-fluoro-(2P)-2-[2-[(1S)-1-methoxyethyl]-5-morpholino-3-pyridyl]indol-3-yl]-2,2-dimethyl-propan-1-ol (compound M3)

To a solution of [3-[5-bromo-1-ethyl-6-fluoro-2-[2-[(1S)-1-methoxyethyl]-5-morpholino-3-pyridyl]indol-3-yl]-2,2-dimethyl-propoxy]-tert-butyl-diphenyl-silane (compound M1, 14.7 g, 18.68 mmol) in DMF (160 mL) was added cesium fluoride (14.2 g, 93.41 mmol). The mixture was stirred at 60° C. for 48 hrs. After being cooled to room temperature, the reaction mixture were added with EtOAc (300 mL) and water (300 mL) and the layers were separated. The aqueous phase was extracted with EtOAc (200 mL, three times). The combined organic layer was washed with brine (200 mL, four times), dried over Na₂SO₄, filtered, and concentrated under vacuum to give a residue. The residue was purified by column chromatography to afford 3-[5-bromo-1-ethyl-6-fluoro-(2M)-2-[2-[(1S)-1-methoxyethyl]-5-morpholino-3-pyridyl]indol-3-yl]-2,2-dimethyl-propan-1-ol (compound M2, 6 g, faster eluted) as colorless foam and 3-[5-bromo-1-ethyl-6-fluoro-(2P)-2-[2-[(1S)-1-methoxyethyl]-5-morpholino-3-pyridyl]indol-3-yl]-2,2-dimethyl-propan-1-ol (compound M3, 4.5 g, slower eluted) as colorless foam. Compound M2: MS calc'd 548.2 (MH⁺), measured 548.2 (MH⁺). ¹H NMR (400 MHz, Methanol-d₄) δ=8.41 (d, J=2.4 Hz, 1H), 7.92 (d, J=6.8 Hz, 1H), 7.37-7.33 (m, 2H), 4.58 (s, 1H), 4.05-3.98 (m, 2H), 3.87-3.82 (m, 5H), 3.27-3.23 (m, 4H), 3.15-3.13 (m, 1H), 3.00 (s, 3H), 2.75-2.71 (m, 1H), 2.24-2.22 (m, 1H), 1.42 (d, J=6.4 Hz, 3H), 1.22 (t, J=7.2 Hz, 3H), 0.76 (s, 3H), 0.76 (s, 3H).

### X-Ray Crystallographic Analysis of Compound M2

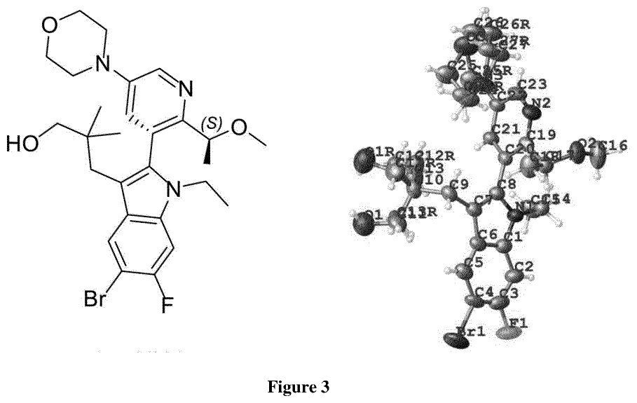

Absolute configuration structure of compound M2 was confirmed by X-ray crystallographic analysis of its single crystal. (FIG. 3).

### Step 3: Preparation of 3-[1-ethyl-6-fluoro-(2M)-2-[2-[(1S)-1-methoxyethyl]-5-morpholino-3-pyridyl]-5-(4,4,5,5-tetramethyl-1,3,2-dioxaborolan-2-yl)indol-3-yl]-2,2-dimethyl-propan-1-ol (compound M4)

To a solution of 3-[5-bromo-1-ethyl-6-fluoro-(2M)-2-[2-[(1S)-1-methoxyethyl]-5-morpholino-3-pyridyl]indol-3-yl]-2,2-dimethyl-propan-1-ol (compound M2, 6 g, 10.94 mmol), bis(pinacolato)diboron (4.2 g, 16.41 mmol) in toluene (60 mL) was added potassium acetate (2.7 g, 27.35 mmol) and Pd(dppf)Cl₂ (0.8 g, 1.09 mmol). The reaction mixture was degassed by bubbling nitrogen for 5 min then stirred at 90° C. for 15 hrs. After being cooled to room temperature, the reaction mixture was filtered and the filtrate was concentrated in vacuo to give a residue. The residue was purified by column chromatography to afford 3-[1-ethyl-6-fluoro-(2M)-2-[2-[(1S)-1-methoxyethyl]-5-morpholino-3-pyridyl]-5-(4,4,5,5-tetramethyl-1,3,2-dioxaborolan-2-yl)indol-3-yl]-2,2-dimethyl-propan-1-ol (compound M4, 4.5 g) as colorless gum. MS calc'd 596.4 (MH⁺), measured 596.4 (MH⁺).

### Step 4: Preparation of methyl (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylate (compound M5)

To a mixture of 3-[1-ethyl-6-fluoro-(2M)-2-[2-[(1S)-1-methoxyethyl]-5-morpholino-3-pyridyl]-5-(4,4,5,5-tetramethyl-1,3,2-dioxaborolan-2-yl)indol-3-yl]-2,2-dimethyl-propan-1-ol (compound M4, 4.5 g, 7.56 mmol) and methyl (3S)-1-[(2S)-3-(4-bromothiazol-2-yl)-2-(tert-butoxycarbonylamino)propanoyl]hexahydropyridazine-3-carboxylate (intermediate C, 3.6 g, 7.56 mmol) in toluene (45 mL), 1,4-dioxane (15 mL) and water (15 mL) were added K₃PO₄ (4.0 g, 18.89 mmol) and Pd(dtbpf)Cl₂ (492.5 mg, 0.75 mmol). The mixture was stirred at 70° C. for 12 hrs under nitrogen atmosphere. After being cooled to room temperature, the reaction mixture was filtered and the filtrate was concentrated in vacuo to give a residue. The residue was purified by column chromatography to afford methyl (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylate (compound M5, 3.8 g) as colorless gum. MS calc'd 866.4 (MH⁺), measured 866.4 (MH⁺).

### Step 5: Preparation of (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylic acid (compound M6)

To a mixture of methyl (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylate (compound M5, 3.8 g, 4.39 mmol) in DCE (76 mL) was added Me₃SnOH (3.2 g, 17.55 mmol). The mixture was stirred at 60° C. for 48 hrs. The reaction mixture was concentrated under vacuum to give a residue. EtOAc (200 mL) and water (100 mL) were added to the residue and the layers were separated. The aqueous phase was extracted with EtOAc (150 mL, twice). The combined organic layer was washed with brine (200 mL), dried over Na₂SO₄, filtered, and concentrated under vacuum to afford (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylic acid (compound M6, 3.7 g) as a brown solid. MS calc'd 852.4 (MH⁺), measured 852.4 (MH⁺).

### Step 6: Preparation of tert-butyl N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound M7)

To a mixture of (3S)-1-[(2S)-2-(tert-butoxycarbonylamino)-3-[4-[1-ethyl-6-fluoro-3-(3-hydroxy-2,2-dimethyl-propyl)-(2M)-2-[2-[(1S)-1-methoxyethyl]-5-morpholino-3-pyridyl]indol-5-yl]thiazol-2-yl]propanoyl]hexahydropyridazine-3-carboxylic acid (compound M6, 2.5 g, 2.93 mmol) in DCM (250 mL) were added DIEA (7.58 mL, 58.68 mmol), EDCl (8.4 g, 44.01 mmol) and HOBt (991.2 mg, 0.91 mmol) at 0° C. After being stirred at 20° C. for 12 hrs, the reaction mixture was poured into water (100 mL) and extracted with EtOAc (100 mL, three times). The combined organic layer was washed with brine (30 mL), dried over Na₂SO₄, filtered and concentrated under vacuum to give a residue which was purified by column chromatography to afford tert-butyl N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound M7, 1.2 g) as a yellow oil. MS calc'd 834.4 (MH⁺), measured 834.4 (MH⁺).

### Step 7: Preparation of (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate M)

To a solution of tert-butyl N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound M7, 1.2 g, 1.44 mmol) in DCM (12 mL) was added TFA (6.0 mL). The mixture was stirred at 20° C. for 3 hrs. After the reaction was completed, the reaction mixture was concentrated under vacuum to give a residue. Sat. NaHCO₃ aq. (60 mL) was added and the mixture was extracted with EtOAc (80 mL, three times). The combined organic layer was washed with brine (100 mL), dried over anhydrous sodium sulfate, filtered and concentrated in vacuo to afford (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate M, 1 g) as a yellow solid. MS calc'd 734.3 (MH⁺), measured 734.3 (MH⁺).

### Intermediate N

### (7S,13S)-7-amino-21-ethyl-24-fluoro-(20M)-20-[2-[(1S)-1-methoxyethyl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

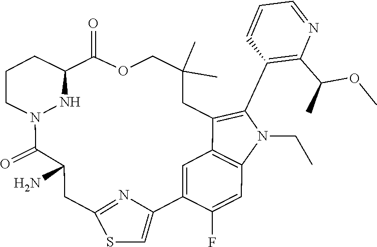

The title compound was prepared in analogy to the preparation of Intermediate I by using by using 2-[(1S)-1-methoxyethyl]-3-(4,4,5,5-tetramethyl-1,3,2-dioxaborolan-2-yl)pyridine (compound N1) instead of 4-[6-[(1S)-1-methoxyethyl]-5-(4,4,5,5-tetramethyl-1,3,2-dioxaborolan-2-yl)-3-pyridyl]morpholine (compound I3).

The compound N1 was prepared in analogy to the preparation of Intermediate A by using 3-bromo-2-[(1S)-1-methoxyethyl]pyridine (compound A1) instead of benzyl 4-[5-bromo-6-[(1S)-1-methoxyethyl]-3-pyridyl]piperazine-1-carboxylate (Intermediate A5).

### Example 1

### (3S)-1-[(2R)-2-chloro-2-fluoro-acetyl]-N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide

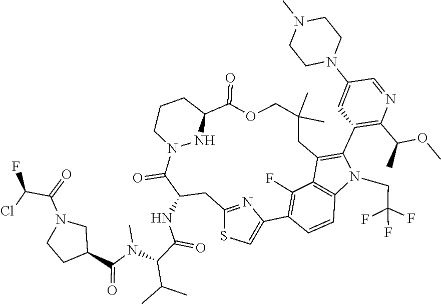

The compound was prepared according to the following scheme:

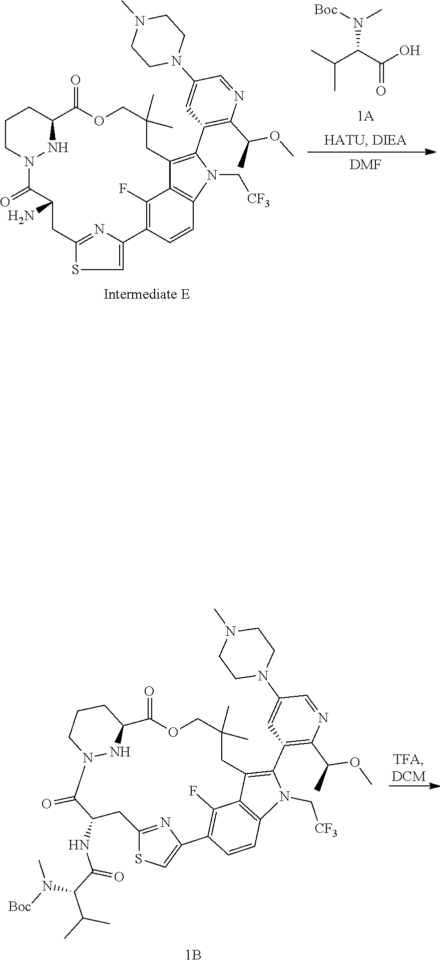

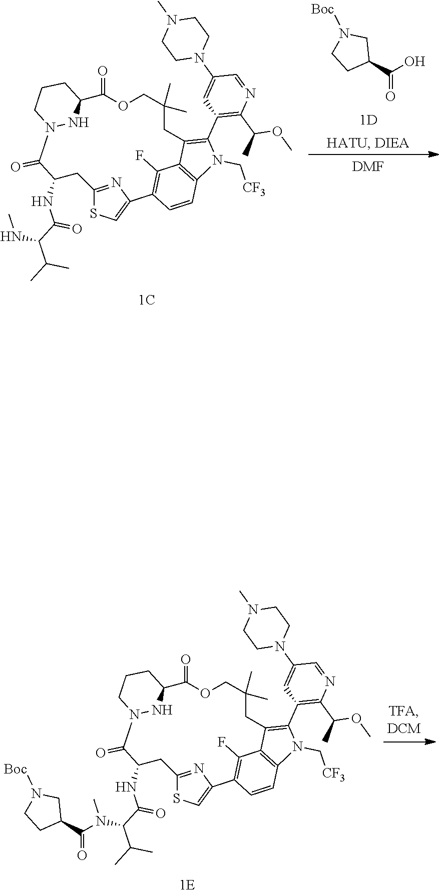

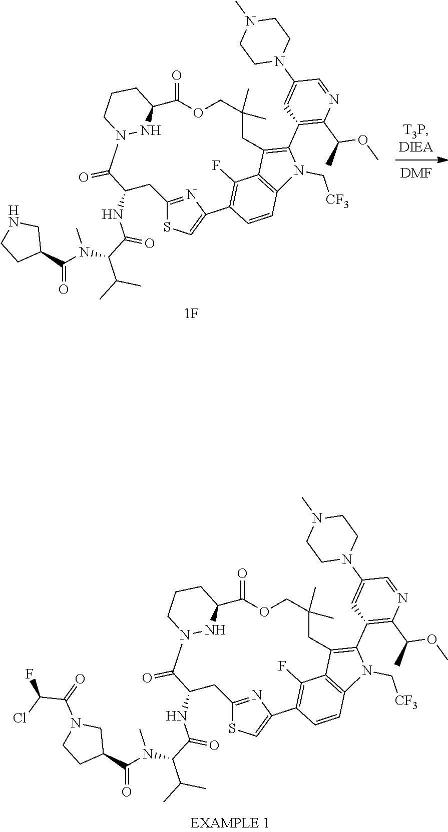

### Step 1: Preparation of tert-butyl N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-carbamate (compound 1B)

To a solution of (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E, 150.0 mg, 0.19 mmol) in DMF (1.5 mL) was added a solution of BOC-N-ME-VAL-OH (compound 1A, 75.0 mg, 0.32 mmol), DIEA (0.1 mL, 0.58 mmol) and HATU (120.0 mg, 0.32 mmol) in DMF (1.5 mL) at 0° C. and the mixture was stirred at 20° C. for 1 h. After the reaction was completed, the reaction mixture was poured into water (30 mL), and extracted with EtOAc (20 mL, three times). The combined organic layer was washed with brine (30 mL, three times), dried over Na₂SO₄, filtered and concentrated under vacuum to give a residue which was purified by prep-HPLC. The eluent was concentrated in vacuo to give tert-butyl N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-carbamate (compound 1B, 150.0 mg) as a yellow solid. MS calc'd 1014.5 (MH⁺), measured 1014.5 (MH⁺).

### Step 2: Preparation of (2S)—N-[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-(methylamino)butanamide (compound 1C)

To a solution of tert-butyl N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-carbamate (compound 1B, 100.0 mg, 0.1 mmol) in DCM (1 mL) was added TFA (1.0 mL) and the mixture was stirred at 20° C. for 2 hrs. After the reaction was completed, the reaction mixture was concentrated under vacuum to give a residue. Sat. NaHCO₃ aqueous solution (20 mL) was added and the mixture was extracted with EtOAc (20 mL, three times). The combined organic layer was washed with brine (30 mL), dried over anhydrous sodium sulfate, filtered and concentrated under vacuum to afford (2S)—N-[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-(methylamino)butanamide (compound 1C, 90.0 mg) as a yellow solid. MS calc'd 914.4 (MH⁺1), measured 914.4 (MH⁺).

### Step 3: Preparation of tert-butyl (3S)-3-[[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-methyl-carbamoyl]pyrrolidine-1-carboxylate (compound 1E)

To a solution of (2S)—N-[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-(methylamino)butanamide (compound 1C, 90.0 mg, 0.1 mmol) in DMF (1.5 mL) was added a solution of (S)-1-Boc-pyrrolidine-3-carboxylic acid (compound 1D, 31.9 mg, 0.15 mmol), DIEA (0.1 mL, 0.33 mmol), HATU (57.2 mg, 0.15 mmol) at 0° C. and then the mixture was stirred at 20° C. for 1 h. After the reaction was completed, the reaction mixture was poured into water (20 mL), and extracted with EtOAc (20 mL, three times). The combined organic layer was washed with brine (20 mL), dried over Na₂SO₄, filtered and concentrated under vacuum to give a residue which was purified by prep-HPLC to give tert-butyl (3S)-3-[[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-methyl-carbamoyl]pyrrolidine-1-carboxylate (compound 1E, 40.0 mg) as a yellow solid. MS calc'd 1111.5 (MH⁺), measured 1111.6 (MH⁺).

### Step 4: Preparation of (3S)—N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide (compound 1F)

To a solution of tert-butyl (3S)-3-[[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-methyl-carbamoyl]pyrrolidine-1-carboxylate (compound 1E, 40.0 mg, 0.04 mmol) in DCM (1 mL) was added TFA (0.3 mL), and the mixture was stirred at 20° C. for 1 h. After the reaction was completed, the reaction mixture was concentrated under vacuum to give a residue. Sat. NaHCO₃ aqueous solution (20 mL) was added and the mixture was extracted with EtOAc (15 mL, three times). The combined organic layer was washed with brine (20 mL), dried over anhydrous sodium sulfate, filtered and concentrated under vacuum to afford (3S)—N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide (compound 1F, 25.0 mg) as a yellow solid. MS calc'd 1011.4 (MH⁺), measured 1011.4 (MH⁺).

### Step 5: Preparation of (3S)-1-[(2R)-2-chloro-2-fluoro-acetyl]-N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo-[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide (Example 1)

To a solution of (2R)-2-chloro-2-fluoro-acetic acid (23.0 mg, 0.2 mmol), (3S)—N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide (compound 1F, 25.0 mg, 0.02 mmol) and DIEA (0.1 mL, 0.77 mmol) in DMF (2 mL) was added T₃P (130.0 mg, 0.2 mmol) at 0° C., and the mixture was stirred at 20° C. for 1 h. After the reaction was completed, the reaction mixture was poured into water (20 mL), and extracted with EtOAc (20 mL, three times). The combined organic layer was washed with brine (30 mL, three times), dried over Na₂SO₄, filtered and concentrated under vacuum to give a residue which was purified by prep-HPLC to afford (3S)-1-[(2R)-2-chloro-2-fluoro-acetyl]-N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo-[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide (Example 1, 9.4 mg) as a white solid. MS calc'd 1105.4 (MH⁺), measured 1105.8 (MH⁺). ¹H NMR (400 MHz, METHANOL-d₄) δ=8.52 (d, J=2.8 Hz, 1H), 7.59-7.41 (m, 4H), 7.01-6.70 (m, 1H), 5.97-5.85 (m, 1H), 5.22-5.04 (m, 1H), 4.68-4.63 (m, 1H), 4.43-4.35 (m, 1H), 4.11-3.91 (m, 3H), 3.81-3.48 (m, 13H), 3.44-3.39 (m, 1H), 3.28-3.24 (m, 1H), 3.23-3.11 (m, 6H), 3.05-2.97 (m, 4H), 2.92-2.80 (m, 1H), 2.66-2.55 (m, 1H), 2.39-1.97 (m, 4H), 1.71-1.60 (m, 1H), 1.46 (d, J=6.4 Hz, 3H), 1.35-1.27 (m, 2H), 1.18 (t, J=7.2 Hz, 3H), 0.98-0.79 (m, 9H), 0.67-0.57 (m, 2H) ppm.

### Example 2

### (3S)-1-[(2R)-2-chloro-2-fluoro-acetyl]-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide

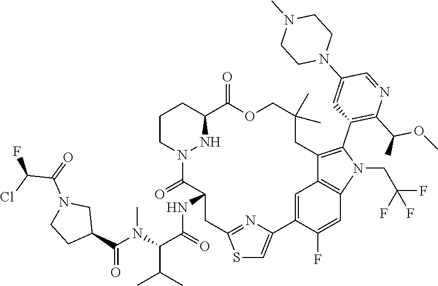

The title compound was prepared in analogy to the preparation of Example 1 by using (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate D) instead of (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E). Example 2 (39.0 mg) was obtained as a white solid. MS calc'd 1105.4 (MH⁺), measured 1105.6 (MH⁺). ¹H NMR (400 MHz, Methanol-d₄) δ=8.68 (d, J=7.6 Hz, 1H), 8.57-8.49 (m, 1H), 7.79-7.66 (m, 1H), 7.56-7.43 (m, 2H), 6.95-6.72 (m, 1H), 5.83-5.61 (m, 1H), 5.30-5.10 (m, 1H), 4.96-4.88 (m, 3H), 4.82 (dd, J=11.2, 3.6 Hz, 1H), 4.52-4.36 (m, 1H), 4.29-4.17 (m, 2H), 4.15-3.93 (m, 2H), 3.90-3.67 (m, 6H), 3.66-3.55 (m, 2H), 3.55-3.41 (m, 2H), 3.36 (d, J=2.8 Hz, 3H), 3.34-3.32 (m, 1H), 3.30-3.23 (m, 2H), 3.19-3.14 (m, 1H), 3.14-3.09 (m, 3H), 3.03-2.96 (m, 3H), 2.89-2.74 (m, 1H), 2.63-2.50 (m, 1H), 2.45-2.21 (m, 3H), 2.20-1.91 (m, 2H), 1.90-1.75 (m, 1H), 1.72-1.57 (m, 1H), 1.45 (d, J=6.4 Hz, 3H), 1.11-0.98 (m, 3H), 0.98-0.94 (m, 3H), 0.94-0.84 (m, 3H), 0.54-0.34 (m, 3H) ppm.

### Example 3

### (3S)-1-[(2R)-2-chloro-2-fluoro-acetyl]-N-[(1S)-1-[[(7S,13S)-(20M)-20-[2-[(1S)-1-methoxyethyl]-5-(4-methylpiperazin-1-yl)-3-pyridyl]-17,17,24-trimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide

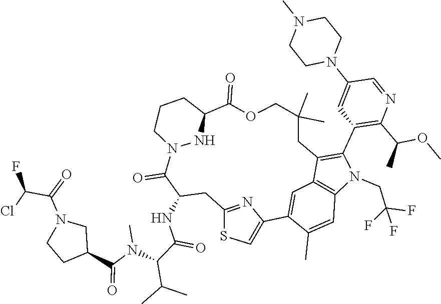

The title compound was prepared in analogy to the preparation of Example 1 by using (7S,13S)-7-amino-(20M)-20-[2-[(1S)-1-methoxyethyl]-5-(4-methylpiperazin-1-yl)-3-pyridyl]-17,17,24-trimethyl-21-(2,2,2-trifluoroethyl)-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate F) instead of (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E). Example 3 (4.7 mg) was obtained as a white solid. MS calc'd 1101.5 (MH⁺), measured 1101.6 (MH⁺). ¹H NMR (400 MHz, Methanol-d₄) δ=8.52-8.47 (m, 1H), 8.15-7.98 (m, 1H), 7.54-7.37 (m, 3H), 6.95-6.70 (m, 1H), 5.85-5.75 (m, 1H), 5.35-5.07 (m, 2H), 4.78-4.67 (m, 1H), 4.50-4.38 (m, 1H), 4.25-4.14 (m, 1H), 4.12-3.95 (m, 2H), 3.91-3.55 (m, 10H), 3.50-3.07 (m, 5H), 3.00 (s, 3H), 2.79-2.64 (m, 1H), 2.62-2.43 (m, 4H), 2.40-2.12 (m, 4H), 2.10-1.84 (m, 3H), 1.72-1.49 (m, 3H), 1.47-1.41 (m, 3H), 1.39-1.32 (m, 2H), 1.29-1.23 (m, 3H), 0.98-0.95 (m, 3H), 0.95-0.91 (m, 3H), 0.88-0.84 (m, 3H), 0.43-0.36 (m, 3H) ppm.

### Example 4 and Example 5

### (2S)-2-[(5R)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 4)

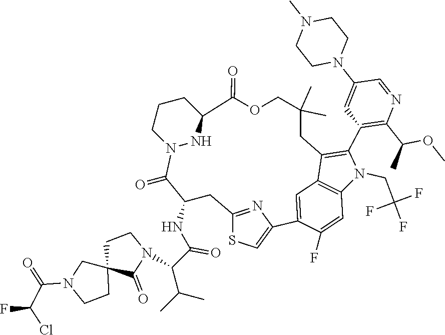

### Example 4

### (2S)-2-[(5S)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 5)

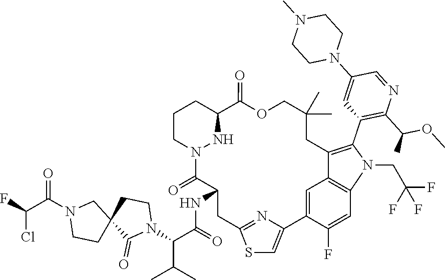

### Example 5

The Example 4 was prepared according to the following scheme:

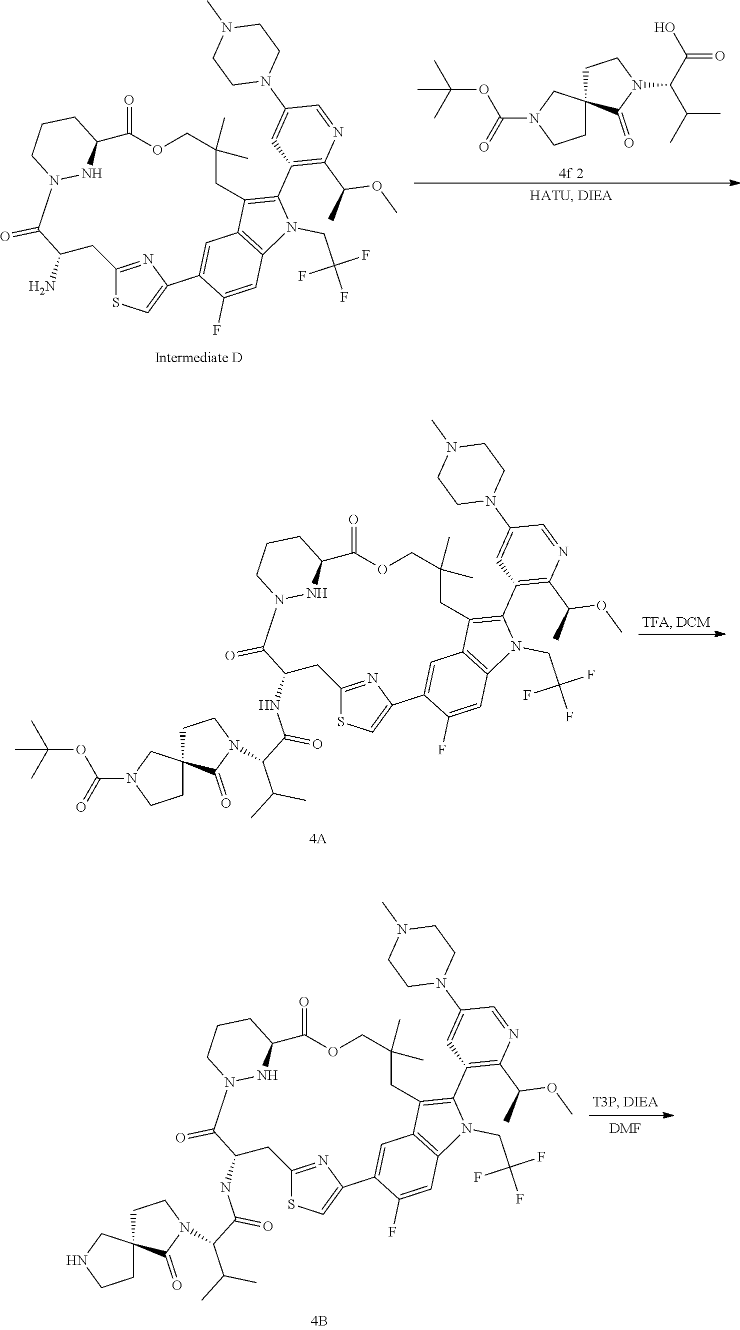

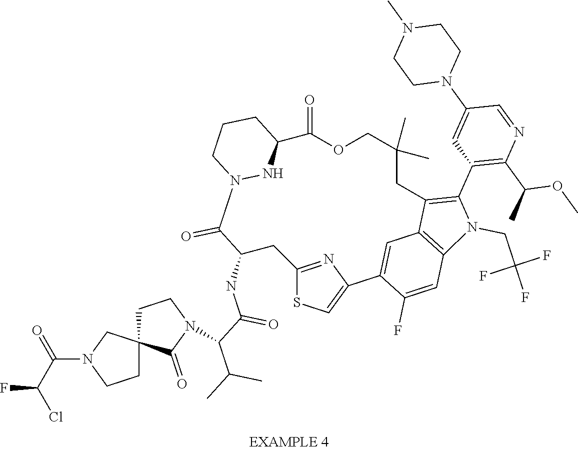

### Step 1: Preparation of tert-butyl (5S)-2-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-1-oxo-2,7-diazaspiro[4.4]nonane-7-carboxylate (compound 4A)

To a solution of (2S)-2-[(5S)-7-tert-butoxycarbonyl-1-oxo-2,7-diazaspiro[4.4]nonan-2-yl]-3-methyl-butanoic acid (compound 4f 2, 85.0 mg, 0.25 mmol) in DMF (3 mL) was added HATU (94.9 mg, 0.25 mmol), DIEA (0.1 mL, 0.37 mmol). After being stirred at 20° C. for 0.2 hour, (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate D, 100.0 mg, 0.12 mmol) was added. The reaction mixture was stirred at 20° C. for another 2 hrs. After the reaction was completed, the reaction mixture was purified directly by reversed phase flash column and the eluent was concentrated under vacuum to remove ACN. The aqueous was neutralized with NaHCO₃ and extracted with EtOAc (10 mL, three times). The combined organic layer was washed with brine (30 mL), dried over Na₂SO₄, filtered and concentrated under vacuum to afford tert-butyl (5S)-2-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-1-oxo-2,7-diazaspiro[4.4]nonane-7-carboxylate (compound 4A, 120.0 mg) as yellow solid. MS calc'd 1123.6 (MH⁺), measured 1123.6 (MH⁺).

### Step 2: Preparation of (2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-[(5R)-1-oxo-2,7-diazaspiro[4.4]nonan-2-yl]butanamide (compound 4B)

To a solution of tert-butyl (5S)-2-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-1-oxo-2,7-diazaspiro[4.4]nonane-7-carboxylate (compound 4A, 120.0 mg, 0.11 mmol) in DCM (3 mL) was added TFA (1.0 mL, 12.98 mmol). The reaction mixture was stirred at 20° C. for 0.5 h. After the reaction was completed, the reaction mixture was concentrated under vacuum. The residue was diluted with EtOAc (10 mL) and neutralized with NaHCO₃ solution. The organic phase was separated and the aqueous phase was then extracted with EtOAc (10 mL, twice). The combined organic layer was washed with brine (30 mL), dried over Na₂SO₄, filtered and concentrated under vacuum to afford (2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-[(5R)-1-oxo-2,7-diazaspiro[4.4]nonan-2-yl]butanamide (compound 4B, 120.0 mg) as yellow solid. MS calc'd 1023.5 (MH⁺), measured 1023.5 (MH⁺).

### Step 3: Preparation of (2S)-2-[(5R)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 4)

To a solution of (2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-[(5R)-1-oxo-2,7-diazaspiro[4.4]nonan-2-yl]butanamide (compound 4B, 110.0 mg, 0.11 mmol), (2R)-2-chloro-2-fluoro-acetic acid (55.0 mg, 0.49 mmol), DIEA (0.1 mL, 0.55 mmol) in DMF (2 mL) was added T₃P (275.0 mg, 0.43 mmol). The reaction mixture was stirred at 20° C. for 1 h. After the reaction was completed, the reaction mixture was added with H₂O (30 mL) and then extracted with EA (10 mL, twice). The combined organic layer was washed with brine (50 mL), dried over Na₂SO₄, filtered and concentrated under vacuum to give the residue. The residue was purified by prep-HPLC to afford (2S)-2-[(5R)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 4, 54.4 mg) as a yellow solid. MS calc'd 1117.4 (MH⁺), measured 1117.4 (MH⁺). 1H NMR (400 MHz, METHANOL-d₄) δ: 8.68 (d, J=7.2 Hz, 1H), 8.50 (d, J=2.8 Hz, 1H), 7.82-7.69 (m, 1H), 7.52-7.45 (m, 2H), 6.95-6.72 (m, 1H), 5.72-5.65 (m, 1H), 5.21-5.14 (m, 1H), 4.46-4.38 (m, 1H), 4.37-4.31 (m, 1H), 4.27-4.20 (m, 2H), 4.16-3.97 (m, 2H), 3.97-3.88 (m, 1H), 3.88-3.72 (m, 4H), 3.72-3.54 (m, 6H), 3.53-3.44 (m, 3H), 3.35 (d, J=4.2 Hz, 2H), 3.19-3.13 (m, 2H), 3.00 (s, 3H), 2.86-2.78 (m, 1H), 2.62-2.51 (m, 1H), 2.34-2.19 (m, 3H), 2.18-2.07 (m, 3H), 2.06-1.93 (m, 2H), 1.89-1.77 (m, 1H), 1.69-1.60 (m, 1H), 1.45 (d, J=6.0 Hz, 3H), 1.37-1.26 (m, 2H), 1.03-0.96 (m, 6H), 0.93-0.89 (m, 3H), 0.46-0.40 (m, 3H) ppm.

Example 5 was prepared in analogy to the preparation of Example 4 by using (2S)-2-[(5R)-7-tert-butoxycarbonyl-1-oxo-2,7-diazaspiro[4.4]nonan-2-yl]-3-methyl-butanoic acid (compound 4f 1) instead of (2S)-2-[(5S)-7-tert-butoxycarbonyl-1-oxo-2,7-diazaspiro[4.4]nonan-2-yl]-3-methyl-butanoic acid (compound 4f 2). Example 5 (44 mg) was obtained as a white solid. MS calc'd 1117.4 (MH⁺), measured 1117.4 (MH⁺). ¹HNMR (400 MHz, METHANOL-d₄) δ=8.74-8.68 (m, 1H), 8.56-8.50 (m, 1H), 7.76-7.69 (m, 1H), 7.62-7.56 (m, 1H), 7.54-7.47 (m, 1H), 6.96-6.73 (m, 1H), 5.75-5.64 (m, 1H), 5.20 (dd, J=8.4, 16.4 Hz, 1H), 4.46-4.40 (m, 1H), 4.35 (dd, J=4.8, 11.1 Hz, 1H), 4.31-4.19 (m, 2H), 4.18-4.02 (m, 2H), 3.95 (dd, J=4.9, 10.1 Hz, 1H), 3.85-3.72 (m, 4H), 3.71-3.61 (m, 3H), 3.56-3.42 (m, 4H), 3.38 (s, 4H), 3.27 (dd, J=5.1, 10.0 Hz, 2H), 3.21-3.14 (m, 1H), 3.21-3.12 (m, 1H), 3.01 (s, 3H), 2.90-2.79 (m, 1H), 2.60 (d, J=14.5 Hz, 1H), 2.33-2.20 (m, 3H), 2.17-2.05 (m, 3H), 2.02-1.92 (m, 1H), 1.90-1.78 (m, 1H), 1.76-1.60 (m, 1H), 1.48 (d, J=6.1 Hz, 3H), 1.37-1.30 (m, 1H), 1.06-0.99 (m, 6H), 0.95-0.87 (m, 3H), 0.47 (s, 3H) ppm.

The compound 4f 1 and 4f 2 were prepared according to the following scheme:

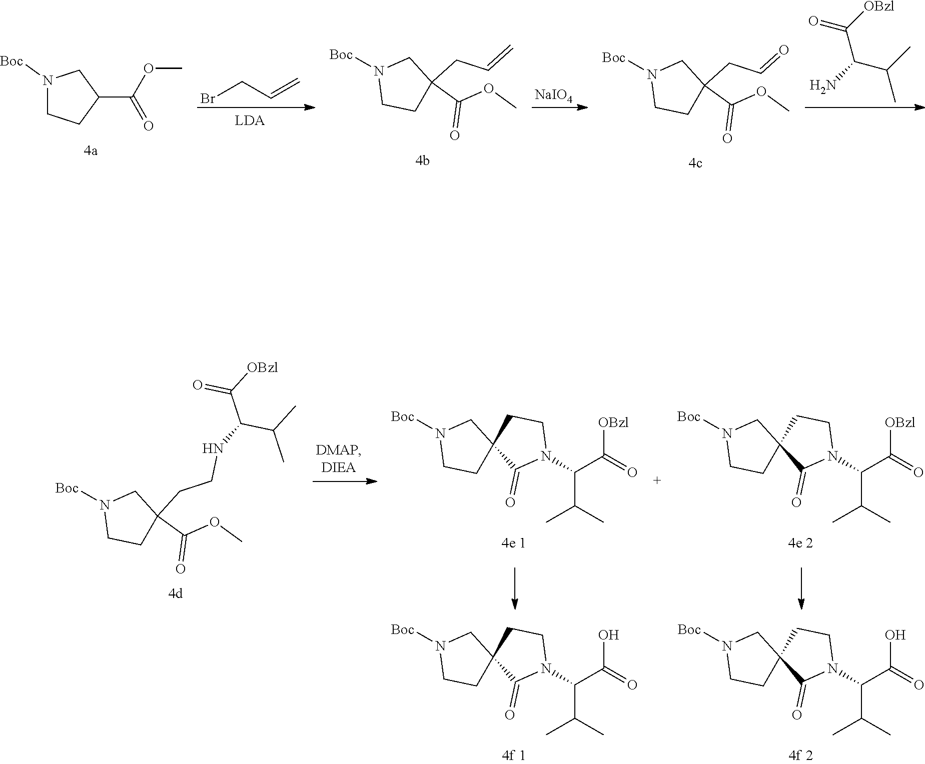

### Step 1: Preparation of 1-(tert-butyl) 3-methyl 3-allylpyrrolidine-1,3-dicarboxylate (compound 4b)

To a solution of 1-(tert-butyl) 3-methyl pyrrolidine-1,3-dicarboxylate (compound 4a, 5.0 g, 21.8 mmol) in THF (60 mL) was added LDA (12 mL, 24 mmol) dropwise at −70° C. under nitrogen atmosphere. After being stirred for 0.5 h, allyl bromide (2.9 g, 23.99 mmol) was added slowly. After the reaction was completed, the reaction mixture was poured into saturated NH₄Cl solution (100 mL) and extracted with EtOAc (70 mL, twice). The combined organic layer was washed with brine (70 mL), dried over Na₂SO₄, filtered and concentrated under vacuum to give a residue, which was purified by silica gel column to afford 1-(tert-butyl) 3-methyl 3-allylpyrrolidine-1,3-dicarboxylate (compound 4b, 2.91 g) as colorless oil.

### Step 2: Preparation of 1-(tert-butyl) 3-methyl 3-(2-oxoethyl)pyrrolidine-1,3-dicarboxylate (compound 4c)

To the mixture of 1-(tert-butyl) 3-methyl 3-allylpyrrolidine-1,3-dicarboxylate (compound 4b, 2.1 g, 7.8 mmol) in 1,4-dioxane (60 mL) and water (6 mL) was added 2,6-Lutidine (1.8 mL, 15.6 mmol) and K₂OsO₄ (0.1 g, 0.39 mmol) in one portion at 0° C. After being stirred at 0° C. for 15 min, sodium metaperiodate (6.6 g, 31.19 mmol) was added portion-wise at 0° C. The resulting mixture was warmed to 20° C. and stirred for another 6 hrs. After the reaction was completed, it was quenched with saturated Na₂S₂O₃ aqueous solution (100 mL) and the reaction mixture was extracted with EtOAc (50 mL, three times). The combined organic layer was washed with brine (50 mL), dried with anhydrous Na₂SO₄, filtered and concentrated under vacuum to afford 1-(tert-butyl) 3-methyl 3-(2-oxoethyl)pyrrolidine-1,3-dicarboxylate (compound 4c, 2.1 g) as yellow oil, which was used in the next step directly.

### Step 3: Preparation of 1-(tert-butyl) 3-methyl 3-(2-(((S)-1-(benzyloxy)-3-methyl-1-oxobutan-2-yl)amino)ethyl)pyrrolidine-1,3-dicarboxylate (compound 4d)

To the mixture of 1-(tert-butyl) 3-methyl 3-(2-oxoethyl)pyrrolidine-1,3-dicarboxylate (compound 4c, 2.1 g, 7.74 mmol) and benzyl (2S)-2-amino-3-methyl-butanoate (1.6 g, 7.74 mmol) in methanol (20 mL) was added zinc chloride (1.05 g, 7.74 mmol) in one portion at 0° C. After being stirred at 0° C. for 1 h, to the mixture was added sodium cyanoborohydride (0.97 g, 15.48 mmol) at 0° C. The resulting mixture was stirred at 0° C. for another 2 hrs. After the reaction was completed, the mixture was poured into saturated NH₄Cl aqueous solution (40 mL) at 0° C. and extracted with EtOAc (50 mL, three times). The combined organic layer was washed with brine (30 mL, four times), dried over anhydrous sodium sulfate, filtered and concentrated under vacuum to give a residue, which was purified by silica gel column to afford 1-(tert-butyl) 3-methyl 3-(2-(((S)-1-(benzyloxy)-3-methyl-1-oxobutan-2-yl)amino)ethyl)pyrrolidine-1,3-dicarboxylate (compound 4d, 2.2 g) as yellow oil. MS calc'd 463.3 (MH⁺), measured 463.2 (MH⁺).

### Step 4: Preparation of tert-butyl 7-((S)-1-(benzyloxy)-3-methyl-1-oxobutan-2-yl)-6-oxo-2,7-diazaspiro[4.4]nonane-2-carboxylate (compound 4e 1& 4e 2)

To the mixture of 1-(tert-butyl) 3-methyl 3-(2-(((S)-1-(benzyloxy)-3-methyl-1-oxobutan-2-yl)amino)ethyl)pyrrolidine-1,3-dicarboxylate (compound 4d, 2.1 g, 4.54 mmol) in toluene (20 mL) was added DIEA (7.9 mL, 45.4 mmol) and DMAP (0.6 g, 4.54 mmol) in one portion. The mixture was heated to 80° C. and stirred for 16 hrs. After the reaction was completed, the mixture was poured into water (30 mL) and extracted with EtOAc (30 mL, three times). The combined organic layer was washed with brine (30 mL), dried with anhydrous Na₂SO₄, filtered and concentrated under vacuum to give a residue, which was purified by reversed phase flash column and the eluent was concentrated under vacuum. The residue was further separated by prep-SFC to afford tert-butyl 7-((S)-1-(benzyloxy)-3-methyl-1-oxobutan-2-yl)-6-oxo-2,7-diazaspiro[4.4]nonane-2-carboxylate (compound 4e 1, faster eluted, 521 mg, compound 4e 2, slower eluted, 525 mg). MS calc'd 453.3 (MNa⁺), measured 453.2 (MNa⁺).

### Step 5: Preparation of (2S)-2-[(5R)-7-tert-butoxycarbonyl-1-oxo-2,7-diazaspiro[4.4]nonan-2-yl]-3-methyl-butanoic acid (compound 4f 1)

To a solution of tert-butyl (R)-7-((S)-1-(benzyloxy)-3-methyl-1-oxobutan-2-yl)-6-oxo-2,7-diazaspiro[4.4]nonane-2-carboxylate (compound 4e 1, 120 mg, 0.28 mmol) in toluene (2 mL) was added wet palladium (12 mg, 10% wt. on activated carbon). The mixture was degassed and purged with hydrogen for 3 times. The reaction mixture was heated to 35° C. and stirred for 3 hrs under hydrogen atmosphere. After the reaction was completed, the solution was filtered and the filtrate was concentrated in vacuo to give (2S)-2-[(5R)-7-tert-butoxycarbonyl-1-oxo-2,7-diazaspiro[4.4]nonan-2-yl]-3-methyl-butanoic acid (compound 4f 1, 94 mg) as a white solid. MS calc'd 363.2 (MNa⁺), measured 363.1 (MNa⁺).

### X-Ray Crystallographic Analysis of Compound 4f 1

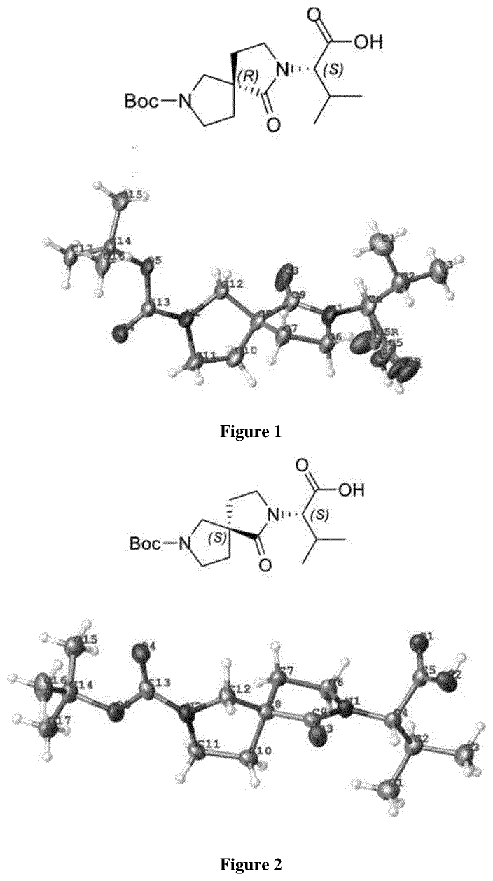

Absolute configuration structure of compound 4f 1 was confirmed by X-ray crystallographic analysis of its single crystal. (FIG. 1)

### Step 6: Preparation of (2S)-2-[(5S)-7-tert-butoxycarbonyl-1-oxo-2,7-diazaspiro[4.4]nonan-2-yl]-3-methyl-butanoic acid (compound 4f 2)

To a solution of tert-butyl (S)-7-((S)-1-(benzyloxy)-3-methyl-1-oxobutan-2-yl)-6-oxo-2,7-diazaspiro[4.4]nonane-2-carboxylate (compound 4e 2, 120 mg, 0.28 mmol) in toluene (2 mL) was added wet palladium (12 mg, 10% wt. on activated carbon). The mixture was degassed and purged with hydrogen for 3 times. The mixture was heated to 35° C. and stirred for 3 h under hydrogen atmosphere. After the reaction was completed, the solution was filtered and the filtrate was concentrated in vacuo to afford (2S)-2-[(5S)-7-tert-butoxycarbonyl-1-oxo-2,7-diazaspiro[4.4]nonan-2-yl]-3-methyl-butanoic acid (compound 4f 2, 79 mg) as a white solid. MS calc'd 363.2 (MNa⁺), measured 363.1 (MNa⁺).

### X-Ray Crystallographic Analysis of Compound 4f 2

Absolute configuration structure of compound 4f 2 was confirmed by X-ray crystallographic analysis of its single crystal. (FIG. 2)

### Example 6

### (2S)-2-[9-[(2R)-2-chloro-2-fluoro-acetyl]-1-oxo-2,9-diazaspiro[5.5]undecan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

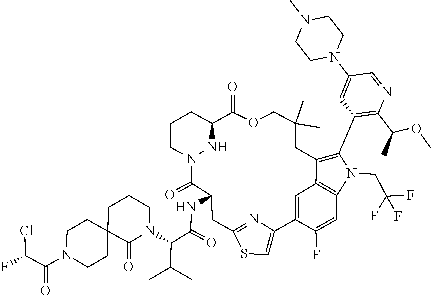

The Example 6 was prepared according to the following scheme:

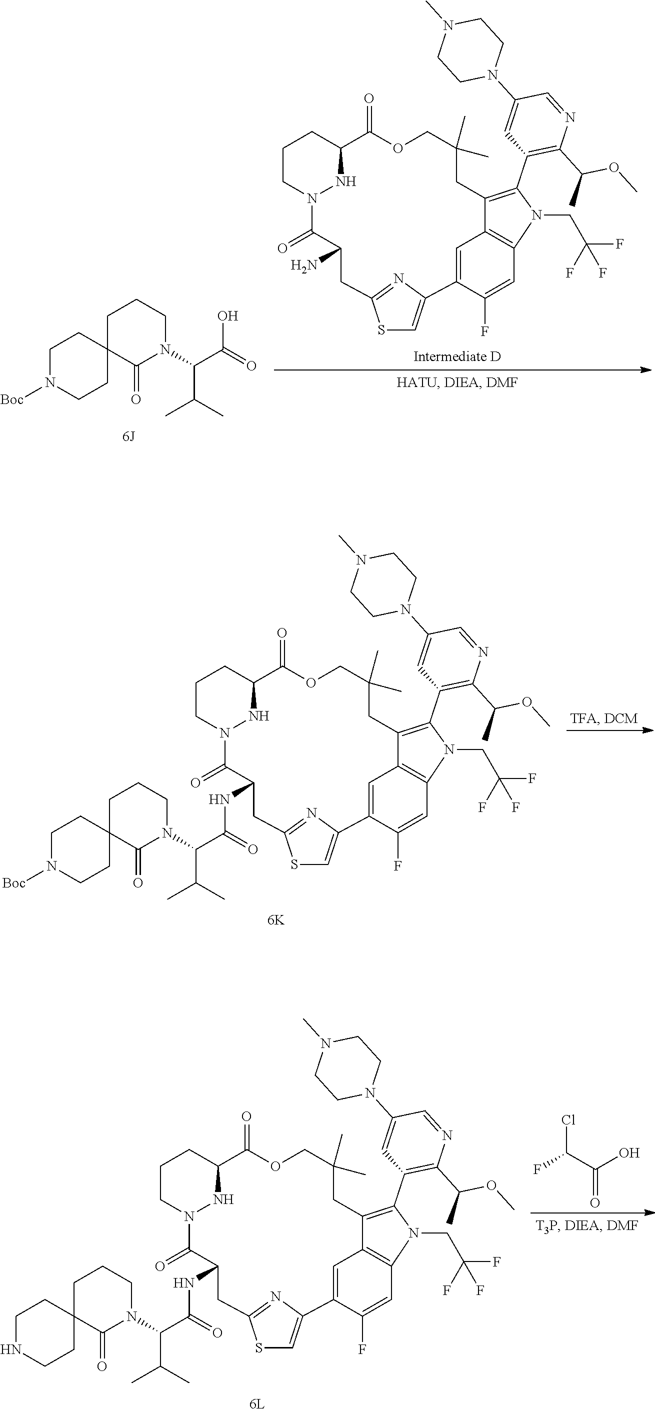

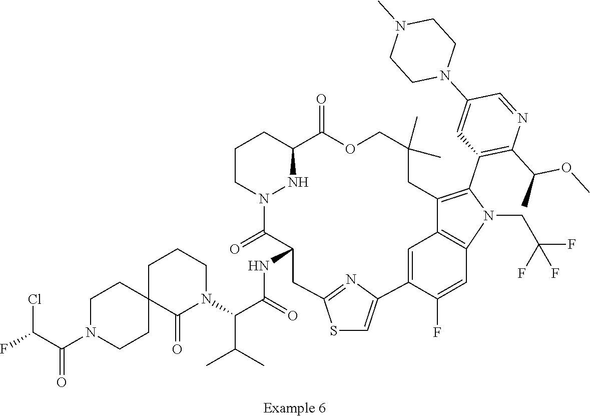

### Step 1: Preparation of tert-butyl 2-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-1-oxo-2,9-diazaspiro[5.5]undecane-9-carboxylate (compound 6K)

To a solution of (2S)-2-(9-tert-butoxycarbonyl-1-oxo-2,9-diazaspiro[5.5]undecan-2-yl)-3-methyl-butanoic acid (compound 6J, 138.0 mg, 0.37 mmol) in DMF (2 mL) was added DIEA (0.2 mL, 0.94 mmol), HATU (106.8 mg, 0.28 mmol). After being stirred at 20° C. for 10 min, the reaction mixture was added with a solution of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D, 150.0 mg, 0.19 mmol) in DMF (2 mL) and the reaction mixture was stirred at 20° C. for 50 min. After the reaction was completed, the reaction mixture was added into water (20 mL) and extracted with EtOAc (20 mL, three times). The combined organic layer was washed with brine (20 mL, three times), dried over anhydrous sodium sulfate, filtered and concentrated under vacuum to give a residue, which was purified by reversed phase flash column to afford tert-butyl 2-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-1-oxo-2,9-diazaspiro[5.5]undecane-9-carboxylate (compound 6K, 125.0 mg) as a yellow solid. MS calc'd 1151.6 (MH⁺), measured 1151.8 (MH⁺).

### Step 2: Preparation of (2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-(1-oxo-2,9-diazaspiro[5.5]undecan-2-yl)butanamide (compound 6L)

To a solution of tert-butyl 2-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-1-oxo-2,9-diazaspiro[5.5]undecane-9-carboxylate (compound 6K, 125.0 mg, 0.11 mmol) in DCM (1.5 mL) was added TFA (0.6 mL, 7.73 mmol). The mixture was stirred at 20° C. for 0.5 h. After the reaction was completed, the reaction mixture was added into sat. NaHCO₃ aq. (20 mL) and extracted with EtOAc (20 mL, three times). The combined organic layer was washed with brine (20 mL, three times), dried over anhydrous sodium sulfate, filtered and concentrated under vacuum to afford (2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-(1-oxo-2,9-diazaspiro[5.5]undecan-2-yl)butanamide (compound 6L, 110.0 mg) as a yellow solid. MS calc'd 1051.5 (MH⁺), measured 1051.7 (MH⁺).

### Step 3: Preparation of (2S)-2-[9-[(2R)-2-chloro-2-fluoro-acetyl]-1-oxo-2,9-diazaspiro[5.5]undecan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 6)

To a solution of (2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-2-(1-oxo-2,9-diazaspiro[5.5]undecan-2-yl)butanamide (compound 6L, 110.0 mg, 0.1 mmol), (R)-2-chloro-2-fluoroacetic acid (58.8 mg, 0.52 mmol) in DMF (3 mL) was added DIEA (0.1 mL, 0.52 mmol) and T₃P (99.9 mg, 0.16 mmol) at 0° C. The mixture was stirred at 20° C. for 1 h. After the reaction was completed, the reaction mixture was poured into water (40 mL) and extracted with EtOAc (20 mL, three times). The combined organic layer was washed with brine (20 mL, three times), dried over anhydrous sodium sulfate, filtered and concentrated under vacuum to give a residue, which was purified by prep-HPLC to afford (2S)-2-[9-[(2R)-2-chloro-2-fluoro-acetyl]-1-oxo-2,9-diazaspiro[5.5]undecan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 6, 17.7 mg) as a white solid. MS calc'd 1145.5 (MH⁺), measured 1145.3 (MH⁺). ¹H NMR (400 MHz, Methanol-d₄) δ=8.69 (d, J=6.4 Hz, 1H), 8.50 (d, J=2.8 Hz, 1H), 7.74-7.70 (m, 1H), 7.53-7.45 (m, 2H), 7.09-6.91 (m, 1H), 5.70-5.63 (m, 1H), 5.22-5.13 (m, 1H), 4.97-4.89 (m, 3H), 4.83-4.77 (m, 1H), 4.44-4.36 (m, 1H), 4.27-4.20 (m, 2H), 4.17-3.89 (m, 4H), 3.80-3.69 (m, 3H), 3.65-3.59 (m, 1H), 3.52-3.41 (m, 4H), 3.36 (s, 5H), 3.24-3.11 (m, 3H), 2.99 (s, 3H), 2.87-2.78 (m, 1H), 2.61-2.54 (m, 1H), 2.31-2.08 (m, 4H), 1.97-1.80 (m, 6H), 1.69-1.59 (m, 3H), 1.45 (d, J=6.0 Hz, 3H), 1.02-0.94 (m, 6H), 0.87 (d, J=6.4 Hz, 3H), 0.44 (s, 3H) ppm.

The compound 6J was prepared according to the following scheme:

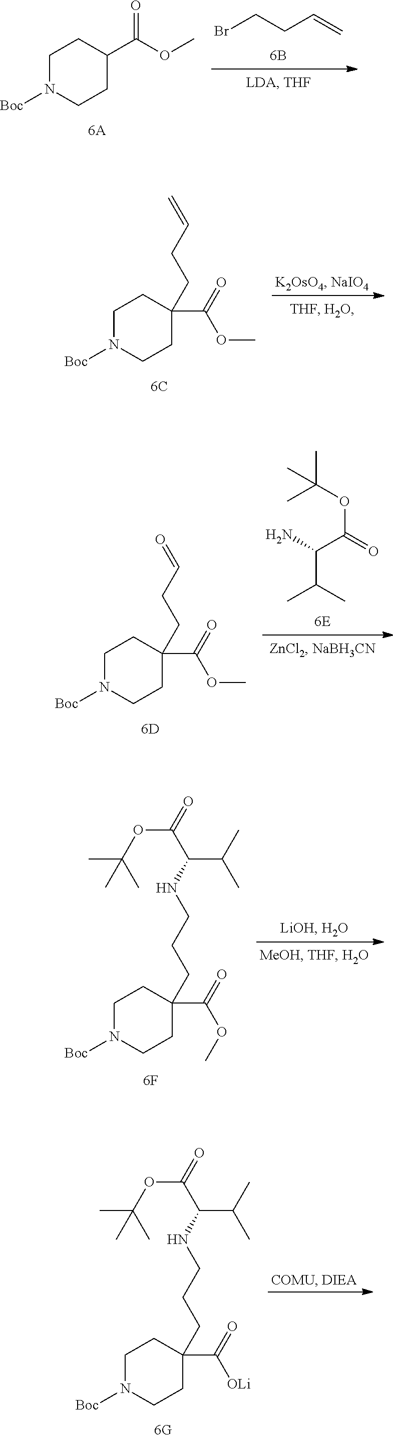

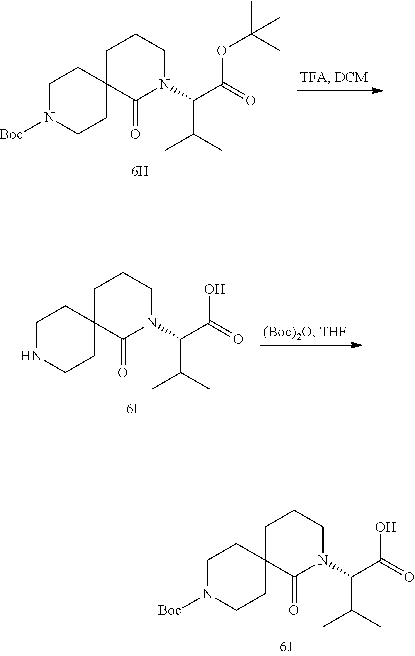 [SMILES: CC(C)[C@@H](C(=O)OC(C)(C)C)N1CCCC2(CCN(C(=O)OC(C)(C)C)CC2)C1=O ; CC(C)[C@@H](C(=O)O)N1CCCC2(CCNCC2)C1=O ; CC(C)[C@@H](C(=O)O)N1CCCC2(CCN(C(=O)OC(C)(C)C)CC2)C1=O]

### Step 1: Preparation of O₁-tert-butyl O₄-methyl 4-but-3-enylpiperidine-1,4-dicarboxylate (compound 6C)

To a solution of O₁-tert-butyl O₄-methyl piperidine-1,4-dicarboxylate (compound 6A, 5.0 g, 20.55 mmol) in THF (80 mL) was added LDA (11.3 mL, 22.61 mmol) at −70° C. under nitrogen atmosphere. After being stirred for 0.5 h, the reaction mixture was added with 4-bromobut-1-ene (compound 6B, 3.0 g, 22.61 mmol) and the resulting mixture was warmed up to 20° C. and stirred for another 2.5 hrs. After the reaction was completed, it was quenched by addition of sat.

NH₄Cl aq. at 0° C. and the reaction mixture was extracted with EtOAc (50 mL, twice). The combined organic layer was washed with brine, dried over Na₂SO₄, filtered and concentrated under vacuum to give a residue, which was purified by column chromatography (EtOAc in PE=5% to 30%) to afford O₁-tert-butyl O₄-methyl 4-but-3-enylpiperidine-1,4-dicarboxylate (compound 6C, 4.13 g) as yellow oil. ¹H NMR (400 MHz, CHLOROFORM-d) δ 5.69-5.81 (m, 1H), 4.91-5.06 (m, 2H), 3.87 (d, J=14.0 Hz, 2H), 3.71 (s, 3H), 2.92-2.82 (m, 2H), 2.11 (d, J=13.6 Hz, 2H), 2.00-1.92 (m, 2H), 1.63-1.58 (m, 2H), 1.45 (s, 9H), 1.40-1.32 (m, 2H) ppm.

### Step 2: Preparation of O₁-tert-butyl O₄-methyl 4-(3-oxopropyl)piperidine-1,4-dicarboxylate (compound 6D)

To a mixture of O₁-tert-butyl O₄-methyl 4-but-3-enylpiperidine-1,4-dicarboxylate (compound 6C, 1.9 g, 6.49 mmol) in 1,4-dioxane (40 mL) and water (4 mL) was added 2,6-Lutidine (1.5 mL, 12.98 mmol) and potassium osmate (VI) dihydrate (119.5 mg, 0.32 mmol) at 0° C. After being stirred at 0° C. for 15 min, the reaction mixture was added with sodium metaperiodate (5.6 g, 25.96 mmol) at 0° C. and the resulting mixture was warmed to 20° C. and stirred for 3 hrs. After the reaction was completed, the reaction mixture was extracted with EtOAc (20 mL, three times), and washed with saturated Na₂SO₃ aqueous solution (20 mL). The combined organic layer was washed with brine (20 mL), dried with anhydrous Na₂SO₄, filtered and concentrated under vacuum to afford O₁-tert-butyl O₄-methyl 4-(3-oxopropyl)piperidine-1,4-dicarboxylate (compound 6D, 2.0 g) as yellow oil. MS calc'd 200.2 (M-Boc+H⁺), measured 200.1 (M-Boc+H⁺).

### Step 3: Preparation of O₁-tert-butyl O₄-methyl 4-[3-[[(1S)-1-tert-butoxycarbonyl-2-methyl-propyl]amino]propyl]piperidine-1,4-dicarboxylate (compound 6F)

To a mixture of O₁-tert-butyl O₄-methyl 4-(3-oxopropyl)piperidine-1,4-dicarboxylate (compound 6D, 1.0 g, 3.34 mmol) and Boc-N-Me-Val-OH (770.5 mg, 3.67 mmol) in methanol (20 mL) was added zinc chloride (500.7 mg, 3.67 mmol) at 0° C. After being was stirred at 0° C. for 0.5 h, the reaction mixture was added with sodium cyanoborohydride (272.8 mg, 4.34 mmol) at 0° C. and the resulting mixture was warmed to 20° C. and stirred for another 1 h. After the reaction was completed, it was quenched with sat. NH₄Cl aq. (20 mL) and the reaction mixture was extracted with EtOAc (20 mL, three times). The combined organic layer was washed with brine (20 mL), dried with anhydrous Na₂SO₄, filtered and concentrated under vacuum to give a residue, which was purified by column chromatography (EtOAc in PE=10% to 30%) to afford O₁-tert-butyl O₄-methyl 4-[3-1[[(1S)-1-tert-butoxycarbonyl-2-methyl-propyl]amino]propyl]piperidine-1,4-dicarboxylate (compound 6F, 1.1 g) as yellow oil. MS calc'd 457.3 (MH⁺), measured 457.2 (MH⁺).

### Step 4: Preparation of lithium;1-tert-butoxycarbonyl-4-[3-[[(1S)-1-tert-butoxycarbonyl-2-methyl-propyl]amino]propyl]piperidine-4-carboxylate (compound 6G)

To a mixture of O₁-tert-butyl O₄-methyl 4-[3-[[(1S)-1-tert-butoxycarbonyl-2-methyl-propyl]amino]propyl]piperidine-1,4-dicarboxylate (compound 6F, 400.0 mg, 0.88 mmol) in methanol (6 mL), THF (0.6 mL) and water (0.6 mL) was added lithium hydroxide monohydrate (367.6 mg, 8.76 mmol). The resulting mixture was heated to 60° C. and stirred for 12 hrs. After the reaction was completed, the reaction mixture was cooled to 20° C. and concentrated under vacuum to afford lithium;1-tert-butoxycarbonyl-4-[3-[[(1S)-1-tert-butoxycarbonyl-2-methyl-propyl]amino]propyl]piperidine-4-carboxylate (compound 6G, 392.0 mg) as a white solid. MS calc'd 443.3 (M-Li+H⁺), measured 443.2 (M-Li+H⁺).

### Step 5: Preparation of tert-butyl 2-[(1S)-1-tert-butoxycarbonyl-2-methyl-propyl]-1-oxo-2,9-diazaspiro[5.5]undecane-9-carboxylate (compound 6H)

To the mixture of lithium;1-tert-butoxycarbonyl-4-[3-[[(1S)-1-tert-butoxycarbonyl-2-methyl-propyl]amino]propyl]piperidine-4-carboxylate (392.0 mg, 0.87 mmol) in DMF (8 mL) was added DIEA (0.5 mL, 2.62 mmol) and COMU (673.7 mg, 1.57 mmol) at 0° C. The mixture was stirred at 20° C. for 1 h. After the reaction was completed, the reaction mixture was poured into water (80 mL) and extracted with EtOAc (20 mL, three times). The combined organic layer was washed with brine (20 mL, three times), dried over anhydrous sodium sulfate, filtered and concentrated under vacuum to give a residue, which was purified by column chromatography (EtOAc in PE=5% to 25%) to afford tert-butyl 2-[(1S)-1-tert-butoxycarbonyl-2-methyl-propyl]-1-oxo-2,9-diazaspiro[5.5]undecane-9-carboxylate (compound 6H, 200.0 mg) as yellow oil. MS calc'd 425.3 (MH⁺), measured 425.3 (MH⁺).

### Step 6: Preparation of (2S)-3-methyl-2-(1-oxo-2,9-diazaspiro[5.5]undecan-2-yl)butanoic acid (compound 61)

To a solution of tert-butyl 2-[(1S)-1-tert-butoxycarbonyl-2-methyl-propyl]-1-oxo-2,9-diazaspiro[5.5]undecane-9-carboxylate (compound 6H, 200.0 mg, 0.47 mmol) in DCM (1.5 mL) was added trifluoroacetic acid (1.0 mL, 12.98 mmol). The mixture was stirred at 20° C. for 1 h. After the reaction was completed, the mixture was concentrated under vacuum to give (2S)-3-methyl-2-(1-oxo-2,9-diazaspiro[5.5]undecan-2-yl)butanoic acid (compound 61, 180.0 mg, TFA salt) as colorless oil. MS calc'd 269.2 (MH⁺), measured 269.2 (MH⁺).

### Step 7: Preparation of (2S)-2-(9-tert-butoxycarbonyl-1-oxo-2,9-diazaspiro[5.5]undecan-2-yl)-3-methyl-butanoic acid (compound 6J)

To a solution of (2S)-3-methyl-2-(1-oxo-2,9-diazaspiro[5.5]undecan-2-yl)butanoic acid (compound 61, 180.0 mg, 0.47 mmol, TFA salt) in THF (3 mL) and water (3 mL) was added sodium carbonate (99.8 mg, 0.94 mmol) and di-tert-butyl dicarbonate (123.3 mg, 0.56 mmol). The mixture was stirred at 20° C. for 1 h. After the reaction was completed, the mixture was concentrated under vacuum to give a residue, which was purified by reversed phase flash column to afford (2S)-2-(9-tert-butoxycarbonyl-1-oxo-2,9-diazaspiro[5.5]undecan-2-yl)-3-methyl-butanoic acid (compound 6J, 400.0 mg) as an off-white solid. MS calc'd 367.2 (MH⁻), measured 367.2 (MH⁻).

### Example 8

### (3S)-1-[(2R)-2-chloro-2-fluoro-acetyl]-N-[(1S)-1-[[(8S,14S)-25-fluoro-4-hydroxy-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl]-2-methyl-propyl]-N-methyl-pyrrolidine-3-carboxamide

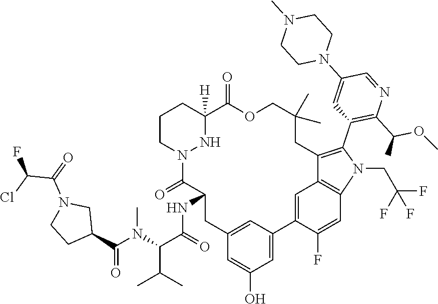

The title compound was prepared in analogy to the preparation of Example 1 by using (8S,14S)-8-amino-25-fluoro-4-hydroxy-(21M)-21-[2-[(1S)-1-methoxyethyl]-5-(4-methylpiperazin-1-yl)-3-pyridyl]-18,18-dimethyl-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaene-9,15-dione (Intermediate G) instead of (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E). Example 8 (21.6 mg) was obtained as a white solid. MS calc'd 1114.5 (MH⁺), measured 1114.8 (MH⁺). ¹H NMR (400 MHz, Methanol-d₄) δ=8.43 (d, J=2.4 Hz, 1H), 7.62 (dd, J=10.8, 7.2 Hz, 1H), 7.55-7.53 (m, 2H), 7.29 (s, 1H), 7.26-7.20 (m, 1H), 6.96-6.90 (m, 1H), 6.65-6.55 (m, 1H), 5.65-5.49 (m, 1H), 4.97-4.68 (m, 2H), 4.67-4.62 (m, 1H), 4.46-4.38 (m, 1H), 4.20-4.12 (m, 1H), 4.00-3.87 (m, 1H), 3.82-3.61 (m, 6H), 3.59-3.43 (m, 5H), 3.38-3.32 (m, 2H), 3.25-3.11 (in, 1H), 3.04 (s, 2H), 2.99-2.96 (m, 1H), 2.93 (s, 4H), 2.90-2.65 (m, 3H), 2.63-2.47 (m, 2H), 2.39-2.11 (m, 3H), 2.10-1.95 (m, 2H), 1.89-1.79 (m, 1H), 1.58-1.27 (m, 6H), 1.03-0.92 (m, 6H), 0.91-0.84 (in, 1H). 0.83-0.75 (in, 3H). 0.47-0.32 (in, 3H) ppm.

### Example 9

### 1-[(2R)-2-chloro-2-fluoro-acetyl]-4-fluoro-N-[(1S)-1-[[(7S,13S)-(20M)-20-[2-[(1S)-1-methoxyethyl]-5-(4-methylpiperazin-1-yl)-3-pyridyl]-17,17,24-trimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-piperidine-4-carboxamide

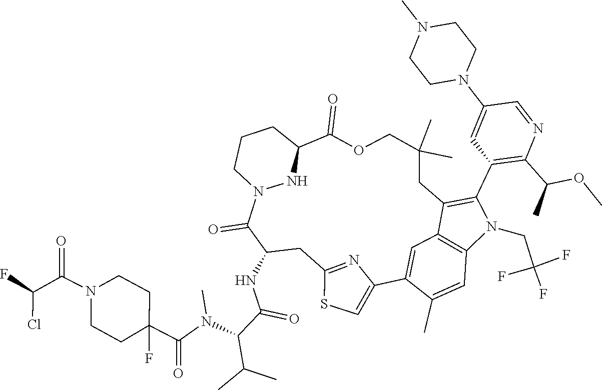

The title compound was prepared in analogy to the preparation of Example 1 by using 1-tert-butoxycarbonyl-4-fluoro-piperidine-4-carboxylic acid and (7S,13S)-7-amino-(20M)-20-[2-[(1S)-1-methoxyethyl]-5-(4-methylpiperazin-1-yl)-3-pyridyl]-17,17,24-trimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate F) instead of (S)-1-Boc-pyrrolidine-3-carboxylic acid (compound 1D) and (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E). Example 9 (2.7 mg) was obtained as a white solid. MS calc'd 1133.5 (MH⁺), measured 1133.5 (MH⁺). ¹H NMR (400 MHz, Methanol-d₄) δ=8.49-8.36 (m, 1H), 8.03 (d, J=13.6 Hz, 1H), 7.58-7.39 (m, 3H), 7.12-6.94 (m, 1H), 5.90-5.77 (m, 1H), 5.26-5.11 (m, 1H), 4.73-4.61 (m, 1H), 4.52-4.31 (m, 2H), 4.21 (m, 1H), 4.14-3.84 (m, 4H), 3.75-3.49 (m, 3H), 3.26-3.07 (m, 10H), 3.00 (s, 4H), 2.52 (m, 6H), 2.15 (m, 8H), 1.97-1.82 (m, 1H), 1.71-1.50 (m, 2H), 1.45 (m, 3H), 1.36-1.24 (m, 3H), 1.02-0.87 (m, 9H), 0.39 (s, 3H) ppm.

### Example 10

### 1-[(2R)-2-chloro-2-fluoro-acetyl]-4-fluoro-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-piperidine-4-carboxamide

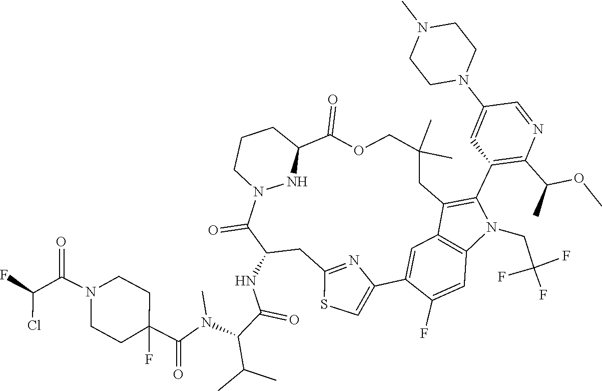

The title compound was prepared in analogy to the preparation of Example 1 by using 1-tert-butoxycarbonyl-4-fluoro-piperidine-4-carboxylic acid and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D) instead of (S)-1-Boc-pyrrolidine-3-carboxylic acid (compound 1D) and (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E). Example 10 (17.5 mg) was obtained as a yellow solid. MS calc'd 1137.5 (MH⁺), measured 1137.5 (MH⁺), ¹H NMR (400 MHz, Methanol-d₄) δ=8.74-8.68 (m, 1H), 8.54-8.51 (m, 1H), 7.74-7.70 (m, 1H), 7.57-7.47 (m, 2H), 7.15-6.97 (m, 1H), 5.75-5.64 (m, 1H), 5.26-5.12 (m, 1H), 4.83-4.73 (m, 1H), 4.51-4.32 (m, 2H), 4.29-4.21 (m, 2H), 4.20-3.89 (m, 3H), 3.88-3.62 (m, 5H), 3.60-3.43 (m, 4H), 3.40-3.36 (m, 4H), 3.26-3.22 (m, 4H), 3.04-3.00 (m, 4H), 2.89-2.81 (m, 1H), 2.64-2.56 (m, 1H), 2.39-2.15 (m, 6H), 1.98-1.95 (m, 1H), 1.89-1.78 (m, 1H), 1.73-1.59 (m, 1H), 1.50-1.43 (m, 4H), 1.38-1.28 (m, 1H), 1.04-0.97 (m, 6H), 0.95-0.88 (m, 3H), 0.51-0.41 (m, 3H) ppm.

### Example 11

### (2S)-2-[2-[(2R)-2-chloro-2-fluoro-acetyl]-5-oxo-2,6-diazaspiro[3.5]nonan-6-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

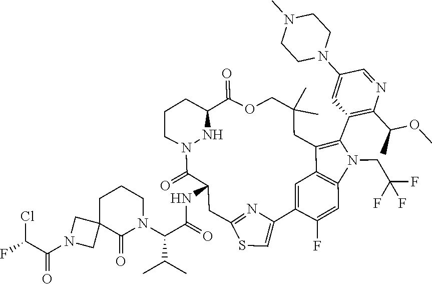

The title compound was prepared in analogy to the preparation of Example 6 by using (2S)-2-(2-tert-butoxycarbonyl-5-oxo-2,6-diazaspiro[3.5]nonan-6-yl)-3-methyl-butanoic acid (compound 11J) instead of (2S)-2-(9-tert-butoxycarbonyl-1-oxo-2,9-diazaspiro[5.5]undecan-2-yl)-3-methyl-butanoic acid (compound 6J). Example 11 (6.6 mg) was obtained as a white solid. MS calc'd 1117.4 (MH⁺), measured 1117.5 (MH⁺). ¹H NMR (400 MHz, Methanol-d₄) δ=8.68 (d, J=7.3 Hz, 1H), 8.50 (d, J=2.8 Hz, 1H), 7.72 (dd, J=2.4, 15.6 Hz, 1H), 7.50-7.45 (m, 2H), 6.79-6.60 (m, 1H), 5.72-5.64 (m, 1H), 5.21-5.10 (m, 3H), 4.44 (br s, 2H), 4.21 (br d, J=5.6 Hz, 3H), 4.05-3.86 (m, 3H), 3.79 (br d, J=11.2 Hz, 1H), 3.74-3.61 (m, 4H), 3.57-3.40 (m, 8H), 3.35 (br s, 2H), 3.15 (br d, J=14.8 Hz, 3H), 3.00 (s, 3H), 2.83 (br t, J=11.6 Hz, 1H), 2.62-2.50 (m, 1H), 2.20 (br s, 3H), 1.99-1.94 (m, 1H), 1.83 (br s, 3H), 1.64 (br dd, J=4.0, 12.0 Hz, 1H), 1.44 (d, J=6.0 Hz, 3H), 1.00 (br dd, J=2.8, 6.4 Hz, 3H), 0.97 (s, 3H), 0.89 (br d, J=6.4 Hz, 3H), 0.44 (s, 3H) ppm.

The compound 11J was prepared according to the following scheme:

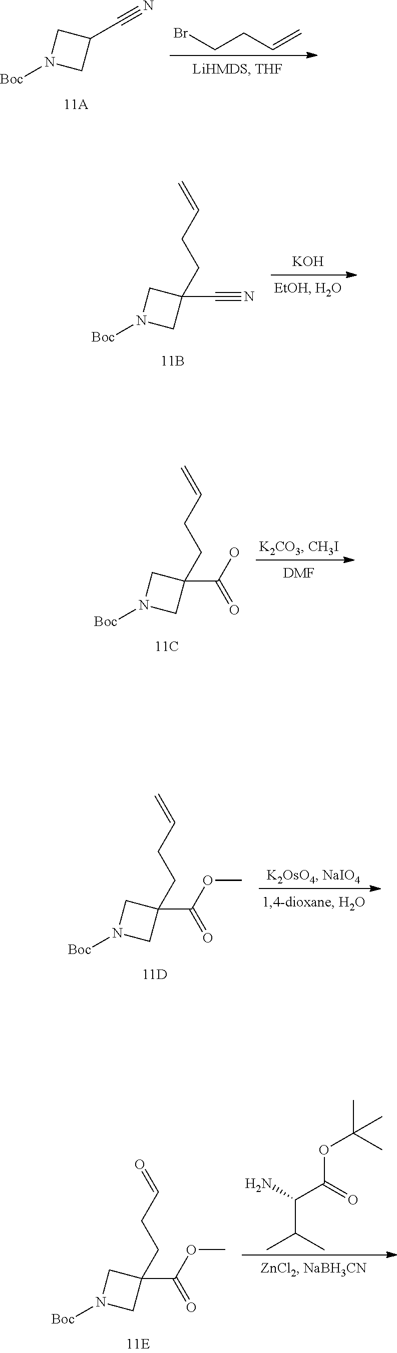

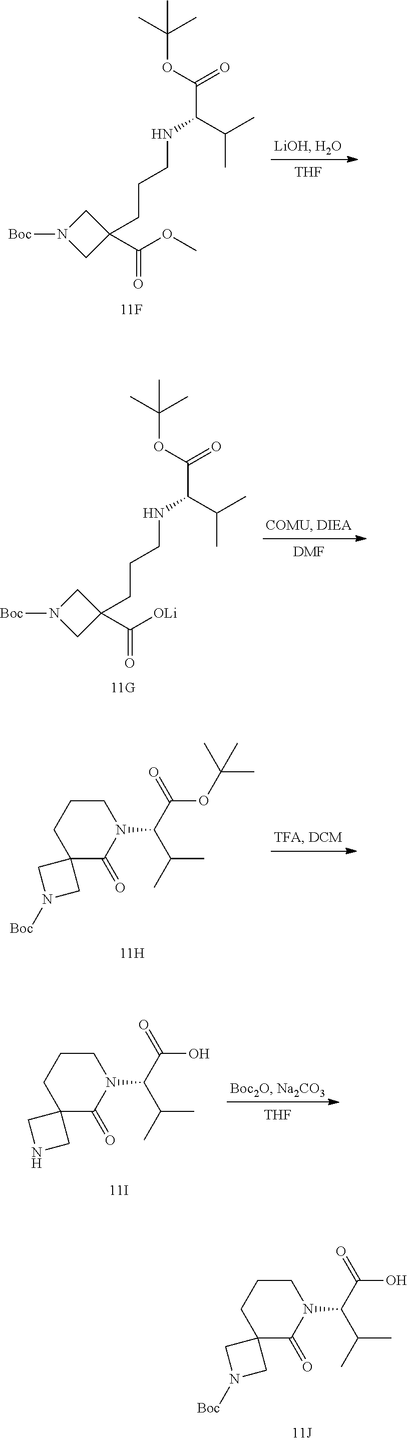

### Step 1: Preparation of tert-butyl 3-but-3-enyl-3-cyano-azetidine-1-carboxylate (compound 11B)

To a solution of tert-butyl 3-cyanoazetidine-1-carboxylate (compound 11A, 5.0 g, 27.44 mmol) in THF (50 mL) cooled to −70° C. was added lithium bis(trimethylsilyl)amide (30.1 mL, 30.18 mmol) dropwise under nitrogen atmosphere. After being stirred for 0.5 h, the reaction mixture was added with 4-bromo-1-butene (4.4 g, 32.93 mmol) slowly at −70° C. The mixture was warmed up to 20° C. and stirred for 3 hrs. After the reaction was completed, it was quenched with saturated NH₄Cl (200 mL) and the reaction mixture was extracted with EtOAc (50 mL, three times). The combined organic layer was washed with brine (100 mL), dried over anhydrous Na₂SO₄, filtered and concentrated under vacuum to give a residue, which was purified by column chromatography (EtOAc in PE=5% to 30%) to afford tert-butyl 3-but-3-enyl-3-cyano-azetidine-1-carboxylate (compound 11B, 5.4 g) as yellow oil. MS calc'd 237.2 (MH⁺), measured 181.6 (M-C₄H₈+H⁺).

### Step 2: Preparation of 3-but-3-enyl-1-tert-butoxycarbonyl-azetidine-3-carboxylic acid (compound 11C)

To the mixture of tert-butyl 3-but-3-enyl-3-cyano-azetidine-1-carboxylate (compound 11B, 5.4 g, 22.85 mmol) in ethanol (60 mL) and water (60 mL) was added potassium hydroxide (5.1 g, 91.41 mmol) in one portion. The mixture was heated at 80° C. for 16 hrs. After the reaction was completed, the pH of the reaction mixture was adjust to pH=3 with 1N HCl aqueous solution and it was extracted with EtOAc (50 mL, three times). The combined organic layer was washed with brine (100 mL), dried with anhydrous Na₂SO₄, filtered and concentrated under vacuum to give 3-but-3-enyl-1-tert-butoxycarbonyl-azetidine-3-carboxylic acid (compound 11C, 6.31 g) as yellow oil. MS calc'd 256.2 (MH⁺), measured 200.3 (M-C₄H₈+H⁺).

### Step 3: Preparation of O₁-tert-butyl O₃-methyl 3-but-3-enylazetidine-1,3-dicarboxylate (compound 11D)

To the mixture of 3-but-3-enyl-1-tert-butoxycarbonyl-azetidine-3-carboxylic acid (compound 11C, 6.3 g, 24.72 mmol) in DMF (60 mL) was added potassium carbonate (10.2 g, 74.15 mmol) and iodomethane (7.0 g, 49.43 mmol) in one portion. The mixture was stirred at 20° C. for 2 hrs. After the reaction was completed, the mixture was poured into water (150 mL) and extracted with EtOAc (70 mL, three times). The combined organic layer was washed with brine (70 mL, three times), dried with anhydrous Na₂SO₄, filtered and concentrated under vacuum. The residue was purified by column chromatography (EtOAc in PE=5% to 30%) to afford O₁-tert-butyl O₃-methyl 3-but-3-enylazetidine-1,3-dicarboxylate (compound 11D, 3.76 g) as yellow oil. MS calc'd 270.2 (MH⁺), measured 214.3 (M-C₄H₈+H⁺).

### Step 4: Preparation of O₁-tert-butyl O₃-methyl 3-(3-oxopropyl)azetidine-1,3-dicarboxylate (compound 11E)

To the mixture of O₁-tert-butyl O₃-methyl 3-but-3-enylazetidine-1,3-dicarboxylate (compound 11D, 3.7 g, 13.96 mmol) in 1,4-dioxane (100 mL) and water (10 mL) was added 2,6-Lutidine (3.2 mL, 27.92 mmol) and potassium osmate(VI) dihydrate (0.3 g, 0.7 mmol) in one portion at 0° C. After being stirred at 0° C. for 15 minutes, the reaction mixture was added with sodium metaperiodate (11.9 g, 55.84 mmol) and the resulting mixture was warmed to 20° C. and stirred for another 3 hrs. After the reaction was completed, the reaction was quenched by addition with saturated Na₂SO₃ aqueous solution (200 mL) and the reaction mixture was extracted with EtOAc (100 mL, three times). The combined organic layer was washed with brine (100 mL), dried with anhydrous Na₂SO₄, filtered and concentrated under vacuum to afford O₁-tert-butyl O₃-methyl 3-(3-oxopropyl)azetidine-1,3-dicarboxylate (compound 11E, 3.8 g) as yellow oil. MS calc'd 272.2 (MH⁺), measured 216.3 (M-C₄H₈+H⁺).

### Step 5: Preparation of O₁-tert-butyl O₃-methyl 3-[3-[[(1S)-1-tert-butoxycarbonyl-2-methyl-propyl]amino]propyl]azetidine-1,3-dicarboxylate (compound 11F)

To the mixture of tert-butyl-L-valinate hydrogen chloride (1.8 g, 10.5 mmol) and O₁-tert-butyl O₃-methyl 3-(3-oxopropyl)azetidine-1,3-dicarboxylate (compound 11E, 1.9 g, 7.0 mmol) in Methanol (20 mL) was added zinc chloride (1.4 g, 10.5 mmol) in one portion at 0° C. After being stirred at 20° C. for 0.5 h, the reaction mixture was added with sodium cyanoborohydride (0.9 g, 14.01 mmol) at 0° C. and the resulting mixture was stirred at 20° C. for another 1 h. After the reaction was completed, it was quenched by addition of saturated NH₄Cl aqueous solution (100 mL) and the reaction mixture was extracted with EtOAc (50 mL, three times). The combined organic layer was washed with brine (50 mL), dried with anhydrous Na₂SO₄, filtered and concentrated under vacuum to give a residue, which was purified by column chromatography (EtOAc in PE=10% to 30%) to afford O₁-tert-butyl O₃-methyl 3-[3-[[(1S)-1-tert-butoxycarbonyl-2-methyl-propyl]amino]propyl]azetidine-1,3-dicarboxylate (compound 11F, 1.2 g) as yellow oil. MS calc'd 429.3 (MH⁺), measured 429.4 (MH⁺).

### Step 6: Preparation of lithium;1-tert-butoxycarbonyl-3-[3-[[(1S)-1-tert-butoxycarbonyl-2-methyl-propyl]amino]propyl]azetidine-3-carboxylate (compound 11G)

To the mixture of O₁-tert-butyl O₃-methyl 3-[3-[[(1S)-1-tert-butoxycarbonyl-2-methyl-propyl]amino]propyl]azetidine-1,3-dicarboxylate (compound 11F, 1.2 g, 2.8 mmol) in methanol (30 mL), water (3 mL) and THF (3 mL) was added lithium hydroxide monohydrate (1.2 g, 28.0 mmol). The mixture was then heated to 60° C. and stirred for 12 hrs. After the reaction was completed, the reaction mixture was concentrated under vacuum to afford lithium;1-tert-butoxycarbonyl-3-[3-[[(1S)-1-tert-butoxycarbonyl-2-methyl-propyl]amino]propyl]azetidine-3-carboxylate (compound 11G, 2.3 g) as a yellow solid. MS calc'd 421.8 (MH⁺), measured 415.2 (M-Li+H⁺).

### Step 7: Preparation of tert-butyl 6-[(1S)-1-tert-butoxycarbonyl-2-methyl-propyl]-5-oxo-2,6-diazaspiro[3.5]nonane-2-carboxylate (compound 11H)

To a solution of lithium;1-tert-butoxycarbonyl-3-[3-[[(1S)-1-tert-butoxycarbonyl-2-methyl-propyl]amino]propyl]azetidine-3-carboxylate (compound 11G, 2.0 g, 2.41 mmol) in DMF (30 mL) was added DIEA (2.1 mL, 12.06 mmol) and COMU (2.0 g, 4.82 mmol) at 0° C. in one portion. The reaction mixture was then warmed up to 20° C. and stirred for 1 h. After the reaction was completed, the mixture was poured into water (100 mL) and extracted with EtOAc (50 mL, three times). The combined organic layer was washed with brine (50 mL, three times), dried with anhydrous Na₂SO₄, filtered and concentrated under vacuum to give a residue, which was purified by column chromatography (EtOAc in PE=20% to 30%) to afford tert-butyl 6-[(1S)-1-tert-butoxycarbonyl-2-methyl-propyl]-5-oxo-2,6-diazaspiro[3.5]nonane-2-carboxylate (compound 11H, 512.0 mg) as a yellow solid. MS calc'd 397.3 (MH⁺), measured 419.4 (MNa⁺).

### Step 8: Preparation of (2S)-3-methyl-2-(5-oxo-2,6-diazaspiro[3.5]nonan-6-yl)butanoic acid (compound 111)

To a solution of tert-butyl 6-[(1S)-1-tert-butoxycarbonyl-2-methyl-propyl]-5-oxo-2,6-diazaspiro[3.5]nonane-2-carboxylate (compound 11H, 512.0 mg, 1.29 mmol) in DCM (3 mL) was added TFA (3.0 mL, 38.94 mmol). The mixture was stirred at 20° C. for 2 hrs. After the reaction was completed, the mixture was concentrated under vacuum to afford (2S)-3-methyl-2-(5-oxo-2,6-diazaspiro[3.5]nonan-6-yl)butanoic acid (compound 11I, 301.0 mg) as yellow oil as TFA salt. MS calc'd 241.2 (MH⁺), measured 241.5 (MH⁺).

### Step 9: Preparation of (2S)-2-(2-tert-butoxycarbonyl-5-oxo-2,6-diazaspiro[3.5]nonan-6-yl)-3-methyl-butanoic acid (compound 11J)

To the mixture of (2S)-3-methyl-2-(5-oxo-2,6-diazaspiro[3.5]nonan-6-yl)butanoic acid (compound 11I, 301.0 mg, 1.25 mmol) in THF (12 mL) was added di-t-butyldicarbonate (326.9 mg, 1.5 mmol) and sodium carbonate (661.6 mg, 6.24 mmol) in one portion, the mixture was stirred at 20° C. for 2 hrs. After the reaction was completed, the mixture was filtered, and the filtrate was concentrated under vacuum. The resulting residue was purified by prep-HPLC to afford (2S)-2-(2-tert-butoxycarbonyl-5-oxo-2,6-diazaspiro[3.5]nonan-6-yl)-3-methyl-butanoic acid (compound 11J, 303.0 mg) as colorless gum. MS calc'd 341.2 (MH⁺), measured 363.2 (MNa⁺).

### Example 13

### (2R)-2-[[1-[(2R)-2-chloro-2-fluoro-acetyl]azetidin-3-yl]oxymethyl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo-[17.5.2.1^{2,5}.1^{9,13}.0^{22,26}]octacosa-1(25),2,5(28),19,22(26),23-hexaen-7-yl]-3-methyl-butanamide

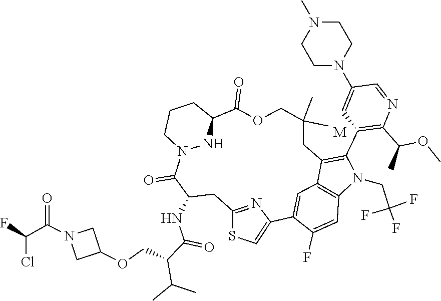

The compound was prepared according to the following scheme:

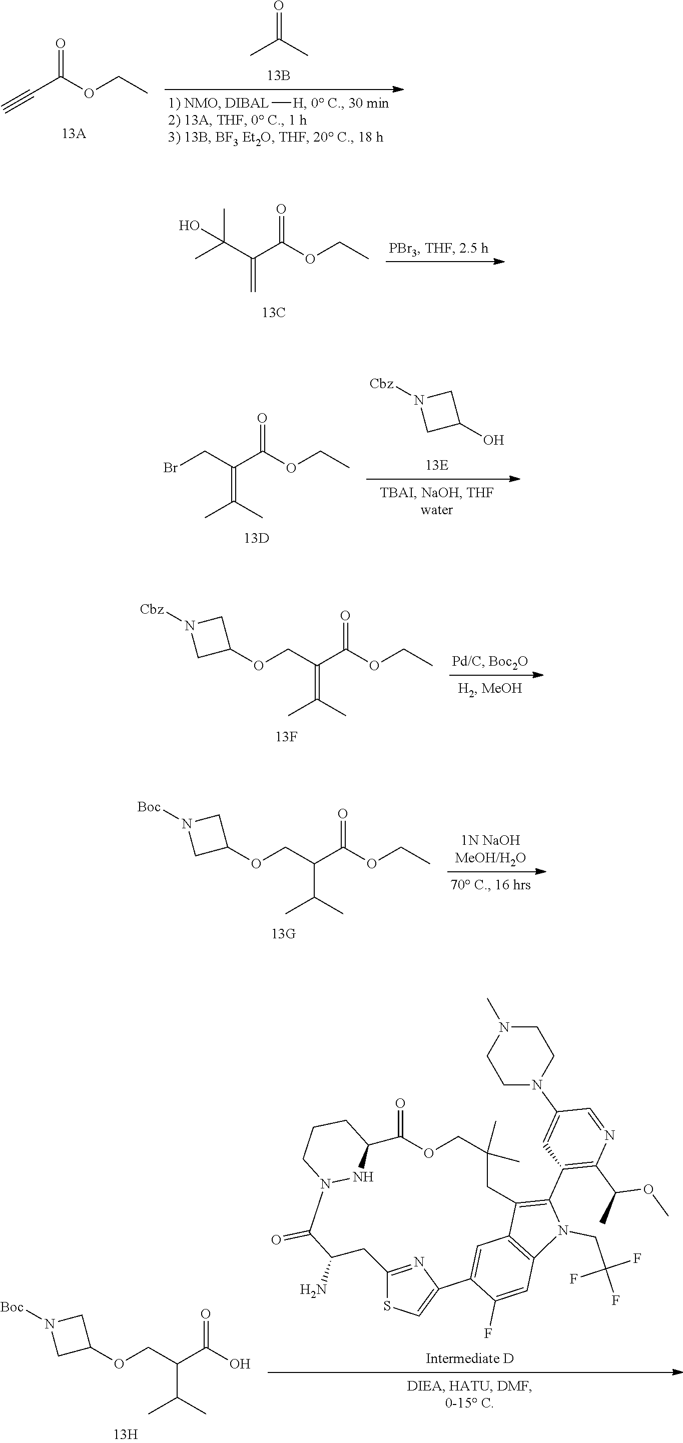

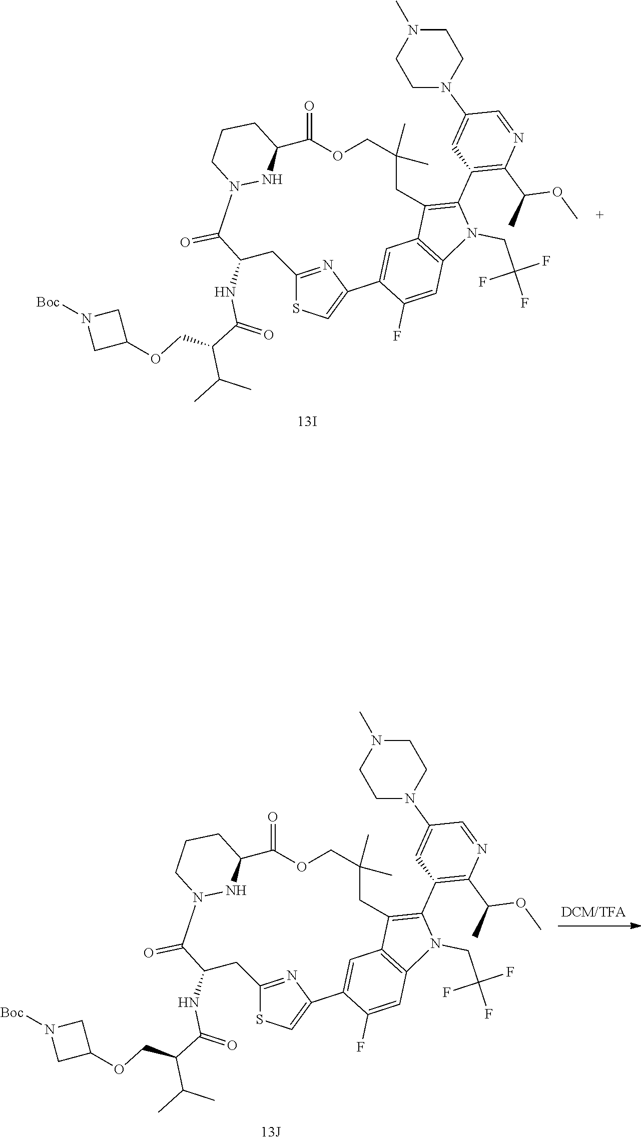

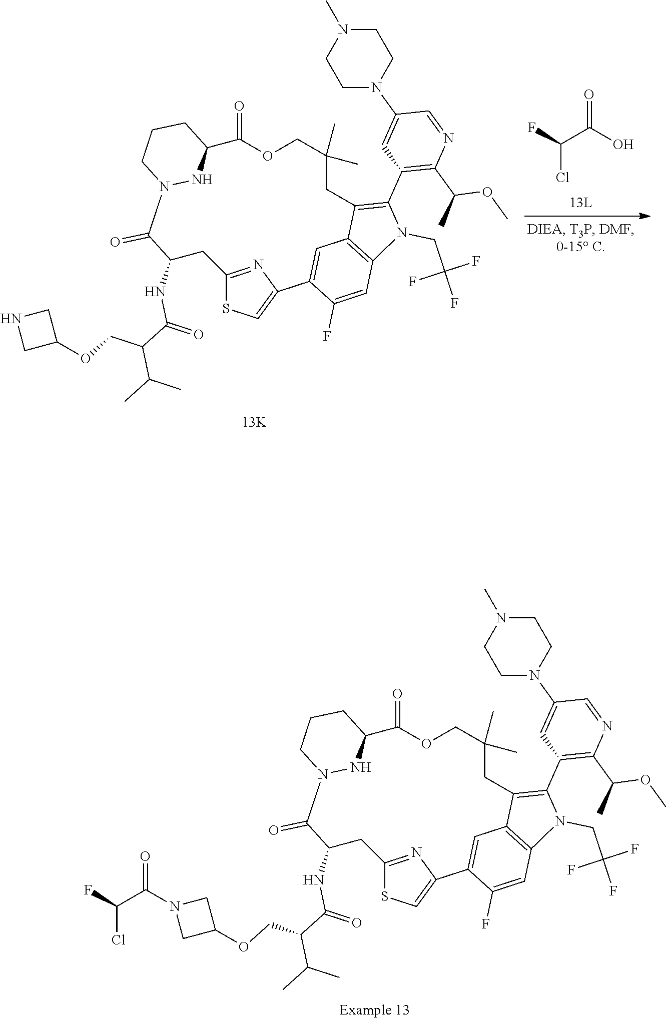

### Step 1: Preparation of ethyl 3-hydroxy-3-methyl-2-methylene-butanoate (compound 13C)

DIBAL-H (152.9 mL, 152.91 mmol) was added dropwise at 0° C. to a solution of NMO (23.8 g, 203.87 mmol) in THF (500 mL). After being stirred at 0° C. for 30 min, the reaction mixture was added with ethyl prop-2-ynoate (compound 13A, 10 g, 101.94 mmol) dropwise at 0° C. After being stirred at 0° C. for another 1 h, the reaction mixture were added with acetone (compound 13B, 5.9 g, 101.94 mmol) and boron trifluoride etherate (12.9 mL, 101.94 mmol) and the resulting mixture was stirred at room temperature for 18 hrs. The reaction was worked up by addition of EA (100 mL). The organic layer was washed with HCl (1N, 37.5 mL), sat. NaHCO₃ (10 mL) and water (10 mL), and then dried over Na₂SO₄ and concentrated under vacuum. The resulting residue was purified by silica gel chromatography (PE:EA=10:1) to afford ethyl 3-hydroxy-3-methyl-2-methylene-butanoate (compound 13C, 4.8 g) as colorless oil. ¹H NMR (400 MHz, CHLOROFORM-d) δ=6.14 (s, 1H), 5.83-5.70 (m, 1H), 4.23 (q, J=7.3 Hz, 2H), 3.93 (br s, 1H), 1.49-1.37 (m, 6H), 1.31 (t, J=7.1 Hz, 3H) ppm.

### Step 2: Preparation of ethyl 2-(bromomethyl)-3-methyl-but-2-enoate (compound 13D)

To a solution of ethyl 3-hydroxy-3-methyl-2-methylene-butanoate (compound 13C, 4.8 g, 30.34 mmol) in THF (100 mL) was added dropwise a solution of Phosphorus tribromide (4.9 g, 18.21 mmol) at 0° C. After being stirred at 15° C. for 2.5 hrs, the reaction mixture was poured into ice water at 0° C., and the aqueous layer was extracted with EA (10 mL). The organic layer was washed with brine, dried over Na₂SO₄, and concentrated to give the crude product, which was purified by silica gel chromatography (petroleum/EtOAc=20/1-10/1) to afford ethyl 2-(bromomethyl)-3-methyl-but-2-enoate (compound 13D, 5.3 g) as colorless oil. ¹H NMR (400 MHz, CHLOROFORM-d) δ=4.30 (s, 2H), 4.24 (d, J=7.2 Hz, 2H), 2.15 (s, 3H), 1.97 (s, 3H), 1.32 (t, J=7.2 Hz, 3H) ppm.

### Step 3: Preparation of benzyl 3-(2-ethoxycarbonyl-3-methyl-but-2-enoxy)azetidine-1-carboxylate (compound 13F)

To a mixture of benzyl 3-hydroxyazetidine-1-carboxylate (compound 13E, 625.0 mg, 3.02 mmol), tetrabutylammonium iodide (116.0 mg, 0.31 mmol) and sodium hydroxide (267.8 mg, 6.7 mmol) in Water (2 mL) and THF (4 mL) was added ethyl 2-(bromomethyl)-3-methyl-but-2-enoate (compound 13D, 1.0 g, 4.52 mmol) at 0° C. After being stirred at 10° C. for 16 hrs, the reaction mixture was dilute with water (5 mL) and the resulting mixture was extracted with EA (10 mL, twice). The combined organic layer was washed with brine (10 mL), dried over Na₂SO₄, filtered and concentrated in vacuo. The residue was purified by silica gel chromatography (petroleum/EtOAc=20/1-10/1) to afford benzyl 3-(2-ethoxycarbonyl-3-methyl-but-2-enoxy)azetidine-1-carboxylate (compound 13F, 590.0 mg) as colorless oil. MS calc'd 348.1 (MH⁺), measured 348.1 (MH⁺).

### Step 4: Preparation of tert-butyl 3-(2-ethoxycarbonyl-3-methyl-butoxy)azetidine-1-carboxylate (compound 13G)

To a solution benzyl 3-(2-ethoxycarbonyl-3-methyl-but-2-enoxy)azetidine-1-carboxylate (compound 13F, 950.0 mg, 2.73 mmol) in Methanol (25 mL) was added Pd/C (240.0 mg, 0.26 mmol) at 15° C. under N₂. The reaction was purged with H₂ and stirred at 50° C. for 2 hrs under H₂ atmosphere. The reaction mixture was filtered and the filter cake was washed with methanol (30 mL). The combined filtrate was concentrated under vacuum and the resulting residue was purified by silica gel chromatography (petroleum/EtOAc=10/1-5/1) to afford tert-butyl 3-(2-ethoxycarbonyl-3-methyl-butoxy)azetidine-1-carboxylate (compound 13G, 700.0 mg) as colorless oil. ¹H NMR (400 MHz, CHLOROFORM-d) δ=4.25-4.11 (m, 3H), 4.08-3.99 (m, 2H), 3.80 (dd, J=4.3, 9.4 Hz, 2H), 3.61-3.46 (m, 2H), 2.45 (ddd, J=4.9, 7.3, 9.5 Hz, 1H), 1.97-1.86 (m, 1H), 1.43 (s, 9H), 1.31-1.24 (m, 3H), 0.99-0.90 (m, 6H) ppm.

### Step 5: Preparation of 2-[(1-tert-butoxycarbonylazetidin-3-yl)oxymethyl]-3-methyl-butanoic acid (compound 13H)

To a mixture of tert-butyl 3-(2-ethoxycarbonyl-3-methyl-butoxy)azetidine-1-carboxylate (compound 13G, 350.0 mg, 1.11 mmol) in methanol (6 mL) was added NaOH aq. (1 N, 6.0 mL, 6.0 mmol). After being stirred at 50° C. for 16 hrs, the reaction mixture was concentrated under vacuum. The resulting residue was diluted with water (10 mL), and the pH of the reaction mixture was adjusted pH=5 by 1N HCl aq., then the reaction mixture was extracted with EA (30 mL, twice). The combined organic layer was washed with brine (50 mL), dried (Na₂SO₄) and concentrated under vacuum to give crude product. The crude was purified by silica gel chromatography (petroleum/EtOAc=5/1-3/1) to afford 2-[(1-tert-butoxycarbonylazetidin-3-yl)oxymethyl]-3-methyl-butanoic acid (compound 13H, 200.0 mg) as colorless oil. ¹H NMR (400 MHz, CHLOROFORM-d) δ=4.22 (tt, J=4.3, 6.4 Hz, 1H), 4.10-4.03 (m, 2H), 3.83 (dd, J=4.3, 9.7 Hz, 2H), 3.63-3.48 (m, 2H), 2.50 (ddd, J=4.8, 7.1, 9.2 Hz, 1H), 1.99 (qd, J=6.8, 13.7 Hz, 1H), 1.44 (s, 9H), 0.99 (dd, J=2.9, 6.8 Hz, 6H) ppm.

### Step 6: Preparation of tert-butyl 3-[(2R)-2-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-3-methyl-butoxy]azetidine-1-carboxylate (compound 131) & tert-butyl 3-[(2S)-2-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-3-methyl-butoxy]azetidine-1-carboxylate (compound 13J)

To a solution of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate D, 225.0 mg, 0.28 mmol) and 2-[(1-tert-butoxycarbonylazetidin-3-yl)oxymethyl]-3-methyl-butanoic acid (compound 13H, 104.9 mg, 0.37 mmol) in DMF (2.5 mL) were added DIEA (0.2 mL, 0.84 mmol) and HATU (213.6 mg, 0.56 mmol) at 0° C. After being stirred at 20° C. for 1 h, the reaction mixture was diluted with water (3 mL), extracted with EtOAc (8 mL, twice). The combined organic layer was washed with brine (10 mL), dried over Na₂SO₄, filtered and concentrated in vacuo. The residue was purified by prep-HPLC to afford tert-butyl 3-[(2R)-2-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-3-methyl-butoxy]azetidine-1-carboxylate (compound 131, faster eluted, 45.0 mg) as a light yellow solid and tert-butyl 3-[(2S)-2-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-3-methyl-butoxy]azetidine-1-carboxylate (compound 13J, slower eluted, 100.0 mg) as a light yellow solid. MS calc'd 1070.6 (MH⁺), measured 1070.6 (MH⁺).

### Step 7: Preparation of (2R)-2-(azetidin-3-yloxymethyl)-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (compound 13K)

To a solution of tert-butyl 3-[(2R)-2-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-3-methyl-butoxy]azetidine-1-carboxylate (compound 131, 45.0 mg, 0.04 mmol) in DCM (0.5 mL) was added TFA (0.2 mL) at 0° C., then the mixture was stirred at 15° C. for 0.5 h. The reaction mixture was concentrated under vacuum to afford (2R)-2-(azetidin-3-yloxymethyl)-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;2,2,2-trifluoroacetic acid (compound 13K, 40.0 mg) as yellow oil. MS calc'd 970.4 (MH⁺), measured 970.4 (MH⁺).

### Step 8: Preparation of (2R)-2-[[1-[(2R)-2-chloro-2-fluoro-acetyl]azetidin-3-yl]oxymethyl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo-[17.5.2.1^{2,5}.1^{9,13}.0^{22,26}]octacosa-1(25),2,5(28),19,22(26),23-hexaen-7-yl]-3-methyl-butanamide (Example 13)

To a solution of (2R)-2-(azetidin-3-yloxymethyl)-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;2,2,2-trifluoroacetic acid (compound 13K, 40.0 mg, 0.04 mmol) in DMF (0.2 mL) were added DIEA (0.1 mL, 0.74 mmol), T₃P (469.6 mg, 0.74 mmol) and (2R)-2-chloro-2-fluoro-acetic acid (compound 13L, 83.0 mg, 0.74 mmol) at 0° C. After being stirred at 20° C. for 1 h, the reaction mixture was concentrated under vacuum and the resulting residue was purified by prep-HPLC to afford (2R)-2-[[1-[(2R)-2-chloro-2-fluoro-acetyl]azetidin-3-yl]oxymethyl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo-[17.5.2.1^{2,5}.1^{9,13}.0^{22,26}]octacosa-1(25),2,5(28),19,22(26),23-hexaen-7-yl]-3-methyl-butanamide (Example 13, 22.6 mg) as a white solid. MS calc'd 1064.5 (MH⁺), measured 1064.5 (MH⁺). ¹H NMR (400 MHz, CHLOROFORM-d) δ=8.65 (br d, J=6.8 Hz, 2H), 7.64 (dd, J=1.9, 4.9 Hz, 1H), 7.26-7.20 (m, 1H), 7.14 (br d, J=11.9 Hz, 1H), 6.77-6.67 (m, 1H), 6.41-6.22 (m, 1H), 5.89 (br t, J=8.1 Hz, 1H), 4.95 (br dd, J=9.0, 16.5 Hz, 1H), 4.65-4.48 (m, 3H), 4.46-4.21 (m, 6H), 4.17-4.09 (m, 1H), 3.86 (br d, J=11.1 Hz, 2H), 3.74-3.69 (m, 3H), 3.60-3.55 (m, 2H), 3.52-3.45 (m, 2H), 3.36 (s, 3H), 3.15-3.09 (m, 2H), 2.94-2.87 (m, 3H), 2.75-2.67 (m, 1H), 2.47-2.34 (m, 1H), 2.30-2.21 (m, 2H), 2.04-1.94 (m, 4H), 1.88-1.79 (m, 1H), 1.70-1.54 (m, 2H), 1.50 (d, J=6.1 Hz, 3H), 1.30-1.20 (m, 1H), 1.02 (dd, J=4.6, 6.3 Hz, 6H), 0.97 (s, 3H), 0.45 (s, 3H) ppm.

### Example 14

### (2S)-2-[[1-[(2R)-2-chloro-2-fluoro-acetyl]azetidin-3-yl]oxymethyl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

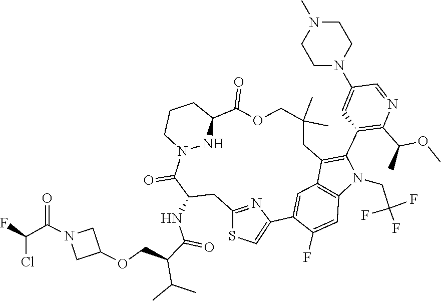

The title compound was prepared in analogy to the preparation of Example 13 by using tert-butyl 3-[(2S)-2-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-3-methyl-butoxy]azetidine-1-carboxylate (compound 13J) instead of tert-butyl 3-[(2R)-2-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-3-methyl-butoxy]azetidine-1-carboxylate (compound 13I). Example 14 (38.0 mg) was obtained as a white solid. MS calc'd 1064.5 (MH⁺), measured 1064.5 (MH⁺). ¹H NMR (400 MHz, CHLOROFORM-d) δ=8.72 (br s, 1H), 8.66 (d, J=7.4 Hz, 1H), 7.65 (s, 1H), 7.14 (d, J=12.0 Hz, 1H), 6.80-6.72 (m, 1H), 6.39-6.23 (m, 1H), 5.97-5.86 (m, 1H), 5.01-4.83 (m, 2H), 4.60-4.49 (m, 5H), 4.36-4.22 (m, 6H), 4.05-3.93 (m, 2H), 3.87 (br d, J=10.4 Hz, 1H), 3.73-3.68 (m, 2H), 3.64-3.53 (m, 4H), 3.45 (s, 1H), 3.36 (s, 3H), 3.20-3.00 (m, 4H), 2.93 (br s, 3H), 2.73 (br t, J=13.2 Hz, 1H), 2.43 (br d, J=14.1 Hz, 1H), 2.30-2.20 (m, 2H), 2.04-1.96 (m, 2H), 1.85-1.78 (m, 1H), 1.69-1.62 (m, 1H), 1.51 (d, J=6.1 Hz, 3H), 1.06 (br dd, J=3.0, 6.6 Hz, 3H), 1.03-1.00 (m, 3H), 0.96 (br s, 3H), 0.46 (s, 3H) ppm.

### Example 15

### (2S)-2-[8-[(2R)-2-chloro-2-fluoro-acetyl]-1-oxo-2,8-diazaspiro[4.5]decan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

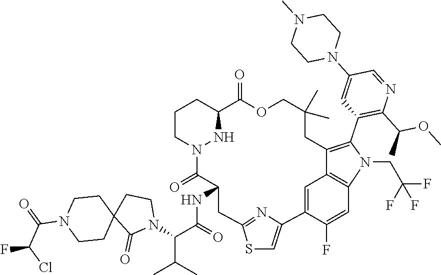

The title compound was prepared in analogy to the preparation of Example 6 by using (2S)-2-(8-tert-butoxycarbonyl-1-oxo-2,8-diazaspiro[4.5]decan-2-yl)-3-methyl-butanoic acid (compound 15F) instead of (2S)-2-(9-tert-butoxycarbonyl-1-oxo-2,9-diazaspiro[5.5]undecan-2-yl)-3-methyl-butanoic acid (compound 6J). Example 15 (56.8 mg) was obtained as a white solid. MS calc'd 1131.5 (MH⁺), measured 1131.5 (MH⁺). ¹H NMR (400 MHz, Methanol-d₄) δ=8.86 (d, J=7.2 Hz, 1H), 8.68 (d, J=7.6 Hz, 1H), 8.50 (d, J=2.8 Hz, 1H), 7.71 (s, 1H), 7.50-7.46 (m, 1H), 7.13-6.92 (m, 1H), 5.68 (br s, 1H), 5.20-5.13 (m, 1H), 4.41 (d, J=14.1 Hz, 1H), 4.36-4.21 (m, 4H), 4.20-3.85 (m, 4H), 3.82-3.68 (m, 3H), 3.61 (d, J=7.3 Hz, 4H), 3.46 (d, J=14.4 Hz, 4H), 3.36-3.35 (m, 2H), 3.24-3.08 (m, 4H), 3.00 (s, 3H), 2.90-2.78 (m, 1H), 2.57 (d, J=14.4 Hz, 1H), 2.28-2.17 (m, 2H), 2.08 (s, 2H), 1.96 (d, J=12.8 Hz, 2H), 1.88-1.77 (m, 2H), 1.76-1.52 (m, 4H), 1.45 (d, J=6.0 Hz, 3H), 1.00 (d, J=6.4 Hz, 3H), 0.97 (s, 3H), 0.92-0.84 (m, 3H), 0.44 (s, 3H) ppm.

The compound 15F was prepared according to the following scheme:

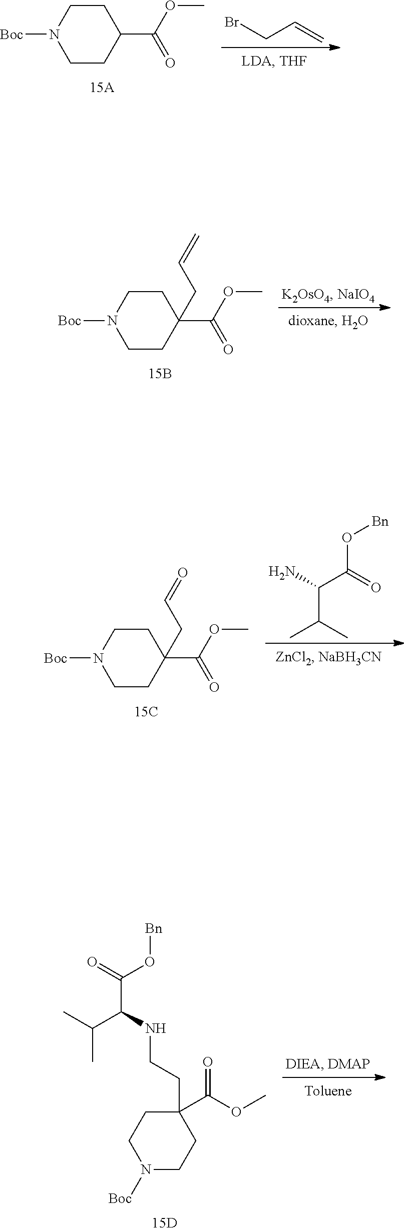

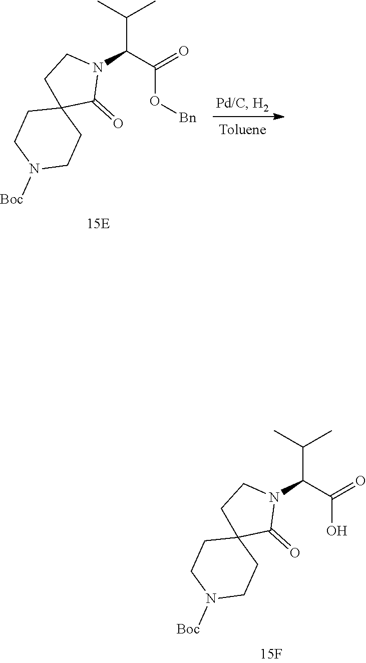 [SMILES: CC(C)[C@@H](C(=O)OCc1ccccc1)N1CCC2(CCN(C(=O)OC(C)(C)C)CC2)C1=O ; CC(C)[C@@H](C(=O)O)N1CCC2(CCN(C(=O)OC(C)(C)C)CC2)C1=O]

### Step 1: Preparation of O₁-tert-butyl O₄-methyl 4-allylpiperidine-1,4-dicarboxylate (compound 15B)

To a solution of O₁-tert-butyl O₄-methyl piperidine-1,4-dicarboxylate (compound 15A, 10.0 g, 41.1 mmol) in THF (100 mL) was added LDA (22.6 mL, 45.21 mmol) dropwise at −70° C. under nitrogen atmosphere. After being stirred for 0.5 h, the reaction mixture was added with allyl bromide (5.5 g, 45.21 mmol) at −70° C., and then the reaction mixture was allowed to warm up to 15° C. and stirred for 2 hrs. After the reaction was completed, it was quenched with saturated NH₄Cl aqueous solution (300 mL) and the reaction mixture was extracted with EtOAc (100 mL, three times). The combined organic layer was washed with brine (200 mL), dried with anhydrous Na₂SO₄, filtered and concentrated under vacuum. The residue was purified by silica gel chromatography (EtOAc in PE=5% to 30%) to afford O₁-tert-butyl O₄-methyl 4-allylpiperidine-1,4-dicarboxylate (compound 15B, 10.8 g) as yellow oil. MS calc'd 284.2 (MH⁺), measured 228.2 (M-C₄H₈+H⁺).

### Step 2: Preparation of O₁-tert-butyl O₄-methyl 4-(2-oxoethyl)piperidine-1,4-dicarboxylate (compound 15C)

To a mixture of O₁-tert-butyl O₄-methyl 4-allylpiperidine-1,4-dicarboxylate (compound 15B, 6.0 g, 21.17 mmol) in 1,4-dioxane (200 mL) and water (20 ML) were added 2,6-Lutidine (4.9 mL, 42.35 mmol) and potassium osmate(VI)dihydrate (0.4 g, 1.06 mmol) in one portion at 0° C. After being stirred at 0° C. for 15 minutes, the reaction mixture was added with sodium metaperiodate (18.1 g, 84.7 mmol) portion-wise at 0° C. and the resulting mixture was warmed to 20° C. and stirred for 2 hrs. The reaction was quenched with saturated Na₂SO₃ aqueous solution (200 mL) and the reaction mixture was extracted with EtOAc (100 mL, three times). The combined organic layer was washed with brine (100 mL), dried with anhydrous Na₂SO₄, filtered and concentrated under vacuum to afford O₁-tert-butyl O₄-methyl 4-(2-oxoethyl)piperidine-1,4-dicarboxylate (compound 15C, 6.0 g) as yellow oil. MS calc'd 286.2 (MH⁺), measured 186.2 (M-C₅H₈O₂+H⁺).

### Step 3: Preparation of O1-tert-butyl O₄-methyl 4-[2-[[(1S)-1-benzyloxycarbonyl-2-methyl-propyl]amino]ethyl]piperidine-1,4-dicarboxylate (compound 15D)

To a mixture of benzyl (2S)-2-amino-3-methyl-butanoate hydrogen chloride (2.2 g, 10.51 mmol) and O₁-tert-butyl O₄-methyl 4-(2-oxoethyl)piperidine-1,4-dicarboxylate (compound 15C, 2.0 g, 7.01 mmol) in methanol (30 mL) was added zinc chloride (1.4 g, 10.51 mmol) at 0° C. After being stirred at 0° C. for 0.5 h, the reaction mixture was added with sodium cyanoborohydride (0.9 g, 14.02 mmol). After being stirred at 20° C. for another 1 h, the reaction was quenched with saturated NH₄Cl aqueous solution (100 mL) and the reaction mixture was extracted with EtOAc (50 mL, three times). The combined organic layer was washed with brine (50 mL), dried with anhydrous Na₂SO₄, filtered and concentrated under vacuum to give a residue, which was purified by column chromatography (EtOAc in PE=10% to 30%) to afford O₁-tert-butyl O₄-methyl 4-[2-1[[(1S)-1-benzyloxycarbonyl-2-methyl-propyl]amino]ethyl]piperidine-1,4-dicarboxylate (compound 15D, 2.46 g) as yellow oil. MS calc'd 477.3 (MH⁺), measured 477.3 (MH⁺).

### Step 4: Preparation of tert-butyl 2-[(1S)-1-benzyloxycarbonyl-2-methyl-propyl]-1-oxo-2,8-diazaspiro[4.5]decane-8-carboxylate (compound 15E)

To a mixture of 1-(tert-butyl) 4-methyl (S)-4-(2-((1-(benzyloxy)-3-methyl-1-oxobutan-2-yl)amino)ethyl)piperidine-1,4-dicarboxylate (compound 15D, 1.2 g, 2.52 mmol) in toluene (20 mL) were added DIEA (4.4 mL, 25.18 mmol) and DMAP (0.3 g, 2.52 mmol). The reaction mixture was heated to 100° C. and stirred for 30 hrs. After the reaction was completed, the reaction mixture was poured into water (30 mL) and extracted with EtOAc (30 mL, three times). The combined organic layer was washed with brine (30 mL), dried with anhydrous Na₂SO₄, filtered and concentrated under vacuum. The residue was purified by reversed phase column to afford tert-butyl 2-[(1S)-1-benzyloxycarbonyl-2-methyl-propyl]-1-oxo-2,8-diazaspiro[4.5]decane-8-carboxylate (compound 15E, 586.0 mg) as yellow oil. MS calc'd 445.3 (MH⁺), measured 467.4 (MNa⁺).

### Step 5: Preparation of (2S)-2-(8-tert-butoxycarbonyl-1-oxo-2,8-diazaspiro[4.5]decan-2-yl)-3-methyl-butanoic acid (compound 15F)

To a solution of tert-butyl 2-[(1S)-1-benzyloxycarbonyl-2-methyl-propyl]-1-oxo-2,8-diazaspiro[4.5]decane-8-carboxylate (compound 15E, 500.0 mg) in toluene (20 mL) was added wet palladium on activated carbon (50.0 mg, 10% wt). The mixture was degassed and purged with H2 for 3 times. The mixture was hydrogenated at 35° C. The reaction solution was filtered and the filter cake was washed with DMF (100 mL). The combined filtrate was concentrated in vacuo to afford (2S)-2-(8-tert-butoxycarbonyl-1-oxo-2,8-diazaspiro[4.5]decan-2-yl)-3-methyl-butanoic acid (compound 15F, 144.0 mg) as a white solid. MS calc'd 355.2 (MH⁺), measured 377.1 (MNa⁺).

### Example 16 and Example 18

### (2S)-2-[(5S)-2-[(2R)-2-chloro-2-fluoro-acetyl]-6-oxo-2,7-diazaspiro[4.5]decan-7-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 16) and (2S)-2-[(5R)-2-[(2R)-2-chloro-2-fluoro-acetyl]-6-oxo-2,7-diazaspiro[4.5]decan-7-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 18)

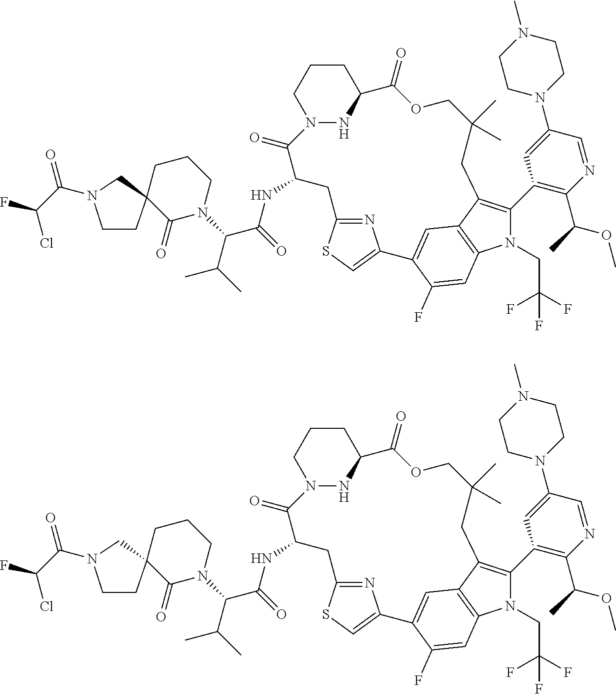

The title compound was prepared in analogy to the preparation of Example 6 by using (2S)-2-[(5S)-2-tert-butoxycarbonyl-6-oxo-2,7-diazaspiro[4.5]decan-7-yl]-3-methyl-butanoic acid (compound 16o 1) instead of (2S)-2-(9-tert-butoxycarbonyl-1-oxo-2,9-diazaspiro[5.5]undecan-2-yl)-3-methyl-butanoic acid (compound 6J). Example 16 (14.0 mg) was obtained as a white solid. MS calc'd 1131.5 (MH⁺), measured 1131.4 (MH⁺). ¹H NMR (400 MHz, Methanol-d₄) δ=8.69 (dd, J=2.0, 7.6 Hz, 1H), 8.50 (d, J=2.8 Hz, 1H), 7.80-7.70 (m, 1H), 7.51-7.45 (m, 2H), 6.91-6.73 (m, 1H), 5.72-5.64 (m, 1H), 5.23-5.13 (m, 2H), 4.42 (d, J=12.0 Hz, 1H), 4.27-4.20 (m, 2H), 4.10-4.00 (m, 3H), 3.93-3.82 (m, 2H), 3.79-3.69 (m, 3H), 3.66-3.56 (m, 3H), 3.52-3.43 (m, 4H), 3.36-3.34 (m, 4H), 3.19-3.12 (m, 2H), 3.00 (s, 3H), 2.87-2.79 (m, 1H), 2.60-2.53 (m, 1H), 2.52-2.41 (m, 1H), 2.39-2.18 (m, 3H), 2.07-1.81 (m, 8H), 1.68-1.61 (m, 1H), 1.45 (d, J=6.0 Hz, 3H), 1.00-0.96 (m, 6H), 0.90 (d, J=6.4 Hz, 3H), 0.47-0.41 (m, 3H) ppm.

The title compound was prepared in analogy to the preparation of Example 6 by using (2S)-2-[(5R)-2-tert-butoxycarbonyl-6-oxo-2,7-diazaspiro[4.5]decan-7-yl]-3-methyl-butanoic acid (compound 16o 2) instead of (2S)-2-(9-tert-butoxycarbonyl-1-oxo-2,9-diazaspiro[5.5]undecan-2-yl)-3-methyl-butanoic acid (compound 6J). Example 18 (25.6 mg) was obtained as a white solid. MS calc'd 1131.4 (MH⁺), measured 1131.5 (MH⁺). ¹H NMR (400 MHz, Methanol-d₄) δ=8.68 (d, J=7.6 Hz, 1H), 8.50 (d, J=2.8 Hz, 1H), 7.74-7.71 (m, 1H), 7.50-7.45 (m, 2H), 6.91-6.76 (m, 1H), 5.72-5.65 (m, 1H), 5.24-5.11 (m, 2H), 4.83-4.79 (m, 3H), 4.47-4.18 (m, 2H), 4.12-3.87 (m, 3H), 3.81-3.60 (m, 6H), 3.56-3.44 (m, 3H), 3.41-3.33 (m, 6H), 3.26-3.12 (m, 3H), 3.00 (s, 3H), 2.87-2.78 (m, 1H), 2.26-2.46 (m, 2H), 2.39-2.19 (m, 2H), 2.05-1.79 (m, 7H), 1.71-1.60 (m, 1H), 1.44 (d, J=6.4 Hz, 3H), 1.02-0.95 (m, 6H), 0.90 (d, J=6.4 Hz, 3H), 0.44 (s, 3H) ppm.

The compound 16o 1 and compound 16o 2 was prepared according to the following scheme:

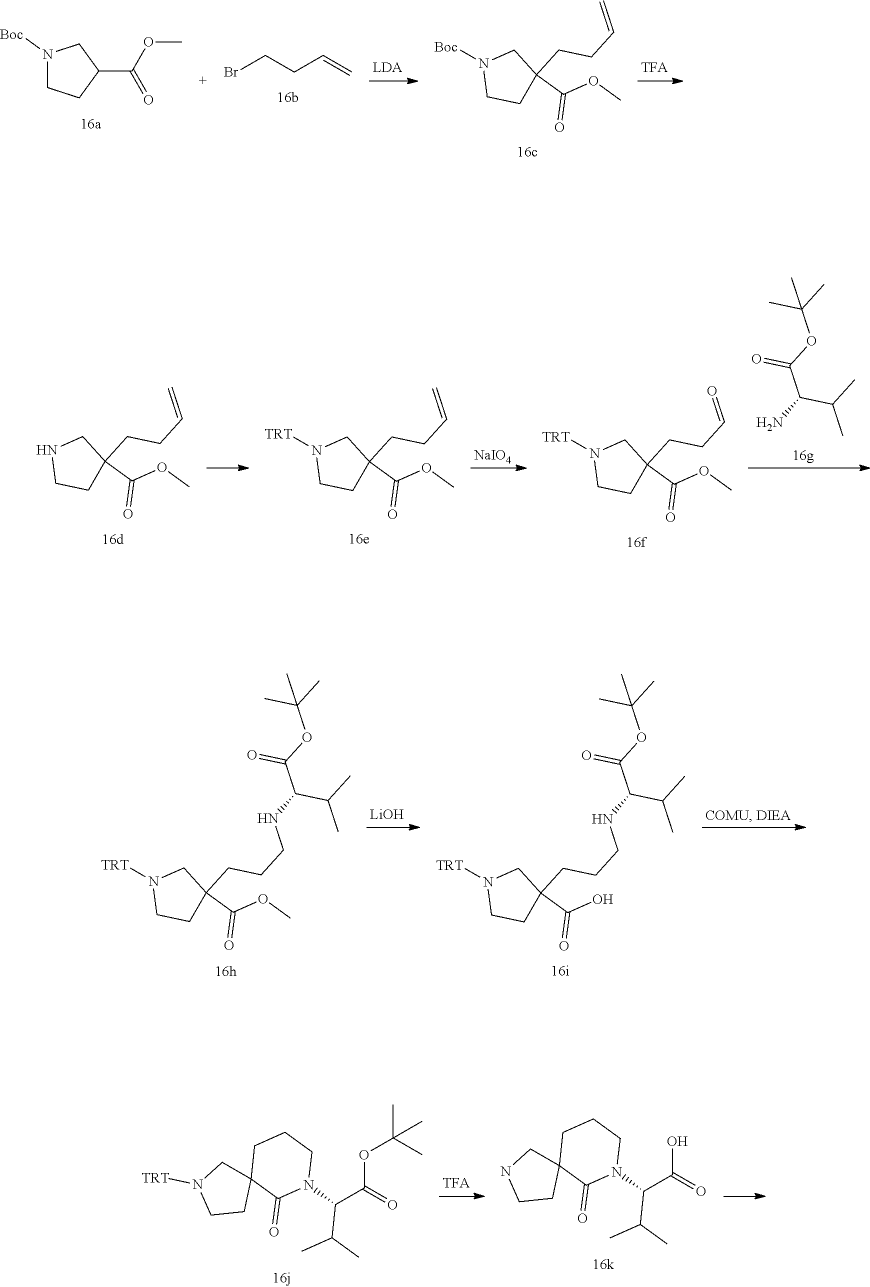

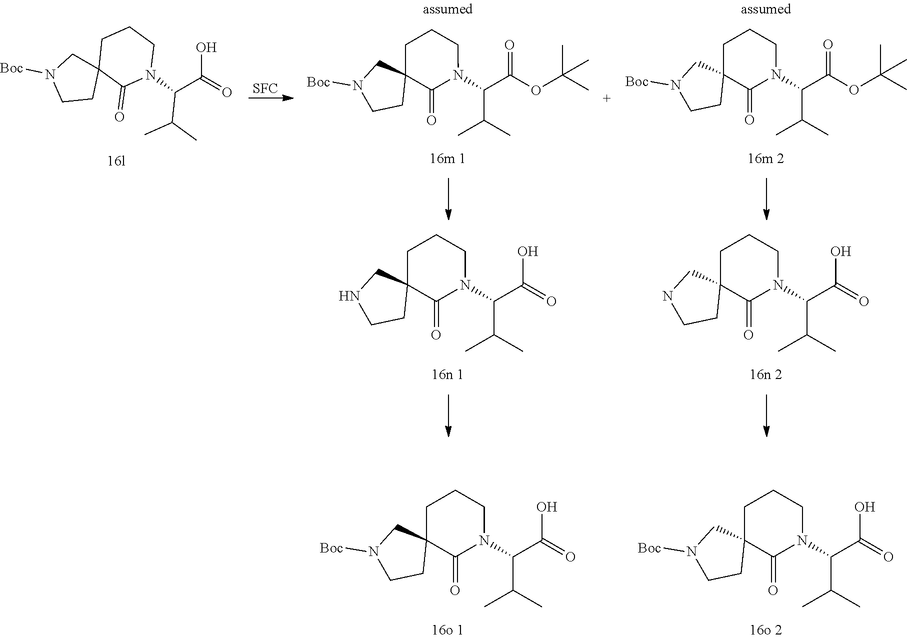

### Step 1: Preparation of 1-(tert-butyl) 3-methyl 3-(but-3-en-1-yl)pyrrolidine-1,3-dicarboxylate (compound 16c)

To a solution of 1-(tert-butyl) 3-methyl pyrrolidine-1,3-dicarboxylate (compound 16a, 25.0 g, 109.04 mmol) in THF (300 mL) was added LDA (59.9 mL, 119.95 mmol) at −70° C. under nitrogen atmosphere. After being stirred for 0.5 h, the reaction mixture was added with 4-bromo-1-butene (compound 16b, 16.1 g, 119.95 mmol). After being stirred at 20° C. for 2.5 hrs, the reaction was quenched with saturated NH₄Cl (100 mL) and the reaction mixture was extracted with EtOAc (150 mL, twice). The combined organic layer was dried over anhydrous sodium sulfate, filtered and concentrated under vacuum to give a residue, which was purified by silica gel column (EtOAc in PE=0%-5%) to afford 1-(tert-butyl) 3-methyl 3-(but-3-en-1-yl)pyrrolidine-1,3-dicarboxylate (compound 16c, 13.2 g) as yellow oil. ¹H NMR (400 MHz, Methanol-d₄) δ=5.83-5.73 (m, 1H), 5.05-4.93 (m, 2H), 3.84 (d, J=10.8 Hz, 1H), 3.71 (s, 3H), 3.46-3.37 (m, 1H), 3.29-3.11 (m, 2H), 2.40-2.32 (m, 1H), 2.01-1.91 (m, 2H), 1.90-1.74 (m, 3H), 1.46 (d, J=3.2 Hz, 9H) ppm.

### Step 2: Preparation of methyl 3-(but-3-en-1-yl)pyrrolidine-3-carboxylate (compound 16d)

To a solution of 1-(tert-butyl) 3-methyl 3-(but-3-en-1-yl)pyrrolidine-1,3-dicarboxylate (compound 16c, 13.2 g, 46.58 mmol) in 1,4-dioxane (50 mL) was added HCl solution (50.0 mL, 4 M in dioxane). After being stirred at 20° C. for 0.5 h, the reaction mixture was concentrated under vacuum to afford methyl 3-(but-3-en-1-yl)pyrrolidine-3-carboxylate hydrochloride salt (compound 16d, 10.2 g) as yellow oil.

### Step 3: Preparation of methyl 3-(but-3-en-1-yl)-1-tritylpyrrolidine-3-carboxylate (compound 16e)

To a solution of methyl 3-(but-3-en-1-yl)pyrrolidine-3-carboxylate hydrochloride salt (compound 16d, 3.0 g, 13.65 mmol) in ACN (70 mL) were added triphenylmethyl chloride (3.81 g, 13.65 mmol) and potassium carbonate (4.72 g, 34.14 mmol). The mixture was stirred at 20° C. for 12 hrs. After the reaction was completed, the mixture was poured into water (100 mL) and extracted with EtOAc (30 mL, three times). The combined organic layer was washed with brine (30 mL), dried over anhydrous sodium sulfate, filtered and concentrated under vacuum to give a residue, which was purified by silica gel column (EtOAc in PE=0%-5%) to afford methyl 3-(but-3-en-1-yl)-1-tritylpyrrolidine-3-carboxylate (compound 16e, 1.3 g) as yellow oil. ¹H NMR (400 MHz, CDCl₃) δ=7.48-7.45 (m, 6H), 7.26-7.23 (m, 6H), 7.15 (t, J=7.2 Hz, 3H), 5.78-5.68 (m, 1H), 5.00-4.89 (m, 2H), 3.74 (s, 3H), 3.00 (d, J=9.6 Hz, 1H), 2.61-2.55 (m, 1H), 2.43-2.35 (m, 1H), 2.20-2.14 (m, 1H), 2.03 (s, 1H), 1.98-1.83 (m, 2H), 1.82-1.61 (m, 2H), 1.52-1.45 (m, 1H) ppm.

### Step 4: Preparation of methyl 3-(3-oxopropyl)-1-tritylpyrrolidine-3-carboxylate (compound 16f)

To a solution of methyl 3-(but-3-en-1-yl)-1-tritylpyrrolidine-3-carboxylate (compound 16e, 660 mg, 1.55 mmol) in THF (20 mL) and water (10 mL) were added potassium osmate (VI) (5 mg, 0.02 mmol) and sodium periodate (663 mg, 3.1 mmol). The reaction mixture was stirred at 20° C. for 2 hrs. After the reaction was completed, it was quenched with sat. NH₄Cl aqueous solution and the reaction mixture was extracted with EtOAc (50 mL, twice). The combined organic layer was washed with brine, dried over anhydrous sodium sulfate, filtered and concentrated under vacuum to give a residue, which was purified by silica gel column (EtOAc in PE=0%-5%) to afford methyl 3-(3-oxopropyl)-1-tritylpyrrolidine-3-carboxylate (compound 16f, 300 mg) as yellow oil. ¹H NMR (400 MHz, CDCl₃) δ=9.75-9.69 (m, 1H), 7.52-7.48 (m, 6H), 7.29 (t, J=4.0 Hz, 6H), 7.19 (t, J=7.2 Hz, 3H), 3.77 (s, 3H), 3.05 (d, J=9.2 Hz, 1H), 2.67-2.57 (m, 1H), 2.47-2.31 (m, 3H), 2.29-2.21 (m, 1H), 2.04-1.87 (m, 2H), 1.62-1.43 (m, 2H) ppm.

### Step 5: Preparation of methyl 3-(3-(((S)-1-(tert-butoxy)-3-methyl-1-oxobutan-2-yl)amino)propyl)-1-tritylpyrrolidine-3-carboxylate (compound 16h)

To a mixture of methyl 3-(3-oxopropyl)-1-tritylpyrrolidine-3-carboxylate (compound 16f, 550.0 mg, 1.29 mmol), H-VAL-OTBU HCl (compound 16g, 296.7 mg, 1.42 mmol) and zinc chloride (192.8 mg, 1.42 mmol) in methanol (6 mL) was added sodium cyanoborohydride (88.9 mg, 1.42 mmol) at 0° C. The mixture was stirred at 25° C. for 2 hrs. After the reaction was completed, water (30 mL) was added to the reaction mixture. The resulting mixture was concentrated under vacuum. The resulting suspension was diluted with water (40 mL) and extracted with EtOAc (30 mL, twice). The combined organic layer was washed with brined, dried over anhydrous sodium sulfate, filtered and concentrated under vacuum to give a residue which was purified by column chromatography (EtOAc in PE=0%-10%) to afford methyl 3-(3-(((S)-1-(tert-butoxy)-3-methyl-1-oxobutan-2-yl)amino)propyl)-1-tritylpyrrolidine-3-carboxylate (compound 16 h, 510 mg) as yellow oil. ¹H NMR (400 MHz, CDCl₃) δ=7.47 (d, J=8.0 Hz, 6H), 7.28-7.27 (m, 1H), 7.27-7.23 (m, 5H), 7.18-7.13 (m, 3H), 3.78-3.73 (m, 3H), 3.05 (t, J=8.4 Hz, 1H), 2.79 (dd, J=2.4, 6.0 Hz, 1H), 2.67-2.59 (m, 1H), 2.55-2.49 (m, 1H), 2.43-2.31 (m, 2H), 2.14-2.09 (m, 1H), 1.99-1.94 (m, 1H), 1.88-1.82 (m, 1H), 1.68-1.64 (m, 3H), 1.60-1.55 (m, 2H), 1.48-1.45 (m, 9H), 1.37-1.33 (m, 1H), 0.94-0.90 (m, 6H) ppm.

### Step 6: Preparation of 3-(3-(((S)-1-(tert-butoxy)-3-methyl-1-oxobutan-2-yl)amino)propyl)-1-tritylpyrrolidine-3-carboxylic acid (compound 16i)

To a solution of methyl 3-(3-(((S)-1-(tert-butoxy)-3-methyl-1-oxobutan-2-yl)amino)propyl)-1-tritylpyrrolidine-3-carboxylate (compound 16 h, 510 mg, 0.8 mmol) in methanol (6 mL), water (0.6 mL) and THF (0.6 mL) was added lithium hydroxide monohydrate (336 mg, 8 mmol). The mixture was stirred at 60° C. for 12 hrs. After the reaction was completed, the mixture was diluted with water (20 mL), neutralized with 1 M HCl aqueous and extracted with EtOAc (20 mL, twice). The combined organic layer was washed with brine, dried over anhydrous sodium sulfate, filtered and concentrated under vacuum to afford 3-(3-(((S)-1-(tert-butoxy)-3-methyl-1-oxobutan-2-yl)amino)propyl)-1-tritylpyrrolidine-3-carboxylic acid (compound 16i, 440 mg) as white gum. ¹H NMR (400 MHz, CDCl₃) δ=7.46 (d, J=8.0 Hz, 6H), 7.26-7.19 (m, 6H), 7.16-7.10 (m, 3H), 3.10-2.86 (m, 4H), 2.54-2.45 (m, 2H), 2.37-2.22 (m, 2H), 2.18-2.10 (m, 1H), 2.04-1.97 (m, 1H), 1.68-1.54 (m, 2H), 1.49-1.44 (m, 9H), 1.41-1.35 (m, 2H), 0.99-0.89 (m, 6H) ppm.

### Step 7: Preparation of tert-butyl (2S)-3-methyl-2-(6-oxo-2-trityl-2,7-diazaspiro[4.5]decan-7-yl)butanoate (compound 16j)

To a solution of 3-(3-(((S)-1-(tert-butoxy)-3-methyl-1-oxobutan-2-yl)amino)propyl)-1-tritylpyrrolidine-3-carboxylic acid (compound 16i, 440 mg, 0.77 mmol) in DMF (5 mL) was added DIEA (0.67 mL, 3.85 mmol) and COMU (594 mg, 1.39 mmol) at 0° C. The mixture was stirred at 20° C. for 1 h. After the reaction was completed, the reaction mixture was poured into water (20 mL) and extracted with EtOAc (20 mL, twice). The combined organic layer was washed with brine, dried over anhydrous sodium sulfate, filtered and concentrated under vacuum to give a residue, which was purified by silica gel column (EtOAc in PE=0%-10%) to afford tert-butyl (2S)-3-methyl-2-(6-oxo-2-trityl-2,7-diazaspiro[4.5]decan-7-yl)butanoate (compound 16j, 420 mg) as yellow oil. MS calc'd 553.3 (MH⁺), measured 553.2 (MH⁺).

### Step 8: Preparation of (2S)-3-methyl-2-(6-oxo-2,7-diazaspiro[4.5]decan-7-yl)butanoic acid (compound 16k)

To a solution of tert-butyl (2S)-3-methyl-2-(6-oxo-2-trityl-2,7-diazaspiro[4.5]decan-7-yl)butanoate (compound 16j, 550 mg, 1 mmol) in DCM (3 mL) was added TFA (3.0 mL). The mixture was stirred at 20° C. for 12 hrs. After the reaction was completed, the mixture was concentrated under vacuum to give a residue, which was dissolved in water (10 mL) and the aqueous phase was washed with EtOAc (20 mL, twice). The aqueous phase was concentrated under vacuum to afford (2S)-3-methyl-2-(6-oxo-2,7-diazaspiro[4.5]decan-7-yl)butanoic acid in TFA salt form (compound 16k, 365 mg) as colorless oil. MS calc'd 255.2 (MH⁺), measured 255.2 (MH⁺)

### Step 9: Preparation of (2S)-2-(2-(tert-butoxycarbonyl)-6-oxo-2,7-diazaspiro[4.5]decan-7-yl)-3-methylbutanoic acid (compound 16l)

To a solution of (2S)-3-methyl-2-(6-oxo-2,7-diazaspiro[4.5]decan-7-yl)butanoic acid in TFA salt form (compound 16k, 365 mg, 0.99 mmol) in THF (10 mL) and water (10 mL) were added sodium carbonate (210 mg, 1.98 mmol) and di-t-butyldicarbonate (259 mg, 1.19 mmol). The mixture was stirred at 20° C. for 2 hrs. After the reaction was completed, the mixture was neutralized with 1 M HCl aq. and the reaction mixture was extracted with EtOAc (20 mL, twice). The combined organic layer was washed with brine, dried over sodium sulfate and concentrated under vacuum to afford (2S)-2-(2-(tert-butoxycarbonyl)-6-oxo-2,7-diazaspiro[4.5]decan-7-yl)-3-methylbutanoic acid (compound 16l, 350 mg) as a white solid.

### Step 10: Preparation of tert-butyl (5S)-7-[(1S)-1-tert-butoxycarbonyl-2-methyl-propyl]-6-oxo-2,7-diazaspiro[4.5]decane-2-carboxylate (compound 16m 1) and tert-butyl (5R)-7-[(1S)-1-tert-butoxycarbonyl-2-methyl-propyl]-6-oxo-2,7-diazaspiro[4.5]decane-2-carboxylate (compound 16m 2)

Tert-butyl 7-[(1S)-1-tert-butoxycarbonyl-2-methyl-propyl]-6-oxo-2,7-diazaspiro[4.5]decane-2-carboxylate (compound 16l, 680.0 mg, 1.66 mmol) was separated by SFC (regis (S,S)whelk-O1 (250 mm, twice 5 mm×10 um), mobile phase: (0.1% NH₃H₂O) IPA, B %: 15%-15%, 4.4 min) to afford tert-butyl (5S)-7-[(1S)-1-tert-butoxycarbonyl-2-methyl-propyl]-6-oxo-2,7-diazaspiro[4.5]decane-2-carboxylate (compound 16m 1, 245.0 mg, faster eluted) as yellow oil and tert-butyl (5R)-7-[(1S)-1-tert-butoxycarbonyl-2-methyl-propyl]-6-oxo-2,7-diazaspiro[4.5]decane-2-carboxylate (compound 16m 2, 223.0 mg, slower eluted) as yellow oil. MS calc'd 411.3 (MH⁺), measured 411.3 (MH⁺).

### Step 11: Preparation of (2S)-3-methyl-2-[(5S)-6-oxo-2,7-diazaspiro[4.5]decan-7-yl]butanoic acid (compound 16n 1)

To a solution of tert-butyl (5S)-7-[(1S)-1-tert-butoxycarbonyl-2-methyl-propyl]-6-oxo-2,7-diazaspiro[4.5]decane-2-carboxylate (compound 16m 1, 245.0 mg, 0.6 mmol) in DCM (1 mL) was added TFA (1.0 mL, 12.98 mmol). The mixture was stirred at 20° C. for 1 h. After the reaction was completed, the mixture was concentrated under vacuum to afford (2S)-3-methyl-2-[(5S)-6-oxo-2,7-diazaspiro[4.5]decan-7-yl]butanoic acid (compound 16n 1, 219.0 mg, TFA salt) as colorless liquid, which was used directly in the next step. MS calc'd 255.2 (MH⁺), measured 255.2 (MH⁺).

### Step 12: Preparation of (2S)-2-[(5S)-2-tert-butoxycarbonyl-6-oxo-2,7-diazaspiro[4.5]decan-7-yl]-3-methyl-butanoic acid (compound 16 ol)

To a solution of (2S)-3-methyl-2-[(5S)-6-oxo-2,7-diazaspiro[4.5]decan-7-yl]butanoic acid (compound 16n 1, 219.0 mg, 0.59 mmol) in THF (2 mL) and water (2 mL) was added sodium carbonate (189.05 mg, 1.78 mmol) and di-t-butyldicarbonate (155.71 mg, 0.71 mmol). The mixture was stirred at 20° C. for 1 h. After the reaction was completed, the mixture was filtered and the filtrate was concentrated under vacuum to give a residue, which was purified by reversed phase flash column to afford (2S)-2-[(5S)-2-tert-butoxycarbonyl-6-oxo-2,7-diazaspiro[4.5]decan-7-yl]-3-methyl-butanoic acid (compound 16o 1, 200.0 mg) as a white solid. MS calc'd 353.2 (MH⁻), measured 353.0 (MH⁻).

### Step 13: Preparation of (2S)-3-methyl-2-[(5R)-6-oxo-2,7-diazaspiro[4.5]decan-7-yl]butanoic acid (compound 16n 2)

To a solution of tert-butyl (5R)-7-[(1S)-1-tert-butoxycarbonyl-2-methyl-propyl]-6-oxo-2,7-diazaspiro[4.5]decane-2-carboxylate (compound 16m 2, 223.0 mg, 0.54 mmol) in DCM (1.5 mL) was added TFA (1.5 mL, 19.47 mmol). The mixture was stirred at 20° C. for 1 h. After the reaction was completed, the mixture was concentrated under vacuum to afford (2S)-3-methyl-2-[(5R)-6-oxo-2,7-diazaspiro[4.5]decan-7-yl]butanoic acid (compound 16n 2, 200.0 mg, TFA salt) as colorless liquid, which was used directly in the next step. MS calc'd 255.2 (MH⁺), measured 255.1 (MH⁺).

### Step 14: Preparation of (2S)-2-[(5R)-2-tert-butoxycarbonyl-6-oxo-2,7-diazaspiro[4.5]decan-7-yl]-3-methyl-butanoic acid (compound 16o 2)

To a solution of (2S)-3-methyl-2-[(5R)-6-oxo-2,7-diazaspiro[4.5]decan-7-yl]butanoic acid (compound 16n 2, 200.0 mg, 0.54 mmol) in THF (2 mL) and water (2 mL) was added sodium carbonate (115.1 mg, 1.09 mmol) and di-t-butyldicarbonate (142.2 mg, 0.65 mmol). The mixture was stirred at 20° C. for 1 h. After the reaction was completed, the mixture was filtered and filtrate was concentrated under vacuum to give a residue, which was purified by reversed phase flash column to afford (2S)-2-[(5R)-2-tert-butoxycarbonyl-6-oxo-2,7-diazaspiro[4.5]decan-7-yl]-3-methyl-butanoic acid (compound 16 o2, 150.0 mg) as a white solid. MS calc'd 355.2 (MH⁺), measured 377.2 (MNa⁺).

### Example 20

### (2S)-2-[9-[(2R)-2-chloro-2-fluoro-acetyl]-1-oxo-2,9-diazaspiro[5.5]undecan-2-yl]-N-[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

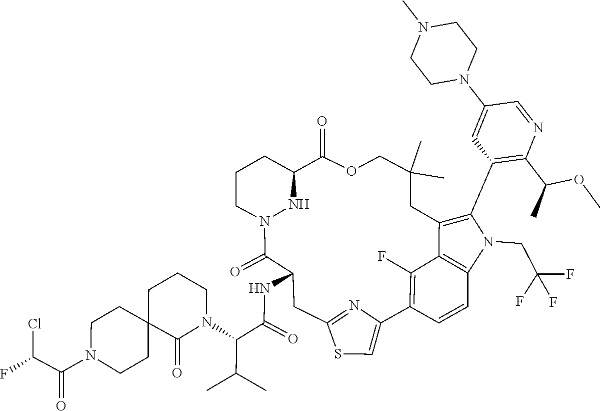

The title compound was prepared in analogy to the preparation of Example 6 by using (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E) instead of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 20 (6.1 mg) was obtained as a white solid. MS calc'd 1145.5 (MH⁺), measured 1145.5 (MH⁺). ¹H NMR (400 MHz, Methanol-d₄) δ=8.52 (s, 1H), 7.61-7.45 (m, 4H), 7.09-6.91 (m, 1H), 5.97-5.84 (m, 1H), 5.24-5.06 (m, 3H), 4.69-4.56 (m, 0.5H), 4.44-4.33 (m, 0.5H), 4.20-3.96 (m, 3H), 3.90-3.40 (m, 10H), 3.27-3.10 (m, 12H), 3.00 (s, 3H), 2.95-2.75 (m, 1H), 2.71-2.53 (m, 1H), 2.25-2.04 (m, 2H), 2.00-1.79 (m, 4H), 1.72-1.53 (m, 3H), 1.44 (d, J=6.0 Hz, 3H), 1.37-1.19 (m, 2H), 0.94 (d, J=6.0 Hz, 3H), 0.89-0.73 (m, 7H), 0.71-0.53 (m, 3H) ppm.

### Example 22 and Example 23

### (2S)-2-[(5R)-2-[(2S)-2-chloro-2-fluoro-acetyl]-6-oxo-2,7-diazaspiro[4.5]decan-7-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 22) and (2S)-2-[(5S)-2-[(2S)-2-chloro-2-fluoro-acetyl]-6-oxo-2,7-diazaspiro[4.5]decan-7-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 23)

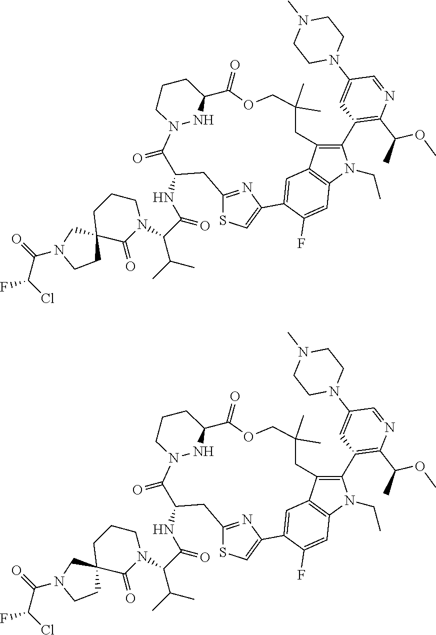

The title compounds were prepared in analogy to the preparation of Example 6 by using (2S)-2-chloro-2-fluoro-acetic acid, (2S)-2-[(5S)-2-tert-butoxycarbonyl-6-oxo-2,7-diazaspiro[4.5]decan-7-yl]-3-methyl-butanoic acid (compound 16o 1) and (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (2R)-2-chloro-2-fluoro-acetic acid, (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 22 (4.1 mg) was obtained as a white solid. MS calc'd 1077 (MH⁺), measured 1077 (MH⁺). ¹HNMR (400 MHz, DMSO-d₆) δ 9.98-10.20 (m, 1H), 8.42-8.62 (m, 2H), 8.10-8.34 (m, 1H), 7.71 (d, J=2.50 Hz, 1H), 7.57 (d, J=13.01 Hz, 1H), 7.39 (br s, 1H), 7.15 (d, J=10.01 Hz, 1H), 7.00-7.07 (m, 1H), 5.41-5.54 (m, 1H), 5.18 (br dd, J=19.51, 11.88 Hz, 1H), 4.81 (br dd, J=12.88, 11.38 Hz, 1H), 4.23-4.28 (m, 2H), 4.13 (br s, 5H), 3.57 (br s, 3H), 3.34-3.34 (m, 3H), 2.82-2.94 (m, 3H), 2.66-2.81 (m, 7H), 2.41 (br s, 1H), 2.31-2.34 (m, 3H), 2.21 (br d, J=6.88 Hz, 1H), 1.69-1.88 (m, 7H), 1.50 (br s, 1H), 1.33 (d, J=6.13 Hz, 3H), 1.18-1.26 (m, 3H), 0.84-0.98 (m, 11H), 0.80 (br t, J=7.32 Hz, 3H), 0.35 (s, 3H) ppm.

Example 23 was prepared in analogy to the preparation of Example 6 by using (2S)-2-chloro-2-fluoro-acetic acid, (2S)-2-[(5R)-2-tert-butoxycarbonyl-6-oxo-2,7-diazaspiro[4.5]decan-7-yl]-3-methyl-butanoic acid (compound 16o 2) and (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (2R)-2-chloro-2-fluoro-acetic acid, (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 23 (2.2 mg) was obtained as a white solid. MS calc'd 1077 (MH⁺), measured 1077 (MH⁺). ¹H NMR (400 MHz, METHANOL-d₄) δ ppm 8.66 (d, J=7.50 Hz, 1H), 8.47 (d, J=2.88 Hz, 1H), 7.66 (br d, J=7.13 Hz, 1H), 7.45 (d, J=2.75 Hz, 1H), 7.33-7.36 (m, 1H), 6.73-6.94 (m, 1H), 5.32-5.38 (m, 2H), 4.37-4.53 (m, 2H), 4.09-4.30 (m, 5H), 3.68-3.81 (m, 4H), 3.61 (br d, J=7.00 Hz, 4H), 3.04 (q, J=7.75 Hz, 4H), 2.94 (br s, 3H), 2.77-2.87 (m, 2H), 2.54-2.66 (m, 2H), 2.16-2.22 (m, 3H), 2.01-2.05 (m, 3H), 1.88 (br s, 2H), 1.63 (dt, J=15.26, 4.31 Hz, 8H), 1.43 (d, J=6.25 Hz, 3H), 0.89-1.00 (m, 15H), 0.49 (br d, J=2.63 Hz, 3H).

### Example 24

### (3S)-1-[(2R)-2-chloro-2-fluoro-acetyl]-N-[(1S)-1-[[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide

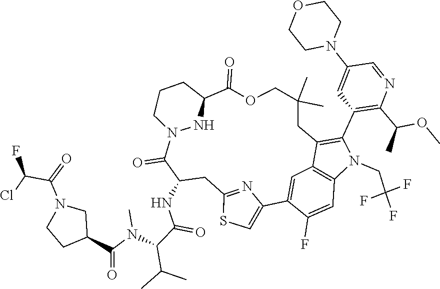

The title compound was prepared in analogy to the preparation of Example 1 by using (7S,13S)-7-amino-24-fluoro-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate I) instead of (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E). Example 24 (18.9 mg) was obtained as a white solid. MS calc'd 1092.4 (MH⁺), measured 1092.5 (MH⁺). ¹H NMR (400 MHz, Methanol-d₄) δ=8.79-8.66 (m, 1H), 8.60-8.35 (m, 1H), 7.81-7.67 (m, 2H), 7.57-7.42 (m, 1H), 6.94-6.72 (m, 1H), 5.76-5.61 (m, 1H), 5.33-5.10 (m, 1H), 4.80-4.60 (m, 2H), 4.43-4.10 (m, 3H), 3.88-3.52 (m, 12H), 3.43-3.36 (m, 8H), 3.11-2.99 (m, 4H), 2.85-2.81 (m, 1H), 2.68-2.64 (m, 1H), 2.34-2.21 (m, 4H), 1.95-1.61 (m, 3H), 1.48-1.47 (m, 3H), 1.07-0.81 (m, 9H), 0.60-0.42 (m, 3H) ppm.

### Example 25

### (2S)-2-[2-[(2R)-2-chloro-2-fluoro-acetyl]-5-oxo-2,6-diazaspiro[3.4]octan-6-yl]-N-[(7S,13S)-24-fluoro-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

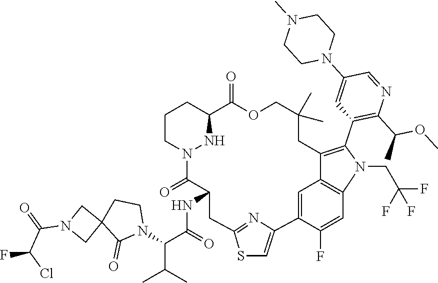

The title compound was prepared in analogy to the preparation of Example 6 by using (2S)-2-(8-tert-butoxycarbonyl-1-oxo-2,8-diazaspiro[4.5]decan-2-yl)-3-methyl-butanoic acid (compound 25H) instead of (2S)-2-(9-tert-butoxycarbonyl-1-oxo-2,9-diazaspiro[5.5]undecan-2-yl)-3-methyl-butanoic acid (compound 6J). Example 25 (20.8 mg) was obtained as a white solid. MS calc'd 1103.4 (MH⁺), measured 1103.7 (MH⁺). ¹H NMR (400 MHz, Methanol-d₄) δ=8.96 (d, J=8.4 Hz, 1H), 8.68 (d, J=7.6 Hz, 1H), 8.50 (d, J=2.8 Hz, 1H), 7.72 (dd, J=2.4, 17.6 Hz, 1H), 7.54-7.41 (m, 2H), 6.80-6.61 (m, 1H), 5.72-5.60 (m, 1H), 5.21-5.14 (m, 1H), 4.65-4.55 (m, 1H), 4.45-4.38 (m, 2H), 4.38-4.28 (m, 2H), 4.26-4.19 (m, 2H), 4.16-4.00 (m, 3H), 3.81-3.67 (m, 3H), 3.66-3.53 (m, 3H), 3.51-3.39 (m, 4H), 3.35 (s, 3H), 3.25-3.10 (m, 3H), 3.00 (s, 3H), 2.89-2.78 (m, 1H), 2.60-2.52 (m, 1H), 2.47-2.36 (m, 2H), 2.28-2.19 (m, 2H), 1.99-1.92 (m, 1H), 1.89-1.78 (m, 1H), 1.70-1.59 (m, 1H), 1.44 (d, J=6.0 Hz, 3H), 1.01 (dd, J=3.6, 6.4 Hz, 3H), 0.97 (s, 3H), 0.92-0.86 (m, 3H), 0.43 (s, 3H) ppm.

The compound 25H was prepared according to the following scheme:

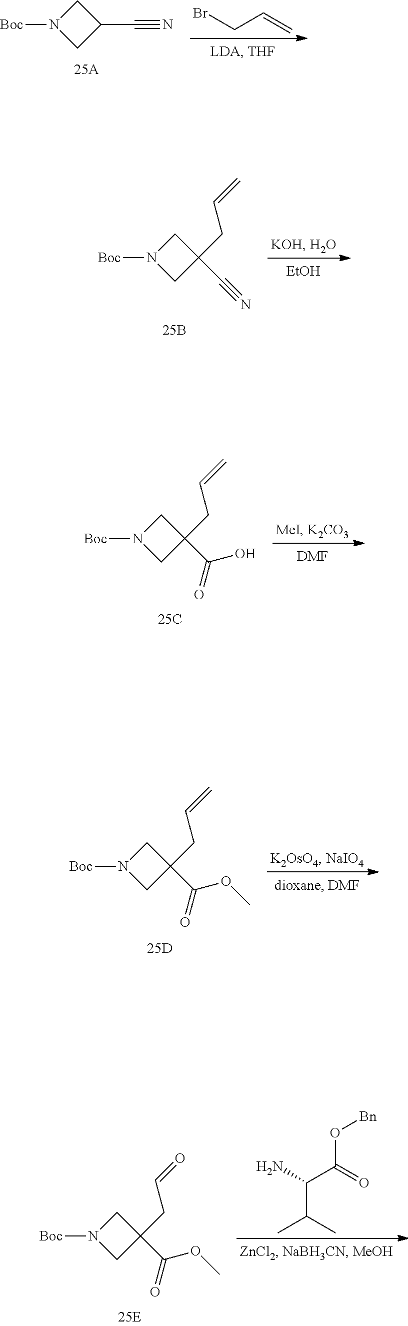

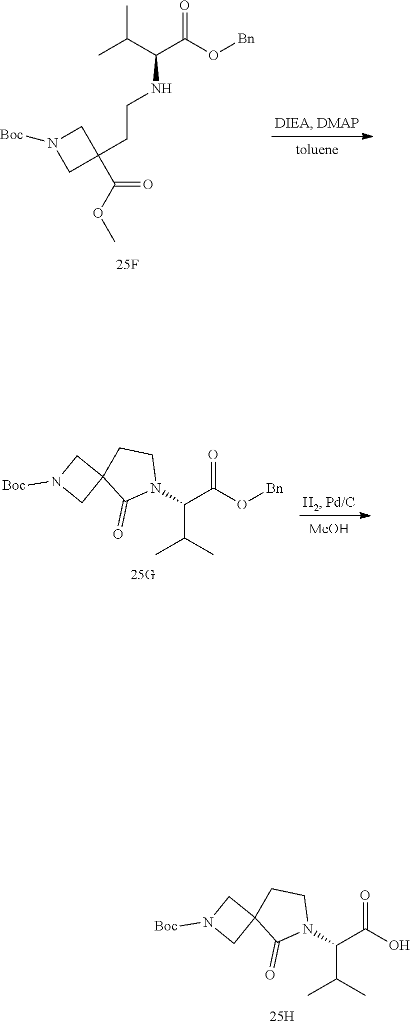

### Step 1: tert-butyl 3-allyl-3-cyano-azetidine-1-carboxylate (compound 25B)

To a solution of tert-butyl 3-cyanoazetidine-1-carboxylate (compound 25A, 10.0 g, 54.88 mmol) in THF (100 mL) was added LDA (60.3 mL, 60.37 mmol) dropwise at −70° C. under nitrogen atmosphere. After being stirred at −70° C. for 0.5 h, the reaction mixture was added with allyl bromide (7.9 g, 65.85 mmol). The mixture was stirred at 16° C. for another 1 h, then concentrated in vacuo and the residue was purified by column chromatography (EtOAc in PE: 3%-10%) to afford tert-butyl 3-allyl-3-cyano-azetidine-1-carboxylate (compound 25B, 12.5 g) as light yellow oil. ¹H NMR (400 MHz, CHLOROFORM-d) δ 5.90-5.70 (m, 1H), 5.32 (s, 1H), 5.28 (d, J=5.2 Hz, 1H), 4.23 (d, J=8.8 Hz, 2H), 3.86 (d, J=8.8 Hz, 2H), 2.63 (d, J=7.2 Hz, 2H), 1.45 (s, 9H) ppm.

### Step 2: 3-allyl-1-tert-butoxycarbonyl-azetidine-3-carboxylic acid (compound 25C)

A mixture of tert-butyl 3-allyl-3-cyano-azetidine-1-carboxylate (compound 25B, 12.5 g, 56.24 mmol) and potassium hydroxide (12.6 g, 224.94 mmol) in ethanol (60 mL) and water (60 mL) was stirred at 100° C. for 16 hrs. After the reaction was completed, the pH of the reaction mixture was acidified by HCl (1 M) aq. to pH=3. The reaction mixture was extracted with EtOAc (120 mL, three times). The combined organic layer was washed with brine (300 mL), dried over Na₂SO₄, filtered and concentrated in vacuo to afford 3-allyl-1-tert-butoxycarbonyl-azetidine-3-carboxylic acid (compound 25C, 14.0 g) as a white solid. MS calc'd 241.1 (MH⁺), measured 186.2 (M-C₄H₈+H⁺).

### Step 3: O₁-tert-butyl O₃-methyl 3-allylazetidine-1,3-dicarboxylate (compound 25D)

To a mixture of 3-allyl-1-tert-butoxycarbonyl-azetidine-3-carboxylic acid (compound 25C, 8.0 g, 33.16 mmol) in DMF (80 mL) was added potassium carbonate (13.7 g, 99.47 mmol) followed by iodomethane (9.4 g, 66.31 mmol). After being stirred at 20° C. for 2 hrs, the reaction mixture was poured into water (400 mL) and the resulting mixture was extracted with EtOAc (100 mL, three times). The combined organic layer was washed with brine (300 mL), dried over Na₂SO₄, filtered and concentrated in vacuo to afford O₁-tert-butyl O₃-methyl 3-allylazetidine-1,3-dicarboxylate (compound 25D, 7.3 g) as yellow oil. ¹H NMR (400 MHz, CHLOROFORM-d) δ 5.80-5.60 (m, 1H), 5.19-5.14 (m, 1H), 5.12 (s, 1H), 4.16 (d, J=8.8 Hz, 2H), 3.82-3.69 (m, 5H), 2.63 (d, J=7.2 Hz, 2H), 1.44 (s, 9H) ppm.

### Step 4: O₁-tert-butyl O₃-methyl 3-(2-oxoethyl)azetidine-1,3-dicarboxylate (compound 25E)

To a mixture of O₁-tert-butyl O₃-methyl 3-allylazetidine-1,3-dicarboxylate (compound 25D, 2 g, 7.83 mmol) in 1,4-dioxane (20 mL) and water (20 mL) was added 2,6-Lutidine (1.8 mL, 15.67 mmol) and K₂OsO₄ (144.3 mg, 0.39 mmol). After being stirred at 16° C. for 15 minutes, the reaction mixture was added with sodium metaperiodate (6702.2 mg, 31.33 mmol) and stirred for another 1 h, then poured into water (120 mL) and the resulting mixture was extracted with EtOAc (40 mL, three times). The combined organic layer was washed with sat. Na₂SO₃ (60 mL), brine (60 mL), dried over Na₂SO₄, filtered and concentrated in vacuo to afford O₁-tert-butyl O₃-methyl 3-(2-oxoethyl)azetidine-1,3-dicarboxylate (compound 25E, 2.8 g) as yellow oil. MS calc'd 257.1 (MH⁺), measured 202.1, (M-C₄H₈+H⁺).

### Step 5: O₁-tert-butyl O₃-methyl 3-[2-[[(1S)-1-benzyloxycarbonyl-2-methyl-propyl]amino]ethyl]azetidine-1,3-dicarboxylate (compound 25F)

To a mixture of L-valine benzyl ester hydrochloride (3.2 g, 13.06 mmol) and O₁-tert-butyl O₃-methyl 3-(2-oxoethyl)azetidine-1,3-dicarboxylate (compound 25E, 2.8 g, 10.88 mmol) in methanol (50 mL) was added zinc chloride (1.8 g, 13.06 mmol). After being stirred at 16° C. for 0.5 h, the reaction mixture was added with sodium cyanoborohydride (1.4 g, 21.77 mmol) and the resulting mixture was stirred for another 1 h. The reaction mixture was concentrated in vacuo and the residue was purified by column chromatography (EtOAc in PE: 30%-50%) to afford O₁-tert-butyl O₃-methyl 3-[2-[[(1S)-1-benzyloxycarbonyl-2-methyl-propyl]amino]ethyl]azetidine-1,3-dicarboxylate (compound 25F, 2.2 g) as colorless oil. MS calc'd 449.3 (MH⁺), measured 449.3 (MH⁺).

### Step 6: tert-butyl 6-[(1S)-1-benzyloxycarbonyl-2-methyl-propyl]-5-oxo-2,6-diazaspiro[3.4]octane-2-carboxylate (compound 25G)

To a mixture of 4-dimethylaminopyridine (544.7 mg, 4.46 mmol), DIEA (7.7 mL, 44.59 mmol) and O₁-tert-butyl O₃-methyl 3-[2-[[(1S)-1-benzyloxycarbonyl-2-methyl-propyl]amino]ethyl]azetidine-1,3-dicarboxylate (compound 25F, 2.0 g, 4.46 mmol) in toluene (2 mL) was stirred at 100° C. for 20 hrs. After the reaction was completed, the reaction mixture was concentrated in vacuo and the residue was dissolved in EtOAc (60 mL). The resulting solution was washed with HCl aq. (1 M, 60 mL), then brine (60 mL), dried over Na₂SO₄, filtered. The filtrate was concentrated in vacuo to afford tert-butyl 6-[(1S)-1-benzyloxycarbonyl-2-methyl-propyl]-5-oxo-2,6-diazaspiro[3.4]octane-2-carboxylate (compound 25G, 1.5 g) as a white solid. MS calc'd 417.2 (MH⁺), measured 361.2 (M-C₄H₈+H⁺).

### Step 7: (2S)-2-(2-tert-butoxycarbonyl-5-oxo-2,6-diazaspiro[3.4]octan-6-yl)-3-methyl-butanoic acid (compound 25H)

A mixture of tert-butyl 6-[(1S)-1-benzyloxycarbonyl-2-methyl-propyl]-5-oxo-2,6-diazaspiro[3.4]octane-2-carboxylate (compound 25G, 1.5 g, 3.6 mmol) and Pd on activated carbon (150.0 mg) in methanol (20 mL) was stirred under H₂ balloon at 16° C. for 2 hrs. The reaction mixture was filtered and the filtrate was concentrated in vacuo to afford (2S)-2-(2-tert-butoxycarbonyl-5-oxo-2,6-diazaspiro[3.4]octan-6-yl)-3-methyl-butanoic acid (compound 25H, 920.0 mg) as a white solid. MS calc'd 327.2 (MH⁺), measured 271.2 (M-C₄H₈+H⁺).

### Example 26

### (3S)-1-[(2R)-2-chloro-2-fluoro-acetyl]-N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide

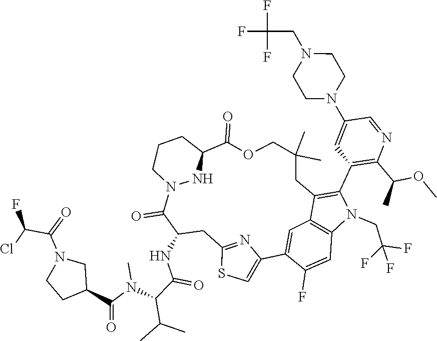

The title compound was prepared in analogy to the preparation of Example 1 by using (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate J) instead of (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E). Example 26 (14.6 mg) was obtained as a white solid. MS calc'd 1173.4 (MH⁺), measured 1173.7 (MH⁺). ¹H NMR (400 MHz, METHANOL-d₄) δ=8.72-8.68 (m, 1H), 8.42 (d, J=2.4 Hz, 1H), 7.77-7.68 m, 1H), 7.57-7.45 (m, 2H), 6.93-6.76 (m, 1H), 5.81-5.63 (m, 1H), 5.27-5.11 (m, 1H), 4.96-4.93 (m, 1H), 4.88-4.79 (m, 2H), 4.47-4.38 (m, 1H), 4.28-4.18 (m, 2H), 3.88-3.53 (m, 7H), 3.47-3.34 (m, 8H), 3.30-3.24 (m, 1H), 3.23-3.07 (m, 6H), 2.92-2.80 (m, 5H), 2.70-2.56 (m, 1H), 2.40-2.06 (m, 4H), 2.00-1.91 (m, 1H), 1.88-1.74 (m, 1H), 1.69-1.57 (m, 1H), 1.49-1.41 (m, 3H), 1.01-0.97 (m, 5H), 0.92-0.82 (m, 3H), 0.53-0.42 (m, 3H) ppm.

### Example 27

### (3S)-1-[(2R)-2-chloro-2-fluoro-acetyl]-N-[(1S)-1-[[(8S,14S)-26-fluoro-4-hydroxy-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl]-2-methyl-propyl]-N-methyl-pyrrolidine-3-carboxamide

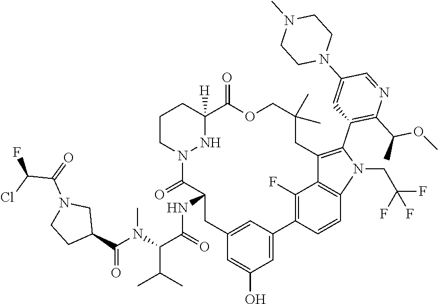

The title compound was prepared in analogy to the preparation of Example 1 by using (8S,14S)-8-amino-26-fluoro-4-hydroxy-(21M)-21-[2-[(1S)-1-methoxyethyl]-5-(4-methylpiperazin-1-yl)-3-pyridyl]-18,18-dimethyl-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaene-9,15-dione (Intermediate K) instead of (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E). Example 27 (5.2 mg) was obtained as a white solid. MS calc'd 1114.5 (MH⁺), measured 1114.6 (MH⁺), ¹H NMR (400 MHz, METHANOL-d₄) δ: 8.50 (d, J=2.8 Hz, 1H), 8.21-8.10 (m, 1H), 7.51 (s, 1H), 7.47 (d, J=8.0 Hz, 1H), 7.42-7.36 (m, 1H), 7.14-7.06 (m, 1H), 6.89-6.86 (m, 1H), 5.34 (t, J=4.4 Hz, 1H), 4.69 (d, J=10.8 Hz, 1H), 4.43-4.34 (m, 0.5H), 4.27-4.17 (m, 0.5H), 4.14-3.95 (m, 1H), 3.79-3.56 (m, 6H), 3.23 (s, 2H), 3.02 (d, J 14 Hz, 6H), 2.91-2.78 (m, 2H), 2.78-2.56 (m, 2H), 2.19 (t, J=7.6 Hz, 2H), 2.06-2.01 (m, 3H), 1.88-1.77 (m, 1H), 1.64-1.56 (m, 1H), 1.50-1.46 (m, 3H), 1.44 (d, J=1.6 Hz, 1H), 1.34-1.29 (m, 10H), 1.18 (t, J=6.8 Hz, 2H), 0.97-0.87 (m, 8H), 0.86-0.82 (m, 3H), 0.41 (s, 3H) ppm.

### Example 28

### (2S)-2-[8-[(2R)-2-chloro-2-fluoro-acetyl]-1-oxo-2,8-diazaspiro[4.5]decan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

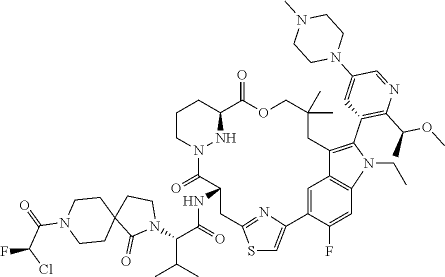

The title compound was prepared in analogy to the preparation of Example 6 by using (2S)-2-(8-tert-butoxycarbonyl-1-oxo-2,8-diazaspiro[4.5]decan-2-yl)-3-methyl-butanoic acid (compound 15F) and (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 28 (11.7 mg) was obtained as a white solid. MS calc'd 1077.5 (MH⁺), measured 1077.5 (MH⁺). ¹H NMR (400 MHz, METHANOL-d₄) δ 8.64 (d, J=7.7 Hz, 1H), 8.43 (d, J=1.7 Hz, 1H), 7.66 (s, 1H), 7.43 (d, J=2.2 Hz, 1H), 7.30 (d, J=12.3 Hz, 1H), 7.13-6.92 (m, 1H), 5.69-5.53 (m, 2H), 5.51-5.36 (m, 1H), 4.58 (s, 11H), 4.54-4.23 (m, 1H), 4.22-3.80 (m, 1H), 3.78-3.46 (m, 3H), 2.75-2.59 (m, 5H), 2.44-2.30 (m, 5H), 2.29-2.19 (m, 2H), 2.18-2.05 (m, 3H), 2.03-1.74 (m, 6H), 1.70-1.55 (m, 3H), 1.30-1.15 (m, 7H), 1.03-0.94 (m, 7H), 0.90 (dd, J=2.6, 6.5 Hz, 3H), 0.61 (s, 3H) ppm.

### Example 29

### (2S)-2-[2-[(2R)-2-chloro-2-fluoro-acetyl]-5-oxo-2,6-diazaspiro[3.4]octan-6-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

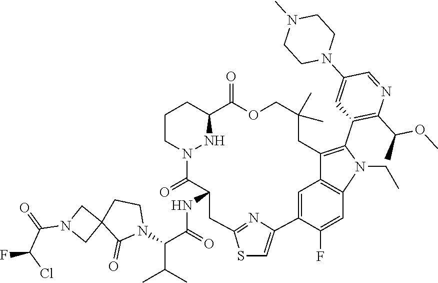

The title compound was prepared in analogy to the preparation of Example 6 by using (2S)-2-(2-tert-butoxycarbonyl-5-oxo-2,6-diazaspiro[3.4]octan-6-yl)-3-methyl-butanoic acid (compound 25H) and (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 29 (63.2 mg) was obtained as a yellow solid. MS calc'd 1049.5 (MH⁺), measured 1049.5 (MH⁺). ¹H NMR (400 MHz, Methanol-d₄) δ=8.76-8.63 (m, 1H), 8.58-8.45 (m, 1H), 7.78-7.56 (m, 2H), 7.44-7.30 (m, 1H), 6.82-6.59 (m, 1H), 5.83-5.67 (m, 1H), 4.65-4.53 (m, 1H), 4.50-3.99 (m, 11H), 3.88-3.67 (m, 3H), 3.61-3.47 (m, 3H), 3.47-3.42 (m, 3H), 3.41-3.35 (m, 4H), 3.32-3.22 (m, 2H), 3.11-3.04 (m, 1H), 3.01 (s, 3H), 2.90-2.78 (m, 1H), 2.69-2.60 (m, 1H), 2.45-2.35 (m, 2H), 2.31-2.16 (m, 2H), 2.03-1.92 (m, 1H), 1.90-1.75 (m, 1H), 1.72-1.58 (m, 1H), 1.52-1.41 (m, 3H), 1.08-0.98 (m, 6H), 0.96 (s, 3H), 0.89 (dd, J=2.8, 6.4 Hz, 3H), 0.62-0.43 (m, 3H) ppm.

### Example 30 and Example 31

### (2S)-2-[(5R)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 30)

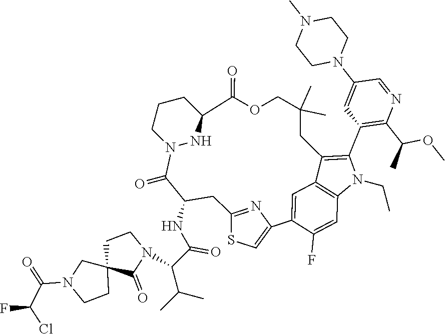

### Example 30

### (2S)-2-[(5S)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 31)

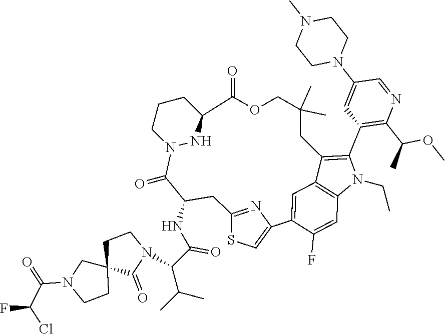

### Example 31

Example 30 was prepared in analogy to the preparation of Example 4 by using (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 30 (42.4 mg) was obtained as a yellow solid. MS calc'd 1063.5 (MH⁺), measured 1063.5 (MH⁺). ¹H NMR (400 MHz, Methanol-d₄) δ 8.72-8.65 (m, 1H), 8.56-8.45 (m, 1H), 7.78-7.73 (m, 0.5H), 7.70-7.64 (m, 0.5H), 7.63-7.57 (m, 1H), 7.41-7.31 (m, 1H), 6.95-6.71 (m, 1H), 5.82-5.67 (m, 1H), 4.51-4.02 (m, 8H), 3.99-3.90 (m, 1H), 3.88-3.70 (m, 5H), 3.69-3.56 (m, 4H), 3.52-3.43 (m, 3H), 3.37 (d, J=6.4 Hz, 4H), 3.30-3.23 (m, 2H), 3.13-3.05 (m, 1H), 3.01 (s, 3H), 2.88-2.79 (m, 1H), 2.69-2.57 (m, 1H), 2.36-2.18 (m, 3H), 2.18-2.04 (m, 3H), 2.01-1.96 (m, 1H), 1.88-1.76 (m, 1H), 1.71-1.59 (m, 1H), 1.46 (d, J=6.0 Hz, 3H), 1.05-0.94 (m, 9H), 0.94-0.89 (d, J=6.6 Hz, 3H), 0.58-0.41 (m, 3H) ppm.

Example 31 was prepared in analogy to the preparation of Example 4 by using (2S)-2-[(5R)-7-tert-butoxycarbonyl-1-oxo-2,7-diazaspiro[4.4]nonan-2-yl]-3-methyl-butanoic acid (compound 4f 1) and (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (2S)-2-[(5S)-7-tert-butoxycarbonyl-1-oxo-2,7-diazaspiro[4.4]nonan-2-yl]-3-methyl-butanoic acid (compound 4f 2) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 31 (56.7 mg) was obtained as a yellow solid. MS calc'd 1063.5 (MH⁺), measured 1063.5 (MH⁺). ¹HNMR (400 MHz, METHANOL-d₄) δ=8.67 (d, J=7.6 Hz, 1H), 8.48 (d, J=2.8 Hz, 1H), 7.65 (s, 1H), 7.57 (s, 1H), 7.34 (d, J=12.8 Hz, 1H), 6.94-6.73 (m, 1H), 5.75 (t, J=8.8 Hz, 1H), 4.45-4.37 (m, 1H), 4.36-4.29 (m, 2H), 4.28-4.08 (m, 4H), 4.07-3.97 (m, 1H), 3.97-3.87 (m, 1H), 3.85-3.72 (m, 4H), 3.71-3.63 (m, 2H), 3.63-3.41 (m, 7H), 3.34 (s, 3H), 3.30-3.23 (m, 2H), 3.07-3.02 (m, 1H), 3.00 (s, 3H), 2.87-2.77 (m, 1H), 2.69-2.57 (m, 1H), 2.33-2.15 (m, 3H), 2.14-2.03 (m, 3H), 1.97-1.91 (m, 1H), 1.84-1.72 (m, 1H), 1.69-1.57 (m, 1H), 1.44 (d, J=6.0 Hz, 3H), 1.03-0.92 (m, 9H), 0.89 (d, J=6.8 Hz, 3H), 0.51 (s, 3H) ppm.

### Example 32

### (3S)-1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide

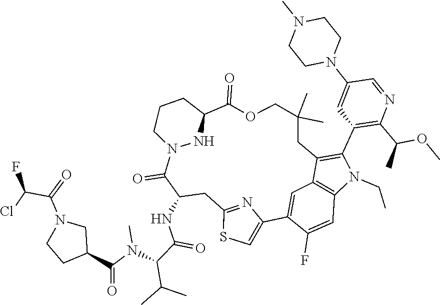

The title compound was prepared in analogy to the preparation of Example 1 by using (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate H) instead of (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E). Example 32 (36.7 mg) was obtained as a yellow solid. MS calc'd 1051.4 (MH⁺), measured 1051.6 (MH⁺). ¹H NMR (400 MHz, MeOD) δ 8.67 (d, J=7.6 Hz, 1H), 8.49 (d, J=2.8 Hz, 1H), 7.73-7.59 (m, 2H), 7.34 (d, J=12.8 Hz, 1H), 6.96-6.70 (m, 1H), 5.82-5.61 (m, 1H), 5.00-4.90 (m, 2H), 4.83-4.77 (m, 1H), 4.49-4.38 (m, 1H), 4.37-4.05 (m, 5H), 4.00-3.89 (m, 1H), 3.89-3.47 (m, 10H), 3.47-3.39 (m, 2H), 3.36 (d, J=4.8 Hz, 3H), 3.28-3.22 (m, 1H), 3.15-3.07 (m, 3H), 3.06-3.02 (m, 1H), 3.00 (s, 3H), 2.89-2.76 (m, 1H), 2.69-2.56 (m, 1H), 2.42-2.01 (m, 4H), 2.00-1.90 (m, 1H), 1.89-1.73 (m, 1H), 1.71-1.56 (m, 1H), 1.44 (d, J=6.4 Hz, 3H), 1.25-0.96 (m, 6H), 0.96-0.92 (m, 3H), 0.92-0.82 (m, 3H), 0.61-0.39 (m, 3H) ppm.

### Example 33

### (3S)-1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide

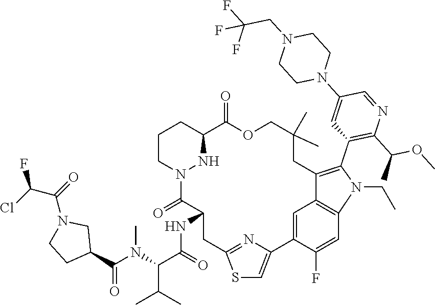

The title compound was prepared in analogy to the preparation of Example 1 by using (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate L) instead of (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E). Example 33 (45.2 mg) was obtained as a yellow solid. MS calc'd 1119.5 (MH⁺), measured 1119.7 (MH⁺). ¹H NMR (400 MHz, METHANOL-d₄) δ=8.73-8.65 (m, 1H), 8.38 (s, 1H), 7.94-7.86 (m, 1H), 7.72-7.62 (m, 1H), 7.37 (d, J=12.6 Hz, 1H), 6.94-6.72 (m, 1H), 5.83-5.69 (m, 1H), 4.84-4.76 (m, 1H), 4.47-4.19 (m, 3H), 4.17-3.92 (m, 2H), 3.89-3.62 (m, 6H), 3.61-3.53 (m, 1H), 3.51-3.46 (m, 4H), 3.43-3.37 (m, 3H), 3.28-3.13 (m, 3H), 3.10-3.03 (m, 3H), 2.93-2.87 (m, 4H), 2.86-2.66 (m, 2H), 2.42-2.11 (m, 4H), 2.00-1.90 (m, 1H), 1.86-1.73 (m, 1H), 1.71-1.57 (m, 1H), 1.46 (d, J=6.4 Hz, 3H), 1.36-1.27 (m, 1H), 1.14-0.78 (m, 13H), 0.69-0.54 (m, 3H) ppm.

### Example 34

### 1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-4-fluoro-N-methyl-piperidine-4-carboxamide

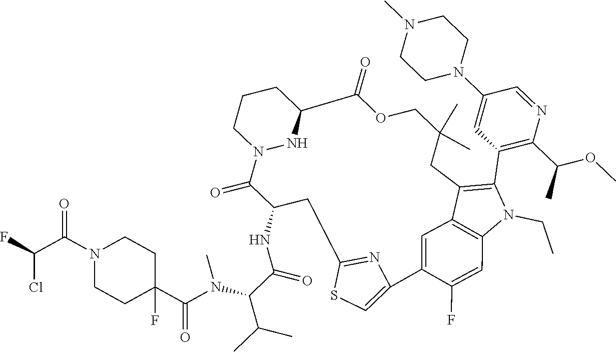

The title compound was prepared in analogy to the preparation of Example 1 by using 1-tert-butoxycarbonyl-4-fluoro-piperidine-4-carboxylic acid and (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (S)-1-Boc-pyrrolidine-3-carboxylic acid (compound 1D) and (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E). Example 34 (12.9 mg) was obtained as a yellow solid. MS calc'd 1083.5 (MH⁺), measured 1083.6 (MH⁺), ¹H NMR (400 MHz, Methanol-d₄) δ=8.65 (d, J=7.6 Hz, 1H), 8.48 (d, J=2.4 Hz, 1H), 7.64 (s, 1H), 7.54 (s, 1H), 7.34 (d, J=12.4 Hz, 1H), 7.11-6.95 (m, 1H), 5.78-5.71 (m, 1H), 4.47-4.37 (m, 2H), 4.35-4.23 (m, 2H), 4.22-4.07 (m, 4H), 4.00-3.88 (m, 2H), 3.80-3.69 (m, 3H), 3.68-3.57 (m, 2H), 3.54-3.41 (m, 4H), 3.34 (s, 3H), 3.20 (t, J=4.8 Hz, 4H), 3.14-3.05 (m, 2H), 3.03-2.95 (m, 5H), 2.86-2.79 (m, 1H), 2.65-2.57 (m, 1H), 2.32-2.13 (m, 5H), 2.03-1.90 (m, 2H), 1.84-1.77 (m, 1H), 1.68-1.59 (m, 1H), 1.44 (d, J=6.0 Hz, 3H), 1.29-1.09 (m, 2H), 1.00-0.97 (m, 3H), 0.93 (s, 3H), 0.91-0.86 (m, 3H), 0.50 (d, J=5.2 Hz, 3H).

### Example 35 and Example 38

### (2S)-2-[(5R)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 35)

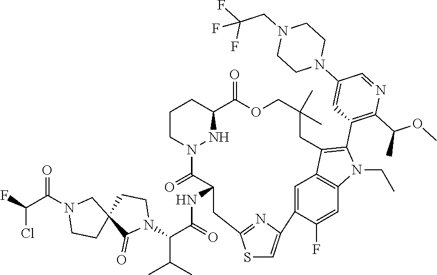

### Example 35

### (2S)-2-[(5S)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 38)

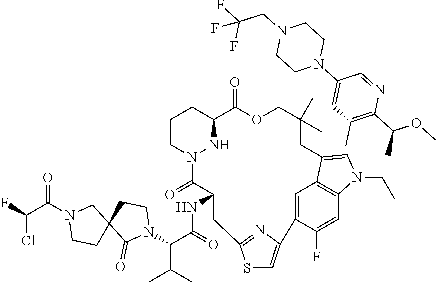

### Example 38

Example 35 was prepared in analogy to the preparation of Example 4 by using (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate L) instead of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 35 (21.1 mg) was obtained as a yellow solid. MS calc'd 1131.5 (MH⁺), measured 1131.6 (MH⁺). ¹H NMR (400 MHz, Methanol-d₄) δ 8.71 (dd, J=2.4, 7.6 Hz, 1H), 8.38 (t, J=2.4 Hz, 1H), 7.87-7.81 (m, 1H), 7.76-7.62 (m, 1H), 7.37 (dd, J=2.8, 12.6 Hz, 1H), 6.92-6.69 (m, 1H), 5.84-5.71 (m, 1H), 4.45-4.12 (m, 5H), 4.10-4.00 (m, 1H), 3.96-3.69 (m, 5H), 3.60-3.54 (m, 2H), 3.51-3.42 (m, 6H), 3.41-3.37 (m, 3H), 3.26-3.12 (m, 3H), 3.09-2.96 (m, 1H), 2.93-2.87 (m, 4H), 2.85-2.66 (m, 2H), 2.34-2.15 (m, 3H), 2.12-1.93 (m, 4H), 1.86-1.73 (m, 1H), 1.71-1.57 (m, 1H), 1.45 (d, J=6.4 Hz, 3H), 1.04-0.95 (m, 9H), 0.89 (d, J=6.8 Hz, 3H), 0.58 (d, J=12.8 Hz, 3H).

Example 38 was prepared in analogy to the preparation of Example 4 by using (2S)-2-[(5R)-7-tert-butoxycarbonyl-1-oxo-2,7-diazaspiro[4.4]nonan-2-yl]-3-methyl-butanoic acid (compound 4f 1) and (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate L) instead of (2S)-2-[(5S)-7-tert-butoxycarbonyl-1-oxo-2,7-diazaspiro[4.4]nonan-2-yl]-3-methyl-butanoic acid (compound 4f 2) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 38 (26.1 mg) was obtained as a yellow solid. MS calc'd 1131.5 (MH⁺), measured 1131.7 (MH⁺). ¹HNMR (400 MHz, METHANOL-d₄) δ=8.88-8.81 (m, 1H), 8.71 (d, J=7.6 Hz, 1H), 8.38 (d, J=2.0 Hz, 1H), 7.96-7.87 (m, 1H), 7.67-7.64 (m, 1H), 7.37 (d, J=12.4 Hz, 1H), 6.91-6.75 (m, 1H), 5.84-5.79 (m, 1H), 4.46-4.36 (m, 2H), 4.33-4.25 (m, 2H), 4.18-4.12 (m, 1H), 4.08-3.84 (m, 2H), 3.81-3.43 (m, 4H), 3.69-3.61 (m, 2H), 3.52-3.48 (m, 5H), 3.40 (s, 3H), 3.23-3.15 (m, 3H), 3.03-2.97 (m, 1H), 2.91-2.88 (m, 4H), 2.83-2.73 (m, 2H), 2.27-2.15 (m, 3H), 2.11-2.04 (m, 3H), 2.00-1.91 (m, 2H), 1.85-1.76 (m, 1H), 1.70-1.61 (m, 1H), 1.46 (d, J=6.4 Hz, 3H), 1.04-0.99 (m, 5H), 0.96 (s, 3H), 0.89 (d, J=6.8 Hz, 3H), 0.61 (s, 3H) ppm.

### Example 36

### (2S)-2-[9-[(2R)-2-chloro-2-fluoro-acetyl]-1-oxo-2,9-diazaspiro[5.5]undecan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

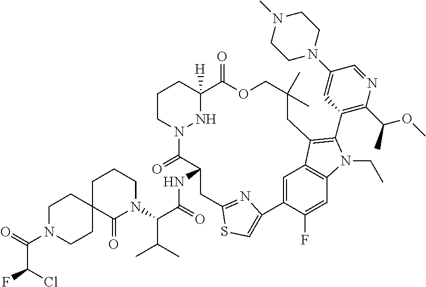

The title compound was prepared in analogy to the preparation of Example 6 by using (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 36 (27.5 mg) was obtained as a yellow solid. MS calc'd 1091.5 (MH⁺), measured 1091.5 (MH⁺). ¹H NMR (400 MHz, Methanol-d₄) δ=8.67 (d, J=7.6 Hz, 1H), 8.48 (d, J=2.8 Hz, 1H), 7.67 (d, J=2.4 Hz, 1H), 7.61-7.51 (m, 1H), 7.34 (d, J=12.8 Hz, 1H), 7.15-6.85 (m, 1H), 5.80-5.68 (m, 1H), 4.86-4.76 (m, 1H), 4.47-4.36 (m, 1H), 4.35-4.05 (m, 6H), 3.97-3.86 (m, 1H), 3.83-3.67 (m, 3H), 3.66-3.51 (m, 2H), 3.50-3.40 (m, 4H), 3.34 (s, 3H), 3.30-3.09 (m, 4H), 3.07-3.01 (m, 1H), 3.00 (s, 3H), 2.88-2.76 (m, 1H), 2.68-2.56 (m, 1H), 2.33-2.08 (m, 4H), 1.97-1.75 (m, 6H), 1.68-1.57 (m, 3H), 1.44 (d, J=6.0 Hz, 3H), 1.37-1.24 (m, 2H), 1.01-0.90 (m, 9H), 0.87 (d, J=6.4 Hz, 3H), 0.51 (s, 3H) ppm.

### Example 37

### (2S)-2-[2-[(2R)-2-chloro-2-fluoro-acetyl]-5-oxo-2,6-diazaspiro[3.4]octan-6-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

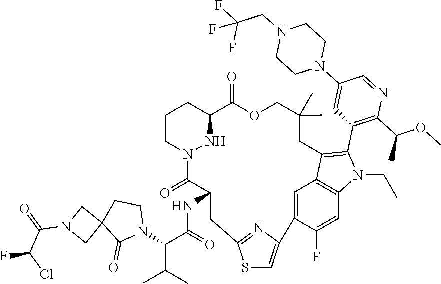

The title compound was prepared in analogy to the preparation of Example 6 by using (2S)-2-(2-tert-butoxycarbonyl-5-oxo-2,6-diazaspiro[3.4]octan-6-yl)-3-methyl-butanoic acid (compound 25H) and (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate L) instead of (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 37 (32.1 mg) was obtained as a yellow solid. MS calc'd 1117.5 (MH⁺), measured 1117.5 (MH⁺). ¹H NMR (400 MHz, Methanol-d₄) δ=8.69 (d, J=7.6 Hz, 1H), 8.38 (d, J=2.4 Hz, 1H), 7.74 (s, 1H), 7.66 (dd, J=4.0 Hz, 24.4 Hz, 1H), 7.35 (d, J=12.8 Hz, 1H), 6.68 (d, J=57.6 Hz, 1H), 5.78 (s, 1H), 4.57 (t, J=5.6 Hz, 1H), 4.45-4.22 (m, 6H), 4.19-4.11 (m, 1H), 4.11-4.01 (m, 2H), 3.82-3.68 (m, 2H), 3.45 (s, 6H), 3.37 (d, J=6.8 Hz, 4H), 3.22-3.12 (m, 2H), 3.07-2.97 (m, 1H), 2.88 (s, 4H), 2.76-2.66 (m, 2H), 2.38 (s, 2H), 2.26-2.13 (m, 2H), 1.99-1.92 (m, 1H), 1.86-1.74 (m, 1H), 1.69-1.61 (m, 1H), 1.45 (d, J=6.4 Hz, 3H), 1.34-1.23 (m, 1H), 1.05-0.98 (m, 6H), 0.95 (s, 3H), 0.87 (dd, J=2.4 Hz, 6.4 Hz, 3H), 0.57 (s, 3H) ppm.

### Example 39

### (2S)-2-[8-[(2R)-2-chloro-2-fluoro-acetyl]-1-oxo-2,8-diazaspiro[4.5]decan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

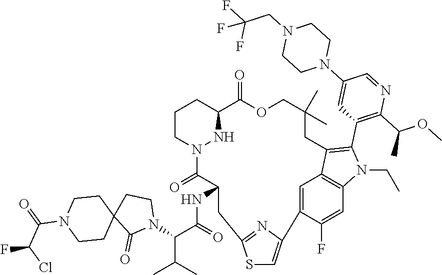

The title compound was prepared in analogy to the preparation of Example 6 by using (2S)-2-(8-tert-butoxycarbonyl-1-oxo-2,8-diazaspiro[4.5]decan-2-yl)-3-methyl-butanoic acid (compound 15F) and (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate L) instead of (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 39 (22.9 mg) was obtained as a yellow solid. MS calc'd 1145.5 (MH⁺), measured 1145.5 (MH⁺). ¹H NMR (400 MHz, METHANOL-d₄) δ=8.85-8.77 (m, 1H), 8.69 (d, J=7.6 Hz, 1H), 8.39 (d, J=2.8 Hz, 1H), 7.72-7.64 (m, 2H), 7.37-7.33 (m, 1H), 7.09-6.93 (m, 1H), 5.83-5.74 (m, 1H), 4.46-4.37 (m, 1H), 4.34-4.24 (m, 4H), 4.18-4.14 (m, 1H), 4.11-4.04 (m, 1H), 3.96-3.89 (m, 1H), 3.80-3.71 (m, 2H), 3.64-3.50 (m, 2H), 3.45-3.41 (m, 5H), 3.36 (s, 3H), 3.20-3.13 (m, 4H), 3.04-2.98 (m, 1H), 2.90-2.86 (m, 4H), 2.83-2.78 (m, 1H), 2.73-2.68 (m, 1H), 2.25-2.16 (m, 2H), 2.06-2.01 (m, 2H), 1.97-1.76 (m, 5H), 1.68-1.53 (m, 3H), 1.44 (d, J=6.4 Hz, 3H), 1.02-0.98 (m, 5H), 0.95 (s, 3H), 0.89-0.86 (m, 3H), 0.57 (s, 3H) ppm.

### Example 40

### (2R)-2-[[1-[(2R)-2-chloro-2-fluoro-acetyl]azetidin-3-yl]oxymethyl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

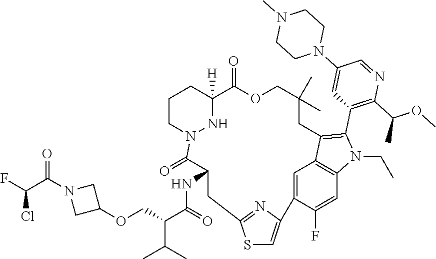

The title compound was prepared in analogy to the preparation of Example 13 by using (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 40 (26.2 mg) was obtained as a yellow solid. MS calc'd 1010.5 (MH⁺), measured 1010.4 (MH⁺). ¹H NMR (400 MHz, METHANOL-d₄) δ=8.66 (t, J=7.8 Hz, 1H), 8.50 (d, J=2.4 Hz, 1H), 7.72 (br s, 1H), 7.63 (t, J=2.9 Hz, 1H), 7.34 (dd, J=2.9, 12.6 Hz, 1H), 6.69-6.44 (m, 1H), 5.83 (br t, J=7.5 Hz, 1H), 5.01-4.92 (m, 1H), 4.63-3.86 (m, 11H), 3.84-3.65 (m, 3H), 3.64-3.53 (m, 3H), 3.45 (br d, J=15.2 Hz, 3H), 3.36 (br d, J=3.4 Hz, 3H), 3.31-3.22 (m, 3H), 3.10-2.94 (m, 4H), 2.81 (br t, J=12.4 Hz, 1H), 2.65 (br t, J=13.4 Hz, 1H), 2.52-2.37 (m, 1H), 2.19 (br d, J=12.2 Hz, 1H), 1.96 (br d, J=12.8 Hz, 1H), 1.90-1.71 (m, 2H), 1.70-1.54 (m, 1H), 1.45 (d, J=6.2 Hz, 3H), 1.17-0.77 (m, 12H), 0.54 (br d, J=9.3 Hz, 3H) ppm.

### Example 41

### (2S)-2-[[1-[(2R)-2-chloro-2-fluoro-acetyl]azetidin-3-yl]oxymethyl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

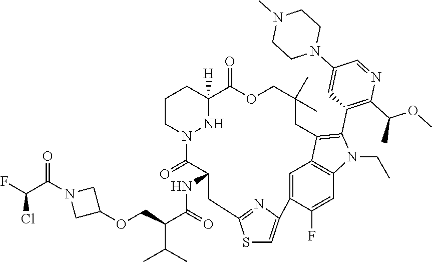

The title compound was prepared in analogy to the preparation of Example 13 by using tert-butyl 3-[(2S)-2-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-3-methyl-butoxy]azetidine-1-carboxylate, compound 40B) instead of tert-butyl 3-[(2R)-2-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]-octacosa-1(25),2,5(28),19,22(26),23-hexaen-7-yl]carbamoyl]-3-methyl-butoxy]azetidine-1-carboxylate (compound 13I). Example 41 (32.9 mg) was obtained as a yellow solid. MS calc'd 1010.5 (MH⁺), measured 1010.4 (MH⁺). ¹H NMR (400 MHz, METHANOL-d₄) δ=8.68 (d, J=7.4 Hz, 1H), 8.51 (d, J=2.8 Hz, 1H), 7.80 (d, J=2.8 Hz, 1H), 7.65 (d, J=2.4 Hz, 1H), 7.35 (d, J=12.6 Hz, 1H), 6.75-6.53 (m, 1H), 5.86 (br d, J=7.1 Hz, 1H), 5.03-4.92 (m, 1H), 4.54-4.01 (m, 11H), 4.01-3.83 (m, 2H), 3.81-3.66 (m, 3H), 3.66-3.50 (m, 4H), 3.50-3.38 (m, 3H), 3.36 (s, 3H), 3.02 (br s, 1H), 2.99 (s, 3H), 2.86-2.74 (m, 1H), 2.74-2.62 (m, 1H), 2.48-2.34 (m, 1H), 2.17 (br d, J=12.1 Hz, 1H), 2.00-1.85 (m, 2H), 1.83-1.70 (m, 1H), 1.68-1.55 (m, 1H), 1.45 (d, J=6.3 Hz, 3H), 1.09-0.85 (m, 12H), 0.57 (s, 3H) ppm.

Compound 40B was prepared in analogy to the preparation of compound 13J by using (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D).

### Example 42

### (2S)-2-[9-[(2R)-2-chloro-2-fluoro-acetyl]-1-oxo-2,9-diazaspiro[5.5]undecan-2-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

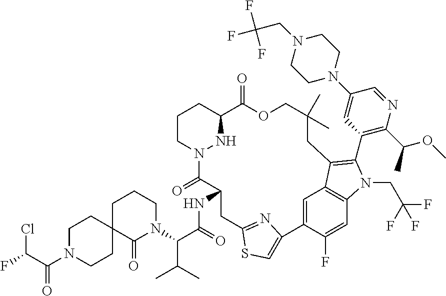

The title compound was prepared in analogy to the preparation of Example 6 by using (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate J) instead of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 42 (7.8 mg) was obtained as a yellow solid. MS calc'd 1213.5 (MH⁺), measured 1213.4 (MH⁺). ¹H NMR (400 MHz, Methanol-d₄) δ=8.70 (d, J=7.6 Hz, 1H), 8.42 (d, J=2.8 Hz, 1H), 7.74-7.67 (m, 1H), 7.52-7.41 (m, 2H), 7.13-6.87 (m, 1H), 5.75-5.65 (m, 1H), 5.20-5.11 (m, 1H), 4.81-4.78 (m, 1H), 4.48-4.34 (m, 1H), 4.25-4.05 (m, 3H), 3.98-3.65 (m, 3H), 3.52-3.33 (m, 11H), 3.30-3.09 (m, 6H), 2.91-2.78 (m, 5H), 2.68-2.57 (m, 1H), 2.32-2.06 (m, 4H), 2.00-1.77 (m, 6H), 1.70-1.57 (m, 3H), 1.44 (d, J=6.0 Hz, 3H), 1.03-0.92 (m, 6H), 0.90-0.84 (m, 3H), 0.47 (s, 3H) ppm.

### Example 43 and Example 45

### (2S)-2-[(5R)-7-[(2R)-2-chloro-2-fluoro-acetyl]-1-oxo-2,7-diazaspiro[4.4]nonan-2-yl]-N-[(7S,13S)-24-fluoro-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 43)

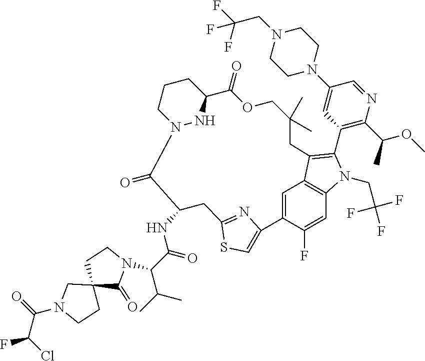

### Example 43

### (2S)-2-[(5S)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 45)

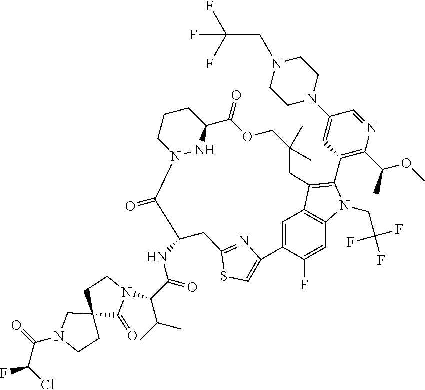

### Example 45

Example 43 prepared in analogy to the preparation of Example 4 by using (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate J) instead of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 43 (43.9 mg) was obtained as a yellow solid. MS calc'd 1185.4 (MH⁺), measured 1185.5 (MH⁺). ¹H NMR (400 MHz, Methanol-d₄) δ 8.82-8.71 (m, 1H), 8.44 (br s, 1H), 7.84-7.66 (m, 1H), 7.56-7.45 (m, 2H), 6.96-6.71 (m, 1H), 5.77-5.65 (m, 1H), 5.26-5.16 (m, 1H), 4.49-4.33 (m, 2H), 4.31-4.19 (m, 2H), 4.07-3.69 (m, 5H), 3.66-3.55 (m, 2H), 3.54-3.34 (m, 10H), 3.25-3.12 (m, 3H), 2.96-2.78 (m, 5H), 2.72-2.57 (m, 1H), 2.40-2.17 (m, 3H), 2.16-1.92 (m, 4H), 1.91-1.76 (m, 1H), 1.75-1.57 (m, 1H), 1.53-1.41 (m, 3H), 1.38-1.14 (m, 1H), 1.06-0.97 (m, 6H), 0.94-0.86 (m, 3H), 0.59-0.42 (m, 3H) ppm.

Example 45 was prepared in analogy to the preparation of Example 4 by using (2S)-2-[(5R)-7-tert-butoxycarbonyl-1-oxo-2,7-diazaspiro[4.4]nonan-2-yl]-3-methyl-butanoic acid (compound 4f 1) and (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate J) instead of (2S)-2-[(5S)-7-tert-butoxycarbonyl-1-oxo-2,7-diazaspiro[4.4]nonan-2-yl]-3-methyl-butanoic acid (compound 4f 2) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 45 (24.2 mg) was obtained as a white solid. MS calc'd 1185.4 (MH⁺), measured 1185.5 (MH⁺). ¹HNMR (400 MHz, METHANOL-d₄) δ=8.70 (d, J=7.6 Hz, 1H), 8.42 (d, J=2.8 Hz, 1H), 7.71 (t, J=2.4 Hz, 1H), 7.57 (s, 1H), 7.48 (d, J=12.8 Hz, 1H), 6.84 (dd, J=10.8 Hz, 48.8 Hz, 1H), 5.72 (s, 1H), 5.24-5.21 (m, 2H), 4.48-4.18 (m, 3H), 3.82-3.68 (m, 4H), 3.68-3.55 (m, 2H), 3.55-3.44 (m, 3H), 3.42 (d, J=3.6 Hz, 4H), 3.37 (s, 3H), 3.30-3.08 (m, 5H), 2.89 (t, J=4.8 Hz, 4H), 2.86-2.77 (m, 1H), 2.64 (d, J=14.4 Hz, 1H), 2.32-2.15 (m, 3H), 2.14-2.01 (m, 3H), 1.98-1.90 (m, 1H), 1.88-1.74 (m, 1H), 1.69-1.57 (m, 1H), 1.45 (d, J=6.4 Hz, 3H), 1.01 (d, J=6.4 Hz, 3H), 0.99 (s, 3H), 0.90 (d, J=6.4 Hz, 3H), 0.49 (s, 3H) ppm.

### Example 44

### 1-[(2R)-2-chloro-2-fluoro-acetyl]-4-fluoro-N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-piperidine-4-carboxamide

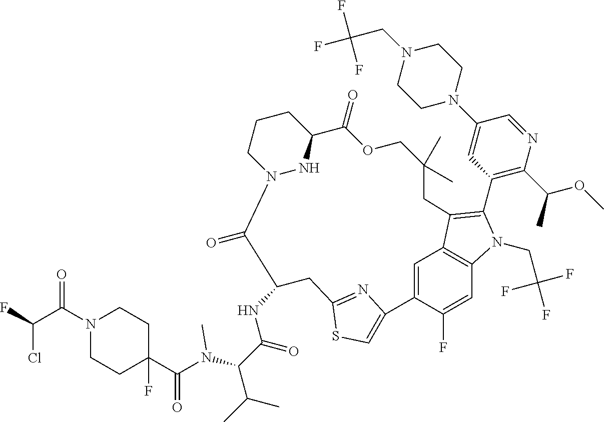

The title compound was prepared in analogy to the preparation of Example 1 by using 1-tert-butoxycarbonyl-4-fluoro-piperidine-4-carboxylic acid and (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate J) instead of (S)-1-Boc-pyrrolidine-3-carboxylic acid (compound 1D) and (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E). Example 44 (18.4 mg) was obtained as an off-white solid. MS calc'd 1205.4 (MH⁺), measured 1205.6 (MH⁺), ¹H NMR (400 MHz, Methanol-d₄) δ=8.69 (d, J=7.2 Hz, 1H), 8.42 (d, J=2.8 Hz, 1H), 7.69 (s, 1H), 7.58-7.51 (m, 1H), 7.48 (d, J=12.4 Hz, 1H), 7.11-6.97 (m, 1H), 5.74-5.68 (m, 1H), 5.24-5.10 (m, 2H), 4.75 (d, J=11.2 Hz, 1H), 4.46-4.34 (m, 2H), 4.27-4.16 (m, 2H), 4.00-3.88 (m, 1H), 3.81-3.69 (m, 2H), 3.57-3.49 (m, 1H), 3.47-3.38 (m, 5H), 3.37 (s, 3H), 3.22-3.17 (m, 4H), 3.16-3.12 (m, 2H), 3.10-3.01 (m, 1H), 2.88 (t, J=4.4 Hz, 5H), 2.84-2.77 (m, 1H), 2.68-2.59 (m, 1H), 2.35-2.15 (m, 6H), 1.99-1.93 (m, 1H). 1.88-1.77 (m, 1H), 1.69-1.59 (m, 1H), 1.45 (d, J=6.0 Hz, 3H), 1.00-0.95 (m, 6H), 0.89 (d, J=6.4 Hz, 3H), 0.48 (d, J=5.6 Hz, 3H).

### Example 46

### (2S)-2-[2-[(2R)-2-chloro-2-fluoro-acetyl]-5-oxo-2,6-diazaspiro[3.4]octan-6-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

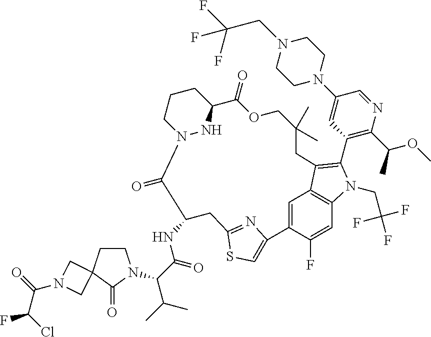

The title compound was prepared in analogy to the preparation of Example 6 by using (2S)-2-(2-tert-butoxycarbonyl-5-oxo-2,6-diazaspiro[3.4]octan-6-yl)-3-methyl-butanoic acid (compound 25H) and (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate J) instead of (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 46 (17.0 mg) was obtained as an off-white solid. MS calc'd 1171.4 (MH⁺), measured 1171.5 (MH⁺). ¹H NMR (400 MHz, Methanol-d₄) δ=8.72 (d, J=7.6 Hz, 1H), 8.42 (d, J=2.4 Hz, 1H), 7.74 (d, J=2.4 Hz, 1H), 7.70 (d, J=2.0 Hz, 1H), 7.50 (d, J=12.4 Hz, 1H), 6.69 (dd, J=1.6 Hz, 48.8 Hz, 1H), 5.74-5.69 (m, 1H), 4.44-4.21 (m, 5H), 3.84-3.67 (m, 2H), 3.59-3.52 (m, 1H), 3.46 (s, 6H), 3.40 (d, J=1.2 Hz, 3H), 3.24-3.14 (m, 3H), 3.14-3.08 (m, 1H), 2.90 (t, J=5.2 Hz, 4H), 2.85-2.77 (m, 1H), 2.71-2.61 (m, 1H), 2.44-2.36 (m, 2H), 2.27-2.14 (m, 2H), 2.01-1.90 (m, 1H), 1.90-1.71 (m, 1H), 1.70-1.55 (m, 1H), 1.47 (d, J=6.4 Hz, 3H), 1.25 (s, 2H), 1.20 (s, 3H), 1.02-0.96 (m, 6H), 0.88 (dd, J=3.2 Hz, 6.4 Hz, 3H), 0.51 (s, 3H) ppm.

### Example 47

### (2S)-2-[8-[(2R)-2-chloro-2-fluoro-acetyl]-1-oxo-2,8-diazaspiro[4.5]decan-2-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

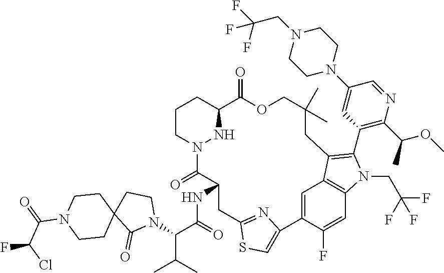

The title compound was prepared in analogy to the preparation of Example 6 by using (2S)-2-(8-tert-butoxycarbonyl-1-oxo-2,8-diazaspiro[4.5]decan-2-yl)-3-methyl-butanoic acid (compound 15F) and (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate J) instead of (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 47 (21.1 mg) was obtained as a white solid. MS calc'd 1199.5 (MH⁺), measured 1199.4 (MH⁺), ¹H NMR (400 MHz, Methanol-d₄) δ=8.86-8.79 (m, 1H), 8.69 (d, J=7.2 Hz, 1H), 8.44-8.40 (m, 1H), 7.69 (s, 1H), 7.51-7.43 (m, 2H), 7.12-6.90 (m, 1H), 5.69 (s, 1H), 5.22-5.11 (m, 1H), 4.44-4.37 (m, 1H), 4.34-4.19 (m, 4H), 3.99-3.87 (m, 1H), 3.81-3.68 (m, 2H), 3.62-3.58 (m, 1H), 3.49-3.42 (m, 2H), 3.41-3.37 (im, 4H), 3.35 (s, 3H), 3.28-3.23 (m, 1H), 3.20-3.09 (m, 4H), 2.90-2.86 (m, 4H), 2.84-2.78 (m, 1H), 2.62 (d, J=14.4 Hz, 1H), 2.26-2.19 (m, 2H), 2.10-2.02 (m, 2H), 2.00-1.90 (m, 2H), 1.87-1.76 (m, 2H), 1.70-1.53 (m, 3H), 1.44 (d, J=6.0 Hz, 3H), 1.18 (t, J=7.2 Hz, 1H), 1.03-0.96 (m, 6H), 0.88 (d, J=5.6 Hz, 3H), 0.47 (s, 3H) ppm.

### Example 48

### (2S)-2-[9-[(2R)-2-chloro-2-fluoro-acetyl]-1-oxo-2,9-diazaspiro[5.5]undecan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

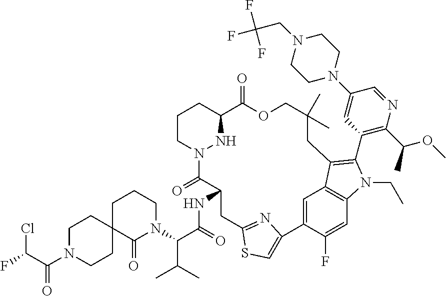

The title compound was prepared in analogy to the preparation of Example 6 by using (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate L) instead of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 48 (10 mg) was obtained as a yellow solid. MS calc'd 1159.5 (MH⁺), measured 1159.5 (MH⁺). ¹H NMR (400 MHz, Methanol-d₄) δ=8.69 (d, J=7.6 Hz, 1H), 8.40 (d, J=2.8 Hz, 1H), 7.68-7.64 (m, 1H), 7.63-7.57 (m, 1H), 7.35 (d, J=12.8 Hz, 1H), 7.11-6.87 (m, 1H), 5.82-5.71 (m, 1H), 4.82-4.75 (m, 2H), 4.45-4.38 (m, 1H), 4.36-4.29 (m, 1H), 4.27-4.03 (m, 4H), 3.97-3.81 (m, 1H), 3.79-3.70 (m, 2H), 3.47-3.42 (m, 6H), 3.36 (s, 3H), 3.30-3.12 (m, 5H), 3.08-2.96 (m, 1H), 2.92-2.79 (m, 5H), 2.73-2.64 (m, 1H), 2.32-2.06 (m, 4H), 2.00-1.90 (m, 1H), 1.90-1.72 (m, 5H), 1.68-1.55 (m, 3H), 1.44 (d, J=6.4 Hz, 3H), 1.05-0.94 (m, 9H), 0.89-0.82 (m, 3H), 0.55 (s, 3H) ppm.

### Example 49

### 1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-4-fluoro-N-methyl-piperidine-4-carboxamide

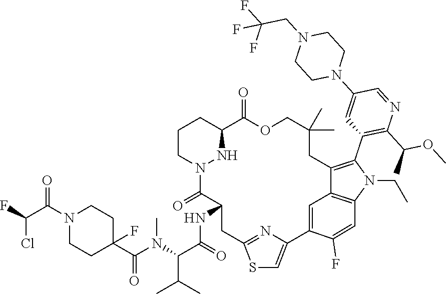

The title compound was prepared in analogy to the preparation of Example 1 by using 1-tert-butoxycarbonyl-4-fluoro-piperidine-4-carboxylic acid and (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate L) instead of (S)-1-Boc-pyrrolidine-3-carboxylic acid (compound 1D) and (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E). Example 49 (25.1 mg) was obtained as a yellow solid. MS calc'd 1151.5 (MH⁺), measured 1151.5 (MH⁺), ¹H NMR (400 MHz, Methanol-d₄) δ=8.70 (d, J=7.6 Hz, 1H), 8.39 (d, J=2.8 Hz, 1H), 7.85-7.78 (m, 1H), 7.67-7.60 (m, 1H), 7.36 (d, J=12.4 Hz, 1H), 7.11-6.94 (d, J=49.2 Hz, 1H), 5.89-5.69 (m, 1H), 4.77-4.67 (m, 1H), 4.46-4.32 (m, 3H), 4.30-4.21 (m, 1H), 4.18-4.12 (m, 1H), 4.10-4.00 (m, 1H), 3.96-3.86 (m, 1H), 3.81-3.71 (m, 2H), 3.51-3.43 (m, 5H), 3.43-3.34 (m, 4H), 3.28-3.13 (m, 7H), 3.03-2.98 (m, 1H), 2.92-2.87 (m, 4H), 2.86-2.78 (m, 1H), 2.76-2.66 (m, 1H), 2.36-2.08 (m, 6H), 1.99-1.90 (m, 1H), 1.85-1.71 (m, 1H), 1.70-1.58 (m, 1H), 1.45 (d, J=6.4 Hz, 3H), 1.04-0.93 (m, 9H), 0.91-0.80 (m, 3H), 0.65-1.53 m, 3H) ppm.

### Example 50

### (2S)-2-[8-[(2R)-2-chloro-2-fluoro-acetyl]-1-oxo-2,8-diazaspiro[4.5]decan-2-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

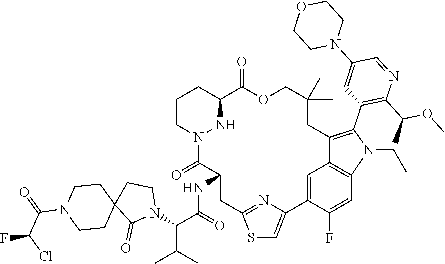

The title compound was prepared in analogy to the preparation of Example 6 by using (2S)-2-(8-tert-butoxycarbonyl-1-oxo-2,8-diazaspiro[4.5]decan-2-yl)-3-methyl-butanoic acid (compound 15F) and (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate M) instead of (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 50 (51.8 mg) was obtained as a yellow solid. MS calc'd 1064.5 (MH⁺), measured 1064.3 (MH⁺), ¹H NMR (400 MHz, CHLOROFORM-d) δ=8.99 (br s, 1H), 8.66-8.64 (d, J=7.5 Hz, 1H), 7.60-7.58 (dd, J=1.8, 8.4 Hz, 1H), 7.45-7.41 (dd, J=2.3, 16.1 Hz, 1H), 7.32-7.30 (br d, J=8.9 Hz, 0.5H), 7.19-7.18 (br d, J=8.8 Hz, 0.5H), 7.14-7.11 (d, J=12.3 Hz, 1H), 6.57-6.54 (m, 0.5H), 6.40-6.28 (m, 0.5H), 5.81 (td, J=9.2, 13.9 Hz, 1H), 4.66-4.52 (m, 1H), 4.40 (q, J=6.4 Hz, 1H), 4.31-4.22 (m, 1H), 4.19-4.03 (m, 5H), 3.92 (br t, J=4.5 Hz, 4H), 3.83 (br d, J=11.2 Hz, 1H), 3.69 (br d, J=11.1 Hz, 1H), 3.62-3.54 (m, 8H), 3.45-3.36 (m, 9H), 3.21-3.07 (m, 2H), 2.78-2.65 (m, 1H), 2.50-2.30 (m, 2H), 2.22 (br d, J=12.0 Hz, 1H), 2.00-1.96 (m, 2H), 1.92-1.76 (m, 1H), 1.61 (br d, J=10.1 Hz, 2H), 1.55-1.54 (d, J=6.4 Hz, 3H), 1.06-0.93 (m, 9H), 0.89-0.87 (dd, J=2.4, 6.4 Hz, 3H), 0.49 (s, 3H) ppm.

### Example 51 and Example 53

### (2S)-2-[(5S)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 51)

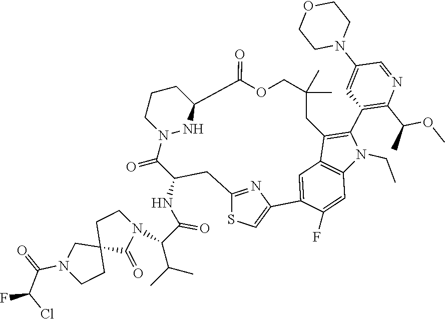

### Example 51

### (2S)-2-[(5R)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide (Example 53)

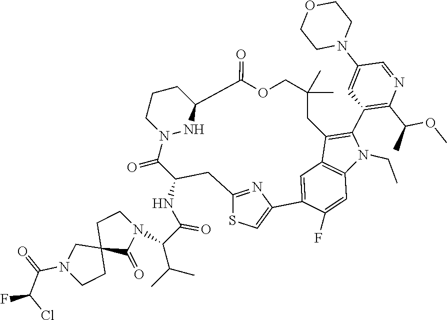

### Example 53

Example 51 was prepared in analogy to the preparation of Example 4 by using (2S)-2-[(5R)-7-tert-butoxycarbonyl-1-oxo-2,7-diazaspiro[4.4]nonan-2-yl]-3-methyl-butanoic acid (compound 4f 1) and (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate M) instead of (2S)-2-[(5S)-7-tert-butoxycarbonyl-1-oxo-2,7-diazaspiro[4.4]nonan-2-yl]-3-methyl-butanoic acid (compound 4f 2) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 51 (21.4 mg) was obtained as a yellow solid. MS calc'd 1050.4 (MH⁺), measured 1050.4 (MH⁺). ¹H NMR (400 MHz, Methanol-d₄) δ=8.70 (d, J=7.6 Hz, 1H), 8.39 (d, J=2.8 Hz, 1H), 7.89 (dd, J=2.8, 9.6 Hz, 1H), 7.65 (s, 1H), 7.38 (d, J=12.8 Hz, 1H), 8.90-6.74 (m, 1H), 5.79 (d, J=9.2 Hz, 1H), 4.44-4.37 (m, 2H), 4.33-4.24 (m, 2H), 4.18-4.12 (m, 1H), 4.08-4.00 (m, 1H), 3.87 (t, J=4.4 Hz, 4H), 3.81-3.70 (m, 4H), 3.68-3.63 (m, 1H), 3.58-3.54 (m, 1H), 3.51-3.47 (m, 1H), 3.45-3.41 (m, 5H), 3.40 (s, 3H), 3.29-3.23 (m, 1H), 3.02 (d, J=14.4 Hz, 1H), 2.86-2.71 (m, 2H), 2.28-2.15 (m, 3H), 2.09-2.00 (m, 3H), 1.98-1.93 (m, 1H), 1.84-1.75 (m, 1H), 1.68-1.60 (m, 1H), 1.46 (d, J=6.4 Hz, 3H), 1.34-1.28 (m, 1H), 1.04-0.99 (m, 6H), 0.97 (s, 3H), 0.88 (d, J=6.4 Hz, 3H), 0.60 (s, 3H).

Example 53 prepared in analogy to the preparation of Example 4 by using (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate M) instead of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 53 (28.8 mg) was obtained as a yellow solid. MS calc'd 1050.4 (MH⁺), measured 1050.5 (MH⁺). ¹H NMR (400 MHz, Methanol-d₄) δ=8.88-8.87 (m, 1H), 8.72-8.68 (m, 1H), 8.38 (t, J=2.4 Hz, 1H), 7.80 (t, J=2.8 Hz, 1H), 7.74-7.64 (m, 1H), 7.39-7.34 (m, 1H), 6.91-6.69 (m, 1H), 5.81-5.73 (m, 1H), 4.45-4.36 (m, 2H), 4.34-4.30 (m, 1H), 4.29-4.23 (m, 1H), 4.21-4.13 (m, 1H), 4.11-4.02 (m, 1H), 3.93-3.85 (m, 5H), 3.81-3.72 (m, 4H), 3.63-3.51 (m, 3H), 3.49-3.43 (m, 2H), 3.42-3.38 (m, 7H), 3.09-2.99 (m 1H), 2.87-2.78 (m, 1H), 2.70 (t, J=14.0 Hz, 1H), 2.33-2.25 (m, 1H), 2.24-2.16 (m, 2H), 2.12-2.06 (m, 2H), 2.02-1.91 (m, 2H), 1.85-1.76 (m, 1H), 1.70-1.61 (m, 1H), 1.46 (d, J=6.4 Hz, 3H), 1.04-0.98 (m, 6H), 0.97-0.96 (m, 2H), 0.89 (d, J=6.8 Hz, 3H), 0.61-0.55 (m, 3H).

### Example 52

### (2S)-2-[2-[(2R)-2-chloro-2-fluoro-acetyl]-5-oxo-2,6-diazaspiro[3.4]octan-6-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

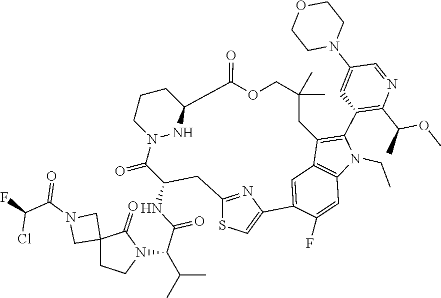

The title compound was prepared in analogy to the preparation of Example 6 by using (2S)-2-(2-tert-butoxycarbonyl-5-oxo-2,6-diazaspiro[3.4]octan-6-yl)-3-methyl-butanoic acid (compound 25H) and (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate M) instead of (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 52 (16.7 mg) was obtained as a yellow solid. MS calc'd 1036.4 (MH⁺), measured 1036.4 (MH⁺). ¹H NMR (400 MHz, Methanol-d₄) δ=8.69 (d, J=7.2 Hz, 1H), 8.38 (d, J=2.4 Hz, 1H), 7.85-7.82 (m, 1H), 7.69-7.63 (m, 1H), 7.37 (d, J=12.4 Hz, 1H), 6.75-6.62 (m, 1H), 5.80-5.74 (m, 1H), 4.57 (t, J=10.0 Hz, 1H), 4.41-4.34 (m, 3H), 4.32-4.26 (m, 2H), 4.19-4.13 (m, 1H), 4.10-4.03 (m, 2H), 3.87 (t, J=4.8 Hz, 4H), 3.80-3.71 (m, 2H), 3.53-3.46 (m, 2H), 3.42-3.41 (m, 3H), 3.39-3.38 (m, 3H), 3.07-2.98 (m, 1H), 2.87-2.79 (m, 1H), 2.75-2.68 (m, 1H), 2.41-2.35 (m, 2H), 2.28-2.14 (m, 3H), 2.07-1.90 (m, 2H), 1.85-1.76 (m, 1H), 1.66-1.60 (m, 1H), 1.46 (d, J=6.4 Hz, 3H), 1.33-1.29 (m, 2H), 1.03-0.98 (m, 6H), 0.98-0.94 (m, 3H), 0.89-0.85 (m, 3H), 0.58 (s, 3H).

### Example 54

### 1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-4-fluoro-N-methyl-piperidine-4-carboxamide

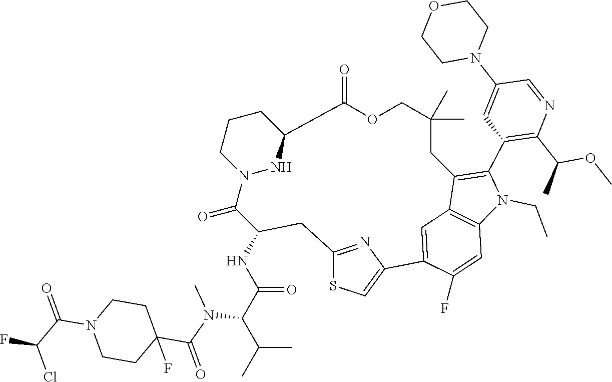

The title compound was prepared in analogy to the preparation of Example 1 by using 1-tert-butoxycarbonyl-4-fluoro-piperidine-4-carboxylic acid and (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate M) instead of (S)-1-Boc-pyrrolidine-3-carboxylic acid (compound 1D) and (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate E). Example 54 (44.2 mg) was obtained as a yellow solid. MS calc'd 1070.5 (MH⁺), measured 1070.3 (MH⁺), ¹H NMR (400 MHz, Methanol-d₄) δ=8.70 (d, J=7.2 Hz, 1H), 8.39 (d, J=2.8 Hz, 1H), 7.90-7.83 (m, 1H), 7.64 (d, J=2.4 Hz, 1H), 7.37 (d, J=12.8 Hz, 1H), 7.12-6.95 (d, J=49.2 Hz, 1H), 5.87-5.72 (m, 1H), 4.85-4.69 (m, 1H), 4.46-4.22 (m, 4H), 4.19-4.12 (m, 1H), 4.10-4.00 (m, 1H), 3.98-3.90 (m, 1H), 3.88 (t, J=4.8 Hz, 4H), 3.81-3.71 (m, 2H), 3.55-3.47 (m, 1H), 3.45-3.38 (m, 8H), 3.30-3.20 (m, 2H), 3.20-3.13 (m, 3H), 3.06-2.98 (m, 1H), 2.89-2.67 (m, 2H), 2.35-2.07 (m, 6H), 1.99-1.90 (m, 1H), 1.86-1.73 (m, 1H), 1.72-1.57 (m, 1H), 1.46 (d, J=6.4 Hz, 3H), 1.05-0.94 (m, 9H), 0.88 (d, J=6.4 Hz, 3H), 0.66-0.55 (m, 3H).

### Example 55

### (3S)-1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide

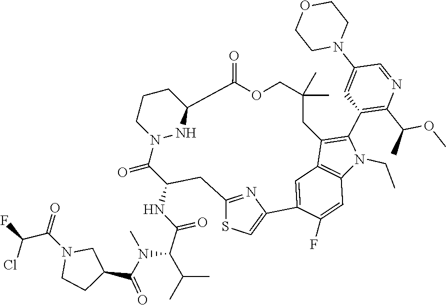

The title compound was prepared in analogy to the preparation of Example 1 by using (7S,13 S)-7-amino-21-ethyl-24-fluoro-(20M)-20-[2-[(1 S)-1-methoxyethyl]-5-morpholino-3-pyridyl]-17,17-dimethyl-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate M) instead of (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E). Example 55 (24.7 mg) was obtained as a yellow solid. MS calc'd 1038.4 (MH⁺), measured 1038.6 (MH⁺). ¹H NMR (400 MHz, METHANOL-d₄) δ=8.73-8.68 (m, 1H), 8.41-8.36 (m, 1H), 7.91-7.82 (m, 1H), 7.70-7.67 (m, 1H), 7.37 (d, J=16.0 Hz, 1H), 6.92-6.72 (m, 1H), 5.81-5.70 (m, 1H), 4.81-4.71 (dd, J=4.0, 12.0 Hz, 1H), 4.47-4.33 (m, 2H), 4.32-4.22 (m, 1H), 4.21-4.11 (m, 1H), 4.09-4.00 (m, 1H), 3.97-3.85 (m, 5H), 3.82-3.72 (m, 4H), 3.69-3.54 (m, 2H), 3.46-3.37 (m, 8H), 3.28-3.21 (m, 1H), 3.15-3.05 (m, 3H), 3.04-2.95 (m, 1H), 2.88-2.67 (m, 2H), 2.41-2.06 (m, 4H), 1.97-1.57 (m, 3H), 1.46 (d, J=8.0 Hz, 3H), 1.08-0.93 (m, 9H), 0.86 (d, J=4.0 Hz, 3H), 0.73-0.56 (m, 3H).

### Example 57

### (2S)-2-[(5S)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

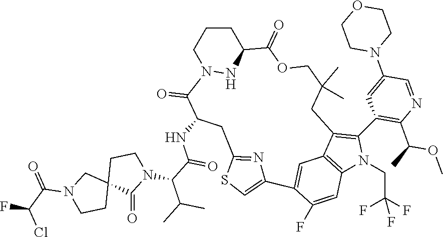

Example 57 was prepared in analogy to the preparation of Example 4 by using (2S)-2-[(5R)-7-tert-butoxycarbonyl-1-oxo-2,7-diazaspiro[4.4]nonan-2-yl]-3-methyl-butanoic acid (compound 4f 1) and (7S,13S)-7-amino-24-fluoro-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate I) instead of (2S)-2-[(5S)-7-tert-butoxycarbonyl-1-oxo-2,7-diazaspiro[4.4]nonan-2-yl]-3-methyl-butanoic acid (compound 4f 2) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 57 (26 mg) was obtained as a white solid. MS calc'd 1104.4 (MH⁺), measured 1104.4 (MH⁺). ¹H NMR (400 MHz, Methanol-d₄) δ=8.86-8.98 (d, J=7.6 Hz, 1H), 8.66-8.75 (d, J=7.2 Hz, 1H), 8.32-8.49 (d, J=2.8 Hz, 1H), 7.66-7.75 (t, J=2.8 Hz, 1H), 7.56-7.63 (m, 1H), 7.40-7.54 (d, J=12.4 Hz, 1H), 6.73-6.94 (dd, J₁=11.2 Hz, J₂=49.2 Hz, 1H), 5.60-5.81 (t, J=8.4 Hz, 1H), 5.11-5.31 (m, 1H), 4.38-4.47 (d, J=12.4 Hz, 1H), 4.30-4.37 (dd, J₁=3.2 Hz, J₂=11.2 Hz, 1H), 4.16-4.30 (m, 2H), 3.82-4.00 (m, 4H), 3.65-3.82 (m, 5H), 3.55-3.64 (m, 2H), 3.42-3.54 (m, 3H), 3.33-3.40 (m, 7H), 3.07-3.18 (m, 1H), 2.76-2.90 (m, 1H), 2.58-2.74 (m, 1H), 2.16-2.33 (m, 3H), 2.02-2.14 (m, 3H), 1.89-2.00 (m, 2H), 1.75-1.88 (m, 1H), 1.56-1.71 (m, 1H), 1.41-1.52 (d, J=6.4 Hz, 3H), 0.94-1.07 (m, 6H), 0.86-0.92 (d, J=6.4 Hz, 3H), 0.43-0.54 (s, 3H).

### Example 58

### (2S)-2-[2-[(2R)-2-chloro-2-fluoro-acetyl]-5-oxo-2,6-diazaspiro[3.4]octan-6-yl]-N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide

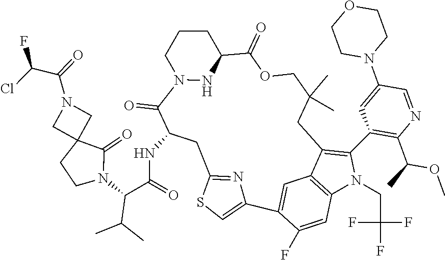

The title compound was prepared in analogy to the preparation of Example 6 by using (2S)-2-(2-tert-butoxycarbonyl-5-oxo-2,6-diazaspiro[3.4]octan-6-yl)-3-methyl-butanoic acid (compound 25H) and (7S,13S)-7-amino-24-fluoro-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate I) instead of (2S)-2-(9-tert-butoxycarbonyl-1-oxo-2,9-diazaspiro[5.5]undecan-2-yl)-3-methyl-butanoic acid (compound 6J) and (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D). Example 58 (24.0 mg) was obtained as a white solid. MS calc'd 1090.4 (MH⁺), measured 1090.4 (MH⁺). ¹H NMR (400 MHz, Methanol-d₄) δ=8.76-8.91 (d, J=8.4 Hz, 1H), 8.56-8.64 (d, J=7.6 Hz, 1H), 8.28-8.36 (d, J=2.4 Hz, 1H), 7.57-7.67 (dd, J=2.4 Hz, 17.6 Hz, 1H), 7.44-7.52 (m, 1H), 7.35-7.42 (d, J=12.8 Hz, 1H), 6.51-6.68 (dd, J=3.2 Hz, 52.4 Hz, 1H), 5.55-5.70 (t, J=8.4 Hz, 1H), 4.99-5.21 (m, 1H), 4.43-4.56 (m, 1H), 4.08-4.38 (m, 6H), 3.91-4.04 (m, 1H), 3.73-3.84 (t, J=4.8 Hz, 4H), 3.66-3.72 (m, 1H), 3.56-3.64 (m, 1H), 3.42-3.50 (m, 1H), 3.32-3.41 (m, 2H), 3.23-3.30 (m, 8H), 2.97-3.07 (m, 1H), 2.65-2.78 (m, 1H), 2.49-2.58 (m, 1H), 2.24-2.35 (m, 2H), 2.06-2.19 (m, 2H), 1.80-1.91 (m, 1H), 1.64-1.79 (m, 1H), 1.45-1.61 (m, 1H), 1.31-1.39 (d, J=8 Hz, 3H), 0.85-0.97 (m, 6H), 0.73-0.81 (m, 3H), 0.34-0.44 (s, 3H).

### Biological Example

Compound RM461 (page 115 of FIG. 1) from WO2020132597, and compound RM351 (page 88 of FIG. 1) from WO2020132597 and compound RM44 (page 11 of FIG. 1) from WO2020132597 were cited as reference compounds for this invention.

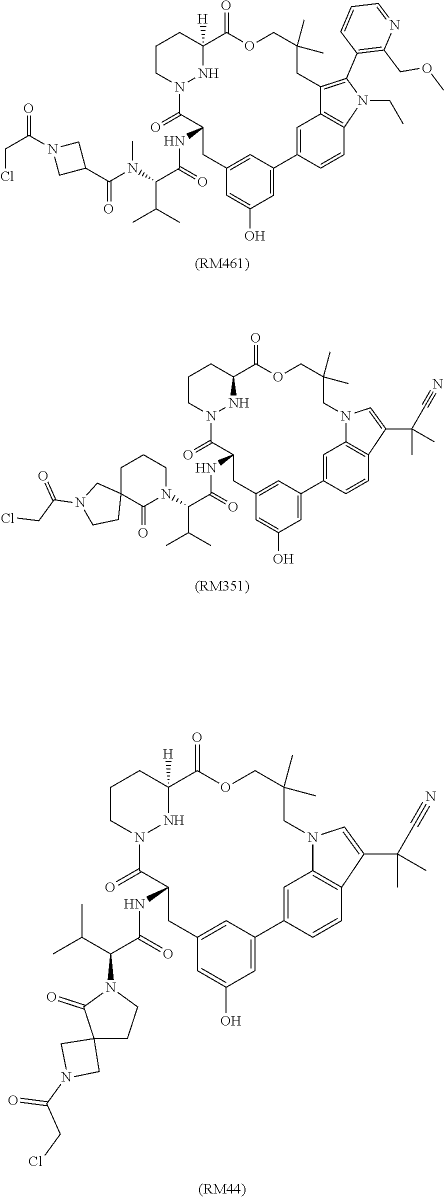

The applicant further synthesized RM461-A and RM44-A with 2-chloro-2-fluoro-acetamide instead of chloroacetamide as the analogue of RM461 and the analogue of RM44 according to the procedure described in WO2020132597, and provided the comparison data to demonstrate the improvement and illustrate the technical problem solved herein.

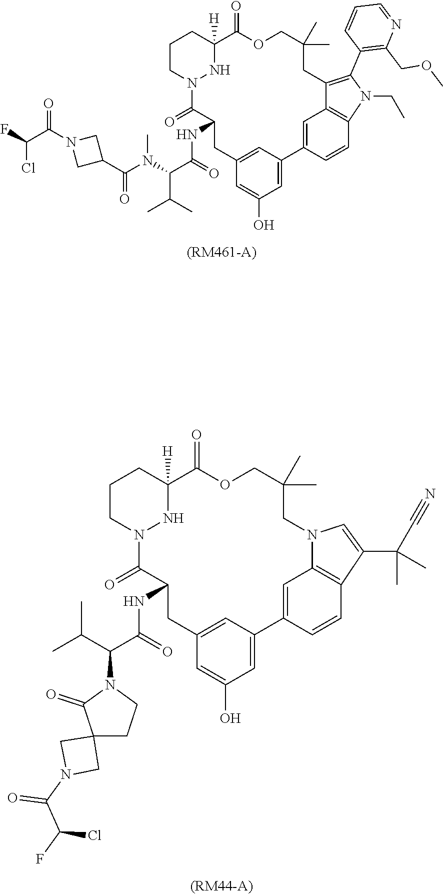

### Example 59

### GSH Reaction Rates

Glutathione (GSH) is a tripeptide found in most of the tissues, especially in high concentrations in the liver, and plays critical roles in protecting cells from oxidative damage and the toxicity of xenobiotic electrophiles, and maintaining redox homeostasis. More specifically, glutathione conjugation helps contribute to detoxification by binding electrophiles that could otherwise bind to proteins or nucleic acids, resulting in cellular damage and genetic mutations.

Many potentially toxic electrophilic xenobiotics and some endogenous compounds are detoxified by conversion to the corresponding glutathione S-conjugate, which consumes inherent GSH and then diminishes detoxification effects. Some drugs and halogenated workplace/environmental contaminants are bioactivated by this mechanism.

On the other hand, conjugation between glutathione and drug molecule in extrahepatic organs as well as in the liver typically leads to the poor PK properties (notably high clearance) of the molecule, and increases its possibility for off-target reactivity (potential liability for various toxicity). Therefore Strategy to minimize the GSH metabolism is very critical. Short T_{1/2} in inherent GSH reaction indicated high GSH reaction rate. Thus T_{1/2} in inherent GSH reaction assay was determined for the screening of candidates.

Reference compounds and compounds of this invention potentially could form conjugation with GSH either through halogenated moieties substitution reaction or direct Michael addition reaction. This test was therefore performed to check GSH reactivity of listed compounds.

For inherent GSH reactivity determination, compounds at 1 M were incubated at 37° C. with and without 5 mM GSH for 0, 0.5, 1, 2, 4 and 6 h in 100 mM potassium phosphate buffer at pH 7.4. At the end of the designated time points, samples were quenched with acetonitrile containing 10 mM N-ethylmaleimide and an internal standard. Quenched samples were centrifuged, and supernatants were analyzed by LC-MS/MS for compound quantification. If % depletion after 6 hour incubation is less than 20%, compounds were reported as stable; if % depletion is greater than 20%, half-life values are reported.

TABLE 1
GSH Reaction Rates of Examples and
Compounds of present invention

| Example | T_{1/2}(hour) in GSH | Remaining at 6 hr in GSH (%) |
|---|---|---|
| RM461 | 5 | 44 |
| RM461-A | stable | 100 |
| RM351 | 4.3 | 37 |
| RM44 | 8.9 | 60 |
| RM44-A | stable | 100 |
| Example 37 | stable | 100 |

Above result clearly shows that reference compounds (RM461, RM351 and RM44) formed conjugation with GSH causing its depletion over 6 hours while compounds of current invention maintained the stability with much less or no conjugation with GSH. Particularly, comparing reference compound RM461 and RM44 with their analogues RM461-A and RM44-A, the only difference is that the chloroacetamide was replaced by 2-chloro-2-fluoro-acetamide like the compounds of this invention, surprisingly such small change could address GSH toxicity issue as found in reference compounds.

### Example 60

### Human Hepatocyte Stability Assay

The hepatocyte stability assay measures the rate of disappearance of a compound from incubations with cryopreserved suspension hepatocytes from human. Positive controls, including Midazolam, Raloxifene and Dextromethorphan, are included in every experiment. Incubations consist of 1 M tested compound and suspension of human hepatocytes (1×10⁶ cells/mL) in supplemented Williams' E Medium with 10% FBS and 0.5% Penicillin-streptomycin. The hepatocyte suspension was incubated with intermittent shaking 900 rpm at 37° C., in a 5% CO₂ incubator. The reaction was stopped by adding methanol containing internal standard (2 μM Tolbutamide) at 2, 10, 20, 40, 60 and 120 minutes after compound addition, depletion of the parent compound was monitored by LC-MS/MS analysis. For human data, CL_hep (mL/min/kg)>16.24 is high clearance, CL_hep (mL/min/kg)<6.96 is low clearance. 16.24>CL_hep (mL/min/kg)>6.96 is medium clearance.

TABLE 2
Human hepatocytes stability of Examples
and Compounds of present invention

| | CL_hep (Human) | |
|---|---|---|
| Example | (mL/min/kg) | Clearance Category |
| RM461 | 16.4 | high |
| RM44 | 14.7 | medium |
| RM44-A | <2.4 | low |
| Example 4 | 6.4 | low |
| Example 5 | 12.5 | medium |
| Example 6 | 13.4 | medium |
| Example 15 | 10.9 | medium |
| Example 37 | 2.4 | low |

Above result clearly shows that reference compounds (RM44) showed medium clearance while Example 37 of current invention maintained the low clearance in human hepatocytes stability assay. Particularly, comparing reference compound RM44 with its analogue RM44-A, the only difference is that the chloroacetamide was replaced by 2-chloro-2-fluoro-acetamide like the compounds of this invention, surprisingly such small change could achieve the clearance reduction in human hepatocytes stability assay.

Achieving low clearance is advantageous to improve in vivo performance of the compound, such as dose reduction, exposure enhancement, and half-life prolongation.

### Example 61

### Cell Viability Assay

The purpose of this cellular assay was to determine the effects of test compounds on the proliferation of human cancer cell lines NCI-H358 (ATCC-CRL5807) cells, AGS (ATCC-CRL-1739) cells, SW620 (ATCC-CCL-227) over a 3-day treatment period by quantifying the amount of NADPH present at endpoint using Cell Counting Kit-8.

Cells were seeded at 5,000 cells/well (NCI-H358), 2,000 cells/well (AGS) 2,000 cells/well (SW620) in 96-well assay plates (Corning-3699) and incubated overnight. On the day of the assay, diluted compounds were then added in a final concentration of 0.5% DMSO. After 72 hrs incubation, a tenth of the volume of cell counting kit 8 (Dnjindo-CK04) was added into each well. Read the signal (OD450 minus OD650) using EnVision after 2 hrs incubation. IC₅₀ was determined by fitting a 4-parameter sigmoidal concentration response model.

Compounds of this invention clearly showed overall much improved cell potency in G12C, G12D and G12V compared with reference compounds RM461 and RM351.

TABLE 3
Activity of Examples and Compounds of present
invention in KRAS Cell viability assay

| Example | G12C IC50 (μM) | G12D IC50 (μM) | G12V IC50 (μM) |
|---|---|---|---|
| RM461 | 0.022 | 2.708 | 4.045 |
| RM461-A | 0.045 | 7.916 | 6.875 |
| RM351 | 1.393 | 2.104 | 4.807 |
| RM44 | 0.799 | 6.230 | >10 |
| Example 1 | 0.005 | 0.390 | 0.092 |
| Example 2 | 0.002 | 0.822 | 0.175 |
| Example 4 | 0.002 | 1.280 | 0.635 |
| Example 5 | 0.002 | 2.240 | 0.042 |
| Example 6 | 0.001 | 0.076 | 0.021 |
| Example 8 | 0.003 | 1.562 | 0.175 |
| Example 9 | 0.007 | 0.505 | 0.028 |
| Example 10 | <0.001 | 0.105 | 0.008 |
| Example 11 | <0.001 | 1.878 | 0.029 |
| Example 13 | 0.002 | 2.389 | 0.023 |
| Example 15 | <0.001 | 3.242 | 0.771 |
| Example 16 | <0.001 | 2.646 | 0.022 |
| Example 18 | 0.001 | 3.704 | 0.019 |
| Example 20 | 0.003 | 0.745 | 0.066 |
| Example 23 | N.A. | 0.322 | 0.090 |
| Example 24 | N.A. | 0.283 | 0.034 |
| Example 25 | N.A. | 1.648 | 0.089 |
| Example 26 | 0.001 | 0.260 | 0.034 |
| Example 27 | N.A. | 0.037 | 0.031 |
| Example 29 | <0.001 | 1.229 | 0.447 |
| Example 30 | <0.001 | 0.435 | 1.047 |
| Example 31 | <0.001 | 0.237 | 0.044 |
| Example 32 | <0.001 | 0.792 | 0.430 |
| Example 33 | <0.001 | 0.338 | 0.039 |
| Example 34 | <0.001 | 0.043 | 0.040 |
| Example 35 | <0.001 | 0.069 | 3.526 |
| Example 36 | <0.001 | 0.030 | 0.132 |
| Example 37 | <0.001 | 0.159 | 0.114 |
| Example 38 | <0.001 | 0.223 | 0.103 |
| Example 39 | <0.001 | 0.112 | 0.184 |
| Example 40 | <0.001 | 1.109 | 0.063 |
| Example 42 | <0.001 | 0.044 | 0.082 |
| Example 43 | <0.001 | 0.067 | 0.171 |
| Example 44 | <0.001 | 0.134 | >5 |
| Example 45 | <0.001 | 0.135 | 0.121 |
| Example 46 | 0.003 | 0.300 | 0.168 |
| Example 47 | 0.030 | >5 | >5 |
| Example 49 | <0.001 | 0.073 | 0.042 |
| Example 51 | <0.001 | 2.502 | 0.052 |
| Example 52 | <0.001 | 4.716 | 0.196 |
| Example 53 | <0.001 | 1.315 | 0.932 |
| Example 54 | <0.001 | 0.072 | 0.013 |
| Example 55 | <0.003 | 0.848 | 0.155 |
| Example 57 | 0.003 | 1.303 | 0.059 |
| Example 58 | 0.005 | 1.498 | 0.130 |

### Example 62

### KRAS G12C-BRAF NanoBit Assay

This assay is to measure the ability of tested compounds in disruption of the KRAS G12C-BRAF complex at the cellular level, we established the NanoBit cellular assay in mammalian HEK293 (ATCC) cells.

HEK293 cells were grown and maintained using DMEM medium (Thermo Fisher Scientific) with 10% fetal bovine serum and 1% penicillin/streptomycin. Both KRAS G12C and BRAF RBD were cloned into the NanoBit vectors (BiBiT vectors system, Promega) with the orientations SnmBit-KRAS G12C and BRAF RBD-LgBit, respectively, and co-transfected into HEK293 cells. Cells were then selected with 100 μg/mL Hygromycin B (10687010, Thermo Fisher) and Blasticidin (5 μg/mL) for 4 weeks to get the stable cell pool.

On the day of the assay, 75 nL of compound solution was presented in a 384-well assay plate as a 16-point 3-fold dilution starting from a final concentration of 30 μM in DMSO. Then cells were seeded at 10,000 cells/25 μL/well in a 384-well plate. After 3 hours of incubation, 6 μL of volume of Nano-Glo® Live Cell Substrate (Promega) was added into each well. Monitor luminescence using ultra384 model in Envision at 20 minutes. Compounds that facilitate disruption of the KRAS G12C-BRAF RBD complex were identified as those eliciting a decrease of luminescence relative to DMSO control wells.

TABLE 4
Activity of Examples and Compounds of present
invention in KRAS G12C-BRAF NanoBit assay

| Example | IC₅₀ (μM) |
|---|---|
| RM461 | 0.440 |
| RM351 | 1.785 |
| RM44 | 11.702 |
| RM44-A | 1.151 |
| Example 8 | 0.043 |
| Example 9 | 0.064 |
| Example 10 | 0.004 |
| Example 11 | 0.005 |
| Example 12 | 0.059 |
| Example 13 | 0.027 |
| Example 14 | 0.106 |
| Example 15 | 0.032 |
| Example 16 | 0.008 |
| Example 17 | 0.037 |
| Example 18 | 0.012 |
| Example 19 | 0.166 |
| Example 20 | 0.047 |
| Example 21 | 0.081 |
| Example 22 | 0.209 |
| Example 29 | 10.978 |
| Example 49 | 0.008 |
| Example 51 | 0.034 |
| Example 52 | 0.070 |
| Example 53 | 0.017 |
| Example 54 | 0.003 |
| Example 55 | 0.010 |
| Example 57 | 0.023 |
| Example 58 | 0.054 |

### Example 63

KRAS-BRAF with CYPA (500 nM) Interaction Assay

In this example, TR-FRET was also used to measure the compound or compound-CYPA dependent disruption of the KRAS G12C-BRAF complex. This protocol was also used to measure disruption of KRAS G12D or KRAS G12V binding to BRAF by a compound of the invention, respectively. In assay buffer containing 25 mM HEPES PH=7.4 (4-(2-hydroxyethyl)-1-piperazineethanesulfonic acid, Thermo, 15630080), 0.002% Tween20, 0.1% BSA, 100 mM NaCl, 5 mM MgCl₂, 10 μM GMPPNP (Guanosine 5′-[β,γ-imido]triphosphate trisodium salt hydrate, Sigma, G0635), tagless CYPA, GMPPNP loaded 6His-KRAS proteins, and GST-BRAF^{RBD} were mixed in a well of a 384-well assay plate at final concentrations of 50 nM, 6.25 nM and 1 nM, respectively. Compound was present in plate wells as a 16-point 3-fold dilution series starting at a final concentration of 10 μM and incubated for 3 hours. A mixture of MAb Anti-6His-XL665 (Cisbio, 61HISXLB) and Mab anti-GST-TB cryptate (Cisbio, 61GSTTLB) was then added at a final concentration of 6.67 nM and 0.21 nM, respectively, and the plate was incubated for an additional 1.5 hours. TR-FRET signal was read on a PHERstar FSX microplate reader (Ex320 nm, Em 665/615 nm). Compounds that facilitate disruption of the KRAS-BRAF complex were identified as those eliciting a decrease in the TR-FRET ratio relative to DMSO control wells.

TABLE 5
Activity of Examples and Compounds of present invention
in KRAS-BRAF with CYPA (500 nM) interaction assay

| Example | G12C IC₅₀ (μM) | G12D IC₅₀ (μM) | G12V IC₅₀ (μM) |
|---|---|---|---|
| RM461 | 0.004 | >10 | >10 |
| RM461-A | 0.027 | >10 | 6.079 |
| RM351 | >10 | >10 | >10 |
| RM44 | 0.017 | >10 | >10 |
| RM44-A | 0.779 | >10 | >10 |
| Example 2 | 0.089 | 3.977 | 1.862 |
| Example 4 | 0.122 | 4.372 | >10 |
| Example 8 | 0.003 | 0.085 | 0.010 |
| Example 9 | 0.176 | >10 | >10 |
| Example 10 | 0.020 | 0.964 | 0.136 |
| Example 11 | 0.045 | >10 | 1.029 |
| Example 16 | 0.100 | >10 | >10 |
| Example 18 | 0.071 | 1.665 | 3.331 |
| Example 20 | 0.148 | 1.240 | 1.779 |
| Example 22 | 0.199 | >10 | >10 |
| Example 23 | 0.064 | 2.085 | 5.392 |
| Example 24 | 0.053 | >10 | 1.392 |
| Example 25 | 0.098 | 6.064 | 3.541 |
| Example 26 | 0.046 | >10 | 0.954 |
| Example 27 | 0.002 | 0.007 | 0.003 |
| Example 30 | 0.038 | >10 | >10 |
| Example 31 | 0.044 | >10 | 1.977 |
| Example 32 | 0.056 | >10 | >10 |
| Example 33 | 0.018 | >10 | >10 |
| Example 34 | 0.047 | 0.909 | 0.724 |
| Example 35 | 0.023 | 3.218 | >10 |
| Example 36 | 0.038 | 3.935 | >10 |
| Example 37 | 0.039 | 0.964 | 1.355 |
| Example 38 | 0.032 | 3.302 | 0.910 |
| Example 39 | 0.104 | >10 | >10 |
| Example 40 | 0.108 | >10 | >10 |
| Example 41 | 0.088 | >10 | 1.175 |
| Example 42 | 0.073 | 3.968 | 7.863 |
| Example 43 | 0.022 | 2.202 | 1.687 |
| Example 44 | 0.034 | 1.340 | 0.607 |
| Example 45 | 0.010 | 4.967 | 0.299 |
| Example 46 | 0.129 | 1.063 | 1.232 |
| Example 47 | 0.079 | >10 | >10 |
| Example 48 | 0.047 | 1.032 | >10 |
| Example 49 | 0.015 | 0.444 | 0.577 |
| Example 50 | 0.124 | >10 | >10 |
| Example 51 | 0.074 | >10 | 6.444 |
| Example 52 | 0.115 | 2.063 | >10 |
| Example 53 | 0.056 | >10 | >10 |
| Example 54 | 0.019 | 0.303 | 0.217 |
| Example 55 | 0.040 | >10 | 2.114 |

### Example 64

pERK Inhibition Assay

This assay is to measure the ability of test compounds in inhibiting the phosphorylation of ERK, the downstream signaling of KRAS G12C in NCJ-H358 cells, KRAS G12D in AGS cells, and KRAS G12V in SW620. NCJ-H358 (ATCC-CRL5807) cells, AGS (ATCC-CRL-1739) cells, SW620 (ATCC-CCL-227) cells were all grown and maintained using RPMI-1640 medium (Thermo Fisher Scientific) with 10% fetal bovine serum and 1% penicillin/streptomycin. On the day prior to compound addition, cells were plated in tissue culture-treated 96 well plates (Corning-3699) at a density of 30,000 cell/well, 20,000 cell/well, 30,000 cell/well for NCJ-H358, AGS and SW620 respectively, and allowed for attachment overnight. Diluted compounds were then added in a final concentration of 0.5% DMSO. After 4 hours of incubation, the medium was removed, 100 μL of 4% formaldehyde was added, and the assay plates were incubated at room temperature for 20 minutes. The plates were then washed once with phosphate buffered saline (PBS), and permeabilized with 100 μL of chilled methanol for 10 minutes. Non-specific antibody binding to the plates was blocked using 50 μL 1×BSA blocking buffer (Thermo-37520, 10-fold dilution by Phosphate-Buffered Saline Tween (PBST) for at least 1 hour at room temperature.

The amount of phosphor-ERK was determined using an antibody specific for phosphorylated form of ERK. Primary antibody (pERK, CST-4370, Cell Signaling Technology) was diluted 1:300 in blocking buffer, with 50 μL aliquoted to each well, and incubated overnight at 4′° C. Cells was washed five times for 5 minutes with PBST. Secondary antibody (IRP-linked anti-rabbit IgG, CST-7074, Cell Signaling Technology) was diluted 1:1000 in blocking buffer, and 50 μL was added to each well and incubated 1-2 hrs at room temperature. Cells was washed 5 times for 5 minutes with PBST, 100 μL TMB ELISA substrate (abcam-ab171523) were added and gently shake for 20 minutes. 50 μL stop solution (abcam-ab171529) were added, and then read the signal (OD450) by EnVision.

IC₅₀ was determined by fitting a 4-parameter sigmoidal concentration response model.

TABLE 6
Activity of Examples and Compounds of present
invention in KRAS pERK inhibition assay

| Example | G12C IC₅₀ (μM) | G12D IC₅₀ (μM) | G12V IC₅₀ (μM) |
|---|---|---|---|
| RM461 | 0.007 | >10 | >10 |
| RM461-A | 0.049 | >10 | 7.022 |
| RM351 | 0.356 | >10 | >10 |
| RM44 | 1.899 | >10 | >10 |
| RM44-A | >10 | >10 | >10 |
| Example 1 | 0.007 | 0.137 | 0.055 |
| Example 2 | 0.002 | 0.038 | 0.020 |
| Example 3 | 0.032 | 0.114 | 0.138 |
| Example 4 | 0.002 | 0.110 | 0.048 |
| Example 5 | 0.002 | 0.048 | 0.026 |
| Example 6 | 0.001 | 0.021 | 0.024 |
| Example 8 | 0.002 | 0.090 | 0.127 |
| Example 9 | 0.003 | 0.023 | 0.022 |
| Example 10 | <0.001 | 0.016 | 0.006 |
| Example 11 | <0.001 | 0.027 | 0.022 |
| Example 14 | 0.033 | 0.170 | 0.078 |
| Example 16 | 0.001 | 0.019 | 0.015 |
| Example 18 | 0.003 | 0.026 | 0.018 |
| Example 20 | 0.005 | 0.098 | 0.073 |
| Example 22 | 0.053 | 1.064 | 1.022 |
| Example 24 | 0.005 | 0.012 | 0.018 |
| Example 26 | 0.007 | 0.015 | 0.014 |
| Example 27 | 0.003 | 0.011 | 0.010 |
| Example 29 | 0.001 | 0.012 | 0.045 |
| Example 30 | 0.002 | 0.021 | 0.100 |
| Example 31 | 0.005 | 0.024 | 0.012 |
| Example 32 | 0.001 | 0.014 | 0.021 |
| Example 33 | 0.004 | 0.019 | 0.008 |
| Example 34 | 0.002 | N.A. | 0.012 |
| Example 35 | 0.003 | N.A. | 0.050 |
| Example 36 | 0.003 | N.A. | 0.013 |
| Example 37 | 0.003 | N.A. | 0.021 |
| Example 38 | 0.001 | N.A. | 0.045 |
| Example 39 | 0.006 | N.A. | 0.044 |
| Example 40 | 0.010 | N.A. | 0.023 |
| Example 41 | 0.107 | N.A. | 0.199 |
| Example 42 | 0.003 | N.A. | 0.024 |
| Example 43 | 0.006 | N.A. | 0.044 |
| Example 44 | 0.010 | N.A. | 0.044 |
| Example 45 | 0.007 | N.A. | 0.020 |
| Example 46 | 0.009 | 0.299 | 0.627 |
| Example 47 | 0.026 | N.A. | 0.176 |
| Example 48 | 0.002 | N.A. | 0.012 |
| Example 49 | 0.003 | 0.065 | 0.020 |
| Example 50 | 0.006 | N.A. | 0.006 |
| Example 51 | 0.002 | 0.062 | 0.057 |
| Example 52 | 0.004 | 0.103 | 0.058 |
| Example 53 | 0.003 | 0.046 | 0.061 |
| Example 54 | 0.002 | 0.025 | 0.015 |
| Example 55 | 0.006 | 0.106 | 0.075 |
| Example 57 | 0.006 | 0.057 | 0.033 |
| Example 58 | 0.012 | 0.124 | 0.041 |

1. A compound of formula (I), 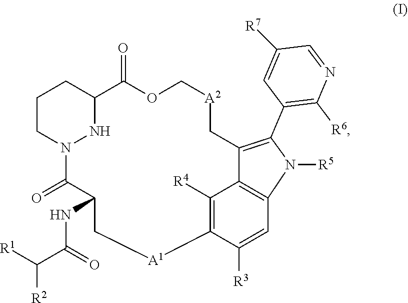
wherein
R¹ is 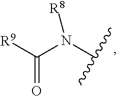
1-oxo-2,7-diazaspiro[4.4]nonanyl substituted by (dihaloC₁₋₆alkyl)carbonyl,
1-oxo-2,8-diazaspiro[4.5]decanyl substituted by (dihaloC₁₋₆alkyl)carbonyl,
1-oxo-2,9-diazaspiro[5.5]undecanyl substituted by (dihaloC₁₋₆alkyl)carbonyl,
5-oxo-2,6-diazaspiro[3.4]octanyl substituted by (dihaloC₁₋₆alkyl)carbonyl,
5-oxo-2,6-diazaspiro[3.5]nonanyl substituted by (dihaloC₁₋₆alkyl)carbonyl,
6-oxo-2,7-diazaspiro[4.5]decanyl substituted by (dihaloC₁₋₆alkyl)carbonyl or
azetidinyloxyC₁₋₆alkyl substituted by (dihaloC₁₋₆alkyl)carbonyl;
wherein R⁸ is C₁₋₆alkyl;
R⁹ is piperidinyl twice substituted by halogen and (dihaloC₁₋₆alkyl)carbonyl, or pyrrolidinyl substituted by (dihaloC₁₋₆alkyl)carbonyl;
R² is C₁₋₆alkyl;
R³ is H, C₁₋₆alkyl or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene or phenylene substituted by hydroxy;
A² is C₁₋₆alkylene;
with the proviso that R³ and R⁴ are not H simultaneously;
or a pharmaceutically acceptable salt thereof.

2. A compound of formula (Ia), 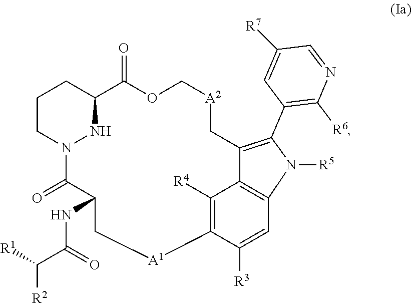
wherein
R¹ is 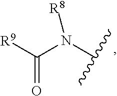
1-oxo-2,7-diazaspiro[4.4]nonanyl substituted by (dihaloC₁₋₆alkyl)carbonyl,
1-oxo-2,8-diazaspiro[4.5]decanyl substituted by (dihaloC₁₋₆alkyl)carbonyl,
1-oxo-2,9-diazaspiro[5.5]undecanyl substituted by (dihaloC₁₋₆alkyl)carbonyl,
5-oxo-2,6-diazaspiro[3.4]octanyl substituted by (dihaloC₁₋₆alkyl)carbonyl,
5-oxo-2,6-diazaspiro[3.5]nonanyl substituted by (dihaloC₁₋₆alkyl)carbonyl,
6-oxo-2,7-diazaspiro[4.5]decanyl substituted by (dihaloC₁₋₆alkyl)carbonyl or
azetidinyloxyC₁₋₆alkyl substituted by (dihaloC₁₋₆alkyl)carbonyl;
wherein R⁸ is C₁₋₆alkyl;
R⁹ is piperidinyl twice substituted by halogen and (dihaloC₁₋₆alkyl)carbonyl, or pyrrolidinyl substituted by (dihaloC₁₋₆alkyl)carbonyl;
R² is C₁₋₆alkyl;
R³ is H, C₁₋₆alkyl or halogen;
R⁴ is H or halogen;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is thiazolylene or phenylene substituted by hydroxy;
A² is C₁₋₆alkylene;
with the proviso that R³ and R⁴ are not H simultaneously;
or a pharmaceutically acceptable salt thereof.

3. A compound according to claim 1 or 2, wherein
R¹ is 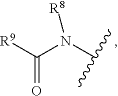
1-oxo-2,7-diazaspiro[4.4]nonanyl substituted by (dihaloC₁₋₆alkyl)carbonyl,
1-oxo-2,8-diazaspiro[4.5]decanyl substituted by (dihaloC₁₋₆alkyl)carbonyl,
1-oxo-2,9-diazaspiro[5.5]undecanyl substituted by (dihaloC₁₋₆alkyl)carbonyl, or
5-oxo-2,6-diazaspiro[3.4]octanyl substituted by (dihaloC₁₋₆alkyl)carbonyl;
wherein R⁸ is C₁₋₆alkyl;
R⁹ is piperidinyl twice substituted by halogen and (dihaloC₁₋₆alkyl)carbonyl, or pyrrolidinyl substituted by (dihaloC₁₋₆alkyl)carbonyl.

4. A compound according to any one of claims 1-3, wherein
R¹ is 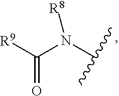
1-oxo-2,7-diazaspiro[4.4]nonan-2-yl substituted by chloro(fluoro)acetyl,
1-oxo-2,8-diazaspiro[4.5]decan-2-yl substituted by chloro(fluoro)acetyl,
1-oxo-2,9-diazaspiro[5.5]undecan-2-yl substituted by chloro(fluoro)acetyl, or
5-oxo-2,6-diazaspiro[3.4]octan-6-yl substituted by chloro(fluoro)acetyl;
wherein R⁸ is methyl;
R⁹ is piperidinyl twice substituted by fluoro and chloro(fluoro)acetyl, or pyrrolidinyl substituted by chloro(fluoro)acetyl.

5. A compound according to any one of claims 1-4, wherein R¹ is (5R)-7-[(2R)-2-chloro-2-fluoro-acetyl]-1-oxo-2,7-diazaspiro[4.4]nonan-2-yl, (5S)-7-[(2R)-2-chloro-2-fluoro-acetyl]-1-oxo-2,7-diazaspiro[4.4]nonan-2-yl, 8-[(2R)-2-chloro-2-fluoro-acetyl]-1-oxo-2,8-diazaspiro[4.5]decan-2-yl, 9-[(2R)-2-chloro-2-fluoro-acetyl]-1-oxo-2,9-diazaspiro[5.5]undecan-2-yl, 2-[(2R)-2-chloro-2-fluoro-acetyl]-5-oxo-2,6-diazaspiro[3.4]octan-6-yl, 1-[(2R)-2-chloro-2-fluoro-acetyl]-4-fluoro-4-piperidinyl or 1-[(2R)-2-chloro-2-fluoro-acetyl]pyrrolidin-3-yl.

6. A compound according to any one of claims 1-5, wherein R² is isopropyl.

7. A compound according to any one of claims 1-6, wherein R³ is H, methyl or fluoro.

8. A compound according to any one of claims 1-7, wherein R³ is fluoro.

9. A compound according to any one of claims 1-8, wherein R⁴ is H or fluoro.

10. A compound according to any one of claims 1-9, wherein R⁴ is H.

11. A compound according to any one of claims 1-10, wherein R⁵ is ethyl or 2,2,2-trifluoroethyl.

12. A compound according to any one of claims 1-11, wherein R⁶ is 1-methoxyethyl.

13. A compound according to any one of claims 1-12, wherein R⁷ is morpholinyl, 4-(2,2,2-trifluoroethyl)piperazin-1-yl or 4-methylpiperazin-1-yl.

14. A compound according to any one of claims 1-13, wherein A¹ is 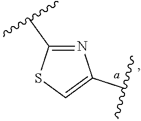 [SMILES: *c1csc(*)n1]
wherein bond “a” connects to indole ring.

15. A compound according to any one of claims 1-14, wherein A² is dimethylmethylene.

16. A compound according to claim 1 or 2, wherein
R¹ is 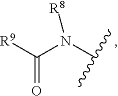
1-oxo-2,7-diazaspiro[4.4]nonanyl substituted by (dihaloC₁₋₆alkyl)carbonyl,
1-oxo-2,8-diazaspiro[4.5]decanyl substituted by (dihaloC₁₋₆alkyl)carbonyl,
1-oxo-2,9-diazaspiro[5.5]undecanyl substituted by (dihaloC₁₋₆alkyl)carbonyl, or
5-oxo-2,6-diazaspiro[3.4]octanyl substituted by (dihaloC₁₋₆alkyl)carbonyl;
wherein R⁸ is C₁₋₆alkyl;
R⁹ is piperidinyl twice substituted by halogen and (dihaloC₁₋₆alkyl)carbonyl, or pyrrolidinyl substituted by (dihaloC₁₋₆alkyl)carbonyl;
R² is C₁₋₆alkyl;
R³ is halogen;
R⁴ is H;
R⁵ is C₁₋₆alkyl or haloC₁₋₆alkyl;
R⁶ is C₁₋₆alkoxyC₁₋₆alkyl;
R⁷ is morpholinyl, (haloC₁₋₆alkyl)piperazinyl or C₁₋₆alkylpiperazinyl;
A¹ is 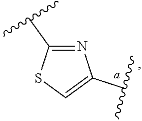 [SMILES: *c1csc(*)n1]
wherein bond “a” connects to indole ring;
A² is C₁₋₆alkylene;
or a pharmaceutically acceptable salt thereof.

17. A compound according to claim 16, wherein
R¹ is (5R)-7-[(2R)-2-chloro-2-fluoro-acetyl]-1-oxo-2,7-diazaspiro[4.4]nonan-2-yl, (5S)-7-[(2R)-2-chloro-2-fluoro-acetyl]-1-oxo-2,7-diazaspiro[4.4]nonan-2-yl, 8-[(2R)-2-chloro-2-fluoro-acetyl]-1-oxo-2,8-diazaspiro[4.5]decan-2-yl, 9-[(2R)-2-chloro-2-fluoro-acetyl]-1-oxo-2,9-diazaspiro[5.5]undecan-2-yl, 2-[(2R)-2-chloro-2-fluoro-acetyl]-5-oxo-2,6-diazaspiro[3.4]octan-6-yl, 1-[(2R)-2-chloro-2-fluoro-acetyl]-4-fluoro-4-piperidinyl or 1-[(2R)-2-chloro-2-fluoro-acetyl]pyrrolidin-3-yl;
R² is isopropyl;
R³ is fluoro;
R⁴ is H;
R⁵ is ethyl or 2,2,2-trifluoroethyl;
R⁶ is (1S)-1-methoxyethyl;
R⁷ is morpholinyl, 4-(2,2,2-trifluoroethyl)piperazin-1-yl or 4-methylpiperazin-1-yl;
A¹ is 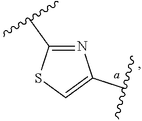 [SMILES: *c1csc(*)n1]
wherein bond “a” connects to indole ring;
A² is dimethylmethylene;
or a pharmaceutically acceptable salt thereof.

18. A compound selected from:
(3S)-1-[(2R)-2-chloro-2-fluoro-acetyl]-N-[(1S)-1-[[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(7S,13S)-(20M)-20-[2-[(1S)-1-methoxyethyl]-5-(4-methylpiperazin-1-yl)-3-pyridyl]-17,17,24-trimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(2S)-2-[(5R)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[9-[(2R)-2-chloro-2-fluoro-acetyl]-1-oxo-2,9-diazaspiro[5.5]undecan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(3S)-1-[(2R)-2-chloro-2-fluoro-acetyl]-N-[(1S)-1-[[(8S,14S)-25-fluoro-4-hydroxy-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl]-2-methyl-propyl]-N-methyl-pyrrolidine-3-carboxamide;
1-[(2R)-2-chloro-2-fluoro-acetyl]-4-fluoro-N-[(1S)-1-[[(7S,13S)-(20M)-20-[2-[(1S)-1-methoxyethyl]-5-(4-methylpiperazin-1-yl)-3-pyridyl]-17,17,24-trimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-piperidine-4-carboxamide;
1-[(2R)-2-chloro-2-fluoro-acetyl]-4-fluoro-N-[(1S)-1-[[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-piperidine-4-carboxamide;
(2S)-2-[2-[(2R)-2-chloro-2-fluoro-acetyl]-5-oxo-2,6-diazaspiro[3.5]nonan-6-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2R)-2-[[1-[(2R)-2-chloro-2-fluoro-acetyl]azetidin-3-yl]oxymethyl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo-[17.5.2.1^{2,5}.1^{9,13}.0^{22,26}]octacosa-1(25),2,5(28),19,22(26),23-hexaen-7-yl]-3-methyl-butanamide;
(2S)-2-[[1-[(2R)-2-chloro-2-fluoro-acetyl]azetidin-3-yl]oxymethyl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[8-[(2R)-2-chloro-2-fluoro-acetyl]-1-oxo-2,8-diazaspiro[4.5]decan-2-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-2-[(2R)-2-chloro-2-fluoro-acetyl]-6-oxo-2,7-diazaspiro[4.5]decan-7-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5R)-2-[(2R)-2-chloro-2-fluoro-acetyl]-6-oxo-2,7-diazaspiro[4.5]decan-7-yl]-N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[9-[(2R)-2-chloro-2-fluoro-acetyl]-1-oxo-2,9-diazaspiro[5.5]undecan-2-yl]-N-[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5R)-2-[(2S)-2-chloro-2-fluoro-acetyl]-6-oxo-2,7-diazaspiro[4.5]decan-7-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-2-[(2S)-2-chloro-2-fluoro-acetyl]-6-oxo-2,7-diazaspiro[4.5]decan-7-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(3S)-1-[(2R)-2-chloro-2-fluoro-acetyl]-N-[(1S)-1-[[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(2S)-2-[2-[(2R)-2-chloro-2-fluoro-acetyl]-5-oxo-2,6-diazaspiro[3.4]octan-6-yl]-N-[(7S,13S)-24-fluoro-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(3S)-1-[(2R)-2-chloro-2-fluoro-acetyl]-N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(8S,14S)-26-fluoro-4-hydroxy-(21M)-21-[2-[(1S)-1-methoxyethyl]-5-(4-methylpiperazin-1-yl)-3-pyridyl]-18,18-dimethyl-9,15-dioxo-22-(2,2,2-trifluoroethyl)-16-oxa-10,22,28-triazapentacyclo[18.5.2.1^{2,6}.1^{10,14}.0^{23,27}]nonacosa-1(26),2,4,6(29),20,23(27),24-heptaen-8-yl]carbamoyl]-2-methyl-propyl]-N-methyl-pyrrolidine-3-carboxamide;
(2S)-2-[8-[(2R)-2-chloro-2-fluoro-acetyl]-1-oxo-2,8-diazaspiro[4.5]decan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[2-[(2R)-2-chloro-2-fluoro-acetyl]-5-oxo-2,6-diazaspiro[3.4]octan-6-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5R)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(3S)-1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(3S)-1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-4-fluoro-N-methyl-piperidine-4-carboxamide;
(2S)-2-[(5R)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[9-[(2R)-2-chloro-2-fluoro-acetyl]-1-oxo-2,9-diazaspiro[5.5]undecan-2-yl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[2-[(2R)-2-chloro-2-fluoro-acetyl]-5-oxo-2,6-diazaspiro[3.4]octan-6-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[8-[(2R)-2-chloro-2-fluoro-acetyl]-1-oxo-2,8-diazaspiro[4.5]decan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2R)-2-[[1-[(2R)-2-chloro-2-fluoro-acetyl]azetidin-3-yl]oxymethyl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[[1-[(2R)-2-chloro-2-fluoro-acetyl]azetidin-3-yl]oxymethyl]-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[9-[(2R)-2-chloro-2-fluoro-acetyl]-1-oxo-2,9-diazaspiro[5.5]undecan-2-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5R)-7-[(2R)-2-chloro-2-fluoro-acetyl]-1-oxo-2,7-diazaspiro[4.4]nonan-2-yl]-N-[(7S,13S)-24-fluoro-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
1-[(2R)-2-chloro-2-fluoro-acetyl]-4-fluoro-N-[(1S)-1-[[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-piperidine-4-carboxamide;
(2S)-2-[2-[(2R)-2-chloro-2-fluoro-acetyl]-5-oxo-2,6-diazaspiro[3.4]octan-6-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[8-[(2R)-2-chloro-2-fluoro-acetyl]-1-oxo-2,8-diazaspiro[4.5]decan-2-yl]-N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[9-[(2R)-2-chloro-2-fluoro-acetyl]-1-oxo-2,9-diazaspiro[5.5]undecan-2-yl]-N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-4-fluoro-N-methyl-piperidine-4-carboxamide;
(2S)-2-[8-[(2R)-2-chloro-2-fluoro-acetyl]-1-oxo-2,8-diazaspiro[4.5]decan-2-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5S)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[(5R)-7-[(2R)-2-chloro-2-fluoro-acetyl]-1-oxo-2,7-diazaspiro[4.4]nonan-2-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
(2S)-2-[2-[(2R)-2-chloro-2-fluoro-acetyl]-5-oxo-2,6-diazaspiro[3.4]octan-6-yl]-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-4-fluoro-N-methyl-piperidine-4-carboxamide;
(3S)-1-[(2R)-2-chloro-2-fluoro-acetyl]-N-[(1S)-1-[[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamoyl]-2-methyl-propyl]-N-methyl-pyrrolidine-3-carboxamide;
(2S)-2-[(5S)-7-[(2R)-2-chloro-2-fluoro-acetyl]-1-oxo-2,7-diazaspiro[4.4]nonan-2-yl]-N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide; and
(2S)-2-[2-[(2R)-2-chloro-2-fluoro-acetyl]-5-oxo-2,6-diazaspiro[3.4]octan-6-yl]-N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-methyl-butanamide;
or a pharmaceutically acceptable salt thereof.

19. A compound which has the following structure:

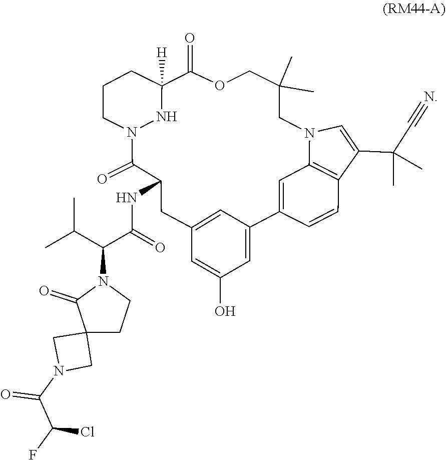 [SMILES: CC(C)[C@@H](C(=O)N[C@H]1Cc2cc(O)cc(c2)-c2ccc3c(C(C)(C)C#N)cn(c3c2)CC(C)(C)COC(=O)[C@@H]2CCCN(N2)C1=O)N1CCC2(CN(C(=O)[C@H](F)Cl)C2)C1=O]

20. A process for the preparation of a compound according to any one of claims 1 to 19 comprising any of the following steps:
a) coupling reaction between compound of formula (II), 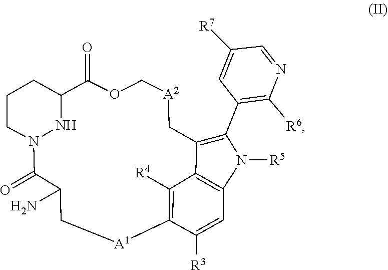
and acid (III), 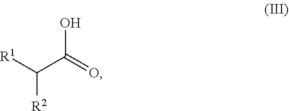
in the presence of a coupling reagent and a base to form the compound of formula (I);
b) coupling reaction between compound of formula (VI), 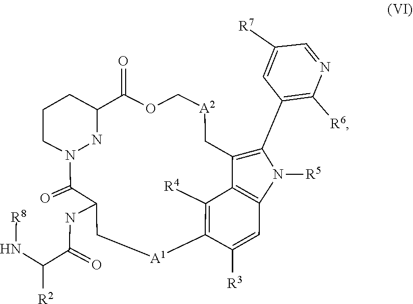
and acid (VII), 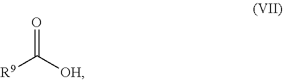
in the presence of a coupling reagent and a base to form the compound of formula (VIII), 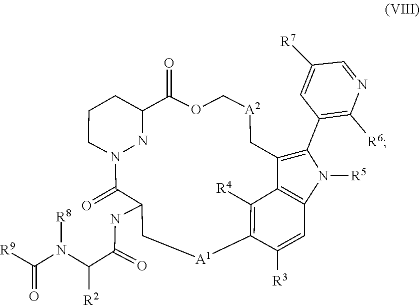
c) coupling reaction between compound of formula (XI), 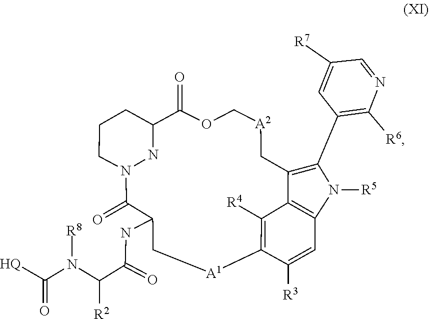
and acid (XII), 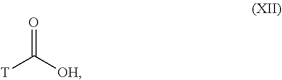
in the presence of a coupling reagent and a base to form the compound of formula (XIII), 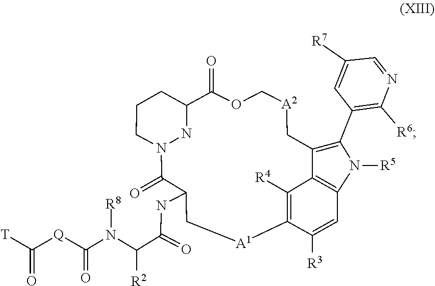
wherein Q is unsubstituted or substituted piperidinylene or pyrrolidinylene; T is dihaloC₁₋₆alkyl; R¹, R², R³, R⁴, R⁵, R⁶, R⁷, A¹ and A² are defined as in any one of claims 1 to 17; the coupling reagent in step a) to c) is T₃P, HATU, PyBOP or EDCl/HOBt; the base in step a) to c) is TEA, DIEPA or DMAP.

21. A compound or pharmaceutically acceptable salt according to any one of claims 1 to 19 for use as therapeutically active substance.

22. A pharmaceutical composition comprising a compound in accordance with any one of claims 1 to 19 and a pharmaceutically acceptable excipient.

23. The use of a compound according to any one of claims 1 to 19 for treating a KRAS G12C protein-related disease.

24. The use of a compound according to any one of claims 1 to 19 for treating a KRAS G12C, G12D and G12V protein-related disease.

25. The use of a compound according to any one of claims 1 to 19 for inhibiting RAS interaction with downstream effectors, wherein the downstream effectors are RAF and PT3K.

26. The use of a compound according to any one of claims 1 to 19 for inhibiting the propagating oncogenic MAPK and PI3K signaling.

27. The use of a compound according to any one of claims 1 to 19 for the treatment or prophylaxis of KRAS mutation driven cancers, wherein the cancer is selected from pancreatic cancer, colorectal cancer, lung cancer, esophageal cancer, gallbladder cancer, melanoma ovarian cancer and endometrial cancer.

28. The use of a compound according to any one of claims 1 to 19 for the treatment or prophylaxis of KRAS mutation driven cancers, wherein the cancer is selected from pancreatic adenocarcinoma, colorectal cancer and non-small cell lung cancer.

29. A compound or pharmaceutically acceptable salt according to any one of claims 1 to 19 for the treatment or prophylaxis of KRAS mutation driven cancers, wherein the cancer is selected from pancreatic adenocarcinoma, colorectal cancer and non-small cell lung cancer.

30. The use of a compound according to any one of claims 1 to 19 for the preparation of a medicament for the treatment or prophylaxis of KRAS mutation driven cancers, wherein the cancer is selected from pancreatic adenocarcinoma, colorectal cancer and non-small cell lung cancer.

31. A method for the treatment or prophylaxis of KRAS mutation driven cancers, wherein the cancer is selected from pancreatic adenocarcinoma, colorectal cancer and non-small cell lung cancer, which method comprises administering a therapeutically effective amount of a compound as defined in any one of claims 1 to 19.

32. A compound or pharmaceutically acceptable salt according to any one of claims 1 to 19, when manufactured according to a process of claim 20.

33. The invention as hereinbefore described.
